(19) United States
(12) Patent Application Publication
(10) Pub. No.: US 2006/0025383 A1
(43) Pub. Date: Feb. 2, 2006

(54) Aminobenzoxazoles as therapeutic agents

(76) Inventors: Neil Wishart, Jefferson, MA (US); Michael Friedman, Newton, MA (US); Lee D. Arnold, Niantic, CT (US); Bryant Yang, Simi Valley, CA (US); Shannon R. Fix-Stenzel, Shrewsbury, MA (US); Anna Ericsson, Shrewsbury, MA (US); Michael R. Michaelides, Libertyville, IL (US); Xiao-Dong Qian, Wellesley, MA (US); James H. Holms, Gurnee, IL (US); Douglas H. Steinman, Morton Grove, IL (US); Zhengping Tian, Grayslake, IL (US); Steven J. Wittenberger, Mundelein, IL (US)

Correspondence Address: ABBOTT BIORESEARCH, 100 RESEARCH DRIVE, WORCESTER, MA, 01605-4314, US

(21) Appl. No.: 11/050,609

(22) Filed: Feb. 3, 2005

Related U.S. Application Data
(60) Provisional application No. 60/541,294, filed on Feb. 3, 2004.
(60) Provisional application No. 60/547,612, filed on Feb. 25, 2004.

(51) Int. Cl. A61K 31/5377; A61K 31/519; A61K 31/695; C07D 487/02

(52) U.S. Cl. 514/63; 514/262.1; 514/265.1; 514/234.5; 544/118; 544/280; 544/229; 544/262; 544/230

(57) ABSTRACT

A compound of Formula (I), 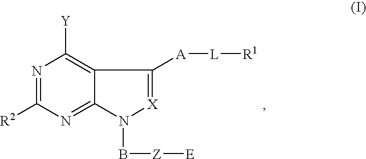 wherein the substituents are as defined herein, which are useful as kinase inhibitors.

### CROSS-REFERENCE TO RELATED APPLICATION

This application claims the benefit of priority to U.S. application No. 60/541,294, filed Feb. 3, 2004 and to U.S. application No. 60/547,612 filed Feb. 25, 2004.

### BACKGROUND OF THE INVENTION

There are at least 400 enzymes identified as protein kinases. These enzymes catalyze the phosphorylation of target protein substrates. The phosphorylation is usually a transfer reaction of a phosphate group from ATP to the protein substrate. The specific structure in the target substrate to which the phosphate is transferred is a tyrosine, serine or threonine residue. Since these amino acid residues are the target structures for the phosphoryl transfer, these protein kinase enzymes are commonly referred to as tyrosine kinases or serine/threonine kinases.

The phosphorylation reactions, and counteracting phosphatase reactions, at the tyrosine, serine and threonine residues are involved in countless cellular processes that underlie responses to diverse intracellular signals (typically mediated through cellular receptors), regulation of cellular functions, and activation or deactivation of cellular processes. A cascade of protein kinases often participate in intracellular signal transduction and are necessary for the realization of these cellular processes. Because of their ubiquity in these processes, the protein kinases can be found as an integral part of the plasma membrane or as cytoplasmic enzymes or localized in the nucleus, often as components of enzyme complexes. In many instances, these protein kinases are an essential element of enzyme and structural protein complexes that determine where and when a cellular process occurs within a cell.

The identification of effective small compounds which specifically inhibit signal transduction and cellular proliferation by modulating the activity of receptor and non-receptor tyrosine and serine/threonine kinases to regulate and modulate abnormal or inappropriate cell proliferation, differentiation, or metabolism is therefore desirable. In particular, the identification of methods and compounds that specifically inhibit the function of a tyrosine kinase which is essential for antiangiogenic processes or the formation of vascular hyperpermeability leading to edema, ascites, effusions, exudates, and macromolecular extravasation and matrix deposition as well as associated disorders would be beneficial.

The present invention provides novel compounds that inhibit one or more receptor and non-receptor and serine/threonine kinases.

### SUMMARY OF THE INVENTION

The present invention provides a compound of Formula (I), 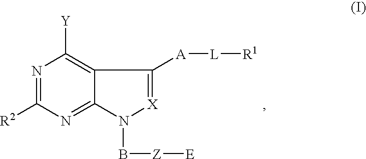
pharmaceutically acceptable salts thereof, metabolites thereof, isomers thereof, or pro-drugs thereof, denoted as Group A, wherein
X is N or CH;
A is optionally substituted phenyl,
or A is 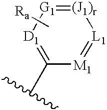
r is 1 and D₁, G₁, J₁, L₁ and M₁ are each independently selected from the group consisting of CRₐ and N, provided that at least two of D₁, G₁, J₁, L₁ and M₁ are CRₐ; or
r is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined below;
L is NH, optionally substituted alkyl, carbonyl, —O-optionally substituted alkyl, NH(optionally substituted aliphatic) or S;
R¹ is —C(═O)—N(R¹⁰⁰)₂ wherein R¹⁰⁰ for each occurrence is independently hydrogen or alkyl; 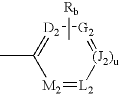
   or R¹ is
or an optionally substituted group selected from the group consisting of an aliphatic group, benzimidazolyl, benzofuranyl, benzoisothiazolyl, benzoisoxazolyl, benzoxazolyl, benzothiazolyl, benzothienyl, cycloalkyl, 2,3-dihydrobenzofuranyl, 1,1-dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, thiadiazolyl, thiazolyl thienyl, 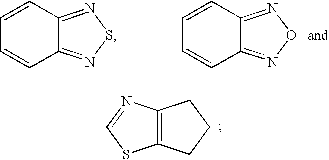
wherein the foregoing optionally substituted groups are optionally substituted by one or more R_{b};
u is 1 and D₂, G₂, J₂, L₂ and M₂ are each independently selected from the group consisting of CRₐ and N, provided that at least two of D₂, G₂, J₂, L₂ and M₂ are CRₐ; or
u is 0, and one of D₂, G₂, L₂ and M₂ is NRₐ, one of D₂, G₂, L₂ and M₂ is CRₐ and the remainder are independently selected from the group consisting of CRₐ and N;
Rₐ and R_{b} each represent one or more substituents and for each occurrence is independently selected from the optionally substituted group consisting of an aliphatic group, alkoxy, alkylamino, aliphatic-carbonyl, aliphatic-cycloalkyl, aliphatic-heterocyclyl, alkyl-S—, alkyl-S(O)ₚ—, amido groups, amino, aminoalkyl, carboxamido, —CF₃, —CN, —C(O)-aliphatic, —C(O)-cycloalkyl, —C(O)-heterocyclyl, —C(O)H, C(O)OH, —C(O)O-aliphatic, C(O)O—C(O)O-heterocyclyl, cycloalkyl, cycloalkyl-aliphatic, cycloalkyl-S, cycloalkyl-S(O)ₚ, cycloalkylthio, dialkylaminoalkoxy, a halo, heterocyclyl, heterocycloalkoxy, heterocycloalkyl, heterocyclyloxy, heterocyclo-S, heterocyclo-S(O)ₚ, heterocyclothio, heterocycloalkyl-S, hydrogen, —NO₂, —OCF₃, —OH, tetrazolyl, trifluoromethylcarbonylamino, trifluoromethylsulfonamido, -Z¹⁰⁵-C(O)N(R)₂, -Z¹⁰⁵-N(R)—C(O)-Z²⁰⁰, -Z¹⁰⁵-N(R)-S(O)₂-Z²⁰⁰, -Z¹⁰⁵-N(R)—C(O)—N(R)-Z²⁰⁰, —N(R)—C(O)R, —N(R)—C(O)OR, O—R—C(O)-heterocyclyl-OR, R_{c} and —CH₂OR_{c};
   where R_{c} for each occurrence is independently hydrogen, optionally substituted aliphatic, optionally substituted heterocyclyl-(C₁-C₆)—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
   Z¹⁰⁵ for each occurrence is independently a covalent bond or an aliphatic group;
   Z²⁰⁰ for each occurrence is independently selected from an optionally substituted group selected from the group consisting of an aliphatic group, aliphatic-phenyl and phenyl;
   R_{d} and Rₑ for each occurrence are independently H, an aliphatic group, alkanoyl or SO₂-alkyl; or R_{d}, Rₑ and the nitrogen atom to which they are attached together form a five- or six-membered heterocyclic ring;
   t for each occurrence is independently an integer from 2 to 6;
   W for each occurrence is independently a bond or O, S, S(O), S(O)₂, or NR_{f}, wherein R_{f} for each occurrence is independently H or an aliphatic group; or
Rₐ is an optionally substituted cycloalkyl or heterocyclyl ring fused with the ring to which it is attached;
B is a bond or a) hydrogen ; b) optionally substituted trityl; c) optionally substituted cycloalkyl; d) azaheterocyclyl substituted with an optionally substituted aliphatic group; e) azacycloalkyl which is substituted with one or more substituents selected from the optionally substituted group consisting of —(C₁-C₆)-alkyl, —(C₁-C₆)-alkyl-OR,—C(O)—(C₁-C₆)-alkyl-N(R)₂,—C₁-C₆)-alkyl-N(R)₂, —(C₁-C₆)-alkyl-cycloalkyl, tetrahydrothienyl, and tetrahydrothiopyranyl; f) a group of the formula 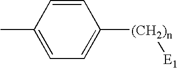
wherein E₁ is selected from an optionally substituted group consisting of amido, amino, imidazolyl, morpholinyl, piperazinyl, piperidinyl, pyrrolidinyl, or tetrahydrothiazolyl, and wherein E₁ is optionally substituted with one or more substituents selected from —(C₀-C₆)-alkyl-OR, —(C₁-C₆)-alkyl-C(O)OR, (C₁-C₆)alkyl-heterocylyl-(C₁-C₆)-alkyl-heterocycloalkyl, —(C₁-C₆)-alkyl-N(R)₂, cyclohexanone, alkoxyalkyl, and pyranyl, g) optionally substituted (C₁-C₆)-alkyl, h) optionally substituted cycloalkyl, i) optionally substituted alkoxyalkoxy, j) optionally substituted alkylamino, k) optionally substituted dialkylamino, l) alkylester, m) alkenyl, n) optionally substituted alkoxy, o) optionally substituted heterocyclyl, p) optionally substituted phenyl, q) optionally substituted 1,4-dioxa-spiro[4.5]decane, r) optionally substituted 1-oxa-2-aza-spiro[4.5]dec-2-ene, s) optionally substituted [1,3]dioxolane, t) —R²⁰⁰—O—(R²⁰⁰)₂—Si(R²⁰⁰)₃, u) a bond, provided that B, Z and E are not each a bond, v) alkoxyalkyl or w) phenylalkyl;
Z is a bond, carbonyl, R²⁰⁰—O—, amino, —O—, —S— or SO₂;
E is a bond or H, or is an optionally substituted group selected from the group consisting of alkoxy, alkoxy-aliphatic, alkoxyamino, alkoxyalkoxy, alkoxycarbonyl-aliphatic, aliphatic group, aliphatic-aminoaliphatic, aliphaticcarbonyl, alkylsulfonyl, amino, amino-aliphatic, amino-aliphatic-carbonyl, aminocarbonyl, aminocarbonyl-aliphatic, aminosulfonyl-aliphatic, CH₂—C(CH₃)₂(OH), —C(CH₃)₂N(CH₃)(H), cycloalkyl, di-aliphatic-amino, di-aliphatic-amino-aliphatic, di-aliphatic-amino-aliphatic-amino, di-aliphatic-aminocarbonyl, di-aliphatic-aminocarbonyl-aliphatic, heterocyclyl, heterocyclo-aliphatic, morpholinocarbonyl-aliphatic, phenyl, piperidinylalkoxy, tetrahydropyranyl-aliphatic, thiopyranyl, tetrahydrothiopyran-1,1-dioxide, triazolyl-aliphatic and urea; or
E is
   —CH(R²⁰⁰)—C(O)—N(C₁-C₆)—N(R²⁰⁰)₂,
   —N(R²⁰⁰)—(C₁-C₆)—C(O)—N(R²⁰⁰)₂,
   —N(R²⁰⁰)—(C₁-C₆)—C(O)—OH,
   —N(R²⁰⁰)—(C₁-C₆)—C(O)-morpholinyl,
   —(C₁-C₆)—S—CH₃,
   —C(R²⁰⁰)(CH₂OH)—(C₁-C₆)—OH,
   —C(R²⁰⁰)₂—N(R²⁰⁰)₂,
   —C(O)—OH,
   —C(R²⁰⁰)₂(OH),
   —C(R²⁰⁰)₂—O—(C₁-C₆)—C(R²⁰⁰)₂(OH),
   —C(R²⁰⁰)₂C(R²⁰⁰)₂(OH),
      wherein R²⁰⁰ is independently hydrogen or alkyl;
R² is H, —NH₂, —S(C₁-C₆) alkyl, —SO₂(C₁-C₆) alkyl, optionally substituted alkyl, —OR⁷, —N(H)SO₂R⁷, —N(R⁷)SO₂R⁷, —N(R⁷)C(O)N(H)R⁷, —N(R⁷)C(O)NR⁷, —N(H)C(O)R⁷, —N(R⁷)₂, —N(R⁷)C(O)R⁷, —NHC(O)NHR⁷, or —NHR⁷;
R⁷ is (C₁-C₆)-aliphatic optionally substituted by one or more substituents each independently selected from the group consisting of (C₁-C₆)alkoxy, heterocyclyl, hydroxyl, —NR⁵R⁶ optionally substituted phenyl, —C(O)R⁴ and heterocyclyl;
   wherein any of said alkoxy, aliphatic and heterocyclyl may be optionally substituted;
wherein R⁵ and R⁶ are independently H or (C₁-C₆)alkyl, —NHS(O)₂R⁴, —NHC(O)R⁴ or —NHC(═NH)R⁴;
   wherein R⁴ is selected from (C₁-C₆)alkyl and H;
Y is H, OR³ or N(R³)₂ wherein R³ is independently selected from H or an optionally substituted group consisting of aliphatic, —(CH₂)₂—C(O)—NH₂, —C(O)-aliphatic, —C(O)-cycloalkyl, and —C(O)-heterocyclyl;
   where R for each occurrence is independently H or selected from an optionally substituted group consisting of aliphatic, heterocyclyl and heterocyclo-aliphatic;
n is an integer from 1 to 6; and
p is 1 or 2;
   provided that
   when A-L-R¹ is or 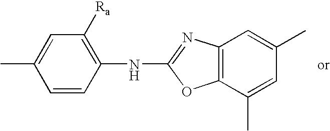 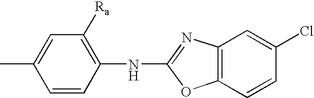
   then B-Z-E is not a pyrrolidinyl which is substituted with 2-methoxyethyl, N,N-dimethylaminomethyl, N,N-dimethylamino-1-oxoethyl, or 2-(N-methylamino)-1-oxopropyl;
   when X is N; Y is NH₂; R² is H; L is NH; A is phenyl optionally substituted with fluoro or methoxy; B is cyclohexyl; Z is a bond and E is piperazinyl substituted with methyl, then R¹ is not:
   phenyl optionally substituted with C₂H₄OH or chloro,
   benzofuranyl optionally substituted with chloro,
   imidazolyl optionally substituted with methyl,
   benzoxazolyl optionally substituted with one or two methyls,
   benzoxazolyl optionally substituted with one or two chloros,
   benzoxazolyl optionally substituted with methoxy,
   benzoxazolyl optionally substituted with ethyl,
   benzoxazolyl optionally substituted with carbonitrile,
   benzoxazolyl optionally substituted with isopropyl,
   benzothiazolyl optionally substituted with one or two methyls,
   benzothiazolyl optionally substituted with propyl,
   benzothiazolyl optionally substituted with isopropyl,
   benzothiazolyl optionally substituted with ethyl and phenyl,
   thiazolyl substituted with ethyl,
   thiazolyl optionally substituted phenyl,
   thiazolyl optionally substituted with phenylmethyl,
   thiazolyl optionally substituted with nitrophenyl,
   thiazolyl optionally substituted with two methyls,
   thiazolyl substituted with phenyl and methyl,
   thiazolyl substituted with phenyl and propyl,
   thiazolyl substituted with phenyl and isopropyl,
   thiazolyl substituted with ethyl and methylphenyl,
   benzoisothiazolyl optionally substituted with CF₃,
   benzoisothiazolyl optionally substituted with one or two oxo,
   benzoisoxazolyl substituted with CF₃,
   indazolyl, or pyrimidinyl; or
   when X is N; Y is NH₂; R² is H; L is NH; A is phenyl optionally substituted with fluoro; R¹ is benzoxazolyl substituted with one or two methyls, benzothiazolyl or ethyl; Z is a bond; and E is COOH, piperazinyl substituted with methyl, piperazinyl substituted with oxo, or ethyl substituted with oxo;
   then B is not ethyl, cyclohexyl, piperidinyl substituted with dimethylamino, or phenyl substituted with CN; or
   when X is N; Y is NH₂; R²is H; L is NH; A is phenyl; B is a bond; Z is a bond; and R¹ is benzofuranyl, benzoisoxazolyl, piperidinyl, pyrrolyl, isooxazolyl substituted with phenyl, isoxazolyl substituted with trifluoromethyl, benzoxazolyl optionally substituted with one or two methyls, benzoxazolyl optionally substituted with ethyl, benzoxazolyl optionally substituted with chloro, or benzoxazolyl optionally substituted with isopropyl then
E is not:
   piperidinyl optionally substituted with substituted alkyl,
   piperazinyl,
   pyrrolidinyl optionally substituted with methoxyethyl,
   piperidinyl optionally substituted with dihydroxypropyl,
   piperidinyl optionally substituted with hydroxyethyl,
   piperidinyl optionally substituted with methoxyethyl,
   piperidinyl optionally substituted with methylsulfanylethyl,
   piperidinyl optionally substituted with optionally substituted ethyl,
   piperidinyl optionally substituted with optionally substituted propyl,
   imidazolyl optionally substituted with methyl,
   imidazolyl optionally substituted with amino,
   aminoalkylcarbonyl,
   cyclohexanecarboxylate, or
   pyrimidinyl substituted with CN; or
when X is N; Y is NH₂; R² is H; A is phenyl; R¹ is phenyl; B is cyclohexyl; Z is a bond; and E is piperazinyl substituted with methyl; then L is not methyl substituted by ═N—OCH₃, ═N—OH, NH₂ or CN; or
when X is N; Y is NH₂; R² is H; L is NH; A is phenyl; R¹ is benzoxazolyl substituted with two methyls; B is pyrrolidinyl optionally substituted with methylaminomethyl and ethyl, or pyrrolidinyl optionally substituted by dimethylamino and ethyl; and Z is carbonyl; then E is not dialkylamino, a bond or alkyl substituted with methylamino; or
when X is N; L is NH; A is phenyl; R¹ is benzoxazolyl optionally substituted with two methyls; B is cyclohexyl; and Z is a bond; then E is not dimethylamino or morpholino; or
when X is N; L is NH; A is phenyl; R¹ is benzoxazolyl optionally substituted with two methyls; B is cyclohexyl; and Z is NH; then E is not methoxyethyl or methyl; or
when X is N; Y is NH₂; R² is H; L is NH; A is phenyl; R¹ is benzoxazolyl substituted with two methyls; B is piperidinyl; and Z is a bond; then E is not a bond; or
when X is N; L is O-alkyl; A is phenyl; B is cyclohexyl or a bond; Z is a bond; and E is cyclopentyl or piperazinyl substituted with methyl; then R¹ is not phenyl optionally substituted with benzenesulfonamide or phenyl optionally substituted with benzylurea; or
when X is N, Y is NH₂, R² is H, L is NH, A is phenyl optionally substituted with fluoro, R¹ is benzoxazolyl substituted with ethyl, benzoxazolyl substituted with chloro, or benzoxazolyl substituted with one or two methyls; B is piperidinyl, azetidinyl, pyrrolyl, or cyclohexyl; and Z is a bond; then E is not:
   methoxyethyl,
   methoxypropyl,
   methyl,
   ethyl optionally substituted with hydroxyl,
   piperazinyl substituted with oxo, or
   imidazolyl optionally substituted with amino; or
when X is N; Y is NH₂; R² is H; L is NH; A is phenyl; B is piperidinyl; Z is carbonyl; and R¹ is benzoxazolyl optionally substituted with two methyls or benzoxazolyl optionally substituted with chloro; then
   E is not:
   morpholinoalkyl,
   dimethylaminomethyl,
   piperidinyl optionally substituted with methyl,
   isopropyl substituted with methylamine,
   pyrrolidinyl,
   ethyl optionally substituted with methyl and methylamino, or
   ethyl optionally substituted with substituted alkyl; or
when X is N; Y is NH₂; R² is H; L is carbonyl; A is phenyl; Z is a bond; E is piperidinyl or pyridinyl; and B is a bond; then
   R¹ is not:
   oxazolyl,
   isoxazolyl optionally substititued with methyl,
   isoxazolyl optionally substituted with phenyl,
   pyrazolyl optionally substituted with benzyl,
   pyrazolyl optionally substituted with benzoyl,
   pyrazolyl optionally substituted with methyl, or
   pyrazolyl optionally substituted with ethanone; or
when X is N; Y is NH₂; R² is H; L is carbonyl; A is phenyl; Z is a bond; R¹ is phenyl; and B is cyclohexyl; then E is not piperazinyl substituted with methyl; or
when X is N; L is alkyl optionally substituted with OH; A is phenyl optionally substituted with methoxy; R¹ is benzoxazolyl or benzimidazolyl; B is cyclohexyl; and Z is a bond; then E is not piperazinyl substituted with methyl.

A preferred embodiment of Formula I, pharmaceutically acceptable salts thereof, metabolites thereof, isomers thereof, or pro-drugs thereof, is where Y is —N(R³)₂.

A more preferred embodiment of the compound of any of the foregoing inventions wherein:
X is N;
A is optionally substituted phenyl;
R¹ is optionally substituted benzoxazolyl or optionally substituted benzothiazolyl;
B is a bond or is selected from an optionally substituted group consisting of alkenyl, alkyl, alkoxyalkyl, (C₃-C₇)cycloalkyl, (C₃-C₇)cycloalkenyl, heterocyclyl, phenyl, 1,4-dioxa-spiro[4.5]dec-2-ene, 2,2-dipropyl[1,3]dixolane, 1-oxa-2-aza-spiro[4.5]dec-2-ene, 1,4-dioxa-spiro[4.5]decane and 2,2-dipropyl[1,3]dioxolane;
E is H or selected from an optionally substituted group consisting of alkoxy, alkoxyalkyl, alkoxyalkoxy, alkoxyamino, alkyl, alkylaminoalkyl, aminoalkyl, aminoalkylcarbonyl, aminocarbonyl, azetidinyl, benzimidazolyl, —C(CH₃)(CH₂OH)—CH₂—OH, —C(CH₃)₂, —NH(CH₃), —C(CH₃)₂—O—CH₂—C(CH₃)₂(OH), —CH₂—C(CH₃)₂(OH), —(CH₂)₂—S—CH₃, COOH, cycloalkyl, diazepanyl, dimethylamino, dimethylaminoalkyl, dimethylaminoalkylamino, dimethylaminocarbonyl, dimethylaminocarbonylalkyl, furanyl, imidazolinyl, imidazolyl, imidazolylalkyl, isoxazolyl, morpholinyl, morpholinylalkyl, —N(CH₃)—CH₂—C(═O)-morpholinyl, —N(CH₃)—CH₂—C(═O)—N(CH₃)₂, —N(CH₃)—CH₂—C(═O)—OH, oxodiazolyl, oxazolyl, piperazinyl, piperidinyl, pyranyl, pyrazinyl, pyrazolyl, pyridinyl, pyrrolidinyl, pyrrolyl, tetrahydropyranyl, tetrazolyl, thiadiazolyl, thiopyranyl, thienyl, triazolyl and triazolylalkyl;
R²is H, SCH₃, NH₂, or S(O)₂—CH₃; and
R³ for each occurrence is independently H or —(CH₂)₂—C(═O)NH₂.

A more preferred embodiment of the compound, pharmaceutically acceptable salts thereof, metabolites thereof, isomers thereof, or pro-drugs thereof, of any of the foregoing inventions wherein:
A is optionally substituted by one or more substituents selected from the group consisting of alkyl, alkoxy, chloro and fluoro;
R¹ is optionally substituted by one or more substituents selected from the group consisting of alkyl, alkenyl, alkoxy, alkoxyalkoxy, alkoxycarbonylpiperidinylalkoxy, alkylcarbonyl, aminocarbonyl, bromo, CF₃, chloro, C(═O)—O(CH₃)₃, dialkylaminoalkoxy, dialkylaminocarbonyl, dialkylaminocarbonylalkoxy, fluoro, —OH, morpholinoalkoxy, NO₂, OCF₃, phenyl-S-alkoxy, optionally substituted piperidinylalkoxy, optionally substituted pyridinylalkoxy, optionally substituted pyrrollidinylalkoxy and optionally substituted thienylalkoxy;
B is a bond or an optionally substituted group selected from the group consisting of alkoxyalkyl, alkyl, azetidinyl, cycloalkenyl, cycloalkyl, isoxazolyl, phenyl, piperidinyl, pyranyl, pyridinyl, pyrrolidinyl, tetrahydropyranyl, tetrahydrothiopyranyl, thiopyranyl, 1,4-dioxa-spiro[4.5]dec-2-ene, [1,3]dioxolane, 1-oxa-2-aza-spiro[4.5]dec-2-ene, and 1,4-dioxa-spiro[4.5]decane;
E is H, dimethylaminoalkyl, dimethylaminocarbonyl or an optionally substituted group selected from the group consisting of alkyl, alkoxyalkyl, azetidinyl, benzimidizolyl, diazepanyl, furanyl, imidazolyidinyl, imidazolyl, isoxazolyl, morpholinyl, oxadiazolyl, oxazolyl, phenyl, piperidinyl, piperazinyl, pyrazinyl, pyrazolyl, pyridinyl, pyrrolidinyl, pyrrolyl, tetrahydrofuranyl, tetrahydropyranyl, tetrahydrothiopyran 1,1-dioxide, tetrazolyl, thiadiazolyl, thienyl, thiopyranyl, and triazolyl; and
wherein the group is optionally substituted by one or more substituents selected from the group consisting of alkoxy, alkoxyalkyl, alkyl, alkylcarbonyl, alkylsulfonyl, dialkylaminosulfonyl, fluoro, hydroxy, hydroxyalkyl, nitrile, oxo, S(O)₂CH₃, and S(O)₂CF₃.

A more preferred embodiment of the compound, pharmaceutically acceptable salts thereof, metabolites thereof, isomers thereof, or pro-drugs thereof, of any of the foregoing inventions wherein L is NH, C(OH)H or carbonyl;
B is a bond or is selected from the optionally substituted group consisting of alkyl, azetidinyl, cycloalkyl, isoxazolyl, phenyl, piperidinyl, pyranyl, tetrahydropyranyl, tetrahydrothiopyranyl, 1,4-dioxa-spiro[4.5]dec-2-ene, [1,3]dioxolane, 1-oxa-2-aza-spiro[4.5]dec-2-ene, and 1,4-dioxa-spiro[4.5]decane;
wherein the group is substituted by one or more substituents selected from the group consisting of alkoxy, alkyl, CF₃, C≡N, cycloalkyl, fluoro, and hydroxyl.

A more preferred embodiment of the compound, pharmaceutically acceptable salts thereof, metabolites thereof, isomers thereof, or pro-drugs thereof, of any of the foregoing inventions wherein R² is H.

A more preferred embodiment of the compound, pharmaceutically acceptable salts thereof, metabolites thereof, isomers thereof, or pro-drugs thereof, of any of the foregoing inventions wherein R³ for each occurrence is H.

An even more preferred embodiment of the compound, pharmaceutically acceptable salts thereof, metabolites thereof, isomers thereof, or pro-drugs thereof, any of the foregoing inventions wherein R¹ is benzoxazolyl or benzothiazolyl, each optionally substituted by one or more substituents selected from the group consisting of alkenyl, alkoxy, alkyl, bromo, CF₃, chloro, dimethylaminocarbonyl, fluoro, hydroxyl, OCF₃ and nitrile.

A most preferred embodiment of the compound, pharmaceutically acceptable salts thereof, metabolites thereof, isomers thereof, or prodrugs thereof, any of the foregoing inventions wherein A is phenyl optionally substituted by fluoro or alkoxy;
L is NH;
R¹ is benzoxazolyl optionally substituted by one or more substituents selected from the group consisting of CF₃, CH₃ and chloro;
Z is a bond, carbonyl, R²⁰⁰—O—, —O— or —S—; and
E is H or selected from the optionally substituted group consisting of alkoxyalkyl alkoxyamino, alkyl, COOH, cycloalkyl, diazepanyl, dimethylaminocarbonyl, furanyl, imidazolylalkyl, imidazolidinyl, imidazolyl, isoxazolyl, morpholinyl, —N(R²⁰⁰)—R²⁰⁰—C(═O)—N(R²⁰⁰)₂, —N(R²⁰⁰)—R²⁰⁰—C(═O)—OH, —N(R²⁰⁰)—R²⁰⁰—C(═O)-morpholinyl, OH, oxazolyl, piperazinyl, piperidinyl, pyrazinyl, pyrazolyl, pyridinyl, pyrrolidinyl, tetrahydrofuranyl, tetrahydropyranyl, tetrazolyl, thiadiazolyl, thienyl, and triazolyl;
wherein R²⁰⁰ is alkyl.

The compound of any of the foregoing inventions wherein the compound is
3-[3-(fluoro-4-(5-trifluoromethyl-benzoxazol-2-ylamino)-phenyl]-1-[4-(2-methoxy-ethoxy)-cyclohexyl]-1H-pyrazolo[3,4-d]pyrimidin-4-ylamine,
3-[4-(7-chloro-5-methyl-benzoxazol-2-ylamino-phenyl]-1-[4-(2-methoxy-ethoxy)-cyclohexyl]-1H-pyrazolo[3,4-d]pyrimidin-4-ylamine, or
1-(4-{4-amino-3-[4-(5-chloro-benzoxazol-2-ylamino)-3-fluoro-phenyl]-pyrazolo[3,4-d]pyrimidin-1-yl}-cyclohexyloxy)-2-methyl-propan-2-ol.

The compound, pharmaceutically acceptable salts thereof, metabolites thereof, isomers thereof, or pro-drugs thereof, of Group A, denoted as Group B, wherein:
X is CH;
A is optionally substituted phenyl;
R¹ is optionally substituted benzoxazolyl;
B is H or selected from the optionally substituted group consisting of alkoxyalkyl, alkyl, cycloalkyl and heterocyclyl;
E is H, or is selected from an optionally substituted group consisting of alkoxy, alkyl, alkylsulfonyl, aminocarbonylalkyl, diazepanyl, dimethylamino, morpholinyl, phenyl, piperazinyl, tetrazolyl and urea;
R² is H, NH₂, SCH₃, or SO₂CH₃; and
R³ for each occurrence is H.

A preferred embodiment of the compound, pharmaceutically acceptable salts thereof, metabolites thereof, isomers thereof, or pro-drugs thereof, of Group B wherein:
A is optionally substituted by fluoro;
R¹ is an optionally substituted benzoxazolyl substituted by one or more substituents selected from the group consisting of alkoxy, alkyl, bromo, chloro, CF₃, dialkylaminoethoxy, fluoro, morpholinylalkoxy, morpholinylalkyl and nitrile;
B is H or is selected from the optionally substituted group consisting of cycloalkyl, alkyl, piperidinyl and pyrrolidinyl;
wherein the substituents are selected from the group consisting of alkyl, hydroxyl, oxo, nitrile and nitro;
E is H or selected from the optionally substituted group consisting of alkyl, alkoxy, alkoxyalkyl, alkylsulfonyl, aminocarbonylalkyl, diazepanyl, dimethylamino, morpholinyl, piperazinyl, phenyl, tetrazolyl and urea;
wherein the group is optionally substituted by one or more substituents selected from the group consisting of alkoxy, alkyl, alkylsulfonyl, cycloalkyl, hydroxyl, nitrile, nitro, NH₂ and oxo; and
Z is a bond, R²⁰⁰—O—, NH or —O—.

A preferred embodiment of any of the foregoing inventions of Group B, pharmaceutically acceptable salts thereof, metabolites thereof, isomers thereof, or pro-drugs thereof wherein L is NH or N(alkenyl).

A preferred embodiment of any of the foregoing inventions of Group B pharmaceutically acceptable salts thereof, metabolites thereof, isomers thereof, or pro-drugs thereof wherein R² is H.

A preferred embodiment of any of the foregoing inventions of Group B, pharmaceutically acceptable salts thereof, metabolites thereof, isomers thereof, or pro-drugs thereof, wherein R¹ is optionally substituted benzoxazolyl substituted by one or more substituents selected from the group consisting of alkyl, bromo, CF₃, chloro, fluoro and nitrile.

A preferred embodiment of any of the foregoing inventions of Group B, pharmaceutically acceptable salts thereof, metabolites thereof, isomers thereof, or pro-drugs thereof, wherein
A is phenyl or phenyl substituted by fluoro;
L is NH;
R¹ is benzoxazolyl substituted by one or more substituents selected from the group consisting of alkyl, bromo, CF₃ and chloro;
Z is a bond or —O—; and
E is optionally substituted alkyl, alkoxyalkyl, diazepanyl, piperazinyl or tetrazolyl.

A preferred embodiment of any of the foregoing inventions of Group B, pharmaceutically acceptable salts thereof, metabolites thereof, isomers thereof, or pro-drugs thereof, wherein:
X is CH;
A is optionally substituted phenyl;
R¹ is optionally substituted benzoxazolyl;
B is H or a bond or selected from the optionally substituted group consisting of alkyl and cycloalkyl;
Z is a bond, —R²⁰⁰—O—, amino or —O—;
E is H, a bond or an optionally substituted group selected from the group consisting of alkoxy, alkyl, alkylsulfonyl, aminocarbonylalkyl, dialkylamino, heterocyclyl, phenyl and urea;
R² is H, NH₂, —S(C₁-C₆)alkyl, or —SO₂(C₁-C₆)alkyl; and
R³ for each occurrence is H.

A more preferred embodiment of any of the foregoing inventions of Group B, pharmaceutically acceptable salts thereof, metabolites thereof, isomers thereof, or pro-drugs thereof, wherein
A is optionally substituted by one or more fluoro;
R¹ is optionally substituted by one or more substituents selected from the group consisting of alkyl, alkoxy, aminoalkoxy, bromo, CF₃, chloro, fluoro, morpholinoalkoxy, morpholinoalkyl and nitrile;
E is H or an optionally substituted group selected from the group consisting of alkoxy, alkyl, alkylsulfonyl, aminocarbonylalkyl, diazapenyl, dimethylamino, morpholinyl, phenyl, piperazinyl, pyridinyl, pyrrolidinyl, tetrazolyl and urea;
wherein the optionally substituted group is optionally substituted by one or more alkoxy, alkyl, amino, bromo, cycloalkyl, dimethylamino, hydroxyl, oxo, nitrile, NO₂ or sulfonyl; and
R is H.

An even more preferred embodiment of any of the foregoing inventions of Group B, pharmaceutically acceptable salts thereof, metabolites thereof, isomers thereof, or pro-drugs thereof, wherein
L is NH or N-alkenyl;
R¹ is substituted by one or more alkyl, bromo, CF₃, chloro, fluoro, or nitrile;
A is phenyl optionally substituted by fluoro;
B is a bond or is selected from the optionally substituted group consisting of alkyl, cycloalkenyl, cyclopentyl or cyclohexyl;
Z is a bond, —O— or —R²⁰⁰—O—; and
E is H, or selected from an optionally substituted group consisting of alkoxy, alkenyl, alkyl, cycloalkyl, diazapenyl, piperazinyl and tetrazolyl.

A most preferred embodiment of any of the foregoing inventions of Group B compound, pharmaceutically acceptable salts thereof, metabolites thereof, isomers thereof, or pro-drugs thereof, wherein:
R¹ is substituted by alkyl, bromo or chloro; L is NH; B is cyclohexyl; Z is a bond or —R²⁰⁰—O—;
   wherein R²⁰⁰ is alkyl;
E is alkoxy or optionally substituted piperazinyl; and Y is NH.

The compound of any of the foregoing inventions of Group B wherein the compound is
4-(4-{4-Amino-5-[4-(5-chloro-benzoxazol-2-ylamino)-3-fluoro-phenyl]-pyrrolyl[2,3-d]pyrimidin-7-yl}-cyclohexyl-1-methyl-piperazin-2-one
5-[4-(5-Chloro-benzooxazol-2-ylamino)-phenyl]-7-[4-(2-methoxy-ethoxy)-cyclohexyll]-7H-pyrrolo[2,3-d]pyrimidin-4-ylamine; or
5-[4-(5-Bromo-7-methyl-benzooxazol-2-ylamino)-phenyl]-7-[4-(2-methoxyethoxy)-cyclohexyl]-7H-pyrrolo[2,3-d]pyrimidin-4-ylamine.

The compounds of this invention are useful for treating a disease or condition in a patient in need thereof, comprising administering a compound of Formula I to said patient, wherein the disease or condition is selected from the group consisting of rheumatoid arthritis, thyroiditis, type 1 diabetes, multiple sclerosis, sarcoidosis, inflammatory bowel disease, Crohn's disease, myasthenia gravis, systemic lupus erythematosus, psoriasis, organ transplant rejection, benign and neoplastic proliferative diseases, lung cancer, breast cancer, stomach cancer, bladder cancer, colon cancer, pancreas cancer, ovarian cancer, prostate cancer, rectal cancer, hematopoietic malignancies, diabetic retinopathy, retinopathy of prematurity, choroidal neovascularization due to age-related macular degeneration, infantile hemangiomas, edema, ascites, effusions, exudates, cerebral edema, acute lung injury, adult respiratory distress syndrome, blood vessel proliferative disorders, fibrotic disorders, mesangial cell proliferative disorders, metabolic diseases, atherosclerosis, restenosis, psoriasis, hemangiomas, myocardial angiogenesis, coronary collaterals, cerebral collaterals, ischemic limb angiogenesis, ischemia/reperfusion injury, wound healing, peptic ulcer Helicobacter related diseases, virally-induced angiogenic disorders, fractures, Crow-Fukase syndrome (POEMS), preeclampsia, menometrorrhagia, cat scratch fever, rubeosis, neovascular glaucoma, retinopathies, malignant ascites, von Hippel Lindau disease, hematopoietic cancers, hyperproliferative disorders, burns, chronic lung disease, stroke, polyps, anaphylaxis, chronic inflammation, allergic inflammation, delayed-type hypersensitivity, ovarian hyperstimulation syndrome, angina, ankylosing spondylitis, asthma, congestive obstructive pulmonary disease (COPD), hepatitis C virus (HCV), idiopathic pulmonary fibrosis, myocardial infarct, psoriatic arthritis, restinosis and sciatica.

A pharmaceutical composition comprising a compound according to Formula I and a pharmaceutically acceptable carrier or excipient.

In a further embodiment, the present invention is directed to a method of making an optionally substituted 2-aminobenzoxazole comprising the step of: reacting an optionally substituted N-(2-hydroxyphenyl)thiourea with an oxidant and a base but not including a toxic metal until the reaction is substantially complete; wherein the oxidant is selected from the group consisting of hydrogen peroxide, oxygen, peracids, chlorine, sodium periodate, potassium periodate, tert-butyl peroxide, tert-butyl hypochlorite, sodium perborate, sodium percarbonate, urea hydrogen peroxide adduct, sodium hypochlorite, potassium hypochlorite, sodium hypobromite, potassium hypobromite, sodium bromate, potassium bromate, potassium permanganate and barium manganate; and the base is selected from the group consisting of metal and tetraalkylammonium hydroxides, metal and tetraalkylammonium carbonates, metal and tetraalkylammonium bicarbonates, metal and tetraalkylammonium alkoxides, metal and tetraalkylammonium phosphates, metal and tetraalkylammonium dibasic phophates.

### DETAILED DESCRIPTION OF THE INVENTION

Protein Tyrosine Kinases. Protein tyrosine kinases (PTKs) are enzymes which catalyse the phosphorylation of specific tyrosine residues in cellular proteins. This post-translational modification of these substrate proteins, often enzymes themselves, acts as a molecular switch regulating cell proliferation, activation or differentiation (for review, see Schiessinger and Ulrich, 1992, Neuron 9:383-391). Aberrant or excessive PTK activity has been observed in many disease states including benign and malignant proliferative disorders as well as diseases resulting from inappropriate activation of the immune system (e.g., autoimmune disorders), allograft rejection, and graft vs. host disease. In addition, endothelial-cell specific receptor PTKs such as KDR and Tie-2 mediate the angiogenic process, and are thus involved in supporting the progression of cancers and other diseases involving inappropriate vascularization (e.g., diabetic retinopathy, choroidal neovascularization due to age-related macular degeneration, psoriasis, arthritis, retinopathy of prematurity, and infantile hemangiomas).

Tyrosine kinases can be of the receptor-type (having extracellular, transmembrane and intracellular domains) or the non-receptor type (being wholly intracellular).

Receptor Tyrosine Kinases (RTKs). The RTKs comprise a large family of transmembrane receptors with diverse biological activities. At present, at least nineteen (19) distinct RTK subfamilies have been identified. The receptor tyrosine kinase (RTK) family includes receptors that are crucial for the growth and differentiation of a variety of cell types (Yarden and Ullrich, Ann. Rev. Biochem. 57:433-478, 1988; Ullrich and Schlessinger, Cell 61:243-254, 1990). The intrinsic function of RTKs is activated upon ligand binding, which results in phosphorylation of the receptor and multiple cellular substrates, and subsequently in a variety of cellular responses (Ullrich & Schlessinger, 1990, Cell 61:203-212). Thus, receptor tyrosine kinase mediated signal transduction is initiated by extracellular interaction with a specific growth factor (ligand), typically followed by receptor dimerization, stimulation of the intrinsic protein tyrosine kinase activity and receptor trans-phosphorylation. Binding sites are thereby created for intracellular signal transduction molecules and lead to the formation of complexes with a spectrum of cytoplasmic signaling molecules that facilitate the appropriate cellular response (e.g., cell division, differentiation, metabolic effects, and changes in the extracellular microenvironment; see Schlessinger and Ullrich, 1992, Neuron 9:1-20).

Proteins with SH2 (src homology-2) or phosphotyrosine binding (PTB) domains bind activated tyrosine kinase receptors and their substrates with high affinity to propagate signals into cells. Both of the domains recognize phosphotyrosine. (Fantl et al., 1992, Cell 69:413-423; Songyang et al., 1994, Mol. Cell. Biol. 14:2777-2785; Songyang et al., 1993, Cell 72:767-778; and Koch et al., 1991, Science 252:668-678; Shoelson, Curr. Opin. Chem. Biol. (1997), 1(2), 227-234; Cowburn, Curr. Opin. Struct. Biol. (1997), 7(6), 835-838). Several intracellular substrate proteins that associate with receptor tyrosine kinases (RTKs) have been identified. They may be divided into two principal groups: (1) substrates which have a catalytic domain; and (2) substrates which lack such a domain but serve as adapters and associate with catalytically active molecules (Songyang et al., 1993, Cell 72:767-778). The specificity of the interactions between receptors or proteins and SH2 or PTB domains of their substrates is determined by the amino acid residues immediately surrounding the phosphorylated tyrosine residue. Thus, phosphorylation provides an important regulatory step which determines the selectivity of signaling pathways recruited by specific growth factor receptors, as well as differentiation factor receptors.

Several receptor tyrosine kinases such as FGFR-1, PDGFR, TIE-2 and c-Met, and growth factors that bind thereto, have been suggested to play a role in angiogenesis, although some may promote angiogenesis indirectly (Mustonen and Alitalo, J. Cell Biol. 129:895-898, 1995). One such receptor tyrosine kinase, known as fetal liver kinase-1 (FLK-1), is a member of the type III subclass of RTKs. An alternative designation for human FLK-1 is kinase insert domain-containing receptor (KDR) (Terman et al., Oncogene 6:1677-83, 1991). Another alternative designation for FLK-1/KDR is vascular endothelial cell growth factor receptor-2 (VEGFR-2) since it binds VEGF with high affinity. The murine version of FLK-1/VEGFR-2 has also been called NYK (Oelrichs et al, Oncogene 8(1):11-15, 1993). Numerous studies such as those reported in Millauer et al., Cell 72:835-846, 1993, suggest that VEGF and FLK-1/KDR/VEGFR-2 are a ligand-receptor pair that play an important role in the proliferation of vascular endothelial cells, and formation and sprouting of blood vessels, termed vasculogenesis and angiogenesis, respectively.

Another type III subclass RTK designated fms-like tyrosine kinase-1 (Flt-1) is related to FLK-1/KDR (DeVries et al. Science 255;989-991, 1992; Shibuya et al., Oncogene 5:519-524, 1990). An alternative designation for Flt-1 is vascular endothelial cell growth factor receptor-1 (VEGFR-1). To date, members of the FLK-1/ KDR/VEGFR-2 and Flt-1/ VEGFR-1 subfamilies have been found expressed primarily on endothelial cells. These subclass members are specifically stimulated by members of the vascular endothelial cell growth factor (VEGF) family of ligands (Klagsburn and D'Amore, Cytokine & Growth Factor Reviews 7: 259-270, 1996). Vascular endothelial cell growth factor (VEGF) binds to Flt-1 with higher affinity than to FLK-1/KDR and is mitogenic toward vascular endothelial cells (Terman et al., 1992, supra; Mustonen et al. supra; DeVries et al., supra). Flt-1 is believed to be essential for endothelial organization during vascular development. Expression of Flt-1 in monocytes, osteoclasts, and osteoblasts, as well as in adult tissues such as kidney glomeruli suggests an additional function for this receptor that is not related to cell growth (Mustonen and Alitalo, supra).

As previously stated, recent evidence suggests that VEGF plays a role in the stimulation of both normal and pathological angiogenesis (Jakeman et al., Endocrinology 133: 848-859, 1993; Kolch et al., Breast Cancer Research and Treatment 36: 139-155, 1995; Ferrara et al., Endocrine Reviews 18(1); 4-25, 1997; Ferrara et al., Regulation of Angiogenesis (ed. L. D. Goldberg and E. M. Rosen), 209-232, 1997). In addition, VEGF has been implicated in the control and enhancement of vascular permeability (Connolly, et al., J. Biol. Chem. 264: 20017-20024, 1989; Brown et al., Regulation of Angiogenesis (ed. L. D. Goldberg and E. M. Rosen), 233-269, 1997). Different forms of VEGF arising from alternative splicing of mRNA have been reported, including the four species described by Ferrara et al. (J. Cell. Biochem. 47:211-218, 1991). Several related homologs of VEGF have recently been identified. Placenta growth factor (PlGF) has an amino acid sequence that exhibits significant homology to the VEGF sequence (Park et al., J. Biol. Chem. 269:25646-54, 1994; Maglione et al. Oncogene 8:925-31, 1993). As with VEGF, different species of PlGF arise from alternative splicing of mRNA, and the protein exists in dimeric form (Park et al., supra). PlGF-1 and PlGF-2 bind to Flt-1 with high affinity, and PlGF-2 also avidly binds to neuropilin-1 (Migdal et al, J. Biol. Chem. 273 (35): 22272-22278), but neither binds to FLK-1/KDR (Park et al., supra). PlGF has been reported to potentiate both the vascular permeability and mitogenic effect of VEGF on endothelial cells when VEGF is present at low concentrations (purportedly due to heterodimer formation) (Park et al., supra).

VEGF-B is produced as two isoforms (167 and 185 residues) that also appear to bind Flt-1/VEGFR-1(Pepper et al, Proc. Natl. Acad. Sci. U.S.A. (1998), 95(20): 11709-11714).

VEGF-C, in its fully processed form, can also bind KDR/VEGFR-2 and stimulate proliferation and migration of endothelial cells in vitro and angiogenesis in in vivo models (Lymboussaki et al, Am. J. Pathol. (1998), 153(2): 395-403; Witzenbichler et al, Am. J. Pathol. (1998), 153(2), 381-394). The transgenic overexpression of VEGF-C causes proliferation and enlargement of only lymphatic vessels, while blood vessels are unaffected. The most recently discovered VEGF-D is structurally very similar to VEGF-C. VEGF-D is reported to bind and activate at least two VEGFRs, VEGFR-3/Flt-4 and KDR/VEGFR-2 (Achen et al, Proc. Natl. Acad. Sci. U.S.A. (1998), 95(2), 548-553 and references therein).

There has been recently reported a virally encoded, novel type of vascular endothelial growth factor, VEGF-E (NZ-7 VEGF), which preferentially utilizes KDR/Flk-1 receptor and carries a potent mitotic activity without heparin-binding domain (Meyer et al, EMBO J. (1999), 18(2), 363-374; Ogawa et al, J. Biol. Chem. (1998), 273(47), 31273-31282.). VEGF-E sequences possess 25% homology to mammalian VEGF and are encoded by the parapoxvirus Orf virus (OV). Like VEGF165, an isoform of VEGF-A, VEGF-E was found to bind with high affinity to VEGF receptor-2 (KDR) resulting in receptor autophosphorylation and a biphasic rise in free intracellular Ca2+ concentrations, while in contrast to VEGF165, VEGF-E did not bind to VEGF receptor-1 (Flt-1).

Based upon emerging discoveries of other homologs of VEGF and VEGFRs and the precedents for ligand and receptor heterodimerization, the actions of such VEGF homologs may involve formation of VEGF ligand heterodimers, and/or heterodimerization of receptors, or binding to a yet undiscovered VEGFR (Witzenbichler et al., supra). Also, recent reports suggest neuropilin-1 (Migdal et al, supra) or VEGFR-3/Flt-4 (Witzenbichler et al., supra), or receptors other than KDR/VEGFR-2 may be involved in the induction of vascular permeability (Stacker, S. A., Vitali, A., Domagala, T., Nice, E., and Wilks, A. F., Angiogenesis and Cancer Conference, Amer. Assoc. Cancer Res., January 1998, Orlando, Fla.; Williams, Diabetelogia 40: S118-120 (1997)).

Tie-2 (TEK) is a member of a recently discovered family of endothelial cell specific receptor tyrosine kinases which is involved in critical angiogenic processes, such as vessel branching, sprouting, remodeling, maturation and stability. Tie-2 is the first mammalian receptor tyrosine kinase for which both agonist ligand(s) (e.g., Angiopoietin1 (“Ang1”), which stimulates receptor autophosphorylation and signal transduction), and antagonist ligand(s) (e.g., Angiopoietin2 (“Ang2”)), have been identified. Knock-out and transgenic manipulation of the expression of Tie-2 and its ligands indicates tight spatial and temporal control of Tie-2 signaling is essential for the proper development of new vasculature. The current model suggests that stimulation of Tie-2 kinase by the Ang1 ligand is directly involved in the branching, sprouting and outgrowth of new vessels, and recruitment and interaction of periendothelial support cells important in maintaining vessel integrity and inducing quiescence. The absence of Ang1 stimulation of Tie-2 or the inhibition of Tie-2 autophosphorylation by Ang2, which is produced at high levels at sites of vascular regression, may cause a loss in vascular structure and matrix contacts resulting in endothelial cell death, especially in the absence of growth/survival stimuli. The situation is however more complex, since at least two additional Tie-2 ligands (Ang3 and Ang4) have recently been reported, and the capacity for heterooligomerization of the various agonistic and antagonistic angiopoietins, thereby modifying their activity, has been demonstrated. Targeting Tie-2 ligand-receptor interactions as an antiangiogenic therapeutic approach is thus less favored and a kinase inhibitory strategy preferred.

Recently, significant upregulation of Tie-2 expression has been found within the vascular synovial pannus of arthritic joints of humans, consistent with a role in the inappropriate neovascularization. Point mutations producing constitutively activated forms of Tie-2 have been identified in association with human venous malformation disorders.

The Non-Receptor Tyrosine Kinases. The non-receptor tyrosine kinases represent a collection of cellular enzymes which lack extracellular and transmembrane sequences. At present, over twenty-four individual non-receptor tyrosine kinases, comprising eleven (11) subfamilies (Src, Frk, Btk, Csk, Abl, Zap70, Fes/Fps, Fak, Jak, Ack and LIMK) have been identified. At present, the Src subfamily of non-receptor tyrosine kinases is comprised of the largest number of PTKs and include Src, Yes, Fyn, Lyn, Lck, Blk, Hck, Fgr and Yrk. The Src subfamily of enzymes has been linked to oncogenesis and immune responses. A more detailed discussion of non-receptor tyrosine kinases is provided in Bohlen, 1993, Oncogene 8:2025-2031, which is incorporated herein by reference.

Many of the tyrosine kinases, whether an RTK or non-receptor tyrosine kinase, have been found to be involved in cellular signaling pathways involved in numerous pathogenic conditions, including cancer, psoriasis, and other hyperproliferative disorders or hyper-immune responses.

Plk-1 Kinase Inhibitors

Plk-1 is a serine/threonine kinase which is an important regulator of cell cycle progression. It plays critical roles in the assembly and the dynamic function of the mitotic spindle apparatus. Plk-1 and related kinases have also been shown to be closely involved in the activation and inactivation of other cell cycle regulators, such as cyclin-dependent kinases. High levels of Plk-1 expression are associated with cell proliferation activities. It is often found in malignant tumors of various origins.

Cdc2/Cyclin B Kinase Inhibitors (Cdc2 is also Known as cdk1)

Cdc2/cyclin B is another serine/threonine kinase enzyme which belongs to the cyclin-dependent kinase (cdks) family. These enzymes are involved in the critical transition between various phases of cell cycle progression.

Inhibitors of kinases involved in mediating or maintaining disease states represent novel therapies for these disorders. Examples of such kinases include, but are not limited to: (1) inhibition of c-Src (Brickell, Critical Reviews in Oncogenesis, 3:401-406 (1992); Courtneidge, Seminars in Cancer Biology, 5:236-246 (1994), raf (Powis, Pharmacology & Therapeutics, 62:57-95 (1994)) and the cyclin-dependent kinases (CDKs) 1, 2 and 4 in cancer (Pines, Current Opinion in Cell Biology, 4:144-148 (1992); Lees, Current Opinion in Cell Biology, 7:773-780 (1995); Hunter and Pines, Cell, 79:573-582 (1994)), (2) inhibition of CDK2 or PDGF-R kinase in restenosis (Buchdunger et al., Proceedings of the National Academy of Science USA, 92:2258-2262 (1995)), (3) inhibition of CDK5 and GSK3 kinases in Alzheimers (Hosoi et al., Journal of Biochemistry (Tokyo), 117:741-749 (1995); Aplin et al., Journal of Neurochemistry, 67:699-707 (1996), (4) inhibition of c-Src kinase in osteoporosis (Tanaka et al., Nature, 383:528-531 (1996), (5) inhibition of GSK-3 kinase in type-2 diabetes (Borthwick et al., Biochemical & Biophysical Research Communications, 210:738-745 (1995), (6) inhibition of the p38 kinase in inflammation (Badger et al., The Journal of Pharmacology and Experimental Therapeutics, 279:1453-1461 (1996)), (7) inhibition of VEGF-R 1-3 and TIE-1 and -2 kinases in diseases which involve angiogenesis (Shawver et al., Drug Discovery Today, 2:50-63 (1997)), (8) inhibition of UL97 kinase in viral infections (He et al., Journal of Virology, 71:405-411 (1997)), (9) inhibition of CSF-1R kinase in bone and hematopoetic diseases (Myers et al., Bioorganic & Medicinal Chemistry Letters, 7:421-424 (1997), and (10) inhibition of Lck kinase in autoimmune diseases and transplant rejection (Myers et al., Bioorganic & Medicinal Chemistry Letters, 7:417-420 (1997)).

It is additionally possible that inhibitors of certain kinases may have utility in the treatment of diseases when the kinase is not misregulated, but it is nonetheless essential for maintenance of the disease state. In this case, inhibition of the kinase activity would act either as a cure or palliative for these diseases.

VEGF's are unique in that they are the only angiogenic growth factors known to contribute to vascular hyperpermeability and the formation of edema. Hence, VEGF-mediated hyperpermeability can significantly contribute to disorders with these etiologic features.

Because blastocyst implantation, placental development and embryogenesis are angiogenesis dependent, certain compounds of the invention are useful as contraceptive agents and antifertility agents.

The compounds of this invention have inhibitory activity against one or more of the protein kinases listed herein, as well as family members thereof that are not specifically listed. That is, these compounds modulate signal transduction by protein kinases. Compounds of this invention inhibit protein kinases from serine/threonine and tyrosine kinase classes. In particular, these compounds selectively inhibit the activity of the Tie-2/Tie-1 tyrosine kinases. Certain compounds of this invention also inhibit the activity of additional tyrosine kinases such as Flt-1/VEGFR-1, Flt-4, Tie-1, Tie-2, FGFR, PDGFR, IGF-1R, c-Met, Src-subfamily kinases such as Lck, Src, hck, fgr, fyn, yes, etc. Additionally, some compounds of this invention significantly inhibit serine/threonine kinases such as PKC, MAP kinases, erk, CDKs, Plk-1, or Raf-1 which play an essential role in cell proliferation and cell-cycle progression. In addition the metabolites and prodrugs of certain compounds may also possess significant protein kinase inhibitory activity.

The compounds of this invention, when administered to individuals in need of such compounds, inhibit vascular hyperpermeability and the formation of edema in these individuals.

In one embodiment, the present invention provides a method of treating a protein kinase-mediated condition in a patient, comprising adiminstering to the patient a therapeutically or prophylactically effective amount of one or more compounds of Formula I. A “protein kinase-mediated condition” or a “condition mediated by protein kinase activity” is a medical condition, such as a disease or other undesirable physical condition, the genesis or progression of which depends, at least in part, on the activity of at least one protein kinase. The protein kinase can be, for example, a protein tyrosine kinase or a protein serine/threonine kinase.

The patient to be treated can be any animal, and is preferably a mammal, such as a domesticated animal or a livestock animal. More preferably, the patient is a human.

The method of the present invention is useful in the treatment of any of the conditions described above. In one embodiment, the condition is characterized by undesired angiogenesis, edema, or stromal deposition. For example, the condition can be one or more ulcers, such as ulcers caused by bacterial or fungal infections, Mooren ulcers and ulcerative colitis. The condition can also be due to a microbial infection, such as Lyme disease, sepsis, septic shock or infections by Herpes simplex, Herpes Zoster, human immunodeficincy virus, protozoa, toxoplasmosis or parapoxvirus; an angiogenic disorders, such as von Hippel Lindau disease, polycystic kidney disease, pemphigoid, Paget's disease and psoriasis; a reproductive condition, such as endometriosis, ovarian hyperstimulation syndrome, preeclampsia or menometrorrhagia; a fibrotic and edemic condition, such as sarcoidosis, fibrosis, cirrhosis, thyroiditis, hyperviscosity syndrome systemic, Osler-Weber-Rendu disease, chronic occlusive pulmonary disease, asthma, and edema following burns, trauma, radiation, stroke, hypoxia or ischemia; or an inflammatory/immunologic condition, such as systemic lupus, chronic inflammation, glomerulonephritis, synovitis, inflammatory bowel disease, Crohn's disease, rheumatoid arthritis, osteoarthritis, multiple sclerosis and graft rejection. Other suitable conditions also include sickle cell anaemia, osteoporosis, osteopetrosis, tumor-induced hypercalcemia and bone metastases. Additional conditions which can be treated by the method of the present invention include ocular conditions such as ocular and macular edema, ocular neovascular disease, scleritis, radial keratotomy, uveitis, vitritis, myopia, optic pits, chronic retinal detachment, post-laser complications, conjunctivitis, Stargardt's disease and Eales disease, in addition to retinopathy and macular degeneration.

The compounds of the present invention are also useful in the treatment of cardiovascular conditions such as atherosclerosis, restenosis, vascular occlusion and carotid obstructive disease.

The compounds of the present invention are also useful in the treatment of cancer related indications such as solid tumors, sarcomas (especially Ewing's sarcoma and osteosarcoma), retinoblastoma, rhabdomyosarcomas, neuroblastoma, hematopoietic malignancies, including leukaemia and lymphoma, tumor-induced pleural or pericardial effusions, and malignant ascites.

The compounds of the present invention are also useful in the treatment of Crow-Fukase (POEMS) syndrome and diabetic conditions such as glaucoma, diabetic retinopathy and microangiopathy.

The Src, Tec, Jak, Map, Csk, NFγB and Syk families of kinases play pivotal roles in the regulation of immune function. The Src family currently includes Fyn, Lck, Fgr, Fes, Lyn, Src, Yrk, Fyk, Yes, Hck, and Blk. The Syk family is currently understood to include only Zap and Syk. The TEC family includes Tec, Btk, Rlk and Itk. The Janus family of kinases is involved in the transduction of growth factor and proinflammatory cytokine signals through a number of receptors. The Csk family is currently understood to include Csk and Chk. The kinases RIP, IRAK-1, IRAK-2, NIK, p38 MAP kinases, Jnk, IKK-1 and IKK-2 are involved in the signal transduction pathways for key pro-inflammatory cytokines, such as TNF and IL-1. Compounds of Formula I may function as immunomodulatory agents useful for the maintenance of allografts, the treatment of autoimmune disorders and treatment of sepsis and septic shock. Through their ability to regulate the migration or activation of T cells, B-cells, mast cells, monocytes and neutrophils, these compounds could be used to treat such autoimmune diseases and sepsis. Prevention of transplant rejection, either host versus graft for solid organs or graft versus host for bone marrow, are limited by the toxicity of currently available immunosuppressive agents and would benefit from an efficacious drug with improved therapeutic index. Gene targeting experiments have demonstrated the essential role of Src in the biology of osteoclasts, the cells responsible for bone resorption. Compounds of formula I, through their ability to regulate Src, may also be useful in the treatment of osteoporosis, osteopetrosis, Paget's disease, tumor-induced hypercalcemia and in the treatment of bone metastases.

The compounds of formula I which inhibit the kinase activity of normal or aberrant c-kit, c-met, c-fms, src-family members, EGFr, erbB2, erbB4, BCR-Abl, PDGFr, FGFr, IGF1-R and other receptor or cytosolic tyrosine kinases may be of value in the treatment of benign and neoplastic proliferative diseases.

In many pathological conditions (for example, solid primary tumors and metastases, Kaposi's sarcoma, rheumatoid arthritis, blindness due to inappropriate ocular neovascularization, psoriasis and atherosclerosis) disease progression is contingent upon persistent angiogenesis. Certain compounds of formula I capable of blocking the kinase activity of endothelial cell specific kinases could therefore inhibit disease progression.

Vascular destabilization of the antagonist ligand of Tie-2 (Ang2) is believed to induce an unstable “plastic” state in the endothelium. In the presence of high VEGF levels a robust angiogenic response may result; however, in the absence of VEGF or a VEGF-related stimulus, frank vessel regression and endothelial apoptosis can occur (Genes and Devel. 13: 1055-1066 (1999)). In an analogous manner a Tie-2 kinase inhibitor can be proangiogenic or antiangiogenic in the presence or absence of a VEGF-related stimulus, respectively. Hence, Tie-2 inhibitors can be employed with appropriate proangiogenic stimuli, such as VEGF, to promote therapeutic angiogenesis in situations such as wound healing, infarct and ischemia.

The compounds of formula I, a salt thereof, a prodrug thereof or pharmaceutical compositions containing a therapeutically effective amount thereof may be used in the treatment of protein kinase-mediated conditions, such as benign and neoplastic proliferative diseases and disorders of the immune system, as described above. For example, such diseases include autoimmune diseases, such as rheumatoid arthritis, thyroiditis, type 1 diabetes, multiple sclerosis, sarcoidosis, inflammatory bowel disease, Crohn's disease, myasthenia gravis and systemic lupus erythematosus; psoriasis, organ transplant rejection (eg. kidney rejection, graft versus host disease), benign and neoplastic proliferative diseases, human cancers such as lung, breast, stomach, bladder, colon, pancreas, ovarian, prostate and rectal cancer and hematopoietic malignancies (leukemia and lymphoma), and diseases involving inappropriate vascularization for example diabetic retinopathy, retinopathy of prematurity, choroidal neovascularization due to age-related macular degeneration, and infantile hemangiomas in human beings. In addition, such inhibitors may be useful in the treatment of disorders such as, edema, ascites, effusions, and exudates, including for example macular edema, cerebral edema, acute lung injury and adult respiratory distress syndrome (ARDS).

The compounds of formula I or a salt thereof or pharmaceutical compositions containing a therapeutically effective amount thereof are additionally useful in the treatment of one or more diseases afflicting mammals which are characterized by cellular proliferation in the areas of blood vessel proliferative disorders, fibrotic disorders, mesangial cell proliferative disorders and metabolic diseases. Blood vessel proliferative disorders includes restenosis. Fibrotic disorders include hepatic cirrhosis and atherosclerosis. Mesangial cell proliferative disorders include glomerulonephritis, diabetic nephropathy, malignant nephrosclerosis, thrombotic microangiopathy syndromes, organ transplant rejection and glomerulopathies. Metabolic disorders include diabetes mellitus, chronic wound healing, inflammation and neurodegenerative diseases.

The compounds of this invention have antiangiogenic properties. For this reason, these compounds can be used as active agents against such disease states as arthritis, atherosclerosis, restenosis, psoriasis, hemangiomas, myocardial angiogenesis, coronary and cerebral collaterals, ischemic limb angiogenesis, ischemia/reperfusion injury, wound healing, peptic ulcer Helicobacter related diseases, virally-induced angiogenic disorders, fractures, Crow-Fukase syndrome (POEMS), preeclampsia, menometrorrhagia, cat scratch fever, rubeosis, neovascular glaucoma and retinopathies such as those associated with diabetic retinopathy, retinopathy of prematurity, or age-related macular degeneration. In addition, some of these compounds can be used as active agents against solid tumors, malignant ascites, von Hippel Lindau disease, hematopoietic cancers and hyperproliferative disorders such as thyroid hyperplasia (especially Grave's disease), and cysts (such as hypervascularity of ovarian stroma characteristic of polycystic ovarian syndrome (Stein-Leventhal syndrome) and polycystic kidney disease since such diseases require a proliferation of blood vessel cells for growth and/or metastasis.

Further, some of these compounds can be used as active agents against burns, chronic lung disease, stroke, polyps, anaphylaxis, chronic and allergic inflammation, delayed-type hypersensitivity, ovarian hyperstimulation syndrome, brain tumor-associated cerebral edema, high-altitude, trauma or hypoxia induced cerebral or pulmonary edema, ocular and macular edema, ascites, glomerulonephritis and other diseases where vascular hyperpermeability, effusions, exudates, protein extravasation, or edema is a manifestation of the disease. The compounds will also be useful in treating disorders in which protein extravasation leads to the deposition of fibrin and extracellular matrix, promoting stromal proliferation (e.g. keloid, fibrosis, cirrhosis and carpal tunnel syndrome). Increased VEGF production potentiates inflammatory processes such as monocyte recruitment and activation. The compounds of this invention will also be useful in treating inflammatory disorders such as inflammatory bowel disease (IBD) and Crohn's disease.

It is additionally possible that inhibitors of certain kinases may have utility in the treatment of diseases when the kinase is not misregulated, but is nonetheless essential for maintenance of the disease state. In this case, inhibition of the kinase activity would act either as a cure or palliative for these diseases. For example, many viruses, such as human papilloma virus, disrupt the cell cycle and drive cells into the S-phase of the cell cycle (Vousden, FASEB Journal, 7:8720879 (1993)). Preventing cells from entering DNA synthesis after viral infection by inhibition of essential S-phase initiating activities such as CDK2, may disrupt the virus life cycle by preventing virus replication. This same principle may be used to protect normal cells of the body from toxicity of cycle-specific chemotherapeutic agents (Stone et al., Cancer Research, 56:3199-3202 (1996); Kohn et al., Journal of Cellular Biochemistry, 54:44-452 (1994)). Inhibition of CDKs 2 or 4 will prevent progression into the cycle in normal cells and limit the toxicity of cytotoxics which act in S-phase, G2 or mitosis. Furthermore, CDK2/cyclin E activity has also been shown to regulate NF-kB. Inhibition of CDK2 activity stimulates NF-kB-dependent gene expression, an event mediated through interactions with the p300 coactivator (Perkins et al., Science, 275:523-527 (1997)). NF-kB regulates genes involved in inflammatory responses (such as hematopoetic growth factors, chemokines and leukocyte adhesion molecules) (Baeuerle and Henkel, Annual Review of Immunology, 12:141-179 (1994)) and may be involved in the suppression of apoptotic signals within the cell (Beg and Baltimore, Science, 274:782-784 (1996); Wang et al., Science, 274:784-787 (1996); Van Antwerp et al., Science, 274:787-789 (1996)). Thus, inhibition of CDK2 may suppress apoptosis induced by cytotoxic drugs via a mechanism which involves NF-kB. This therefore suggests that inhibition of CDK2 activity may also have utility in other cases where regulation of NF-kB plays a role in etiology of disease. A further example may be take from fungal infections: Aspergillosis is a common infection in immune-compromised patients (Armstrong, Clinical Infectious Diseases, 16:1-7 (1993)). Inhibition of the Aspergillus kinases Cdc2/CDC28 or Nim A (Osmani et al., EMBO Journal, 10:2669-2679 (1991); Osmani et al., Cell, 67:283-291 (1991)) may cause arrest or death in the fungi, improving the therapeutic outcome for patients with these infections.

The compounds of the present invention may also be useful in the prophylaxis of the above diseases.

In another aspect, the present invention provides compounds of formula I as defined initially above for use as medicaments, particularly as inhibitors of protein kinase activity for example tyrosine kinase activity, serine kinase activity and threonine kinase activity. In yet another aspect the present invention provides the use of compounds of formula I as defined initially above in the manufacture of a medicament for use in the inhibition of protein kinase activity.

In this invention, the following definitions are applicable:

A “therapeutically effective amount” is an amount of a compound of Formula I or a combination of two or more such compounds, which inhibits, totally or partially, the progression of the condition or alleviates, at least partially, one or more symptoms of the condition. A therapeutically effective amount can also be an amount which is prophylactically effective. The amount which is therapeutically effective will depend upon the patient's size and gender, the condition to be treated, the severity of the condition and the result sought. For a given patient, a therapeutically effective amount can be determined by methods known to those of skill in the art.

“Physiologically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g. (+) or (−)-tartaric acid or mixtures thereof), amino acids (e.g. (+) or (−)-amino acids or mixtures thereof), and the like. These salts can be prepared by methods known to those skilled in the art.

Certain compounds of formula I which have acidic substituents may exist as salts with pharmaceutically acceptable bases. The present invention includes such salts. Example of such salts include sodium salts, potassium salts, lysine salts and arginine salts. These salts may be prepared by methods known to those skilled in the art.

Certain compounds of formula I and their salts may exist in more than one crystal form and the present invention includes each crystal form and mixtures thereof.

Certain compounds of formula I and their salts may also exist in the form of solvates, for example hydrates, and the present invention includes each solvate and mixtures thereof.

Certain compounds of formula I may contain one or more chiral centers, and exist in different optically active forms. When compounds of formula I contain one chiral center, the compounds exist in two enantiomeric forms and the present invention includes both enantiomers and mixtures of enantiomers, such as racemic mixtures. The enantiomers may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a compound of formula I contains more than one chiral center it may exist in diastereoisomeric forms. The diastereoisomeric pairs may be separated by methods known to those skilled in the art, for example chromatography or crystallization and the individual enantiomers within each pair may be separated as described above. The present invention includes each diastereoisomer of compounds of formula I and mixtures thereof.

Certain compounds of formula I may exist in different tautomeric forms or as different geometric isomers, and the present invention includes each tautomer and/or geometric isomer of compounds of formula I and mixtures thereof.

Certain compounds of formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of formula I and mixtures thereof.

Certain compounds of formula I may exist in zwitterionic form and the present invention includes each zwitterionic form of compounds of formula I and mixtures thereof.

As used herein the term “prodrug” refers to an agent which is converted into the parent drug in vivo by some physiological chemical process (e.g., a prodrug on being brought to the physiological pH is converted to the desired drug form). Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. An example, without limitation, of a prodrug would be a compound of the present invention wherein it is administered as an ester (the “prodrug”) to facilitate transmittal across a cell membrane where water solubility is not beneficial, but then it is metabolically hydrolyzed to the carboxylic acid once inside the cell where water solubility is beneficial

Prodrugs have many useful properties. For example, a prodrug may be more water soluble than the ultimate drug, thereby facilitating intravenous administration of the drug. A prodrug may also have a higher level of oral bioavailability than the ultimate drug. After administration, the prodrug is enzymatically or chemically cleaved to deliver the ultimate drug in the blood or tissue.

Exemplery prodrugs upon cleavage release the corresponding free acid, and such hydrolyzable ester-forming residues of the compounds of this invention include but are not limited to carboxylic acid substituents (e.g., R¹ is —(CH₂)_{q}C(O)X⁶ where X⁶ is hydrogen, or R² or A¹ contains carboxylic acid) wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary prodrugs release an alcohol of Formula I wherein the free hydrogen of the hydroxyl substituent (e.g., R¹ contains hydroxyl) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

The term “heterocyclic” or “heterocyclyl”, as used herein, include aromatic and non-aromatic, ring systems, including, but not limitied to, monocyclic, bicyclic and tricyclic rings, which can be completely saturated or which can contain one or more units of unsaturation and have 3 to 12 atoms including at least one heteroatom, such as nitrogen, oxygen, or sulfur. For purposes of exemplification, which should not be construed as limiting the scope of this invention: azaindole, azetidinyls, benzo(b)thienyl, benzimidazolyl, benzoxazolyl, benzothiazolyl, benzothiadiazolyl, benzoxadiazolyl, furans, imidazoles, imidazopyridine, indole, indazoles, isoxazoles, isothiazoles, oxadiazoles, oxazoles piperazines, piperidines, purine, pyrans, pyrazines, pyrazoles, pyridines, pyrimidines, pyrroles, pyrrolidines, pyrrolo[2,3-d]pyrimidine, pyrazolo[3,4-d]pyrimidine), quinolines, quinazolines, triazoles, thiazoles, tetrahydroindole, tetrazoles, thiadiazoles, thienyls, thiomorpholinos or triazles.

When the term “substituted heterocyclic” (or heterocyclyl) is used, what is meant is that the heterocyclic group is substituted with one or more substituents that can be made by one of ordinary skill in the art and results in a molecule that is a kinase inhibitor. For purposes of exemplification, which should not be construed as limiting the scope of this invention, preferred substituents for the heterocyclyls of this invention are each independently selected from the optionally substituted group consisting of alkenyl, alkoxy, alkoxyalkoxy, alkoxyalkyl, alkoxycarbonyl, alkoxycarbonylheterocycloalkoxy, alkyl, alkylcarbonyl, alkylester, alkyl-O—C(O)—, alkyl-heterocyclyl, alkyl-cycloalkyl, alkyl-nitrile, alkynyl, amido groups, amino, aminoalkyl, aminocarbonyl, carbonitrile, carbonylalkoxy, carboxamido, CF₃, CN, —C(O)OH, —C(O)H, —C(O)—)(CH₃)₃, —OH, —C(O)O-alkyl, —C(O)O-cycloalkyl, —C(O)O-heterocyclyl, —C(O)-alkyl, —C(O)-cycloalkyl, —C(O)-heterocyclyl, cycloalkyl, dialkylaminoalkoxy, dialkylaminocarbonylalkoxy, dialkylaminocarbonyl, halogen, heterocyclyl, a heterocycloalkyl group, heterocyclyloxy, hydroxy, hydroxyalkyl, nitro, NO₂, OCF₃, oxo, phenyl, —SO₂CH₃, —SO₂CR₃, tetrazolyl, thienylalkoxy, trifluoromethylcarbonylamino, trifluoromethylsulfonamido, heterocyclylalkoxy, heterocyclyl-S(O)ₚ, cycloalkyl-S(O)ₚ, alkyl-S-, heterocyclyl-S, heterocycloalkyl, cycloalkylalkyl, heterocycolthio, cycloalkylthio, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)—C(O)R, —N(R)—C(O) OR, OR—C(O)-heterocyclyl-OR, R_{c} and —CH₂OR_{c};
where R_{c} for each occurrence is independently hydrogen, optionally substituted alkyl, optionally substituted aryl, —(C₁-C₆)—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond, alkyl, alkenyl or alkynyl; and
Z²⁰⁰ for each occurrence is independently selected from an optionally substituted group selected from the group consisting of alkyl, alkenyl, alkynyl, phenyl, alkyl-phenyl, alkenyl-phenyl or alkynyl-phenyl.

An “heterocycloalkyl” group, as used herein, is a heterocyclic group that is linked to a compound by an aliphatic group having from one to about eight carbon atoms. For example, a preferred heterocycloalkyl group is an imidazolylethyl group.

As used herein, “aliphatic” or “an aliphatic group” or notations such as “(C₀-C₈)” include straight chained or branched hydrocarbons which are completely saturated or which contain one or more units of unsaturation, and, thus, includes alkyl, alkenyl, alkynyl and hydrocarbons comprising a mixture of single, double and triple bonds. When the group is a C₀ it means that the moiety is not present or in other words, it is a bond. As used herein, “alkyl” means C₁-C₈ and includes straight chained or branched hydrocarbons which are completely saturated. Preferred alkyls are methyl, ethyl, propyl, butyl, pentyl, hexyl and isomers thereof. As used herein, “alkenyl” and “alkynyl” means C₂-C₈ and includes straight chained or branched hydrocarbons which contain one or more units of unsaturation, one or more double bonds for alkenyl and one or more triple bonds for alkynyl.

As used herein, cycloalkyl means C₃-C₁₂ monocyclic or multicyclic (e.g., bicyclic, tricyclic, etc.) hydrocarbons which is completely saturated or has one or more unsaturated bonds but does not amount to an aromatic group. Preferred examples of a cycloalkyl group are cyclopropyl, cyclobutyl, cyclopentyl, cyclopentenyl, cyclohexyl and cyclohexenyl.

As used herein, amido group means —NHC(═O)—.

As used herein, acyloxy groups are —OC(O)R.

As used herein, many moieties or substituents are termed as being either “substituted” or “optionally substituted. When a moiety is modified by one of these terms, it denotes that any portion of the moiety that is known to one skilled in the art as being available for substitution can be substituted, which includes one or more substituents, where if more than one substituent then each substituent is independently selected. Such means for substitution are well-known in the art and/or taught by the instant disclosure. For purposes of exemplification, which should not be construed as limiting the scope of this invention, some examples of groups that are substituents are: alkenyl groups, alkoxy group (which itself can be substituted, such as —O—C₁-C₆-alkyl-OR, —O—C₁-C₆-alkyl-N(R)₂, and OCF₃), alkoxyalkoxy, alkoxycarbonyl, alkoxycarbonylpiperidinylalkoxy, alkyl groups (which itself can also be substituted, such as —C₁-C₆-alkyl-OR, —C₁-C₆-alkyl-N(R)₂, and —CF₃), alkylamino, alkylcarbonyl, alkylester, alkylnitrile, alkylsulfonyl, amino, aminoalkoxy, CF₃, COH, COOH, CN, cycloalkyl, dialkylamino, dialkylaminoalkoxy, dialkylaminocarbonyl, dialkylaminocarbonylalkoxy, dialkylaminosulfonyl, esters (—C(O)—OR, where R is groups such as alkyl, heterocycloalkyl (which can be substituted), heterocyclyl, etc., which can be substituted), halogen or halo group (F, Cl, Br, I), hydroxy, morpholinoalkoxy, morpholinoalkyl, nitro, oxo, OCF₃, optionally substituted phenyl, S(O)₂CH₃, S(O)₂CF₃, and sulfonyl, N-alkylamino or N,N-dialkylamino (in which the alkyl groups can also be substituted).

As used herein, toxic metal means a metal that is considered to be toxic to animals in trace amounts.

Phamaceutical Formulations

One or more compounds of this invention can be administered to a human patient by themselves or in pharmaceutical compositions where they are mixed with biologically suitable carriers or excipient(s) at doses to treat or ameliorate a disease or condition as described herein. Mixtures of these compounds can also be administered to the patient as a simple mixture or in suitable formulated pharmaceutical compositions. A therapeutically effective dose refers to that amount of the compound or compounds sufficient to result in the prevention or attenuation of a disease or condition as described herein. Techniques for formulation and administration of the compounds of the instant application may be found in references well known to one of ordinary skill in the art, such as “Remington's Pharmaceutical Sciences,” Mack Publishing Co., Easton, Pa., latest edition.

Routes of Administration

Suitable routes of administration may, for example, include oral, eyedrop, rectal, transmucosal, topical, or intestinal administration; parenteral delivery, including intramuscular, subcutaneous, intramedullary injections, as well as intrathecal, direct intraventricular, intravenous, intraperitoneal, intranasal, or intraocular injections.

Alternatively, one may administer the compound in a local rather than a systemic manner, for example, via injection of the compound directly into an edematous site, often in a depot or sustained release formulation.

Furthermore, one may administer the drug in a targeted drug delivery system, for example, in a liposome coated with endothelial cell-specific antibody.

Composition/Formulation

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation.

Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combining the active compound with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds can be formulated for parenteral administration by injection, e.g. bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly or by intramuscular injection). Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An example of a pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethysulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free base forms.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art.

For any compound used in a method of the present invention, the therapeutically effective dose can be estimated initially from cellular assays. For example, a dose can be formulated in cellular and animal models to achieve a circulating concentration range that includes the IC₅₀ as determined in cellular assays (i.e., the concentration of the test compound which achieves a half-maximal inhibition of a given protein kinase activity). In some cases it is appropriate to determine the IC₅₀ in the presence of 3 to 5% serum albumin since such a determination approximates the binding effects of plasma protein on the compound. Such information can be used to more accurately determine useful doses in humans. Further, the most preferred compounds for systemic administration effectively inhibit protein kinase signaling in intact cells at levels that are safely achievable in plasma.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the maximum tolerated dose (MID) and the ED₅₀ (effective dose for 50% maximal response). The dose ratio between toxic and therapeutic effects is the therapeutic index and it can be expressed as the ratio between MID and ED₅₀. Compounds which exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g. Fingl et al., 1975, in “The Pharmacological Basis of Therapeutics”, Ch. 1 p 1). In the treatment of crises, the administration of an acute bolus or an infusion approaching the MTD may be required to obtain a rapid response.

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g. the concentration necessary to achieve 50-90% inhibition of protein kinase using the assays described herein. Dosages necessary to achieve the MEC will depend on individual characteristics and route of administration. However, HPLC assays or bioassays can be used to determine plasma concentrations.

Dosage intervals can also be determined using the MEC value. Compounds should be administered using a regimen which maintains plasma levels above the MEC for 10-90% of the time, preferably between 30-90% and most preferably between 50-90% until the desired amelioration of symptoms is achieved. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

In some formulations it may be beneficial to use the compounds of the present invention in the form of particles of very small size, for example as obtained by fluid energy milling.

The use of compounds of the present invention in the manufacture of pharmaceutical compositions is illustrated by the following description. In this description the term “active compound” denotes any compound of the invention but particularly any compound which is the final product of one of the preceding Examples.

a) Capsules

In the preparation of capsules, 10 parts by weight of active compound and 240 parts by weight of lactose can be de-aggregated and blended. The mixture can be filled into hard gelatin capsules, each capsule containing a unit dose or part of a unit dose of active compound.

b) Tablets

Tablets can be prepared, for example, from the following ingredients.

Parts by weight

| | |
|---|---|
| Active compound | 10 |
| Lactose | 190 |
| Maize starch | 22 |
| Polyvinylpyrrolidone | 10 |
| Magnesium stearate | 3 |

The active compound, the lactose and some of the starch can be de-aggregated, blended and the resulting mixture can be granulated with a solution of the polyvinyl-pyrrolidone in ethanol. The dry granulate can be blended with the magnesium stearate and the rest of the starch. The mixture is then compressed in a tabletting machine to give tablets each containing a unit dose or a part of a unit dose of active compound.

c) Enteric Coated Tablets

Tablets can be prepared by the method described in (b) above. The tablets can be enteric coated in a conventional manner using a solution of 20% cellulose acetate phthalate and 3% diethyl phthalate in ethanol:dichloromethane (1:1).

d) Suppositories

In the preparation of suppositories, for example, 100 parts by weight of active compound can be incorporated in 1300 parts by weight of triglyceride suppository base and the mixture formed into suppositories each containing a therapeutically effective amount of active ingredient.

In the compositions of the present invention the active compound may, if desired, be associated with other compatible pharmacologically active ingredients. For example, the compounds of this invention can be administered in combination with another therapeutic agent that is known to treat a disease or condition described herein. For example, with one or more additional pharmaceutical agents that inhibit or prevent the production of VEGF or angiopoietins, attenuate intracellular responses to VEGF or angiopoietins, block intracellular signal transduction, inhibit vascular hyperpermeability, reduce inflammation, or inhibit or prevent the formation of edema or neovascularization. The compounds of the invention can be administered prior to, subsequent to or simultaneously with the additional pharmaceutical agent, whichever course of administration is appropriate. The additional pharmaceutical agents include, but are not limited to, anti-edemic steroids, NSAIDS, ras inhibitors, anti-TNF agents, anti-IL1 agents, antihistamines, PAF-antagonists, COX-1 inhibitors, COX-2 inhibitors, NO synthase inhibitors, Akt/PTB inhibitors, IGF-1R inhibitors, PKC inhibitors, PI3 kinase inhibitors, calcineurin inhibitors and immunosuppressants. The compounds of the invention and the additional pharmaceutical agents act either additively or synergistically. Thus, the administration of such a combination of substances that inhibit angiogenesis, vascular hyperpermeability and/or inhibit the formation of edema can provide greater relief from the deletrious effects of a hyperproliferative disorder, angiogenesis, vascular hyperpermeability or edema than the administration of either substance alone. In the treatment of malignant disorders combinations with antiproliferative or cytotoxic chemotherapies or radiation are included in the scope fo the present invention.

The present invention also comprises the use of a compound of formula I as a medicament.

A further aspect of the present invention provides the use of a compound of formula I or a salt thereof in the manufacture of a medicament for treating vascular hyperpermeability, angiogenesis-dependent disorders, proliferative diseases and/or disorders of the immune system in mammals, particularly human beings.

The present invention also provides a method of treating vascular hyperpermeability, inappropriate neovascularization, proliferative diseases and/or disorders of the immune system which comprises the administration of a therapeutically effective amount of a compound of formula I to a mammal, particularly a human being, in need thereof.

Biological Assays

The in vitro potency of compounds in inhibiting one or more of the protein kinases discussed herein or described in the art may be determined by the procedures detailed below.

The potency of compounds can be determined by the amount of inhibition of the phosphorylation of an exogenous substrate (e.g., synthetic peptide (Z. Songyang et al., Nature. 373:536-539) by a test compound relative to control.

KDR Tyrosine Kinase Production Using Baculovirus System:

The coding sequence for the human KDR intra-cellular domain (aa789-1354) was generated through PCR using cDNAs isolated from HUVEC cells. A poly-His6 sequence was introduced at the N-terminus of this protein as well. This fragment was cloned into transfection vector pVL1393 at the Xba 1 and Not 1 site. Recombinant baculovirus (BV) was generated through co-transfection using the BaculoGold Transfection reagent (PharMingen). Recombinant BV was plaque purified and verified through Western analysis. For protein production, SF-9 cells were grown in SF-900-II medium at 2×106/ml, and were infected at 0.5 plaque forming units per cell (MOI). Cells were harvested at 48 hours post infection.

Purification of KDR

SF-9 cells expressing (His)₆KDR(aa789-1354) were lysed by adding 50 ml of Triton X-100 lysis buffer (20 mM Tris, pH 8.0, 137 mM NaCl, 10% glycerol, 1% Triton X-100, 1 mM PMSF, 10 μg/ml aprotinin, 1 μg/ml leupeptin) to the cell pellet from 1 L of cell culture. The lysate was centrifuged at 19,000 rpm in a Sorval SS-34 rotor for 30 min at 4° C. The cell lysate was applied to a 5 ml NiCl₂ chelating sepharose column, equilibrated with 50 mM HEPES, pH 7.5, 0.3 M NaCl. KDR was eluted using the same buffer containing 0.25 M imidazole. Column fractions were analyzed using SDS-PAGE and an ELISA assay (below) which measures kinase activity. The purified KDR was exchanged into 25 mM HEPES, pH 7.5, 25 mM NaCl, 5 mM DTT buffer and stored at −80° C.

Human Tie-2 Kinase Production and Purification

The coding sequence for the human Tie-2 intra-cellular domain (aa775-1124) was generated through PCR using cDNAs isolated from human placenta as a template. A poly-His₆ sequence was introduced at the N-terminus and this construct was cloned into transfection vector pVL 1939 at the Xba 1 and Not 1 site. Recombinant BV was generated through co-transfection using the BaculoGold Transfection reagent (PharMingen). Recombinant BV was plaque purified and verified through Western analysis. For protein production, SF-9 insect cells were grown in SF-900-II medium at 2×106/ml, and were infected at MOI of 0.5. Purification of the His-tagged kinase used in screening was analogous to that described for KDR.

Human Flt-1 Tyrosine Kinase Production and Purification

The baculoviral expression vector pVL1393 (Phar Mingen, Los Angeles, Calif.) was used. A nucleotide sequence encoding poly-His6 was placed 5′ to the nucleotide region encoding the entire intracellular kinase domain of human Flt-1 (amino acids 786-1338). The nucleotide sequence encoding the kinase domain was generated through PCR using cDNA libraries isolated from HUVEC cells. The histidine residues enabled affinity purification of the protein as a manner analogous to that for KDR and ZAP70. SF-9 insect cells were infected at a 0.5 multiplicity and harvested 48 hours post infection.

EGFR Tyrosine Kinase Source

EGFR was purchased from Sigma (Cat #E-3641; 500 units/50 μl) and the EGF ligand was acquired from Oncogene Research Products/Calbiochem (Cat # PF011-100).

Expression of ZAP70

The baculoviral expression vector used was pVL1393. (Pharmingen, Los Angeles, Calif.) The nucleotide sequence encoding amino acids M(H)6 LVPR₉S was placed 5′ to the region encoding the entirety of ZAP70 (amino acids 1-619). The nucleotide sequence encoding the ZAP70 coding region was generated through PCR using cDNA libraries isolated from Jurkat immortalized T-cells. The histidine residues enabled affinity purification of the protein (vide infra). The LVPR₉S bridge constitutes a recognition sequence for proteolytic cleavage by thrombin, enabling removal of the affinity tag from the enzyme. SF-9 insect cells were infected at a multiplicity of infection of 0.5 and harvested 48 hours post infection.

Extraction and Purification of ZAP70

SF-9 cells were lysed in a buffer consisting of 20 mM Tris, pH 8.0, 137 mM NaCl, 10% glycerol, 1% Triton X-100, 1 mM PMSF, 1 μg/ml leupeptin, 10 μg/ml aprotinin and 1 mM sodium orthovanadate. The soluble lysate was applied to a chelating sepharose HiTrap column (Pharmacia) equilibrated in 50 mM HEPES, pH 7.5, 0.3 M NaCl. Fusion protein was eluted with 250 mM imidazole. The enzyme was stored in buffer containing 50 mM HEPES, pH 7.5, 50 mM NaCl and 5 mM DTT.

Protein Kinase Source

Lck, Fyn, Src, Blk, Csk, and Lyn, and truncated forms thereof may be commercially obtained (e.g. from Upstate Biotechnology Inc. (Saranac Lake, N.Y.) and Santa Cruz Biotechnology Inc. (Santa Cruz, Calif.)) or purified from known natural or recombinant sources using conventional methods.

Enzyme Linked Immunosorbent Assay (ELISA) for PTKs

Enzyme linked immunosorbent assays (ELISA) were used to detect and measure the presence of tyrosine kinase activity. The ELISA were conducted according to known protocols which are described in, for example, Voller, et al., 1980, “Enzyme-Linked Immunosorbent Assay,” In: Manual of Clinical Immunology, 2d ed., edited by Rose and Friedman, pp 359-371 Am. Soc. of Microbiology, Washington, D.C.

The disclosed protocol was adapted for determining activity with respect to a specific PTK. For example, preferred protocols for conducting the ELISA experiments is provided below. Adaptation of these protocols for determining a compound's activity for other members of the receptor PTK family, as well as non-receptor tyrosine kinases, are well within the abilities of those in the art. For purposes of determining inhibitor selectivity, a universal PTK substrate (e.g., random copolymer of poly(Glu₄ Tyr), 20,000-50,000 MW) was employed together with ATP (typically 5 μM) at concentrations approximately twice the apparent Km in the assay.

The following procedure was used to assay the inhibitory effect of compounds of this invention on KDR, Flt-1, Flt-4/VEGFR-3, Tie-1, Tie-2, EGFR, FGFR, PDGFR, IGF-1-R, c-Met, Lck, Blk, Csk, Src, Lyn, Fyn and ZAP70 tyrosine kinase activity:
Buffers and Solutions:
PGTPoly (Glu,Tyr) 4:1
Store powder at −20° C. Dissolve powder in phosphate buffered saline (PBS) for 50 mg/ml solution. Store 1 ml aliquots at −20° C. When making plates dilute to 250 μg/ml in Gibco PBS.
Reaction Buffer: 100 mM Hepes, 20 mM MgCl₂, 4 mM MnCl₂, 5 mM DTT, 0.02% BSA, 200 μM NaVO₄, pH 7.10
ATP: Store aliquots of 100 mM at −20° C. Dilute to 20 μM in water
Washing Buffer: PBS with 0.1% Tween 20
Antibody Diluting Buffer: 0.1% bovine serum albumin (BSA) in PBS
TMB Substrate: mix TMB substrate and Peroxide solutions 9:1 just before use or use K-Blue Substrate from Neogen
Stop Solution: 1M Phosphoric Acid
   Procedure

1. Plate Preparation:
Dilute PGT stock (50 mg/ml, frozen) in PBS to a 250 μg/ml. Add 125 μl per well of Corning modified flat bottom high affinity ELISA plates (Coming #25805-96). Add 125 μl PBS to blank wells. Cover with sealing tape and incubate overnight 37° C.
Wash 1× with 250 μl washing buffer and dry for about 2 hrs in 37° C. dry incubator.
Store coated plates in sealed bag at 4° C. until used.

2. Tyrosine Kinase Reaction:
Prepare inhibitor solutions at a 4× concentration in 20% DMSO in water.
Prepare reaction buffer
Prepare enzyme solution so that desired units are in 50 μl, e.g. for KDR make to 1 ng/μl for a total of 50 ng per well in the reactions. Store on ice.
Make 4×ATP solution to 20 μM from 100 mM stock in water. Store on ice.
Add 50 μl of the enzyme solution per well (typically 5-50 ng enzyme/well depending on the specific activity of the kinase)
Add 25 μl 4× inhibitor
Add 25 μl 4× ATP for inhibitor assay
Incubate for 10 minutes at room temperature
Stop reaction by adding 50 μl 0.05N HCl per well
Wash plate
   **Final Concentrations for Reaction: 5 μM ATP, 5% DMSO

3. Antibody Binding
Dilute 1 mg/ml aliquot of PY20-HRP (Pierce) antibody (a phosphotyrosine antibody) to 50 ng/ml in 0.1% BSA in PBS by a 2 step dilution (100×, then 200×)
Add 100 μl Ab per well. Incubate 1 hr at room temp. Incubate 1 hr at 4° C.
Wash 4× plate

4. Color reaction
Prepare TMB substrate and add 100 μl per well
Monitor OD at 650 nm until 0.6 is reached
Stop with 1M Phosphoric acid. Shake on plate reader.
Read OD immediately at 450 nm

Optimal incubation times and enzyme reaction conditions vary slightly with enzyme preparations and are determined empirically for each lot.

For Lck, the Reaction Buffer utilized was 100 mM MOPSO, pH 6.5, 4 mM MnCl₂, 20 mM MgCl₂, 5 mM DTT, 0.2% BSA, 200 mM NaVO₄ under the analogous assay conditions.

Compounds of formula I may have therapeutic utility in the treatment of diseases involving both identified, including those not mentioned herein, and as yet unidentified protein tyrosine kinases which are inhibited by compounds of formula I. All compounds exemplified herein significantly inhibit either FGFR, PDGFR, KDR, Tie-2, Lck, Fyn, Blk, Lyn or Src at concentrations of 50 micromolar or below. Some compounds of this invention also significantly inhibit other tyrosine or serine/threonine kinases such as cdc2 (cdk1) at concentrations of 50 micromolar or below.

Cdc2 Source

The human recombinant enzyme and assay buffer may be obtained commercially (New England Biolabs, Beverly, Mass. USA) or purified from known natural or recombinant sources using conventional methods.

Cdc2 Assay

The protocol used was that provided with the purchased reagents with minor modifications. In brief, the reaction was carried out in a buffer consisting of 50 mM Tris pH 7.5, 100 mM NaCl, 1 mM EGTA, 2 mM DTT, 0.01% Brij, 5% DMSO and 10 mM MgCl₂ (commercial buffer) supplemented with fresh 300 μM ATP (31 μCi/ml) and 30 μg/ml histone type IIIss final concentrations. A reaction volume of 80 μL, containing units of enzyme, was run for 20 minutes at 25° C. in the presence or absence of inhibitor. The reaction was terminated by the addition of 120 μL of 10% acetic acid. The substrate was separated from unincorporated label by spotting the mixture on phosphocellulose paper, followed by 3 washes of 5 minutes each with 75 mM phosphoric acid. Counts were measured by a betacounter in the presence of liquid scintillant.

Certain compounds of this invention significantly inhibit cdc2 at concentrations below 50 uM.

PKC Kinase Source

The catalytic subunit of PKC may be obtained commercially (Calbiochem). PKC kinase assay

A radioactive kinase assay was employed following a published procedure (Yasuda, I., Kirshimoto, A., Tanaka, S., Tominaga, M., Sakurai, A., Nishizuka, Y. Biochemical and Biophysical Research Communication 3:166, 1220-1227 (1990)). Briefly, all reactions were performed in a kinase buffer consisting of 50 mM Tris-HCl pH 7.5, 10 mM MgCl₂, 2 mM DTT, 1 mM EGTA, 100 μM ATP, 8 μM peptide, 5% DMSO and ³³P ATP (8 Ci/mM). Compound and enzyme were mixed in the reaction vessel and the reaction initiated by addition of the ATP and substrate mixture. Following termination of the reaction by the addition of 10 μL stop buffer (5 mM ATP in 75 mM phosphoric acid), a portion of the mixture was spotted on phosphocellulose filters. The spotted samples were washed 3 times in 75 mM phosphoric acid at room temperature for 5 to 15 minutes. Incorporation of radiolabel was quantified by liquid scintillation counting.

Erk2 Enzyme Source

The recombinant murine enzyme and assay buffer may be obtained commercially (New England Biolabs, Beverly Mass. USA) or purified from known natural or recombinant sources using conventional methods.

Erk2 Enzyme Assay

In brief, the reaction was carried out in a buffer consisting of 50 mM Tris pH 7.5, 1 mM EGTA, 2 mM DTT, 0.01% Brij, 5% DMSO and 10 mM MgCl₂ (commercial buffer) supplemented with fresh 100 μM ATP (31 μCi/ml) and 30 μM myelin basic protein under conditions recommended by the supplier. Reaction volumes and method of assaying incorporated radioactivity were as described for the PKC assay (vide supra).

In Vitro Models for T-Cell Activation

Upon activation by mitogen or antigen, T-cells are induced to secrete IL-2, a growth factor that supports their subsequent proliferative phase. Therefore, one may measure either production of IL-2 from or cell proliferation of, primary T-cells or appropriate T-cell lines as a surrogate for T-cell activation. Both of these assays are well described in the literature and their parameters well documented (in Current Protocols in Immunology, Vol 2, 7.10.1-7.11.2).

In brief, T-cells may be activated by co-culture with allogenic stimulator cells, a process termed the one-way mixed lymphophocyte reaction. Responder and stimulator peripheral blood mononuclear cells are purified by Ficoll-Hypaque gradient (Pharmacia) per directions of the manufacturer. Stimulator cells are mitotically inactivated by treatment with mitomycin C (Sigma) or gamma irradiation. Responder and stimulator cells are co-cultured at a ratio of two to one in the presence or absence of the test compound. Typically 10⁵ responders are mixed with 5×10⁴ stimulators and plated (200 μl volume) in a U bottom microtiter plate (Costar Scientific). The cells are cultured in RPMI 1640 supplemented with either heat inactivated fetal bovine serum (Hyclone Laboratories) or pooled human AB serum from male donors, 5×10⁻⁵ M 2mercaptoethanol and 0.5% DMSO. The cultures are pulsed with 0.5 μCi of ³H thymidine (Amersham) one day prior to harvest (typically day three). The cultures are harvested (Betaplate harvester, Wallac) and isotope uptake assessed by liquid scintillation (Betaplate, Wallac).

The same culture system may be used for assessing T-cell activation by measurement of IL-2 production. Eighteen to twenty-four hours after culture initiation, the supernatants are removed and the IL-2 concentration is measured by ELISA (R and D Systems) following the directions of the manufacturer.

HUVEC KDR Autophosphorylation Assay Protocol

Culturing of HUVEC Cells from Frozen Stock:

1. Thaw one vial of HUVEC cells (Clonetics, cc-2519) into one 100 mm plate (Falcon for tissue culture) containing 10 ml of complete (all supplements added) EBM media (Clonetics, cc-4143). This is passage one (P1).

2. Next day, change media.

3. Two-three days later, plate should be 90-100% confluent. Split into 3-4 100 mm plates (P2). To split either use the Clonetics trypsin or dilute our trypsin/EDTA (Gibco, 25500-056) 1:5 in PBS1X, add 2 ml per plate and watch cells under microscope. Most will lift off in 1 min.

4. 3-4 days later, split the 3 or 4 plates into 12-14 100 mm plates (P3).

5. Continue to passage cells in this manner through P8.

HUVEC VEGF-Induced Autophosphorylation:

1. 3-4 days after plating P3, plates should be 90-100% confluent.

2. Serum starve overnight, all but 2 or 3 plates for use in assay (non-starved plates are used for the next passage).
To serum starve plates: Aspirate off media; rinse in 5-10 ml of PBS; add 10 ml of EBM base media (no supplements added).

3. DAY OF ASSAY: make up all drug dilutions and RIPA buffer just prior to use. Treatment Conditions;
(a) 0=untreated, serum starved
(b) 10′ VEGF (1×)—2 ml at 50 ng/ml*
   *VEGF is kept in the −80° C. freezer in 100 ul aliquots at 10 ug/ml PBS/1% BSA for 1× VEGF—add 100 ul aliquot to 20 ml of EBM base media for 2× VEGF—add 100 μl aliquot to 10 ml of EBM base media
(c) Inhibitors—(1) add 2 ml of either 25 μM or 5 μM of drug dilution for 1 hour at 37° C.
   (2) after 1 hour, add 2 ml of 2× VEGF for 10 min.
(d) Aspirate off, rinse in 5-10 ml of PBS+1 mM Na orthoVanadate
(e) Lyse plate in 500 μl of cRIPA** and scrape, put on ice for 15 min.
   ** Modified RIPA BUFFER
(f) after 15 min, put lysate in a labeled eppendorf tube and continue to lyse on ice for 2-4 hours.
(g) Spin at 14,000 rpm for 30 min, to pellet nuclei
(h) Pour off lysate into fresh tube
(i) Take 10 ul for a BCA Protein Determination (kit by Pierce). Can freeze at this point until ready to (immuno)precipitate or Western blot.
(j) Protein Precipitation is done by adding 3 times the volume (of lysate) of ice cold Ethanol. Place samples in the freezer overnight. Samples can be stored this way until ready for use.

4. Day to run gels:
(a) Spin samples at 14,000 rpms for 30 min. @ 4° C.
(b) Pour off supernate. Air Dry Pellet of 1-2 hrs.
(c) Add 2× Sample Buffer +2 Me (Sigma M7154).
(d) Boil Samples 5 min.
(e) Run gels.
FINAL CONCENTRATION
50 mM Tris-HCl pH 7.5
1% NP-40
0.25% NaDOC (deoxycholic acid) (Sigma D4297)
1 mM EDTA(Sigma E1644)

QS TO 1000 ml

| | | | Final | |
|---|---|---|---|---|
| Add just prior to use | for 10 ml | | Conc. | |
| 100 mM PMSF(diluted in EtOH) | 100 | ul | 1 | mM |
| (Sigma P7626) | | | | |
| 1 mg/ml Aprotinin (Sigma A3428) | 10 | μl | 1 | μg/ml |
| 1 mg/ml Pepstatin (Sigma P5318) | 10 | μl | 1 | μg/ml |
| 1 mg/ml Leupeptin (Sigma L9783) | 10 | μl | 1 | μg/ml |
| 100 mM Na orthoVanadate (Sigma S9265) | 100 | μl | 1 | mM |
| 100 mM Na Fluoride (Sigma S7920) | 100 | μl | 1 | mM |
| 10 mg/ml DNAase (Sigma D4527) | 10 | μl | 1 | μg/ml |
| VEGF Induced Intracellular Calcium Flux Assay | | | | |
| Protocol (& Counterscreen stimuli) | | | | |

Buffers:

Buffer A Hanks Balanced Salt Solution (Gibco/BRL#14175-095 without phenol red)+1% Hepes 1M (10 mM final concentration) (Gibco/BRL #15630-080)

Buffer B Buffer A+5% BSA (Sigma #A-7030)

Buffer C Buffer B+10 ug/ml DNase (Sigma #D-4527).

FACS Buffer 0.1% BSA in HBSS (+0.01% Soduim Azide (Sigma S2002))

Versene Gibco/BRL #15040-066

Fluo4/AM 5 uM(Molecular Probes #F-14201) in Buffer A+0.025% P127 (#P-3000 Molecular Probes)

HUVEC Cells Pooled donors (Clonetics # cc-2519)

EC media EBM media (Clonetics # cc-3121)+supplements (Clonetics # cc-4133)

VEGF R&D Systems (#293-VE050) 10 μg/ml Stock in PBS

Ionomycin Sigma (#I-0634) 10 mM DMSO Stock

Histamine Sigma (#H-7125) 10 mM DMSO Stock

Thrombin Sigma (#T-6884) 1000 units/ml PBS Stock

Bovine Insulin Gibco/BRL (#13007-018) stock in dH₂O

HUVEC Cell Culture:

1. Thaw one vial of HUVEC cells into 10 ml of complete EC media in a 100 mm Tissue Culture plate (Falcon #35-3003).

2. Next day, aspirate media, re-feed.

3. 3-4 days later, once plate is 80-90% confluent, split cells.

4. Dilute Trypsin/EDTA (Gibco/BRL #252000-056) 1:5 in PBS IX without calcium and magnesium (Gibco/BRL #14190-144).

5. Leave on cells that have been rinsed once in PBS, for 1-2 minutes. Tap plate against edge of hood to release cells.

6. Split into 4 100 mm TC plates.

7. 3-4 days later, expand into 4-6 T150 cm² flasks (Corning #430825).

8. Split every 3-4 days, one T150 into 5-6 T150 cm².

9. Use only through passage 10 from the thaw.

Fluo4/AM Labeling of Cells:

1. Rinse cells with PBS 1×.

2. Add 5 ml of Versene per flask.

3. Let cells lift off at room temp., 3-5 min.

4. Add Buffer A to cells to double the cell volume.

5. Count cells using the Trypan Blue Exculsion method, want 1×10⁶ cells/ml.

6. Calculate number of mls needed of dye and spin down cells. Resuspend to 1×10⁶ cells/ml in 5 uM FLuo4/AM+0.025% P127.

7. Leave at RT 20 min

8. Add equal volume of Buffer B, incubate at RT for 10 min.

9. Spin down cells at 1000 rpms for 5-10 min.

10. Aspirate.

11. Cells can be washed 1× in Buffer C prior to the addition of the FACS Buffer. HUVECS do not tolerate this additional step and it can be left out, without causing increased background problems.

12. Resuspend 1×10⁶ cells/ml in FACS Buffer. Put 1 ml in Falcon #35-2025 5 ml polystryrene, round bottom tubes to read on FACS machine.

13. Cells are “live” and will only last for about one hour, therfore have everything organized and the FACSscan machine warmed up by the time cells are labeled.

14. Read using the Becton Dickinson FACSscan machine with Cellquest software using a density plot vs time.

Intracellular Calcium Flux Assay (by FACS):

1. FACScan machine (Beckton Dickenson) should be turned on to warm up 10-20 minutes prior to use.

2. Check buffer level before beginning, a full buffer reservoir is recomended and an empty waste container.

3. Fluo4 has a similar emission to FITC, therefore read on FL1 at ˜350 nm.

4. Once FACS machine has been set up of a density plot (flouresence vs time), begin by reading the controls.

| | |
|---|---|
| Specific for KDR | VEGF 50 ng/ml |
| Nonspecific | Ionomycin 10 μM |
| | Histamine 10 μM |
| | Thrombin 1unit |
| | Bovine Insulin 500 μg/ml |

5. To test compounds, make dilutions of the 10 mM DMSO stock into Buffer A, just prior to the experiment. Add the compound to the tube for a 5 min pre-incubation before taking a 10-15 second background reading. Add VEGF and read for 3 minutes. If the compound inhibits, will not see a shift as with VEGF alone. Do a dose titration until no inhibition is seen.

6. Specificity testing is done by adding the compound, at a concentration that gives complete inhibition, simultaneously with each nonspecific stimulant. Next test by adding VEGF read for 2.5-3 min, until the peak flux is seen; add the compound, at a concentration that gives complete inhibition; read for 2-3 minutes, then add Ionomycin to see if the cells can still flux calcium.

PDGF-β Cellular Assay Protocol

Media: cDMEM=DMEM+10% HI-FBS+1% Hepes+1% L-glutarnine+1% non-essential amino acids+1% Sodium pyruvate
Plate NIH/3T3 cells @ 3×10⁵ cells/well in a 12 well plate (cQstar #3513) and incubate overnight @ 37° C./5% CO₂.
The next day, serum starve the cells by aspirating the media from plates and replacing it with pre-warmed (37° C.) cDMEM without FBS and incubate for one hour at 37° C./5% CO₂.
Make up desired drug dilutions in DMEM+1% DMSO making sure you have 1 ml of each dilution for cell coverage.

After serum starvation, add 1 ml of serial dilutions to each well and incubate cells with drug for 30 mins @ 37° C./5% CO₂.

| | |
|---|---|
| During incubation prepare | 50 mL RIPA base (keep on ice) |
| lysis buffer: | 500 μL 100 mM vanadate |
| | 500 μL 100 mM NaF |
| | 50 μL 1 mg/ml Leupeptin |
| | 50 μL 1 mg/ml A-protinin |
| | 50 μL 1 mg/ml Pepstatin A |
| | 500 μL PMSF (add just before lysis) |

Thaw PDGF-BB (Peprotech #100-14B 2 μg) (This is made to a 10 μg/mL in 200 μL 10 mM acetic acid). For each well you need 100 ng/mL (5 μL of above solution)
After drug incubation, add 5 μL/well PDGF-BB and incubate @ 37° C./5% CO₂ for 10 mins.
Carefully remove media from wells and add 500 μL lysis buffer. Mix on ice for 30 mins.
Place lysates in Eppendorf tubes and spin @ 14,000 rpm for 20 mins.
Conduct a BCA assay to determine protein concentration
Place 150 μg of protein in new Eppendorf tubes and bring up total volume to 500 μL (can store @−20° C. and continue later)
Pre-clear: To each tube add 30 μL protein G/agarose and mix @ 4° C. for 30 mins.
Spin at 14,000 rpm for 2 min @ 4° C. Place supernatant in new Eppendorfs and add 10 μL of IP antibody. (Santa Cruz SC-432 PDGFR-β rabbit polyclonal) and mix @ 4° C. for 2 hrs (or overnight)
Add 50 μL protein G/agarose to each tube and mix for 2 hrs (or overnight)
Wash beads ×3 @ 14,00 rpm, 2 mins, 4° C. with 800 μL PBS+1 mM vanadate+Sigma PI cocktail (in 50ml of PBS add 500 μL 100 mM vanadate and 500 μL Sigma PI cocktail)
After last wash add 50 μL of 5× sample buffer and heat samples @ 95° C. for 5 mins.
Spin @ 14,000 rpm, 2 mins, 4° C. and transfer supurnatent to new Eppendorfs (this can be stored in −20° C. until ready to run gels).
Run 10 μL of each sample on 8-16% Tris-glycine gels (Novex 1.5 mm, 15 well), run duplicate gels to check for equal loading. Run @ 40 mA per gel for ˜1.5 hrs using Novex 1× running buffer.
Equilibrate gels into transfer buffer: 1600 mL MeOH
   800 mL 10× tris-glycine
   5600 mL H₂O
Transfer onto ECL hybond nitrocellulose membrane for 1 hr @ 100 volts/4° C.
Block overnight @ 4° C. PDGF-β blots block in 5% milk/PBST
   p-Tyr blots block in 3% BSA/PBST
Rinse blots 2× quick and 5× over an hour in PBS/0.1% tween-20
Primary antibody: p-Tyr blots-4g10-HRP (amount determined by lot number in 10 mL PBST (Upstate Biotech #16-105)
   PDGF-B blots-use PDGFR-P from Santa Cruz (SC432)—use 10 μl in 10 mL PBST per blot.
Incubate in primary antibody for one hour at room temp. rocking gently
Wash 5× over one hour in PBST
Secondary Antibody: PDGF-β blots—Use Amersham's anti-rabbit IgG HRP (NA#934) 1 uL in 10 ml PBST per blot
   p-Tyr—no secondary needed—keep rocking in PBST
Incubate in secondary antibody for one hour at room temp. rocking gently.
Wash 5× over one hour in PBST
Develop using ECL detection Kit
CSF1-R Cellular ELISA Protocol
   Day #1

Cell Plating: plate 25,000 Clone 5.5 cells (see Nature (1986) 320, 277-80) per well in Costar #3799 96 well round bottom plates, in 150 ul/well of growth media. Need 2 cell plates per set of compounds to be tested. Media is DMEM+10% FBS+1% L-glutamine+1% HEPES+500 ug/ml G418.

Antibody plating: Plate 1 ug/well of Oncogene GR12L (anti-cfms/CSF1R rat monoclonal antibody) in 150 ul of Pierce (#28382) Na carbonate/bicarbonate buffer pH 9.0.

Can coat overnight at 4° C. in refrig or 1 hr at 37° C. in the incubator.

Day #2

Antibody plate: wash using the TECAN plate washer (in 2047) in PBST(PBS+Tween 20 from in-house media kitchen).

Add 200 ul of 5% NFDM(nonfat dry milk, Carnation) in PBS to block plate, incubating at RT until ready to add lysate.

Can incubate overnight at 4° C. in refrig.

2× Drug plate: prepare one drug plate for every 2 cell plates.

Dilute compounds in DMEM+1% DMSO (aka media).

Working stock(WS) is 200 μM which is a 1:5 dilution of 10 mM DMSO stock.
Serial Dilution Scheme:
20 μl WS+180 μl media=20 μM
20 μl 20 μM+180 μl media=2 μM
20 μl 2 μM+180 μl media=0.2 μM
20 μl 0.2 μM+180 μl media=0.02 μM
20 μl 0.02 μM+180 μl media=0.002 μM

2× MCSF (R&D Systems 216-MC): 200 ng/ml is the concentration needed.
200 wells×25 ul/well=5 ml
5 ml×200 ng/ml=1000 ng=1 μg

Therefore 1 μg MCSF in 5 ml media add 25 ul/well to both cell plates.

Assay Protocol:

Remove media from plates, by aspirating with plate washer or flicking the media into the sink.

Add 25 μl/well from 2× Drug Plate. Incubate 20 min. 37° C. in incubator.

Add 25 μl/well 2× MCSF, incubate 10 min 37° C.

Add 50 μl/well Lysis buffer (see below).

Incubate 10 min. RT.

Wash antibody plate ×2 with plate washer in PBST.

Add 170 μl of combined cell lysate (combine the 100 ul from each plate into one of the cell plates) to washed antibody plate.

Incubate 2 hrs RT.

Wash plate 5× in PBST.

To Detect p-Tyr:

Add 150 μl/well of 4G10-Biotin antibody (Upstate #16-103) diluted 1:2000 in PBS.

Incubate 1.5 hrs RT.

Wash ×5 in PBST.

Add 150 μl/well of Streptavidin-HRP (Upstate #18-152) diluted in PBS.

Incubate 1 hr, RT.

Add 100 μl/well Enhanced K-Blue Substrate (Neogen #308177).

Color reaction is blue.

Read plate at 650 nm until (+) control wells read 0.6 OD.

Add 100 μl 1M Phosphoric Acid (Sigma #P6560).

Read plate at 450 nm.

Lysis Buffer

Pierce Lysis buffer (M-PER Mammalian Protein Extraction Reagent #78501)

| Add just prior to use: | for 10 ml | | Final Conc. | |
|---|---|---|---|---|
| 100 mM PMSF(diluted in EtOH) | 100 | μl | 1 | mM |
| (Sigma P7626) | | | | |
| 1 mg/ml Aprotinin (Sigma A3428) | 10 | μl | 1 | μg/ml |
| 1 mg/ml Pepstatin (Sigma P5318) | 10 | μl | 1 | μg/ml |
| 1 mg/ml Leupeptin (Sigma L9783) | 10 | μl | 1 | μg/ml |
| 100 mM Na orthoVanadate (Sigma S9265) | 100 | μl | 1 | mM |
| 100 mM Na Fluoride (Sigma S7920) | 100 | μl | 1 | mM |
| 10 mg/ml DNAase (Sigma D4527) | 10 | μl | 1 | μg/ml |

c-Kit Cellular Assay

1. 2×10⁷ H526 cells (SCLC; ATCC) were serum starved overnight in 0.1% Fetal Calf Serum. (Premium)

2. Incubated with dilutions of compounds for an hour (except two controls).

3. Stimulated with SCF (Stem Cell factor; R&D cat#255-SC) @ 250 ng/ml for 10 minutes (including one control).

4. Cell lysates were immunoprecipitated with 1DC3 @ 10 μg/mg protein

5. Immune complexes were harvested using Protein G+A agarose beads. (100 μl)

6. Used 8% Tris glycine gels for the separation of proteins.

7. Transferred protein on (PVDF or Nitrocellulose ) membrane using 40 volts for 2 hrs.

8. Blocked the membrane in 3% BSA in 1× PBS.

9. Western blotting was done using an anti c-Kit (1:500; AF332 anti human SCFR from R&D Systems ) as a primary antibody & anti-goat (1:2000) as a secondary antibody for detection of c-Kit protein.

4G10 as an anti-phosphotyrosine (1:5000) as a primary antibody & anti mouse (1:10000) as a secondary antibody for the detection of phosphorylation.

Blots were developed by using Cell signaling Lumiglo chemiluminiscent kit.

c-Kit Western Blot using KDR-Kit Chimera
cKit-KDR cells are plated @ 0.5×106 in 6 well plates
Next day cells are serum starved in 0.1% FCS in DMEM overnight
Following day compound added for one hour
50 ng/ml of VEGF added for 30 minutes
protein lysates made
protein assay done
Western blots run 20 μg/lane.

Antibodies used:

| Phospho c-kit (Tyr719) | catalogue number 3391 |
|---|---|
| (1:500) | Cell Signaling Technology |
| Mouse anti-human Flk-1/ | catalogue number RDI-FLK1Eabmx |
| KDR//VEGFR2 | Research Diagnostics, Inc |
| (1:500) | |

Homogenous time-resolved fluorescence (HTRF) in vitro kinase assay (Mathis, G., HTRF(R) Technology. J Biomol Screen, 1999. 4(6): p. 309-314):

For example, purified enzyme was mixed with 4 μM N-biotinylated substrate (e.g., poly(Glu₄Tyr)) and various concentrations of inhibitor in reaction buffer (50 mM HEPES, pH 7.0, 10 mM MgCl₂, 2 mM MnCl₂, 0.1% BSA and 1 mM DTT, 40 L final volume). The kinase reaction was initiated by addition of ATP (1 mM final conc.) in a black 96-well plate (Packard). After 30-60 minutes incubation at room temperature, the reaction was quenched by addition of a buffered EDTA solution (final approximate concentrations: 30 mM EDTA, 0.1% BSA, 0.1% Triton X-100 and 0.24M KF) and a solution of revelation agents (to give 0.084 ng/well streptavidin-XL-665 (Cis-Bio) and 6.5 ng/well antiphsophotyrosine mAb PT66-K Europinium kryptate) was added to the reaction mixture. The quenched reaction was allowed to stand at room temperature for 3 hour and then read in a time-resolved fluorescence detector (Discovery, Packard) at 620 nm and 665 nm simultaneously. A 337 nm nitrogen laser was used for excitation. The ratio between the signal of 620 nm and 665 nm was used in the calculation of the IC₅₀.

More specific details for the various enzymes are included below:

| | | | HTRF ASSAYS | | | | | | | | |
|---|---|---|---|---|---|---|---|---|---|---|---|
| | | | Enz. | | | | | | | | |
| | | | Reaction | | | | Peptide | | | DMSO | |
| | | | Conc. | | Assay | | Substrate | | ATP | Conc. | Reaction |
| Enzyme | Construct | MW (kD) | (ng/well) | | Buffer | Substrate | Conc. (μM) | | Conc. (mM) | (%) | Time (min) |
| Lck (Truncated) | 62-509 | 52 | 2.1 | | MOPSO | bio-LCK | 4 | | 1 | 5 | 60 |
| | | | | | | peptide | | | | | |
| Src (UBI) | NA | 60 | 0.15 | U/well | MOPSO | bio-LCK | 4 | | 1 | 5 | 60 |
| | | | | | | peptide | | | | | |
| Lyn | His6-Tag | 52 | 0.5 | | MOPSO | bio-LCK | 4 | | 1 | 5 | 60 |
| | | | | | | peptide | | | | | |
| Fyn (Catalytic | His6-Tag (257-534) | 34 | 0.15 | | MOPSO | bio-LCK | 4 | | 1 | 5 | 60 |
| Domain) | | | | | | peptide | | | | | |
| Csk | His6-Tag | 50 | 0.33 | | MOPSO | bio-PGT | 4 | | 1 | 5 | 10 |
| Lck (Catalytic | His6-Tag | 35 | 1 | | MOPSO | bio-LCK | 4 | | 1 | 5 | 60 |
| Domain) | | | | | | peptide | | | | | |
| Blk (Catalytic | His6-Tag | 60 | 0.15 | | MOPSO | bio-LCK | 4 | | 1 | 5 | 60 |
| Domain) | | | | | | peptide | | | | | |
| KDR | His6-KDR 789-1354 | 63 | 7 | | HEPES | bio-FGFR | 4 | | 1 | 5 | 60 |
| | | | | | | peptide | | | | | |
| Tie2 | His6-Tag | 40 | 12.6 | | HEPES | bio-PGT | 10 | ng/well | 1 | 5 | 10 |
| cKIT | GST-Fusion | 70 | 4* | | HEPES | bio-FGFR | 0.5* | | 1 | 5 | 60 |
| | | | | | | peptide | | | | | |
| Flt1 | His6-Tag | 65 | | | HEPES | bio-FGFR | 4 | | 1 | 5 | 60 |
| | | | | | | peptide | | | | | |
| CSF-1r | M-His(6)-CSF- | 50 | 10 | | HEPES | bio-Lck | 4 | | 1 | 5 | 60 |
| | 1R Q547-C972 | | | | | peptide | | | | | |

Buffers

| MOPSO Buffer: | HEPES Buffer: |
|---|---|
| 50 mM MOPSO pH6.5 | 50 mM HEPES pH7.1 |
| 2.5 mM DTT | 2.5 mM DTT |
| 10 mM MgCl₂ | 10 mM MgCl₂ |
| 2 mM MnCl₂ | 2 mM MnCl₂ |
| 0.01% BSA | 0.01% BSA |
| 100 μM Na₃VO₄ | 100 μM Na₃VO₄ |

Substrates

Bio-fgfr peptide (Biotin-Ahx-AEEEYFFLFA-amide)

Bio-lck peptide (Biotin-Ahx-GAEEEIYAAFFA-COOH)

Bio-PGT purchased from Cis-bio

One well contains a total of 40 μL reagents

PDGFRβ Enzyme ELISA Protocol

ELISA plates (Costar #3369 EIA/RIA 96 well easy wash high binding plates) pre-coated with 0.0625 μg/well anti-PDGFRβ antibody (Santa Cruz #SC-432) are washed four times in TPBS then blocked with 2% dry milk in PBS. After blocking, plates are blotted dry. 30 μl 0.667 ng/μl PDGFR enzyme (20 ng/well final) is added along with 20 μl drug solution at concentrations ranging from 200 μM to 0.0128 μM. Drug samples are diluted in 20% DMSO with Reaction buffer (50 mM Hepes pH 7.1, 100 mM MgCl₂, 20 mM MnCl₂, 2.5mM DTT, 0.01% BSA, 0.1 mM sodium vanadate). Enzyme and drug solution are incubated for 30 minutes. 30 μl 2.67 mM ATP (1 mM final) is added to initiate the reaction. After 8 minutes, the reaction is stopped with 20 μl 0.5 M EDTA pH 7.0 and plates are incubated for an additional 1.5 hours at room temperature. The plates are washed four times with TPBS. 100 μl anti-phosphotyrosine HRP conjugated antibody diluted 1/1000 in 2% milk/PBS is added to wells and plates are incubated for one hour at room temperature. Plates are washed four times with TPBS then 100 μl K-Blue substrate is added to wells. 10 μl 2N sulfuric acid is added after 10 minutes and the OD is determined at 450-570 nm. Background OD from a minus PDGFRβ control is subtracted from all data, and data are converted to percent activity by division by the OD of PDGFRβ samples lacking inhibitor. IC₅₀ values are determined by fitting the percent activity vs. inhibitor concentration data set to Percent Activity=1/(1+[I]/IC₅₀) by non-linear least-means-squares curve fitting.

In-vivo Models of T-Cell Activation

The in vivo efficacy of compounds can be tested in animal models known to directly measure T-cell activation or for which T-cells have been proven the effectors. T-cells can be activated in vivo by ligation of the constant portion of the T-cell receptor with a monoclonal anti-CD3 antibody (Ab). In this model, BALB/c mice are given 10 μg of anti-CD3 Ab intraperitoneally two hours prior to exsanguination. Animals to receive a test drug are pre-treated with a single dose of the compound one hour prior to anti-CD3 Ab administration. Serum levels of the proinflammatory cytokines interferon-γ (IFN-γ) and tumor necrosis factor-α (TNF-α), indicators of T-cell activation, are measured by ELISA. A similar model employs in vivo T-cell priming with a specific antigen such as keyhole limpet hemocyanin (KLH) followed by a secondary in vitro challenge of draining lymph node cells with the same antigen. As previously, measurement of cytokine production is used to assess the activation state of the cultured cells. Briefly, C57BL/6 mice are immunized subcutaneously with 100 μg KLH emulsified in complete Freund's adjuvant (CFA) on day zero. Animals are pre-treated with the compound one day prior to immunization and subsequently on days one, two and three post immunization. Draining lymph nodes are harvested on day 4 and their cells cultured at 6×10⁶ per ml in tissue culture medium (RPMI 1640 supplemented with heat inactivated fetal bovine serum (Hyclone Laboratories) 5×10⁻⁵ M 2-mercaptoethanol and 0.5% DMSO) for both twenty-four and forty-eight hours. Culture supernatants are then assessed for the autocrine T-cell growth factor Interleukin-2 (IL-2) and/or IFN-γ levels by ELISA.

Compounds can also be tested in animal models of human disease. These are exemplified by experimental auto-immune encephalomyelitis (EAE) and collagen-induced arthritis (CIA). EAE models which mimic aspects of human multiple sclerosis have been described in both rats and mice (reviewed FASEB J. 5:2560-2566, 1991; murine model: Lab. Invest. 4(3):278, 1981; rodent model:J. Immunol 146(4):1163-8, 1991). Briefly, mice or rats are immunized with an emulsion of myelin basic protein (MBP), or neurogenic peptide derivatives thereof, and CFA. Acute disease can be induced with the addition of bacterial toxins such as bordetella pertussis. Relapsing/remitting disease is induced by adoptive transfer of T-cells from MBP/ peptide immunized animals.

CIA may be induced in DBA/1 mice by immunization with type R collagen (J. Immunol:142(7):2237-2243). Mice will develop signs of arthritis as early as ten days following antigen challenge and may be scored for as long as ninety days after immunization. In both the EAE and CIA models, a compound may be administered either prophylactically or at the time of disease onset. Efficacious drugs should reduce severity and/or incidence.

Certain compounds of this invention which inhibit one or more angiogenic receptor PTK, and/or a protein kinase such as lck involved in mediating inflammatory responses can reduce the severity and incidence of arthritis in these models.

Compounds can also be tested in mouse allograft models, either skin (reviewed in Ann. Rev. Immunol., 10:333-58, 1992; Transplantation: 57(12): 1701-17D6, 1994) or heart (Am. J. Anat.:113:273, 1963). Briefly, full thickness skin grafts are transplanted from C57BL/6 mice to BALB/c mice. The grafts can be examined daily, beginning at day six, for evidence of rejection. In the mouse neonatal heart transplant model, neonatal hearts are ectopically transplanted from C57BL/6 mice into the ear pinnae of adult CBA/J mice. Hearts start to beat four to seven days post transplantation and rejection may be assessed visually using a dissecting microscope to look for cessation of beating.

Cellular Receptor PTK Assays

The following cellular assay was used to determine the level of activity and effect of the different compounds of the present invention on KDR/VEGFR2. Similar receptor PTK assays employing a specific ligand stimulus can be designed along the same lines for other tyrosine kinases using techniques well known in the art.

VEGF-Induced KDR Phosphorylation in Human Umbilical Vein Endothelial Cells (HUVEC) as Measured by Western Blots:

1. HUVEC cells (from pooled donors) were purchased from Clonetics (San Diego, Calif.) and cultured according to the manufacturer directions. Only early passages (3-8) were used for this assay. Cells were cultured in 100 mm dishes (Falcon for tissue culture; Becton Dickinson; Plymouth, England) using complete EBM media (Clonetics).

2. For evaluating a compound's inhibitory activity, cells were trypsinized and seeded at 0.5-1.0×10⁵ cells/well in each well of 6-well cluster plates (Costar; Cambridge, Mass.).

3. 3-4 days after seeding, plates were 90-100% confluent. Medium was removed from all the wells, cells were rinsed with 5-10 ml of PBS and incubated 18-24 h with 5 ml of EBM base media with no supplements added (i.e., serum starvation).

4. Serial dilutions of inhibitors were added in 1 ml of EBM media (25 μM, 5 μM, or 1 μM final concentration to cells and incubated for one hour at 37° C. Human recombinant VEGF₁₆₅ ( R & D Systems) was then added to all the wells in 2 ml of EBM medium at a final concentration of 50ng/ml and incubated at 37° C. for 10 minutes. Control cells untreated or treated with VEGF only were used to assess background phosphorylation and phosphorylation induction by VEGF.

All wells were then rinsed with 5-10 ml of cold PBS containing 1 mM Sodium Orthovanadate (Sigma) and cells were lysed and scraped in 200 μl of RIPA buffer (50 mM Tris-HCl) pH 7, 150 mM NaCl, 1% NP-40, 0.25% sodium deoxycholate, 1 mM EDTA) containing protease inhibitors (PMSF 1 mM, aprotinin 1 μg/ml, pepstatin 1 μg/ml, leupeptin 1 μg/ml, Na vanadate 1 mM, Na fluoride 1 mM) and 1 μg/ml of Dnase (all chemicals from Sigma Chemical Company, St Louis, Mo.). The lysate was spun at 14,000 rpm for 30 min, to eliminate nuclei.

Equal amounts of proteins were then precipitated by addition of cold (−20° C.) Ethanol (2 volumes) for a minimum of 1 hour or a maximum of overnight. Pellets were reconstituted in Laemli sample buffer containing 5% -mercaptoethanol (BioRad; Hercules, Calif.) and boiled for 5 min. The proteins were resolved by polyacrylamide gel electrophoresis (6%, 1.5 mm Novex, San Deigo, Calif.) and transferred onto a nitrocellulose membrane using the Novex system. After blocking with bovine serum albumin (3%), the proteins were probed overnight with anti-KDR polyclonal antibody (C20, Santa Cruz Biotechnology; Santa Cruz, Calif.) or with anti-phosphotyrosine monoclonal antibody (4G10, Upstate Biotechnology, Lake Placid, N.Y.) at 4° C. After washing and incubating for 1 hour with HRP-conjugated F(ab)₂ of goat-anti-rabbit or goat-anti-mouse IgG the bands were visualized using the emission chemiluminescience (ECL) system (Amersham Life Sciences, Arlington Height, Ill.). Certain examples of the present invention significantly inhibit cellular VEGF-induced KDR tyrosine kinase phosphorylation at concentrations of less than 50 μM.

In vivo Uterine Edema Model

This assay measures the capacity of compounds to inhibit the acute increase in uterine weight in mice which occurs in the first few hours following estrogen stimulation. This early onset of uterine weight increase is known to be due to edema caused by increased permeability of uterine vasculature. Cullinan-Bove and Koss (Endocrinology (1993), 133:829-837) demonstrated a close temporal relationship of estrogen-stimulated uterine edema with increased expression of VEGF mRNA in the uterus. These results have been confirmed by the use of neutralizing monoclonal antibody to VEGF which significantly reduced the acute increase in uterine weight following estrogen stimulation (WO 97/42187). Hence, this system can serve as a model for in vivo inhibition of VEGF signalling and the associated hyperpermeability and edema.

Materials: All hormones were purchased from Sigma (St. Louis, Mo.) or Cal Biochem (La Jolla, Calif.) as lyophilized powders and prepared according to supplier instructions.

Vehicle components (DMSO, Cremaphor EL) were purchased from Sigma (St. Louis, Mo.).
Mice (Balb/c, 8-12 weeks old) were purchased from Taconic (Germantown, N.Y.) and housed in a pathogen-free animal facility in accordance with institutional Animal Care and Use Committee Guidelines.
   Method:

Day 1: Balb/c mice were given an intraperitoneal (i.p.) injection of 12.5 units of pregnant mare's serum gonadotropin (PMSG).

Day 3: Mice received 15 units of human chorionic gonadotropin (hCG) i.p.

Day 4: Mice were randomized and divided into groups of 5-10. Test compounds were administered by i.p., i.v. or p.o. routes depending on solubility and vehicle at doses ranging from 1-100 mg/kg. Vehicle control group received vehicle only and two groups were left untreated.

Thirty minutes later, experimental, vehicle and one of the untreated groups were given an i.p. injection of 17-estradiol (500 g/kg). After 2-3 hours, the animals were sacrificed by CO₂ inhalation. Following a midline incision, each uterus was isolated and removed by cutting just below the cervix and at the junctions of the uterus and oviducts. Fat and connective tissue were removed with care not to disturb the integrity of the uterus prior to weighing (wet weight). Uteri were blotted to remove fluid by pressing between two sheets of filter paper with a one liter glass bottle filled with water. Uteri were weighed following blotting (blotted weight). The difference between wet and blotted weights was taken as the fluid content of the uterus. Mean fluid content of treated groups was compared to untreated or vehicle treated groups. Significance was determined by Student's test. Non-stimulated control group was used to monitor estradiol response.

Results demonstrate that certain compounds of the present invention inhibit the formation of edema when administered systemically by various routes.

Certain compounds of this invention which are inhibitors of angiogenic receptor tyrosine kinases can also be shown to be active in a Matrigel implant model of neovascularization. The Matrigel neovascularization model involves the formation of new blood vessels within a clear marble of extracellular matrix implanted subcutaneously which is induced by the presence of proangiogenic factor producing tumor cells (for examples see: Passaniti, A., et al, Lab. Investig. (1992), 67(4), 519-528; Anat. Rec. (1997), 249(1), 63-73; Int. J. Cancer (1995), 63(5), 694-701; Vasc. Biol. (1995), 15(11), 1857-6). The model preferably runs over 3-4 days and endpoints include macroscopic visual/image scoring of neovascularization, microscopic microvessel density determinations, and hemoglobin quantitation (Drabkin method) following removal of the implant versus controls from animals untreated with inhibitors. The model may alternatively employ bFGF or HGF as the stimulus.

Certain compounds of this invention which inhibit one or more oncogenic, protooncogenic, or proliferation-dependent protein kinases, or angiogenic receptor PTK also inhibit the growth of primary murine, rat or human xenograft tumors in mice, or inhibit metastasis in murine models.

### EXAMPLES

### ABBREVIATIONS

Boc tert-Butoxycarbonyl
dba dibenzylidene acetone
DCM Dichloromethane
DEAD Diethyl azodicarboxylate
DIAD Diisopropyl azodicarboxylate
DME 1,2-Dimethoxyethane
DMF N,N-dimethylformamide
EtOAc Ethyl acetate
Ms Methanesulfonyl
NMP N-Methylpyrrolidin-2-one
r.t. room temperature
TBAF tert-Butylammonium fluoride
TEA Triethylamine
THF Tetrahydrofuran
Ts para-Toluenesulfonyl

### General Procedures and Examples

The majority of the following examples are ordered according to the ultimate final general procedure used in their preparation. The synthetic routes to any novel intermediates are detailed by sequentially listing the general procedures (letter codes) in parenthesis after their name. A worked example of this protocol is given below. Analytical data is defined either within the experimental conditions or the tables of examples. Unless otherwise stated, all ¹H or ¹³C NMR data was collected on a Varian Mercury plus 400 MHz or a Bruker DRX 400 MHz instrument, chemical shifts are quoted in parts per million (ppm). High pressure liquid chromatography analytical data is either detailed within the experimental or referenced to the table of HPLC conditions using the lower case method letter in parenthesis (Table 1).

Table 1. List of HPLC methods

TABLE 1
List of HPLC methods

| Method | HPLC Conditions |
|---|---|
| a | RP-HPLC (5% to 95% acetonitrile/0.05M aqueous ammonium acetate, |
| | buffered to pH 4.5, over 10 min at 1.7 mL/min; λ = 254 nm; Hypersil |
| | C18, 100 Å, 5 μm, 250 × 4.6 mm column). |
| b | RP-HPLC (5% to 95% acetonitrile/0.05M aqueous ammonium acetate, |
| | buffered to pH 4.5, over 10 min at 1 mL/min; λ = 254 nm; Hypersil |
| | C18, 100 Å, 5 μm, 250 × 4.6 mm column). |
| c | RP-HPLC (5% to 85% acetonitrile/0.05M aqueous ammonium acetate, |
| | buffered to pH 4.5, over 20 min at 1.7 mL/min; λ = 254 nm; Hypersil |
| | C18, 100 Å, 5 μm, 250 × 4.6 mm column). |
| d | RP-HPLC (5% to 95% acetonitrile/0.05M aqueous ammonium acetate, |
| | buffered to pH 4.5, over 20 min at 1.7 mL/min; λ = 254 nm; Hypersil |
| | C18, 100 Å, 5 μm, 250 × 4.6 mm column). |
| e | RP-HPLC (25% to 100% acetonitrile/0.1 M aqueous ammonium |
| | acetate, buffered to pH 4.5, over 20 min at 1.0 mL/min; λ = 254 nm; |
| | Hypersil C18, 100 Å, 5 μm, 250 × 4.6 mm column). |
| f | RP-HPLC (30% to 95% acetonitrile/0.01M aqueous ammonium |
| | acetate, buffered to pH 4.5, over 4.5 min at 0.8 mL/min; λ = 190-700 nm; |
| | Genesis C18, 120 Å, 4 μm, 33 × 4.6 mm column. |
| g | RP-HPLC (10% to 80% acetonitrile/0.01M aqueous ammonium |
| | acetate, buffered to pH 4.5, over 6 min at 0.8 mL/min; λ = 190-700 nm; |
| | Genesis C18, 120 Å, 3 μm, 30 × 4.6 mm column). |
| h | RP-HPLC (5% to 95% acetonitrile/0.1% H₃PO₄ (aq), over 7minutes at |
| | 1.5 mL/min; λ = 190-700 nm; Zorbax SB-C8 rapid resolution, 4.6 mm × 75 mm, |
| | 3.5 μm column). |
| i | RP-HPLC (50% to 100% acetonitrile/0.05M aqueous ammonium |
| | acetate, buffered to pH 4.5, over 15 min at 1.7 mL/min; λ = 254 nm; |
| | Hypersil C18, 100 Å, 5 μm, 250 × 4.6 mm column). |
| j | RP-HPLC (5% to 85% acetonitrile/0.05M aqueous ammonium acetate, |
| | buffered to pH 4.5, over 20 min at 1 mL/min; λ = 254 nm; Hypersil |
| | C18, 100 Å, 5 μm, 250 × 4.6 mm column). |
| k | RP-HPLC (5% to 95% acetonitrile/0.05 M ammonium acetate, buffered |
| | to pH 4.5, over 3.5 min at 2 mL/min, λ = 250-470 nm; Pecosphere |
| | C18, 3 μm, 33 × 4.6 mm column). |
| l | RP-HPLC (25% to 100% acetonitrile/0.1 M aqueous ammonium |
| | acetate, buffered to pH 4.5, over 10 min at 1.0 mL/min; λ = 254 nm; |
| | Hypersil C18, 100 Å, 5 μm, 250 × 4.6 mm column). |

General Synthetic Routes

The general synthetic schemes that were utilized to construct the majority of compounds enclosed in this application are described below (Schemes 1-3).

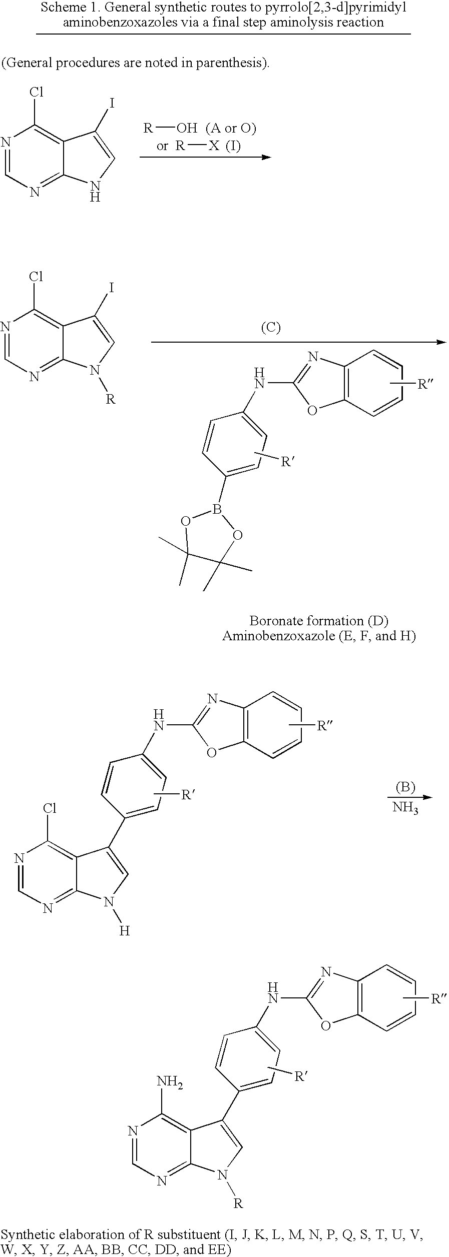

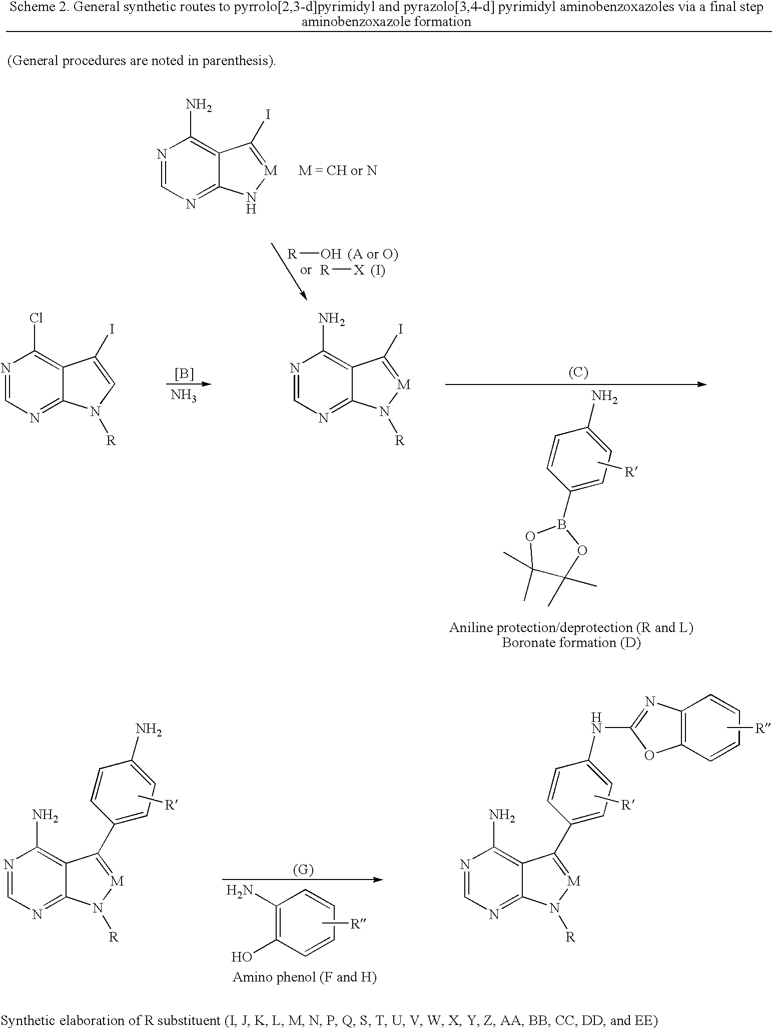

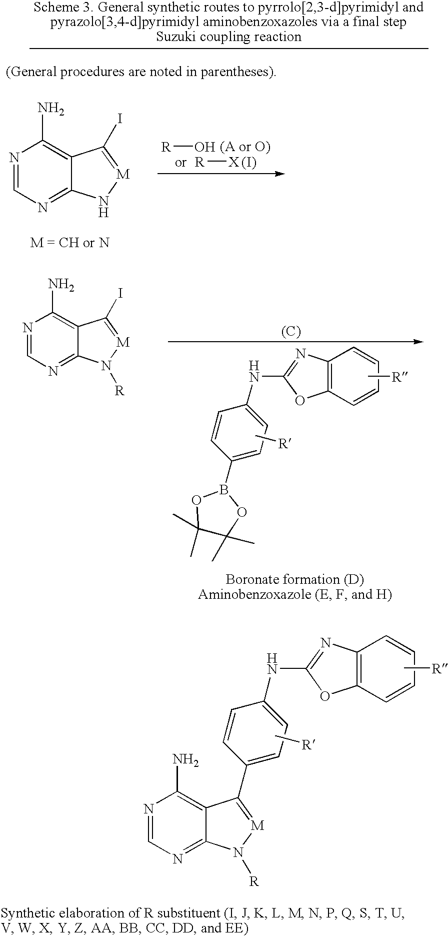

### LIST OF GENERAL PROCEDURES

General procedure A: Mitsunobu coupling of a pyrazolo[3,4-d]pyrimidine or pyrrolo[2,3-d]pyrimidine with an alcohol.
General procedure B: Aminolysis of a chloropyrimidine
General procedure C: Suzuki coupling of a halide with a boronate ester or a boronic acid
General Procedure D: Conversion of a bromide to a boronate
General procedure E: Cyclization of an aminoalcohol to a benzoxazole
General procedure F: Nitration of phenols
General Procedure G: Transformation of an aniline to an aminobenzoxazole
General procedure H: Reduction of a nitroaromatic compound to an aniline
General procedure I: Alkylation of nitrogen-based nucleophile
General procedure J: Reductive coupling of an amine with a ketone
General procedure K: Ketalization of a ketone
General procedure L: Removal of a Boc-protecting group
General procedure M: N-alkylation of lactam
General procedure N: Debenzylation of a benzyl ether compound
General Procedure O: Mitsunobu coupling of a pyrazolo[3,4-d]pyrimidine or a pyrrolo[2,3-d]pyrimidine with an alcohol using a resin bound phosphine source.
General Procedure P: Ester Hydrolysis
General Procedure Q: EDC-coupling of an acid with an amine
General Procedure R: Boc-protection of an amine
General Procedure S: α-Alkylation of a hydroxy alkyl carboxylate.
General Procedure T: Deketalization of a protected cyclohexanone
General procedure U: Reduction of ketone or ester to an alcohol
General Procedure V: Mesylation of an alcohol and subsequent displacement of the mesylate group
General procedure W: Acylation of an amine with an acid chloride, sulfonyl chloride or an anhydride.
General procedure X: 0-alkylation of an alcohol
General procedure Y: 2,5-Diketopiperazine synthesis
General Procedure Z: Homoketopiperazine synthesis
General procedure AA: Carbonylative cyclization of diamines and aminoalcohols
General Procedure BB: Ketomorpholine synthesis
General procedure CC: Deprotection of a silyl-protected alcohol
General procedure DD: Synthesis of a trifluoromethoxy ether
General procedure EE: Oxidation of a sulfide to a sulfoxide or a sulfone
General procedure FF: Ring closure to form substituted aminobenzoxazoles in a one step protocol

### Worked Example Using General Procedures

The general procedure letter codes constitute a synthetic route to the final product. A worked example of how the route is determined is given below using Example #1 as the test case. The synthesis of Example #1 was completed using general procedure B as detailed in Table 2, i.e.

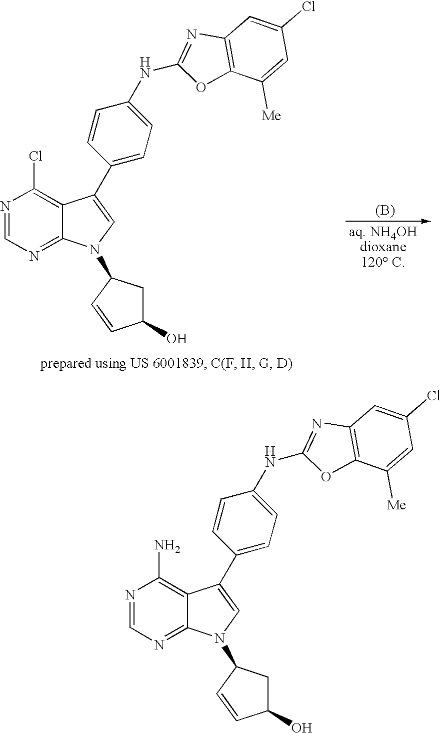

In this case, the chloropyrimidine starting material, compound A, was prepared using the route U.S. Pat. No. 6,001,839, C (F, H, G, D) (as detailed in Table 2). This translates into the following sequence, where the reagent used in general procedure C is the product of following the procedures F, H, G, and D, hence these steps are designated in additional parentheses.

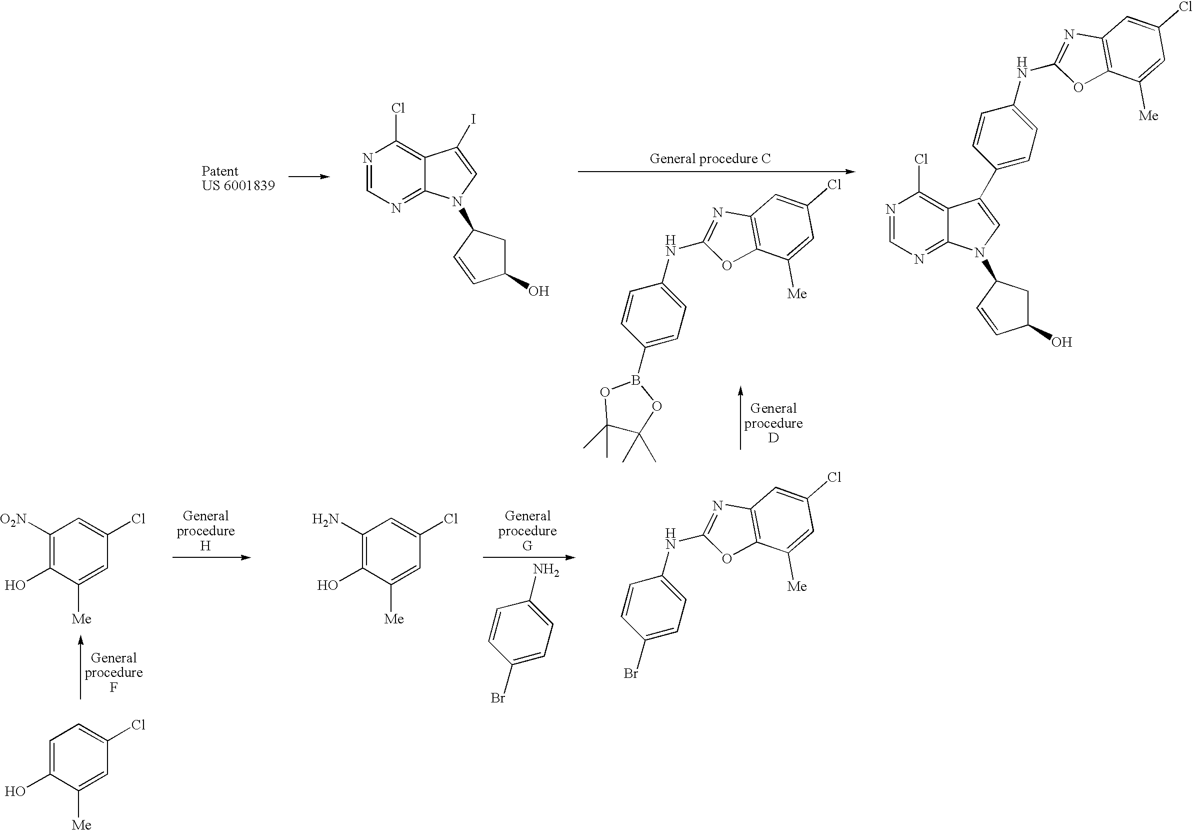

### General Procedure A: Mitsunobu Coupling of a pyrazolo[3,4 d]pyrimidine or pyrrolo[2,3-d]pyrimidine with an Alcohol

A mixture of pyrazolo[3,4-d]pyrimidine or pyrrolo[2,3-d]pyrimidine (preferably 1 equivalent), an alcohol (1-5 equivalents, preferably 3 equivalents), a phosphine (for example, triphenylphosphine) (1-5 equivalents, preferably 3 equivalents), and an azodicarboxylate (for example, diisopropylazodicarboxylate) (1-5 equivalents, preferably 3 equivalents) is stirred in an anhydrous solvent (preferably tetrahydrofuran) at about 0-100° C. (preferably about 20° C.) for about 0.5-24 hours (preferably about 4 hours) under an inert atmosphere. The solvent is removed under reduced pressure. The resulting residue is partitioned between an organic solvent and an aqueous solution. The organic layer is separated and the aqueous layer is further extracted with an organic solvent. The combined organic extracts are dried over a desiccant. The solvent is evaporated under reduced pressure to afford the product, which can be further purified by crystallization or chromatography.

### Illustration of General Procedure A

### Preparation #1: cis -3-Iodo-1-[4-(2-methoxyethoxy)-cyclohexyl]-1H-pyrazolo[3,4-d]pyrimidin-4-ylamine

### Preparation #2: trans-3-Iodo-1-[4-(2-methoxyethoxy)-cyclohexyl]-1H-pyrazolo[3,4-4pyrimidin-4-ylamine

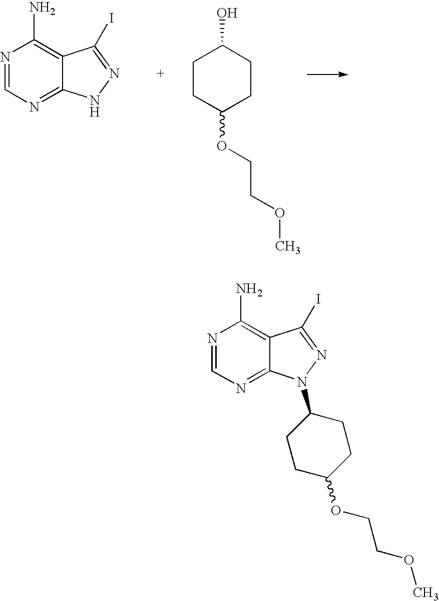

To a solution of 3-iodo-1H-pyrazolo[3,4-d]pyrimidin-4-ylamine (0.626 g, 2.40 mmol) in anhydrous tetrahydrofuran (25 mL) was added triphenylphosphine (1.51 g, 5.76 mmol) and diisopropylazodicarboxylate (1.16 g, 5.76 mmol). The mixture was stirred for about five minutes at ambient temperature under a nitrogen atmosphere and 4-(2-methoxyethoxy)-cyclohexanol (JP 61229865, mixture of cis- and trans-isomers, 1.04 g, 5.98 mmol) was added. The reaction mixture was stirred at ambient temperature for about three hours. The tetrahydrofuran was removed under reduced pressure and the crude mixture was stirred in a mixture of acetone (15 mL) and aqueous hydrochloric acid (2 N, 15 mL) for two hours at ambient temperature. The acetone was removed under reduced pressure and the aqueous mixture was neutralized by the addition of saturated aqueous sodium bicarbonate solution such that the pH was approximately 8. The aqueous mixture was extracted with ethyl acetate (3×25 mL) and the combined organic fractions were dried over anhydrous magnesium sulfate. The crude mixture was purified by flash column chromatography on silica gel using ethyl acetate as the mobile phase to provide pure white solids of both trans-3-iodo-1-[4-(2-methoxyethoxy)-cyclohexyl]-1H-pyrazolo[3,4-d]pyrimidin-4-ylamine (200 mg, 0.480 mmol); ¹H NMR (DMSO-d₆ 400 MHz) δ8.18 (s, 1H), 4.59 (m, 1H), 3.56 (dd, 2H), 3.46 (dd 2H), 3.36 (tt 1H), 3.25 (s, 3H), 2.08 (d, 2H), 1.92 (m, 4H), 1.33-1.37 (qd, 2H); m/z: (M+H)⁺ 418, and cis-3-iodo-1-[4-(2-methoxyethoxy)-cyclohexyl]-1H-pyrazolo[3,4-d]pyrimidin-4-ylamine (120 mg, 0.288 mmol); ¹H NMR (DMSO-d_{6,} 400 MHz) δ8.18 (s, 1H), 4.63 (tt, 1H), 3.58 (t, 1H), 3.52 (td, 2H), 3.50 (td, 2H), 3.29 (s, 3H), 2.15 (q, 2H), 1.95 (d, 2H), 1.61 (m, 4H); m/z: (M+H)⁺ 418.

### General procedure B: Aminolysis of a chloropyrimidine

A mixture of a 4-chloro-5-iodo-7H-pyrrolo[2,3-d]pyrimidine (preferably 1 equivalent) and aqueous ammonium hydroxide (28% ammonia by weight) (100-300 equivalents, preferably 300 equivalents) is heated in dioxane in a Parr mini-reactor at about 80-150° C. (preferably about 120° C.) for about 1-48 hours (preferably about 12 hours). The mixture is allowed to cool to ambient temperature and the solvents are removed under reduced pressure to afford the product that can be further purified by crystallization or chromatography.

### Illustration of General Procedure B

### Preparation #3: trans-7-(4-Cyclopropylmethoxy-cyclohexyl)-5-iodo-7H-pyrrolo[2,3-d]pyrimidin-4-ylamine

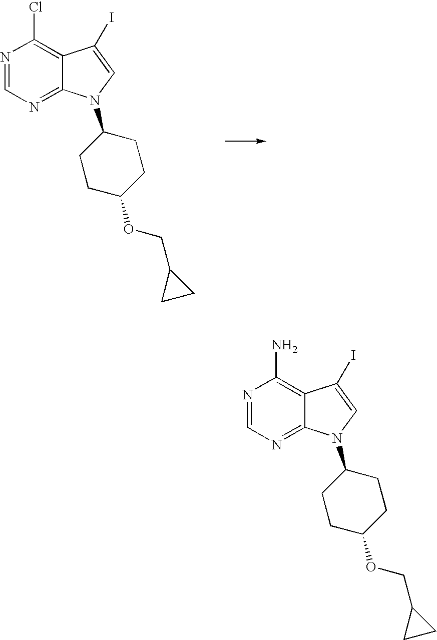

A mixture of trans-4-chloro-7-(4-cyclopropylmethoxy-cyclohexyl)-5-iodo-7H-pyrrolo[2,3-d]pyrimidine (prepared by general procedures X, T, U, and A) (0.357 g, 0.00083 mol) in aqueous ammonium hydroxide (28% ammonia by weight, 15 mL, 0.247 mol, 298 equivalents) was heated in dioxane (15 mL) in a Parr mini-reactor at about 120° C. for about 12 hours. The mixture was allowed to cool to ambient temperature and the solvents were removed under reduced pressure to give trans-7-(4-cyclopropylmethoxy-cyclohexyl)-5-iodo-7H-pyrrolo[2, 3-d]pyrimidin-4-ylamine as a white solid (0.509 g, 0.00083 mol, containing ammonium chloride); LC/MS (30% to 95% acetonitrile/0.01M aqueous ammonium acetate over 4.5 min at 0.8 mL/min; λ=190-700 nm; Genesis C18, 120 Å, 3 μm, 30×4.6 mm column; Electrospray ionization method observing both positive and negative ions) Rₜ 2.80 min; m/z: (M+H)⁺ 413.

Other products obtained using general procedure B are shown (Table 2). The method used to determine the HPLC retention time is given in a lower-case letter in parentheses (see Table 1).

TABLE 2
Examples synthesized using general procedure B

| Chloropyrimidine | | | HPLC RT | |
|---|---|---|---|---|
| Precursor | Product | Example # | (Method) | m/z |
| cis-4-{4-Chloro-5-[4-(5- | cis-4-{4-Amino-5-[4-(5- | 1 | 11.0 min (b) | 473 (M + H)⁺ |
| chloro-7-methyl- | chloro-7-methyl- | | | |
| benzoxazol-2- | benzoxazol-2- | | | |
| ylamino)-phenyl]- | ylamino)-phenyl]- | | | |
| pyrrolo[2,3-d]pyrimidin- | pyrrolo[2,3- | | | |
| 7-yl}-cyclopent-2-enol | d]pyrimidin-7-yl}- | | | |
| ((US 6001839), | cyclopent-2-enol | | | |
| C(F,H,G,D)) | | | | |
| cis-4-{4-Chloro-5-[4- | cis-4-{4-Amino-5-[4- | 2 | 10.6 min (b) | 453 (M + H)⁺ |
| (5,7-dimethyl- | (5,7-dimethyl- | | | |
| benzoxazol-2- | benzoxazol-2- | | | |
| ylamino)-phenyl]- | ylamino)-phenyl]- | | | |
| pyrrolo[2,3-d]pyrimidin- | pyrrolo[2,3- | | | |
| 7-yl}-cyclopent-2-enol | d]pyrimidin-7-yl}- | | | |
| ((US 6001839), | cyclopent-2-enol | | | |
| C(G,D)) | | | | |

### General Procedure C: Suzuki Coupling of a Halide with a Boronate Ester or a Boronic Acid

A mixture of a boronate ester or a boronic acid (1-5 equivalents, preferably 1.5 equivalents), a halide (for example a bromide or an iodide, preferably an iodide) (preferably 1 equivalent) and a base (for example, sodium carbonate or cesium carbonate, preferably sodium carbonate) (1-10 equivalents, preferably 2 equivalents) is heated in a mixture of an organic solvent (for example, ethylene glycol dimethyl ether, N,N-dimethylformamide, or toluene, preferably ethylene glycol dimethyl ether) and water at about 20-120° C. (preferably about 80° C.). A palladium catalyst (for example, palladium(II) acetate, tris(dibenzylideneacetone)dipalladium(0), tetrakis(triphenylphosphine)palladium(0), preferably tetrakis(triphenylphosphine)-palladium(0)) (0.01-0.2 equivalents, preferably 0.05 equivalents) is added and the reaction mixture is allowed to stir for about 1-48 hours (preferably about 12 hours) under an inert atmosphere. The mixture is allowed to cool to ambient temperature and the solvents are removed under reduced pressure. The residue is partitioned between water and an organic solvent, the organic layer is separated and the aqueous layer is further extracted with organic solvent. The combined organic extracts are dried over a desiccant. The solvents are evaporated under reduced pressure to afford the product that can be further purified by crystallization or chromatography.

### Illustration of General Procedure C

### Example #3

cis-{4-(4-{4-Amino-3-[4-(5,7-dimethyl-benzoxazol-2-ylamino)-phenyl]-pyrazolo[3,4-d]pyrimidin-1-yl}-cyclohexyl)}-1-methyl-piperazin-2-one

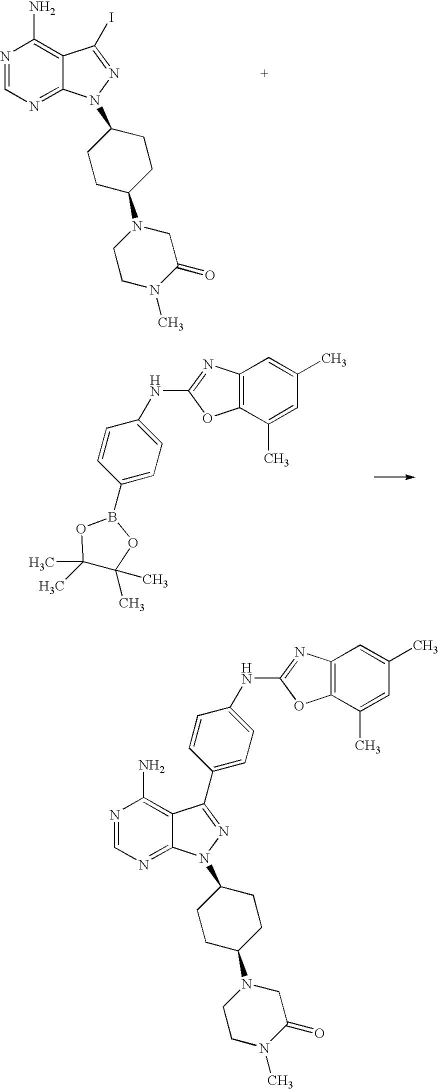

To a mixture of cis-{4-[4-(4-amino-3-iodo-pyrazolo[3,4-d]pyrimidin-1-yl)-cyclohexyl]}-1-methyl-piperazin-2-one (prepared using general procedures A, T, and J (employing a ketopiperazine described in U.S. Pat. No. 4,251,438)) (80 mg, 0.18 mmol), (5,7-dimethyl-benzoxazol-2-yl)-[4-(4,4,5,5-tetramethyl-[1,3,2]dioxaborolan-2-yl)-phenyl]-amine (G,D) (80 mg, 0.22,mmol) and sodium carbonate (47 mg, 0.44 mmol) in N,N-dimethylformamide (4 mL) and water (2 mL) was added tetrakis(triphenylphosphine)palladium(0) (20 mg, 0.017 mmol), at room temperature, under an atmosphere of nitrogen. The reaction mixture was heated at about 80° C for about 16 hours. The mixture was allowed to cool to ambient temperature and solvents were removed under the reduced pressure. The residue was partitioned between water (25 mL) and dichloromethane (25 mL), the organic layer was separated and the aqueous layer further was extracted with dichloromethane (2×25 mL). The combined organic extracts were dried over magnesium sulfate, then evaporated under reduced pressure. The residue was purified by flash column chromatography on silica using dichloromethane/methanol/ammonium hydroxide (28-30% solution) (95:4.95:0.05) mixture as the mobile phase to give cis-{4-(4-{4-amino-3-[4-(5, 7-dimethyl-benzoxazol-2-ylamino)-phenyl]-pyrazolo[3,4-d]pyrimidin-1-yl}-cyclohexyl)}-1-methyl-piperazin-2-one as a white solid (56.0 mg, 0.099 mmol); ¹H NMR (DMSO-d₆, 400 MHz) δ 10.85, 8.23, 7.92, 7.64, 7.11, 6.80, 4.81, 3.17, 3.07, 2.84, 2.72, 2.40, 2.35, 2.12-2.08 and 1.72-1.60; RP-HPLC (5% to 85% acetonitrile/0.05M aqueous ammonium acetate, buffered to pH 4.5, over 20 min at 1 mL/min; λ=254 nm; Hypersil C18, 100 Å, 5 μm, 250×4.6 mm column)Rₜ 16.73 min.

Other products obtained using general procedure C are shown (Table 3). The method used to determine the HPLC retention time is given in a lower-case letter in parentheses (see Table 1).

TABLE 3
Examples synthesized using general procedure C

| Iodide or Bromide | | | | HPLC RT | m/z or ¹H |
|---|---|---|---|---|---|
| Precursor | Boronate Precursor | Product | Example # | (Method) | NMR δ |
| trans-3-Iodo-1-[4-(4- | (5,7-Dimethyl- | trans-3-[4-(5,7- | 4 | 2.27 min (f) | 566 (M + H)⁺ |
| methyl-piperazin-1-yl)- | benzoxazol-2-yl)-[2- | Dimethyl-benzoxazol- | | | |
| cyclohexyl]-1H- | methyl-4-(4,4,5,5- | 2-ylamino)-3-methyl- | | | |
| pyrazolo[3,4- | tetramethyl- | phenyl]-1-[4-(4-methyl- | | | |
| d]pyrimidin-4-ylamine | [1,3,2]dioxaborolan-2- | piperazin-1-yl)- | | | |
| (A, T, J) | yl)-phenyl]-amine | cyclohexyl]-1H- | | | |
| | (G, D) | pyrazolo[3,4- | | | |
| | | d]pyrimidin-4-ylamine | | | |
| trans-3-Iodo-1-[4-(4- | (5,7-Dimethyl- | trans-3-[4-(5,7- | 5 | 3.00 min (f) | 580 (M + H)⁺ |
| methyl-piperazin-1-yl)- | benzoxazol-2-yl)-[2- | Dimethyl-benzoxazol- | | | |
| cyclohexyl]-1H- | ethyl-4-(4,4,5,5- | 2-ylamino)-3-ethyl- | | | |
| pyrazolo[3,4- | tetramethyl- | phenyl]-1-[4-(4-methyl- | | | |
| d]pyrimidin-4-ylamine | [1,3,2]dioxaborolan-2- | piperazin-1-yl)- | | | |
| (A, T, J) | yl)-phenyl]-amine | cyclohexyl]-1H- | | | |
| | (G, D) | pyrazolo[3,4- | | | |
| | | d]pyrimidin-4-ylamine | | | |
| trans-4-(4-Amino-3- | (5,7-Dimethyl- | trans-4-{4-Amino-3-[4- | 6 | 13.90 min | δ (DMSO-d₆) |
| iodo-pyrazolo[3,4- | benzoxazol-2-yl)-[4- | (5,7-dimethyl- | | (k) | 10.85, 8.24, |
| d]pyrimidin-1-yl)-1- | (4,4,5,5-tetramethyl- | benzoxazol-2- | | | 7.92, 7.67, |
| methyl-cyclohexane- | [1,3,2]dioxaborolan-2- | ylamino)-phenyl]- | | | 7.11, 6.80, |
| carboxylic acid ethyl | yl)-phenyl]-amine | pyrazolo[3,4- | | | 4.67, 4.10, |
| ester (S, A) | (G, D) | d]pyrimidin-1-yl}-1- | | | 2.40, 2.35, |
| | | methyl-cyclohexane- | | | 2.20, 1.83, |
| | | carboxylic acid ethyl | | | 1.29, 1.21 |
| | | ester | | | |
| trans-4-(4-Amino-3- | (5,7-Dimethyl- | trans-4-{4-Amino-3-[4- | 7 | 4.33 min (f) | 554 (M + H)⁺ |
| iodo-pyrazolo[3,4- | benzoxazol-2-yl)-[4- | (5,7-dimethyl- | | | |
| d]pyrimidin-1-yl)-1- | (4,4,5,5-tetramethyl- | benzoxazol-2- | | | |
| ethyl- | [1,3,2]dioxaborolan-2- | ylamino)-phenyl]- | | | |
| cyclohexanecarboxylic | yl)-phenyl]-amine | pyrazolo[3,4- | | | |
| acid ethyl ester (S, A) | (G, D) | d]pyrimidin-1-yl}-1- | | | |
| | | ethyl- | | | |
| | | cyclohexanecarboxylic | | | |
| | | acid ethyl ester | | | |
| trans-7-(4- | (5,7-Dimethyl- | trans-7-(4- | 8 | 3.80 min (f) | 523 (M + H)⁺ |
| Cyclopropylmethoxy- | benzoxazol-2-yl)-[4- | Cyclopropylmethoxy- | | | |
| cyclohexyl)-5-iodo-7H- | (4,4,5,5-tetramethyl- | cyclohexyl)-5-[4-(5,7- | | | |
| pyrrolo[2,3- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (X, T, U, A, B) | (G, D) | 7H-pyrrolo[2,3- | | | |
| | | d]pyrimidin-4-ylamine | | | |
| cis-7-(4- | (5,7-Dimethyl- | cis-7-(4- | 9 | 3.96 min (f) | 523 (M + H)⁺ |
| Cyclopropylmethoxy- | benzoxazol-2-yl)-[4- | Cyclopropylmethoxy- | | | |
| cyclohexyl)-5-iodo-7H- | (4,4,5,5-tetramethyl- | cyclohexyl)-5-[4-(5,7- | | | |
| pyrrolo[2,3- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (X, T, U, A, B) | (G, D) | 7H-pyrrolo[2,3- | | | |
| | | d]pyrimidin-4-ylamine | | | |
| cis-2-[4-(4-Amino-5- | (5,7-Dimethyl- | cis-2-(4-{4-Amino-5-[4- | 10 | 2.26 min (f) | 525 (M + H)⁺ |
| iodo-pyrrolo[2,3- | benzoxazol-2-yl)-[4- | (5,7-dimethyl- | | | |
| d]pyrimidin-7-yl)- | (4,4,5,5-tetramethyl- | benzoxazol-2- | | | |
| cyclohexylamino]- | [1,3,2]dioxaborolan-2- | ylamino)-phenyl]- | | | |
| acetamide (A, T, J) | yl)-phenyl]-amine | pyrrolo[2,3- | | | |
| | (G, D) | d]pyrimidin-7-yl}- | | | |
| | | cyclohexylamino)- | | | |
| | | acetamide | | | |
| trans-7-(4- | (5-Chloro-benzoxazol- | trans-5-[4-(5-Chloro- | 11 | 3.62 min (f) | 547 (M + H)⁺ |
| Cyclopropylmethoxy- | 2-yl)-[2-fluoro-4- | benzoxazol-2- | | | |
| cyclohexyl)-5-iodo-7H- | (4,4,5,5-tetramethyl- | ylamino)-3-fluoro- | | | |
| pyrrolo[2,3- | [1,3,2]dioxaborolan-2- | phenyl]-7-(4- | | | |
| d]pyrimidin-4-ylamine | yl)-phenyl]-amine | cyclopropylmethoxy- | | | |
| (X, T, U, A, B) | (G, D) | cyclohexyl)-7H- | | | |
| | | pyrrolo[2,3- | | | |
| | | d]pyrimidin-4-ylamine | | | |
| trans-2-[4-(4-Amino-5- | (5,7-Dimethyl- | trans-2-(4-{4-Amino-5- | 12 | 2.30 min (f) | 525 (M + H)⁺ |
| iodo-pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexylamino]- | [1,3,2]dioxaborolan-2- | ylamino)-phenyl]- | | | |
| acetamide (A, T, J) | yl)-phenyl]-amine | pyrrolo[2,3- | | | |
| | (G, D) | d]pyrimidin-7-yl}- | | | |
| | | cyclohexylamino)- | | | |
| | | acetamide, bis acetic | | | |
| | | acid salt | | | |
| cis-7-(4- | [2-Fluoro-4-(4,4,5,5- | cis-7-(4- | 13 | 3.85 min (f) | 527 (M + H)⁺ |
| Cyclopropylmethoxy- | tetramethyl- | Cyclopropylmethoxy- | | | |
| cyclohexyl)-5-iodo-7H- | [1,3,2]dioxaborolan-2- | cyclohexyl)-5-[3- | | | |
| pyrrolo[2,3- | yl)-phenyl]-(5-methyl- | fluoro-4-(5-methyl- | | | |
| d]pyrimidin-4-ylamine | benzoxazol-2-yl)- | benzoxazol-2- | | | |
| (X, T, U, A B) | amine (G, D) | ylamino)-phenyl]-7H- | | | |
| | | pyrrolo[2,3- | | | |
| | | d]pyrimidin-4-ylamine | | | |
| trans-1-[4-(4-Amino-5- | (5,7-Dimethyl- | trans-1-(4-{4-Amino-5- | 14 | 2.30 min | 565 (M + H)⁺ |
| iodo-pyrrolo[2,3- | benzoxazol-2-yl)-[4- | [4-(5,7-dimethyl- | | (f) | |
| d]pyrimidin-7-yl)- | (4,4,5,5-tetramethyl- | benzoxazol-2- | | | |
| cyclohexyl]- | [1,3,2]dioxaborolan-2- | ylamino)-phenyl]- | | | |
| piperazine-2,5-dione | yl)-phenyl]-amine | pyrrolo[2,3- | | | |
| (A, T, J, W, Y, B) | (G, D) | d]pyrimidin-7-yl}- | | | |
| | | cyclohexyl)- | | | |
| | | piperazine-2,5-dione | | | |
| trans-1-[4-(4-Amino-5- | [2-Fluoro-4-(4,4,5,5- | trans-1-(4-{4-Amino-5- | 15 | 2.13 min (f) | 569 (M + H)⁺ |
| iodo-pyrrolo[2,3- | tetramethyl- | [3-fluoro-4-(5-methyl- | | | |
| d]pyrimidin-7-yl)- | [1,3,2]dioxaborolan-2- | benzoxazol-2- | | | |
| cyclohexyl]- | yl)-phenyl]-(5-methyl- | ylamino)-phenyl]- | | | |
| piperazine-2,5-dione | benzoxazol-2-yl)- | pyrrolo[2,3- | | | |
| (A, T, J, W, Y, B) | amine (G, D) | d]pyrimidine-7-yl}- | | | |
| | | cyclohexyl)- | | | |
| | | piperazine-2,5-dione, | | | |
| | | acetic acid salt | | | |
| trans-1-[4-(4-Amino-5- | (5-Chloro-benzoxazol- | trans-1-(4-{4-Amino-5- | 16 | 2.20 min (f) | 588 (M − H)⁺ |
| iodo-pyrrolo[2,3- | 2-yl)-[2-fluoro-4- | [4-(5-chloro- | | | |
| d]pyrimidin-7-yl)- | (4,4,5,5-tetramethyl- | benzoxazol-2- | | | |
| cyclohexyl]- | [1,3,2]dioxaborolan-2- | ylamino)-3-fluoro- | | | |
| piperazine-2,5-dione | yl)-phenyl]-amine | phenyl]-pyrrolo[2,3- | | | |
| (A, T, J, W, Y, B) | (G, D) | d]pyrimidin-7-yl}- | | | |
| | | cyclohexyl)- | | | |
| | | piperazine-2,5-dione | | | |
| trans-1-[4-(4-Amino-5- | (5,7-Dimethyl- | trans-1-(4-{4-Amino-5- | 17 | 2.37 min (f) | 583 (M + H)⁺ |
| iodo-pyrrolo[2,3- | benzoxazol-2-yl)-[2- | [4-(5,7-dimethyl- | | | |
| d]pyrimidin-7-yl)- | fluoro-4-(4,4,5,5- | benzoxazol-2- | | | |
| cyclohexyl]- | tetramethyl- | ylamino)-3-fluoro- | | | |
| piperazine-2,5-dione | [1,3,2]dioxaborolan-2- | phenyl]-pyrrolo[2,3- | | | |
| (A, T, J, W, Y, B) | yl)-phenyl]-amine | d]pyrimidin-7-yl}- | | | |
| | (G, D) | cyclohexyl)- | | | |
| | | piperazine-2,5-dione, | | | |
| | | acetic acid salt | | | |
| trans-1-[4-(4-Amino-5- | (5,7-Dimethyl- | trans-1-(4-{4-Amino-5- | 18 | 2.52 min (f) | 607 (M + H)⁺ |
| iodo-pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexyl]-4- | [1,3,2]dioxaborolan-2- | ylamino)-phenyl]- | | | |
| isopropyl-piperazine- | yl)-phenyl]-amine | pyrrolo[2,3- | | | |
| 2,5-dione | (G, D) | d]pyrimidin-7-yl}- | | | |
| (A, T, J, W, Y, B) | | cyclohexyl)-4- | | | |
| | | isopropyl-piperazine- | | | |
| | | 2,5-dione | | | |
| trans-1-[4-(2- | (5,7-Dimethyl- | trans-1-[4-(2- | 19 | 3.52 min (f) | 554 (M + H)⁺ |
| Cyclopropoxy-ethoxy)- | benzoxazol-2-yl)-[4- | Cyclopropoxy-ethoxy)- | | | |
| cyclohexyl]-3-iodo-1H- | (4,4,5,5-tetramethyl- | cyclohexyl]-3-[4-(5,7- | | | |
| pyrazolo[3,4- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (X, N, A) | (G, D) | 1H-pyrazolo[3,4- | | | |
| | | d]pyrimidin-4-ylamine | | | |
| trans-1-[4-(4-Amino-3- | (5-Chloro-7-methyl- | trans-1-(4-{4-Amino-3- | 20 | 3.28 min | 562 (M + H)⁺ |
| iodo-pyrazolo[3,4- | benzoxazol-2-yl)-[4- | [4-(5-chloro-7-methyl- | | (f) | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| trans-1-[4-(4-Amino-3- | (7-Chloro-5-methyl- | trans-1-(4-{4-Amino-3- | 21 | 3.35 min (f) | 562 (M + H)⁺ |
| iodo-pyrazolo[3,4- | benzoxazol-2-yl)-[4- | [4-(7-chloro-5-methyl- | | | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| trans-1-[4-(4-Amino-3- | (5-Chloro-benzoxazol- | trans-1-(4-{4-Amino-3- | 22 | 3.10 min | 566 (M + H)⁺ |
| iodo-pyrazolo[3,4- | 2-yl)-[2-fluoro-4- | [4-(5-chloro- | | (f) | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-3-fluoro- | | | |
| methyl-propan-2-ol | yl)-phenyl]-amine | phenyl]-pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| trans-1-[4-(4-Amino-5- | (5,7-Dimethyl- | trans-1-(4-{4-Amino-5- | 23 | 2.40 min (f) | 579 (M + H)⁺ |
| iodo-pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexyl]-4-methyl- | [1,3,2]dioxaborolan-2- | ylamino)-phenyl]- | | | |
| piperazine-2,5-dione | yl)-phenyl]-amine | pyrrolo[2,3- | | | |
| (A, T, J, W, Y, B) | (G, D) | d]pyrimidin-7-yl}- | | | |
| | | cyclohexyl)-4-methyl- | | | |
| | | piperazine-2,5-dione | | | |
| trans-1-[4-(2- | (5,7-Dimethyl- | trans-1-[4-(2- | 24 | 3.72 min (f) | 572 (M + H)⁺ |
| Cyclopropoxy-ethoxy)- | benzoxazol-2-yl)-[2- | Cyclopropoxy-ethoxy)- | | | |
| cyclohexyl]-3-iodo-1H- | fluoro-4-(4,4,5,5- | cyclohexyl]-3-[4-(5,7- | | | |
| pyrazolo[3,4- | tetramethyl- | dimethyl-benzoxazol- | | | |
| d]pyrimidin-4-ylamine | [1,3,2]dioxaborolan-2- | 2-ylamino)-3-fluoro- | | | |
| (X, N, A) | yl)-phenyl]-amine | phenyl]-1H- | | | |
| | (G, D) | pyrazolo[3,4- | | | |
| | | d]pyrimidin-4-ylamine | | | |
| trans-1-[4-(4-Amino-3- | (5-Chloro- | trans-1-(4-{4-Amino-3- | 25 | 3.02 min (f) | 548 (M + H)⁺ |
| iodo-pyrazolo[3,4- | benzoxazol-2-yl)-[4- | [4-(5-chloro- | | | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| trans-1-[4-(4-Amino-3- | (7-Chloro-5-fluoro- | trans-1-(4-{4-Amino-3- | 26 | 3.18 min (f) | 566 (M + H)⁺ |
| iodo-pyrazolo[3,4- | benzoxazol-2-yl)-[4- | [4-(7-chloro-5-fluoro- | | | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| trans-1-[4-(4-Amino-3- | (5-Fluoro-7-methyl- | trans-1-(4-{4-Amino-3- | 27 | 2.85 min (f) | 546 (M + H)⁺ |
| iodo-pyrazolo[3,4- | benzoxazol-2-yl)-[4- | [4-(5-fluoro-7-methyl- | | | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy)-2- | | | |
| | | methyl-propan-2-ol | | | |
| 3-Bromo-1-tert-butyl- | (5,7-Dimethyl- | 1-tert-Butyl-3-[4-(5,7- | 28 | 3.90 min (f) | 428 (M + H)⁺ |
| 1H-pyrazolo[3,4- | benzoxazol-2-yl)-[4- | dimethyl-benzoxazol- | | | |
| d]pyrimidin-4-ylamine | (4,4,5,5-tetramethyl- | 2-ylamino)-phenyl]- | | | |
| (preparation #27) | [1,3,2]dioxaborolan-2- | 1H-pyrazolo[3,4- | | | |
| | yl)-phenyl]-amine | d]pyrimidin-4-ylamine | | | |
| | (G, D) | | | | |
| 3-Bromo-1-tert-butyl- | (5,7-Dimethyl- | 1-tert-Butyl-3-[4-(5,7- | 29 | 4.12 min (f) | 446 (M + H)⁺ |
| 1H-pyrazolo[3,4- | benzoxazol-2-yl)-[2- | dimethyl-benzoxazol- | | | |
| d]pyrimidin-4-ylamine | fluoro-4-(4,4,5,5- | 2-ylamino)-3-fluoro- | | | |
| (preparation #27) | tetramethyl- | phenyl]-1H- | | | |
| | [1,3,2]dioxaborolan-2- | pyrazolo[3,4- | | | |
| | yl)-phenyl]-amine | d]pyrimidin-4-ylamine | | | |
| | (G, D) | | | | |
| 3-Iodo-1-[2-(2-methyl- | (5-Chloro-7-methyl- | 3-[4-(5-Chloro-7- | 30 | 2.58 min (f) | 500 (M + H)⁺ |
| imidazol-1-yl)-ethyl]- | benzoxazol-2-yl)-[4- | methyl-benzoxazol-2- | | | |
| 1H-pyrazolo[3,4- | (4,4,5,5-tetramethyl- | ylamino)-phenyl]-1-[2- | | | |
| d]pyrimidin-4-ylamine | [1,3,2]dioxaborolan-2- | (2-methyl-imidazol-1- | | | |
| (A) | yl)-phenyl]-amine | yl)-ethyl]-1H- | | | |
| | (G, D) | pyrazolo[3,4- | | | |
| | | d]pyrimidin-4-ylamine | | | |
| 1-(2-Imidazol-1-yl- | (5-Chloro-7-methyl- | 3-[4-(5-Chloro-7- | 31 | 2.57 min (f) | 486 (M + H)⁺ |
| ethyl)-3-iodo-1H- | benzoxazol-2-yl)-[4- | methyl-benzoxazol-2- | | | |
| pyrazolo[3,4- | (4,4,5,5-tetramethyl- | ylamino)-phenyl]-1-(2- | | | |
| d]pyrimidin-4-ylamine | [1,3,2]dioxaborolan-2- | imidazol-1-yl-ethyl)- | | | |
| (A) | yl)-phenyl]-amine | 1H-pyrazolo[3,4- | | | |
| | (G, D) | d]pyrimidin-4-ylamine | | | |
| 3-Iodo-1-[2-(1-methyl- | (5-Chloro-7-methyl- | 3-[4-(5-Chloro-7- | 32 | 2.87 min (f) | 532 (M + H)⁺ |
| 1H-imidazol-2- | benzoxazol-2-yl)-[4- | methyl-benzoxazol-2- | | | |
| ylsulfanyl)-ethyl]-1H- | (4,4,5,5-tetramethyl- | ylamino)-phenyl]-1-[2- | | | |
| pyrazolo[3,4- | [1,3,2]dioxaborolan-2- | (1-methyl-1H-imidazol- | | | |
| d]pyrimidin-4-ylamine | yl)-phenyl]-amine | 2-ylsulfanyl)-ethyl]-1H- | | | |
| (A) | (G, D) | pyrazolo[3,4- | | | |
| | | d]pyrimidin-4-ylamine | | | |
| 3-Iodo-1-(tetrahydro- | (5-Chloro-7-methyl- | 3-[4-(5-Chloro-7- | 33 | 3.20 min (f) | 474 (M − H)⁻ |
| pyran-4-yl)-1H- | benzoxazol-2-yl)-[4- | methyl-benzoxazol-2- | | | |
| pyrazolo[3,4- | (4,4,5,5-tetramethyl- | ylamino)-phenyl]-1- | | | |
| d]pyrimidin-4-ylamine | [1,3,2]dioxaborolan-2- | (tetrahydro-pyran-4- | | | |
| (A) | yl)-phenyl]-amine | yl)-1H-pyrazolo[3,4- | | | |
| | (G, D) | d]pyrimidin-4-ylamine | | | |
| 3-Iodo-1-(tetrahydro- | (5,7-Dimethyl- | 3-[4-(5,7-Dimethyl- | 34 | 3.00 min | 454 (M − H)⁻ |
| pyran-4-yl)-1H- | benzoxazol-2-yl)-[4- | benzoxazol-2- | | (f) | |
| pyrazolo[3,4- | (4,4,5,5-tetramethyl- | ylamino)-phenyl]-1- | | | |
| d]pyrimidin-4-ylamine | [1,3,2]dioxaborolan-2- | tetrahydro-pyran-4-yl)- | | | |
| (A) | yl)-phenyl]-amine | 1H-pyrazolo[3,4- | | | |
| | (G, D) | d]pyrimidin-4-ylamine | | | |
| 1-[2-(1H- | (5,7-Dimethyl- | 1-[2-(1H- | 35 | 2.77 min (f) | 514 (M − H)⁻ |
| Benzoimidazol-2-yl)- | benzoxazol-2-yl)-[4- | Benzoimidazol-2-yl)- | | | |
| ethyl]-3-iodo-1H- | (4,4,5,5-tetramethyl- | ethyl]-3-[4-(5,7- | | | |
| pyrazolo[3,4- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (A) | (G, D) | 1H-pyrazolo[3,4- | | | |
| | | d]pyrimidin-4-ylamine | | | |
| 1-Cyclohexyl-3-iodo- | (5,7-Dimethyl- | 1-Cyclohexyl-3-[4- | 36 | 4.10 min (f) | 452 (M − H)⁻ |
| 1H-pyrazolo[3,4- | benzoxazol-2-yl)-[4- | (5,7-dimethyl- | | | |
| d]pyrimidin-4-ylamine | (4,4,5,5-tetramethyl- | benzoxazol-2- | | | |
| (A) | [1,3,2]dioxaborolan-2- | ylamino)-phenyl]-1H- | | | |
| | yl)-phenyl]-amine | pyrazolo[3,4- | | | |
| | (G, D) | d]pyrimidin-4-ylamine | | | |
| 1-[2-(5,6-Dimethyl-1H- | (5,7-Dimethyl- | 1-[2-(5,6-Dimethyl-1H- | 37 | 2.90 min (f) | 542 (M − H)⁻ |
| benzimidazol-2-yl)- | benzoxazol-2-yl)-[4- | benzimidazol-2-yl)- | | | |
| ethyl]-3-iodo-1H- | (4,4,5,5-tetramethyl- | ethyl]-3-[4-(5,7- | | | |
| pyrazolo[3,4- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (A) | (G, D) | 1H-pyrazolo[3,4- | | | |
| | | d]pyrimidin-4-ylamine | | | |
| trans-4-[4-(4-Amino-5- | (5-Chloro-benzoxazol- | trans-4-(4-{4-Amino-5- | 38 | 2.37 min (f) | 537 (M − H)⁻ |
| iodo-pyrrolo[2,3- | 2-yl)-[2-fluoro-4- | [4-(5-chloro- | | | |
| d]pyrimidin-7-yl)- | (4,4,5,5-tetramethyl- | benzoxazol-2- | | | |
| cyclohexyl]-1-methyl- | [1,3,2]dioxaborolan-2- | ylamino)-3-fluoro- | | | |
| piperazin-2-one (A, T, | yl)-phenyl]-amine | phenyl]-pyrrolo[2,3- | | | |
| J (M)) | (G, D) | d]pyrimidin-7-yl}- | | | |
| | | cyclohexyl)-1-methyl- | | | |
| | | piperazin-2-one | | | |
| trans-4-[4-(4-Amino-5- | (5-Fluoro-benzoxazol- | trans-4-(4-{4-Amino-5- | 39 | 2.14 min (f) | 571 (M − H)⁻ |
| iodo-pyrrolo[2,3- | 2-yl)-[2-fluoro-4- | [3-fluoro-4-(5-fluoro- | | | |
| d]pyrimidin-7-yl)- | (4,4,5,5-tetramethyl- | benzoxazol-2- | | | |
| cyclohexyl]-1-methyl- | [1,3,2]dioxaborolan-2- | ylamino)-phenyl]- | | | |
| piperazin-2-one (A, T, | yl)-phenyl]-amine | pyrrolo[2,3- | | | |
| J (M)) | (G, D) | d]pyrimidin-7-yl}- | | | |
| | | cyclohexyl)-1-methyl- | | | |
| | | piperazin-2-one | | | |
| 4-(4-Amino-3-iodo- | (5,7-Dimethyl- | 4-{4-Amino-3-[4-(5,7- | 40 | 4.47 min (f) | 518 (M − H)⁻ |
| pyrazolo[3,4- | benzoxazol-2-yl)-[4- | dimethyl-benzoxazol- | | | |
| d]pyrimidin-1-yl)- | (4,4,5,5-tetramethyl- | 2-ylamino)-phenyl]- | | | |
| benzoic acid ethyl | [1,3,2]dioxaborolan-2- | pyrazolo[3,4- | | | |
| ester (I) | yl)-phenyl]-amine | d]pyrimidin-1-yl}- | | | |
| | (G, D) | benzoic acid ethyl | | | |
| | | ester | | | |
| 3-Iodo-1-(2- | (5,7-Dimethyl- | 3-[4-(5,7-Dimethyl- | 41 | 3.18 min | 456 (M − H)⁻ |
| isopropoxy-ethyl)-1H- | benzoxazol-2-yl)-[4- | benzoxazol-2- | | (f) | |
| pyrazolo[3,4- | (4,4,5,5-tetramethyl- | ylamino)-phenyl]-1-(2- | | | |
| d]pyrimidin-4-ylamine | [1,3,2]dioxaborolan-2- | isopropoxy-ethyl)-1H- | | | |
| (O) | yl)-phenyl]-amine | pyrazolo[3,4- | | | |
| | (G, D) | d]pyrimidin-4-ylamine | | | |
| 3-Iodo-1-(2-methoxy- | (5-Chloro-7-methyl- | 3-[4-(5-Chloro-7- | 42 | 3.00 min (f) | 448 (M − H)⁻ |
| ethyl)-1H- | benzoxazol-2-yl)-[4- | methyl-benzoxazol-2- | | | |
| pyrazolo[3,4- | (4,4,5,5-tetramethyl- | ylamino)-phenyl]-1-(2- | | | |
| d]pyrimidin-4-ylamine | [1,3,2]dioxaborolan-2- | methoxy-ethyl)-1H- | | | |
| (O) | yl)-phenyl]-amine (G, | pyrazolo[3,4- | | | |
| | D) | d]pyrimidin-4-ylamine | | | |
| 3-Iodo-1-(2-methoxy- | (5,7-Dimethyl- | 3-[4-(5,7-Dimethyl- | 43 | 2.83 min | 428 (M − H)⁻ |
| ethyl)-1H- | benzoxazol-2-yl)-[4- | benzoxazol-2- | | (f) | |
| pyrazolo[3,4- | (4,4,5,5-tetramethyl- | ylamino)-phenyl]-1-(2- | | | |
| d]pyrimidin-4-ylamine | [1,3,2]dioxaborolan-2- | methoxy-ethyl)-1H- | | | |
| (O) | yl)-phenyl]-amine | pyrazolo[3,4- | | | |
| | (G, D) | d]pyrimidin-4-ylamine | | | |
| 3-Iodo-1-(2-methoxy- | (5-Chloro-7-methyl- | 3-[4-(5-Chloro-7- | 44 | 3.08 min (f) | 494 (M + H)⁺ |
| 1-methoxymethyl- | benzoxazol-2-yl)-[4- | methyl-benzoxazol-2- | | | |
| ethyl)-1H- | (4,4,5,5-tetramethyl- | ylamino)phenyl]-1-(2- | | | |
| pyrazolo[3,4- | [1,3,2]dioxaborolan-2- | methoxy-1- | | | |
| d]pyrimidin-4-ylamine | yl)-phenyl]-amine | methoxymethyl-ethyl)- | | | |
| (O) | (G, D) | 1H-pyrazolo[3,4- | | | |
| | | d]pyrimidin-4-ylamine | | | |
| 3-Iodo-1-(2- | (5-Chloro-7-methyl- | 3-[4-(5-Chloro-7- | 45 | 3.33 min (f) | 478 (M + H)⁺ |
| isopropoxy-ethyl)-1H- | benzoxazol-2-yl)-[4- | methyl-benzoxazol-2- | | | |
| pyrazolo[3,4- | (4,4,5,5-tetramethyl- | ylamino)-phenyl]-1-(2- | | | |
| d]pyrimidin-4-ylamine | [1,3,2]dioxaborolan-2- | isopropoxy-ethyl)-1H- | | | |
| (O) | yl)-phenyl]-amine | pyrazolo[3,4- | | | |
| | (G, D) | d]pyrimidin-4-ylamine | | | |
| 1-[4-(4-Amino-5-iodo- | (5,7-Dimethyl- | cis-1-(4-{4-Amino-5-[4- | 46 | 2.68 min (f) | 579 (M + H)⁺ |
| pyrrolo[2,3- | benzoxazol-2-yl)-[4- | (5,7-dimethyl- | | | |
| d]pyrimidin-7-yl)- | (4,4,5,5-tetramethyl- | benzoxazol-2- | | | |
| cyclohexyl]-4-methyl- | [1,3,2]dioxaborolan-2- | ylamino)-phenyl]- | | | |
| [1,4]diazepan-5-one | yl)-phenyl]-amine | pyrrolo[2,3- | | | |
| (B, A, T, M, J) | (E, D) | d]pyrimidin-7-yl}- | | | |
| | | cyclohexyl)-4-methyl- | | | |
| | | [1,4]diazepan-5-one | | | |
| | | acetate salt | | | |
| 1-[4-(4-Amino-5-iodo- | (5,7-Dimethyl- | trans-1-(4-{4-Amino-5- | 47 | 2.47 min (f) | 579 (M + H)⁺ |
| pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexyl]-4-methyl- | [1,3,2]dioxaborolan-2- | ylamino)-phenyl]- | | | |
| [1,4]diazepan-5-one | yl)-phenyl]-amine | pyrrolo[2,3- | | | |
| (B, A, T, M, J) | (E, D) | d]pyrimidin-7-yl}- | | | |
| | | cyclohexyl)-4-methyl- | | | |
| | | [1,4]diazepan-5-one | | | |
| | | acetate salt | | | |
| 1-[4-(4-Amino-5-iodo- | (5,7-Dimethyl- | cis-1-(4-{4-Amino-5-[4- | 48 | 2.70 min (f) | 597 (M + H)⁺ |
| pyrrolo[2,3- | benzoxazol-2-yl)-[2- | (5,7-dimethyl- | | | |
| d]pyrimidin-7-yl)- | fluoro-4-(4,4,5,5- | benzoxazol-2- | | | |
| cyclohexyl]-4-methyl- | tetramethyl- | ylamino)-3-fluoro- | | | |
| [1,4]diazepan-5-one | [1,3,2]dioxaborolan-2- | phenyl]-pyrrolo[2,3- | | | |
| (B, A, T, M, J) | yl)-phenyl]-amine | d]pyrimidin-7-yl}- | | | |
| | (G, D) | cyclohexyl)-4-methyl- | | | |
| | | [1,4]diazepan-5-one | | | |
| | | acetate salt | | | |
| 1-[4-(4-Amino-5-iodo- | (5,7-Dimethyl- | trans-1-(4-{4-Amino-5- | 49 | 2.55 min (f) | 597 (M + H)⁺ |
| pyrrolo[2,3- | benzoxazol-2-yl)-[2- | [4-(5,7-dimethyl- | | | |
| d]pyrimidin-7-yl)- | fluoro-4-(4,4,5,5- | benzoxazol-2- | | | |
| cyclohexyl]-4-methyl- | tetramethyl- | ylamino)-3-fluoro- | | | |
| [1,4]diazepan-5-one | [1,3,2]dioxaborolan-2- | phenyl]-pyrrolo[2,3- | | | |
| (B, A, T, M, J) | yl)-phenyl]-amine | d]pyrimidin-7-yl}- | | | |
| | (G, D) | cyclohexyl)-4-methyl- | | | |
| | | [1,4]diazepan-5-one | | | |
| | | acetate salt | | | |
| trans-3-Iodo-1-(4- | (5,7-Dimethyl- | trans-3-[4-(5,7- | 50 | 4.38 min (f) | 468 (M + H)⁺ |
| methyl-cyclohexyl)- | benzoxazol-2-yl)-[4- | Dimethyl-benzoxazol- | | | |
| 1H-pyrazolo[3,4- | (4,4,5,5-tetramethyl- | 2-ylamino)-phenyl]-1- | | | |
| d]pyrimidin-4-ylamine | [1,3,2]dioxaborolan-2- | (4-methyl-cyclohexyl)- | | | |
| (A) | yl)-phenyl]-amine | 1H-pyrazolo[3,4- | | | |
| | (E, D) | d]pyrimidin-4-ylamine | | | |
| cis-3-Iodo-1-(4-methyl- | (5,7-Dimethyl- | cis-3-[4-(5,7-Dimethyl- | 51 | 4.33 min (f) | 468 (M + H)⁺ |
| cyclohexyl)-1H- | benzoxazol-2-yl)-[4- | benzoxazol-2- | | | |
| pyrazolo[3,4- | (4,4,5,5-tetramethyl- | ylamino)-phenyl]-1-(4- | | | |
| d]pyrimidin-4-ylamine | [1,3,2]dioxaborolan-2- | methyl-cyclohexyl)- | | | |
| (A) | yl)-phenyl]-amine | 1H-pyrazolo[3,4- | | | |
| | (E, D) | d]pyrimidin-4-ylamine | | | |
| 1-Bicyclo[2.2.1]hept-2- | (5,7-Dimethyl- | 1-Bicyclo[2.2.1]hept-2- | 52 | 4.35 min (f) | 466 (M + H)⁺ |
| yl-3-iodo-1H- | benzoxazol-2-yl)-[4- | yl-3-[4-(5,7-dimethyl- | | | |
| pyrazolo[3,4- | (4,4,5,5-tetramethyl- | benzoxazol-2- | | | |
| d]pyrimidin-4-ylamine | [1,3,2]dioxaborolan-2- | ylamino)-phenyl]-1H- | | | |
| (A) | yl)-phenyl]-amine | pyrazolo[3,4- | | | |
| | (E, D) | d]pyrimidin-4-ylamine | | | |
| 1-(4,4-Dimethyl- | (5,7-Dimethyl- | 3-[4-(5,7-Dimethyl- | 53 | 4.57 min (f) | 482 (M + H)⁺ |
| cyclohexyl)-3-iodo-1H- | benzoxazol-2-yl)-[4- | benzoxazol-2- | | | |
| pyrazolo[3,4- | (4,4,5,5,-tetramethyl- | ylamino)-phenyl]-1- | | | |
| d]pyrimidin-4-ylamine | [1,3,2]dioxaborolan-2- | (4,4-dimethyl- | | | |
| (A) | yl)-phenyl]-amine | cyclohexyl)-1H- | | | |
| | (E, D) | pyrazolo[3,4- | | | |
| | | d]pyrimidin-4-ylamine | | | |
| 1-Bicyclo[2.2.1]hept-5- | (5,7-Dimethyl- | 1-Bicyclo[2.2.1]hept-5- | 54 | 4.08 min (f) | 464 (M + H)⁺ |
| en-2-yl-3-iodo-1H- | benzoxazol-2-yl)-[4- | en-2-yl-3-[4-(5,7- | | | |
| pyrazolo[3,4- | (4,4,5,5-tetramethyl- | dimethyl-benzoxazol- | | | |
| d]pyrimidin-4-ylamine | [1,3,2]dioxaborolan-2- | 2-ylamino)-phenyl]- | | | |
| (A) | yl)-phenyl]-amine | 1H-pyrazolo[3,4- | | | |
| | (E, D) | d]pyrimidin-4-ylamine | | | |
| 3-Iodo-1-(4- | (5,7-Dimethyl- | trans 3[4-(5,7- | 55 | 3.93 min (f) | 522 (M + H)⁺ |
| trifluoromethyl- | benzoxazol-2-yl)-[4- | Dimethyl-benzoxazol- | | | |
| cyclohexyl)-1H- | (4,4,5,5-tetramethyl- | 2-ylamino)-phenyl]-1- | | | |
| pyrazolo[3,4- | [1,3,2]dioxaborolan-2- | (4-trifluoromethyl- | | | |
| d]pyrimidin-4-ylamine | yl)-phenyl]-amine | cyclohexyl)-1H- | | | |
| (A) | (E, D) | pyrazolo[3,4- | | | |
| | | d]pyrimidin-4-ylamine | | | |
| 4-[4-(4-Amino-5-iodo- | (5,7-Dimethyl- | trans-4-(4-{4-Amino-5- | 56 | 2.62 min (f) | 579 (M + H)⁺ |
| pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexyl]-1-methyl- | [1,3,2]dioxaborolan-2- | ylamino)-phenyl]- | | | |
| [1,4]diazepan-2-one | yl)-phenyl]-amine | pyrrolo[2,3- | | | |
| (B, A, T, Z, M, J) | (E, D) | d]pyrimidin-7-yl}- | | | |
| | | cyclohexyl)-1-methyl- | | | |
| | | [1,4]diazepan-2-one | | | |
| 3-Iodo-1-(4-isopropyl- | (5,7-Dimethyl- | cis-3-[4-(5,7-Dimethyl- | 57 | 5.27 min (f) | 496 (M + H)⁺ |
| cyclohexyl)-1H- | benzoxazol-2-yl)-[4- | benzoxazol-2- | | | |
| pyrazolo[3,4- | (4,4,5,5-tetramethyl- | ylamino)-phenyl]-1-(4- | | | |
| d]pyrimidin-4-ylamine | [1,3,2]dioxaborolan-2- | isopropyl-cyclohexyl)- | | | |
| (A) | yl)-phenyl]-amine | 1H-pyrazolo[3,4- | | | |
| | (E, D) | d]pyrimidin-4-ylamine | | | |
| trans-4-[4-(4-Amino-3- | (5,7-Dimethyl- | trans-4-(4-{4-Amino-3- | 58 | 2.32 min | 566 (M + H)⁺ |
| iodo-pyrazolo[3,4- | benzoxazol-2-yl)-[4- | [4-(5,7-dimethyl- | | (f) | |
| d]pyrimidin-1-yl)- | (4,4,5,5-tetramethyl- | benzoxazol-2- | | | |
| cyclohexyl]- | [1,3,2]dioxaborolan-2- | ylamino)-phenyl]- | | | |
| [1,4]diazepan-2-one | yl)-phenyl]-amine | pyrazolo[3,4- | | | |
| (A, T, Z, J) | (E, D) | d]pyrimidin-1-yl}- | | | |
| | | cyclohexyl)- | | | |
| | | [1,4]diazepan-2-one | | | |
| 1-[4-(4-Amino-3-iodo- | (5,7-Dimethyl- | trans-1-(4-{4-Amino-3- | 59 | 2.35 min (f) | 584 (M + H)⁺ |
| pyrazolo[3,4- | benzoxazol-2-yl)-[2- | [4-(5,7-dimethyl- | | | |
| d]pyrimidin-1-yl)- | fluoro-4-(4,4,5,5- | benzoxazol-2- | | | |
| cyclohexyl]- | tetramethyl- | ylamino)-3-fluoro- | | | |
| [1,4]diazepan-5-one | [1,3,2]dioxaborolan-2- | phenyl]-pyrazolo[3,4- | | | |
| (A, T, J) | yl)-phenyl]-amine | d]pyrimidin-1-yl}- | | | |
| | (G, D) | cyclohexyl)- | | | |
| | | [1,4]diazepan-5-one | | | |
| 3-[4-(4-Amino-3-iodo- | (5-Chloro-7-methyl- | trans-3-(4-{4-Amino-3- | 60 | 2.83 min (f) | 560 (M + H)⁺ |
| pyrazolo[3,4- | benzoxazol-2-yl)-[4- | [4-(5-chloro-7-methyl- | | | |
| d]pyrimidin-1-yl)- | (4,4,5,5-tetramethyl- | benzoxazol-2- | | | |
| cyclohexyl]-oxazolidin- | [1,3,2]dioxaborolan-2- | ylamino)-phenyl]- | | | |
| 2-one (A, T, J, AA) | yl)-phenyl]-amine | pyrazolo[3,4- | | | |
| | (E, D) | d]pyrimidin-1-yl}- | | | |
| | | cyclohexyl)-oxazolidin- | | | |
| | | 2-one | | | |
| 3-[4-(4-Amino-3-iodo- | (5,7-Dimethyl- | trans-3-(4-{4-Amino-3- | 61 | 2.73 min (f) | 539 (M + H)⁺ |
| pyrazolo[3,4- | benzoxazol-2-yl)-[4- | [4-(5,7-dimethyl- | | | |
| d]pyrimidin-1-yl)- | (4,4,5,5-tetramethyl- | benzoxazol-2- | | | |
| cyclohexyl]-oxazolidin- | [1,3,2]dioxaborolan-2- | ylamino)-phenyl]- | | | |
| 2-one (A, T, J, AA) | yl)-phenyl]-amine | pyrazolo[3,4- | | | |
| | (E, D) | d]pyrimidin-1-yl}- | | | |
| | | cyclohexyl)-oxazolidin- | | | |
| | | 2-one | | | |
| trans-1-[4-(4-Amino-3- | (5-Chloro-7-methyl- | trans-1-(4-{4-Amino-3- | 62 | 2.53 min (f) | 615 (M + H)⁺ |
| iodo-pyrazolo[3,4- | benzoxazol-2-yl)-[4- | [4-(5-chloro-7-methyl | | | |
| d]pyrimidin-1-yl)- | (4,4,5,5-tetramethyl- | benzoxazol-2- | | | |
| cyclohexyl]-5,5- | [1,3,2]dioxaborolan-2- | ylamino)-phenyl]- | | | |
| dimethyl-piperazine- | yl)-phenyl]-amine | pyrazolo[3,4- | | | |
| 2,3-dione (A, T, J, AA) | (E, D) | d]pyrimidin-1-yl}- | | | |
| | | cyclohexyl)-5,5- | | | |
| | | dimethyl-piperazine- | | | |
| | | 2,3-dione | | | |
| 1-[4-(4-Amino-3-iodo- | (5-Chloro-7-methyl- | trans-1-(4-{4-Amino-3- | 63 | 2.97 min (f) | 587 (M + H)⁺ |
| pyrazolo[3,4- | benzoxazol-2-yl)-[4- | [4-(5-chloro-7-methyl- | | | |
| d]pyrimidin-1-yl)- | (4,4,5,5-tetramethyl- | benzoxazol-2- | | | |
| cyclohexyl]-4,4- | [1,3,2]dioxaborolan-2- | ylamino)-phenyl]- | | | |
| dimethyl-imidazolidin- | yl)-phenyl]-amine | pyrazolo[3,4- | | | |
| 2-one (A, T, J, AA) | (E, D) | d]pyrimidin-1-yl}- | | | |
| | | cyclohexyl)-4,4- | | | |
| | | dimethyl-imidazolidin- | | | |
| | | 2-one | | | |
| trans-1-[4-(4-Amino-5- | (5-Chloro-7-methyl- | trans-1-(4-{4-Amino-5- | 64 | 2.42 min (f) | 585 (M + H)⁺ |
| iodo-pyrrolo[2,3- | benzoxazol-2-yl)-[4- | [4-(5-chloro-7-methyl- | | | |
| d]pyrimidin-7-yl)- | (4,4,5,5-tetramethyl- | benzoxazol-2- | | | |
| cyclohexyl]- | [1,3,2]dioxaborolan-2- | ylamino)-phenyl]- | | | |
| piperazine-2,5-dione | yl)-phenyl]-amine | pyrrolo[2,3- | | | |
| (A, T, J, W, Y, B) | (G, D) | d]pyrimidin-7-yl}- | | | |
| | | cyclohexyl)- | | | |
| | | piperazine-2,5-dione | | | |
| 4-(4-Amino-3-iodo- | (5-Chloro-7-methyl- | 4-{4-Amino-3-[4-(5- | 65 | 3.00 min (f) | 486 (M − H)⁻ |
| pyrazolo-[3,4- | benzoxazol-2-yl)-[4- | chloro-7-methyl- | | | |
| d]pyrimidin-1-yl)- | (4,4,5,5-tetramethyl- | benzoxazol-2- | | | |
| cyclohexanone (A, T) | [1,3,2]dioxa-borolan-2- | ylamino)-phenyl]- | | | |
| | yl)-phenyl]amine | pyrazolo[3,4- | | | |
| | (F, H, G, D) | d]pyrimidin-1-yl}- | | | |
| | | cyclohexanone | | | |
| cis-3-Iodo-1-(4- | (5,7-Dimethyl- | cis-3-[4-(5,7-Dimethyl | 66 | 16.13 min | 521 (M + H)⁺ |
| [1,2,4]triazol-1-yl-cyclohexyl)- | benzoxazol-2-yl)-[4- | benzoxazol-2- | | (c) | |
| 1H- | (4,4,5,5-tetramethyl | ylamino)-phenyl]-1-(4- | | | |
| pyrazolo[3,4- | [1,3,2]dioxaborolan-2- | [1,2,4]triazol- | | | |
| d]pyrimidin-4-ylamine | yl)-phenyl]-amine | 1-yl-cyclohexyl)-1H | | | |
| (A, T, U, V) | (G, D) | pyrazolo[3,4- | | | |
| | | d]pyrimidin-4-ylamine | | | |
| trans-3-Iodo-1-(4- | (5,7-Dimethyl- | trans-3-[4-(5,7- | 67 | 16.28 min | 521 (M + H)⁺ |
| [1,2,4]triazol-1-yl-cyclohexyl)- | benzoxazol-2-yl)-[4- | Dimethyl benzoxazol- | | (c) | |
| 1H- | (4,4,5,5-tetramethyl | 2-ylamino)-phenyl]-1- | | | |
| pyrazolo[3,4- | [1,3,2]dioxaborolan-2- | (4-[1,2,4]triazol- | | | |
| d]pyrimidin-4-ylamine | yl)-phenyl]-amine | 1-yl-cyclohexyl)-1H | | | |
| (A, T, U, V) | (G, D) | pyrazolo[3,4- | | | |
| | | d]pyrimidin-4-ylamine | | | |
| trans-3-Iodo-1-(4- | (5,7-Dimethyl- | trans-3-[4-(5,7- | 68 | 18.54 min | δ (DMSO-d₆) |
| pyrazol-1-yl- | benzoxazol-2-yl)-[4- | Dimethyl-benzoxazol- | | (c) | 10.86, 8.26, |
| cyclohexyl)-1H- | (4,4,5,5-tetramethyl | 2-ylamino)-phenyl]-1- | | | 7.95, 7.81, |
| pyrazolo[3,4- | [1,3,2]dioxaborolan-2- | (4-pyrazol-1-yl-cyclohexyl)- | | | 7.67, 7.45, |
| d]pyrimidin-4-ylamine | yl)-phenyl]-amine | 1H- | | | 7.13, 6.80, |
| (A, T, U, V) | (G, D) | pyrazolo[3,4-d]- | | | 6.26, 4.81, |
| | | pyrimidin-4-ylamine | | | 4.37, 2.40, |
| | | | | | 2.34, 2.23 |
| trans-3-Iodo-1-[4- | (5,7-Dimethyl- | trans-3-[4-(5,7- | 69 | 19.97 min | δ (DMSO-d₆) |
| (pyrazol-1-yloxy)- | benzoxazol-2-yl)-[2- | Dimethyl-benzoxazol- | | (c) | 10.59, 8.51, |
| cyclohexyl]-1H- | fluoro-4-(4,4,5,5- | 2-ylamino)-3-fluoro- | | | 8.27, 7.81, |
| pyrazolo[3,4- | tetramethyl- | phenyl]-1-[4-(pyrazol- | | | 7.55, 7.50, |
| d]pyrimidin-4-ylamine | [1,3,2]dioxaborolan-2- | 1-yloxy)-cyclohexyl]- | | | 7.30, 7.13, |
| (A, T, U, V) | yl)-phenyl]-amine | 1H-pyrazolo[3,4- | | | 6.83, 6.28, |
| | (F, H, G, D) | d]pyrimidin-4-ylamine | | | 4.78, 4.50, |
| | | | | | 2.40, 2.33, |
| | | | | | 1.96 |
| trans-3-Iodo-1-(4- | (5,7-Dimethyl- | trans-3-[4-(5,7- | 70 | 19.83 min | δ (DMSO-d₆) |
| [1,2,3]triazol-2-yl- | benzoxazol-2-yl)-[2- | Dimethyl-benzoxazol- | | (c) | 10.62, 8.56, |
| cyclohexyl)-1H- | fluoro-4-(4,4,5,5- | 2-ylamino)-3-fluoro- | | | 8.29, 7.82, |
| pyrazolo[3,4- | tetramethyl- | phenyl]-1-(4- | | | 7.58, 7.14, |
| d]pyrimidin-4-ylamine | [1,3,2]dioxaborolan-2- | [1,2,3]triazol-2-yl- | | | 6.83, 4.86, |
| (A, T, U, V) | yl)-phenyl]-amine | cyclohexyl)-1H- | | | 4.74, 2.43, |
| | (F, H, G, D) | pyrazolo[3,4- | | | 2.36, 2.27, |
| | | d]pyrimidin-4-ylamine | | | 2.15 |
| trans-3-Iodo-1- | (5,7-Dimethyl- | trans-3-[4-(5,7- | 71 | 17.23 min | δ (DMSO-d₆) |
| (4[1,2,3]triazol-1-yl- | benzoxazol-2-yl)-[2- | Dimethyl-benzoxazol- | | (c) | 10.53, 8.47, |
| cyclohexyl)-1H- | fluoro-4-(4,4,5,5- | 2-ylamino)-3-fluoro- | | | 8.20, 7.69, |
| pyrazolo[3,4- | tetramethyl- | phenyl]-1-(4- | | | 7.05, 6.74, |
| d]pyrimidin-4-ylamine | [1,3,2]dioxaborolan-2- | [1,2,3]triazol-1-yl- | | | 4.84, 4.69, |
| (A, T, U, V) | yl)-phenyl]-amine | cyclohexyl)-1H- | | | 2.43, 2.36, |
| | (F, H, G, D) | pyrazolo[3,4- | | | 2.27, 2.15 |
| | | d]pyrimidin-4-ylamine | | | |
| trans-1-[4-(3,5- | (5,7-Dimethyl- | trans-3-[4-(5,7- | 72 | 2.51 min | 549 (M + H)⁺ |
| Dimethyl-[1,2,4]triazol- | benzoxazol-2-yl)-[4- | Dimethyl-benzoxazol- | | (e) | |
| 1-yl)-cyclohexyl]-3- | (4,4,5,5-tetramethyl | 2-ylamino)-phenyl]-1- | | | |
| iodo-1H-pyrazolo[3,4- | [1,3,2]dioxaborolan-2- | [4-(3,5-dimethyl- | | | |
| d]pyrimidin-4-ylamine | yl)-phenyl]-amine | [1,2,4]triazol-1-yl)- | | | |
| (A, T, U, V) | (G, D) | cyclohexyl]-1H- | | | |
| | | pyrazolo[3,4- | | | |
| | | d]pyrimidin-4-ylamine | | | |
| 1-[4-(2-Fluoro-ethyl)- | (5,7-Dimethyl- | 3-[4-(5,7-Dimethyl- | 73 | 10.01 min | δ (DMSO-d₆) |
| cyclohexyl]-3-iodo-1H- | benzoxazol-2-yl)-[4- | benzoxazol-2- | | (a) | 10.89, 8.26, |
| pyrazolo[3,4- | (4,4,5,5-tetramethyl | ylamino)-phenyl]-1-[4- | | | 7.94, 7.68, |
| d]pyrimidin-4- | [1,3,2]dioxaborolan-2- | (2-fluoro-ethyl)- | | | 7.12, 6.80, |
| -ylamine (A) | yl)-phenyl]-amine | cyclohexyl]-1H- | | | 4.94, 4.83, |
| | (G, D) | pyrazolo[3,4- | | | 4.68, 4.62, |
| | | d]pyrimidin-4-ylamine | | | 2.40, 2.34 |
| 1-(4- | (5,7-Dimethyl- | 1-(4- | 74 | 3.14 min (f) | 466 (M + H)⁺ |
| Dicyclopropylmethyl- | benzoxazol-2-yl)-[4- | Dicyclopropylmethyl- | | | |
| cyclohexyl)-3-iodo-1H- | (4,4,5,5-tetramethyl | cyclohexyl)-3-[4-(5,7- | | | |
| pyrazolo[3,4- | [1,3,2]dioxaborolan-2- | dimethyl-benzoxazol- | | | |
| d]pyrimidin-4-ylamine | yl)-phenyl]-amine | 2-ylamino)-phenyl]- | | | |
| (A) | (G, D) | 1H-pyrazolo[3,4- | | | |
| | | d]pyrimidin-4-ylamine | | | |
| trans-3-Iodo-1-[4-(2- | (5-Chloro-benzoxazol- | trans-3-[4-(5-Chloro- | 75 | 6.79 min | 532 (M − H)⁻ |
| methoxy-ethoxy)- | 2-yl)-[4-(4,4,5,5- | benzoxazol-2- | | (g) | |
| cyclohexyl]- | tetramethyl- | ylamino)-phenyl]-1-[4- | | | |
| 1Hpyrazolo[3,4- | [1,3,2]dioxaborolan-2- | (2-methoxy-ethoxy)- | | | |
| d]pyrimidin-4-ylamine | yl)-phenyl]-amine | cyclohexyl]-1H- | | | |
| (A) | (F, H, G, D) | pyrazolo[3,4- | | | |
| | | d]pyrimidin-4-ylamine | | | |
| trans-3-Iodo-1-[4-(2- | (5-Chloro-benzoxazol- | trans-3-[4-(5-Chloro- | 76 | 6.84 min | 550 (M − H)⁻ |
| methoxy-ethoxy)- | 2-yl)-[2-fluoro-4- | benzoxazol-2- | | (g) | |
| cyclohexyl]- | (4,4,5,5-tetramethyl- | ylamino)-3-fluoro- | | | |
| 1Hpyrazolo[3,4- | [1,3,2]di- | phenyl]-1-[4-(2- | | | |
| d]pyrimidin-4-ylamine | oxaborolan-2-yl)- | methoxy-ethoxy)- | | | |
| (A) | phenyl]-amine | cyclohexyl)]-1H- | | | |
| | (F, H, G, D) | pyrazolo[3,4- | | | |
| | | d]pyrimidin-4-ylamine | | | |
| trans-4-[4-(4-Amino-3- | (7-Chloro-5-methyl- | trans-4-(4-{4-Amino-3- | 77 | 2.93 min (f) | 600 (M + H)⁺ |
| iodo-pyrazolo[3,4- | benzoxazol-2-yl)-[4- | [4-(7-chloro-5-methyl- | | | |
| d]pyrimidin-1-yl)- | (4,4,5,5-tetramethyl- | benzoxazol-2- | | | |
| cyclohexyl]-6,6- | [1,3,2]dioxaborolan-2- | ylamino)-phenyl]- | | | |
| dimethyl-piperazin-2- | yl)-phenyl]-amine | pyrazolo[3,4- | | | |
| one (A, T, J) | (F, H, G, D) | d]pyrimidin-1-yl}- | | | |
| | | cyclohexyl)-6,6- | | | |
| | | dimethyl-piperazin-2- | | | |
| | | one | | | |
| trans-4-[4-(4-Amino-3- | (5-Fluoro-7-methyl- | trans-4-(4-{4-Amino-3- | 78 | 16.66 min | δ (DMSO-d₆) |
| iodo-pyrazolo[3,4- | benzoxazol-2-yl)-[4- | [4-(5-fluoro-7-methyl- | | (c) | 11.04, 8.23, |
| d]pyrimidin-1-yl)- | (4,4,5,5-tetramethyl- | benzoxazol-2- | | | 7.91, 7.68, |
| cyclohexyl]-6,6- | [1,3,2]dioxaborolan-2- | ylamino)-phenyl]- | | | 7.09, 6.92, |
| dimethyl-piperazin-2- | yl)-phenyl]-amine | pyrazolo [3,4- | | | 4.66, 3.04, |
| one (A, T, J) | (F, H, G, D) | d]pyrimidin-1-yl}- | | | 2.46, 2.00 |
| | | cyclohexyl)-6,6- | | | 1.52, 1.16 |
| | | dimethyl-piperazin-2- | | | |
| | | one | | | |
| 3-Iodo-1-(tetrahydro- | 5-Chloro-2-[4-(4,4,5,5- | 2-{4-[4-Amino-1- | 79 | 2.28 min (f) | 531 (M − H)⁻ |
| pyran-4-yl)-1H- | tetramethyl- | (tetrahydro-pyran-4- | | | |
| pyrazolo[3,4- | [1,3,2]dioxaborolan-2- | yl)-1H-pyrazolo[3,4- | | | |
| d]pyrimidin-4-ylamine | yl)-phenylamino]- | d]pyrimidin-3-yl]- | | | |
| (A) | benzoxazole-7- | phenylamino}-5- | | | |
| | carboxylic acid | chloro-benzoxazole-7- | | | |
| | dimethylamide | carboxylic acid | | | |
| | (F, H, G, P, Q, D) | dimethylamide | | | |
| trans-3-Iodo-1-(4- | 5-Chloro-2-[4-(4,4,5,5- | trans-2-{4-[4-Amino-1- | 80 | 16.65 min | δ (DMSO-d₆) |
| morpholin-4-yl- | tetramethyl- | (4-morpholin-4-yl- | | (c) | 11.24, 8.23, |
| cyclohexyl)-1H- | [1,3,2]dioxaborolan-2- | cyclohexyl)-1H- | | | 7.90, 7.66 |
| pyrazolo[3,4- | yl)-phenylamino]- | pyrazolo[3,4- | | | 7.19, 4.66, |
| d]pyrimidin-4-yl amine | benzoxazole-7- | d]pyrimidin-3-yl]- | | | 3.59, 3.05, |
| (A, T, J) | carboxylic acid | phenylamino}-5- | | | 2.95, 2.36, |
| | dimethylamide | chloro-benzo-oxazole- | | | 2.00, 1.47 |
| | (F, H, G, P, Q, D) | 7-carboxylic acid | | | |
| | | dimethylamide | | | |
| trans-3-Iodo-1-(4- | (5,7-Dimethyl- | trans-3-[4-(5,7- | 81 | 20.63 min | 483 (M + H)⁺ |
| methoxy-cyclohexyl)- | benzoxazol-2-yl)-[4- | Dimethyl-benzoxazol- | | (d) | |
| 1H-pyrazolo[3,4- | (4,4,5,5-tetramethyl- | 2-ylamino)-phenyl]-1- | | | |
| d]pyrimidin-4-ylamine | [1,3,2]dioxaborolan-2- | (4-methoxy- | | | |
| (A) | yl)-phenyl]-amine | cyclohexyl)-1H- | | | |
| | (G, D) | pyrazolo[3,4- | | | |
| | | d]pyrimidin-4-ylamine | | | |
| cis-3-Iodo-1-(4- | (5,7-Dimethyl- | cis-3-[4-(5,7-Dimethyl- | 82 | 21.25 min | 483 (M + H)⁺ |
| methoxy-cyclohexyl)- | benzoxazol-2-yl)-[4- | benzoxazol-2- | | (d) | |
| 1H-pyrazolo[3,4- | (4,4,5,5-tetramethyl- | ylamino)-phenyl]-1-(4- | | | |
| d]pyrimidin-4-ylamine | [1,3,2]dioxaborolan-2- | methoxy-cyclohexyl)- | | | |
| (A) | yl)-phenyl]-amine | 1H-pyrazolo[3,4- | | | |
| | (G, D) | d]pyrimidin-4-ylamine | | | |
| trans-3-Iodo-1-[4-(2- | (5,7-Dimethyl- | trans-3-[4-(5,7- | 83 | 10.94 min | 528 (M + H)⁺ |
| methoxy-ethoxy)- | benzoxazol-2-yl)-[4- | Dimethyl-benzoxazol- | | (a) | |
| cyclohexyl]-1H- | (4,4,5,5-tetramethyl- | 2-ylamino)-phenyl]-1- | | | |
| pyrazolo[3,4- | [1,3,2]dioxaborolan-2- | [4-(2-methoxy-ethoxy)- | | | |
| d]pyrimidin-4-ylamine | yl)-phenyl]-amine | cyclohexyl]-1H- | | | |
| (A) | (G, D) | pyrazolo[3,4- | | | |
| | | d]pyrimidin-4-ylamine | | | |
| cis-3-Iodo-1-[4-(2- | (5,7-Dimethyl- | cis-3-[4-(5,7-Dimethyl- | 84 | 11.33 min | 528 (M + H)⁺ |
| methoxy-ethoxy)- | benzoxazol-2-yl)-[4- | benzoxazol-2- | | (a) | |
| cyclohexyl]-1H- | (4,4,5,5-tetramethyl- | ylamino)-phenyl]-1-[4- | | | |
| pyrazolo[3,4- | [1,3,2]dioxaborolan-2- | (2-methoxy-ethoxy)- | | | |
| d]pyrimidin-4-ylamine | yl)-phenyl]-amine | cyclohexyl]-1H- | | | |
| (A) | (G, D) | pyrazolo[3,4- | | | |
| | | d]pyrimidin-4-ylamine | | | |
| trans-3-Iodo-1-[4-(2- | (5,7-Dimethyl- | trans-3-[4-(5,7- | 85 | 11.10 min | 546 (M + H)⁺ |
| methoxy-ethoxy)- | benzoxazol-2-yl)-[2- | Dimethyl-benzoxazol- | | (a) | |
| cyclohexyl]-1H- | fluoro-4-(4,4,5,5- | 2-ylamino)-3-fluoro- | | | |
| pyrazolo[3,4- | tetramethyl- | phenyl]-1-[4-(2- | | | |
| d]pyrimidin-4-ylamine | [1,3,2]dioxaborolan-2- | methoxy-ethoxy)- | | | |
| (A) | yl)-phenyl]-amine | cyclohexyl]-1H- | | | |
| | (G, D) | pyrazolo[3,4- | | | |
| | | d]pyrimidin-4-ylamine | | | |
| 1-Cycloheptyl-3-iodo- | (5,7-Dimethyl- | 1-Cycloheptyl-3-[4- | 86 | 13.92 min | 566 (M + H)⁺ |
| 1H-pyrazolo[3,4- | benzoxazol-2-yl)-[4- | (5,7-dimethyl- | | (a) | |
| d]pyrimidin-4-ylamine | (4,4,5,5-tetramethyl- | benzoxazol-2- | | | |
| (O) | [1,3,2]dioxaborolan-2- | ylamino)-phenyl]-1H- | | | |
| | yl)-phenyl]-amine | pyrazolo[3,4- | | | |
| | (G, D) | d]pyrimidin-4-ylamine | | | |
| 1-Cycloheptyl-3-iodo- | (5,7-Dimethyl- | 1-Cycloheptyl-3-[4- | 87 | 14.29 min | 486 (M + H)⁺ |
| 1H-pyrazolo[3,4- | benzoxazol-2-yl)-[2- | (5,7-dimethyl- | | (h) | |
| d]pyrimidin-4-ylamine | fluoro-4-(4,4,5,5- | benzoxazol-2- | | | |
| (O) | tetramethyl- | ylamino)-3-fluoro- | | | |
| | [1,3,2]dioxaborolan-2- | phenyl]-1H- | | | |
| | yl)-phenyl]-amine | pyrazolo[3,4- | | | |
| | (G, D) | d]pyrimidin-4-ylamine | | | |
| 1-sec-Butyl-3-iodo-1H- | (5,7-Dimethyl- | 1-sec-Butyl-3-[4-(5,7- | 88 | 12.09 min | 428 (M + H)⁺ |
| pyrazolo[3,4- | benzoxazol-2-yl)-[4- | dimethyl-benzoxazol- | | (a) | |
| d]pyrimidin-4-ylamine | (4,4,5,5-tetramethyl- | 2-ylamino)-phenyl]- | | | |
| (O) | [1,3,2]dioxaborolan-2- | 1H-pyrazolo[3,4- | | | |
| | yl)-phenyl]-amine | d]pyrimidin-4-ylamine | | | |
| | (G, D) | | | | |
| 3-Iodo-1-(1-methyl- | (5,7-Dimethyl- | 3-[4-(5,7-Dimethyl- | 89 | 12.77 min | 442 (M + H)⁺ |
| butyl)-1H- | benzoxazol-2-yl)-[4- | benzoxazol-2- | | (a) | |
| pyrazolo[3,4- | (4,4,5,5-tetramethyl- | ylamino)-phenyl]-1-(1- | | | |
| d]pyrimidin-4-ylamine | [1,3,2]dioxaborolan-2- | methyl-butyl)-1H- | | | |
| (O) | yl)-phenyl]-amine | pyrazolo[3,4- | | | |
| | (G, D) | d]pyrimidin-4-ylamine | | | |
| 1-(2-Fluoro-1- | (5,7-Dimethyl- | 3-[4-(5,7-Dimethyl- | 90 | 16.63 min | 450 (M + H)⁺ |
| fluoromethyl-ethyl)-3- | benzoxazol-2-yl)-[4- | benzoxazol-2- | | (d) | |
| iodo-1H-pyrazolo[3,4- | (4,4,5,5-tetramethyl- | ylamino)-phenyl]-1-(2- | | | |
| d]pyrimidin-4-ylamine | [1,3,2]dioxaborolan-2- | fluoro-1-fluoromethyl- | | | |
| (O) | yl)-phenyl]-amine | ethyl)-1H- | | | |
| | (G, D) | pyrazolo[3,4- | | | |
| | | d]pyrimidin-4-ylamine | | | |
| 1-sec-Butyl-3-iodo-1H- | (5,7-Dimethyl- | 1-sec-Butyl-3-[4-(5,7- | 91 | 12.10 min | 426 (M − H)⁻ |
| pyrazolo[3,4- | benzoxazol-2-yl)-[4- | dimethyl-benzoxazol- | | (a) | |
| d]pyrimidin-4-ylamine | (4,4,5,5-tetramethyl- | 2-ylamino)-phenyl]- | | | |
| (O) | [1,3,2]dioxaborolan-2- | 1H-pyrazolo[3,4- | | | |
| | yl)-phenyl]-amine | d]pyrimidin-4-ylamine | | | |
| | (G, D) | | | | |
| trans-3-Iodo-1-[4-(2- | [2-Fluoro-4-(4,4,5,5- | trans-3-[3-Fluoro-4-(5- | 92 | 11.03 min | 584 (M − H)⁻ |
| methoxy-ethoxy)- | tetramethyl- | trifluoromethyl- | | (a) | |
| cyclohexyl]-1H- | [1,3,2]dioxaborolan-2- | benzoxazol-2- | | | |
| pyrazolo[3,4- | yl)-phenyl]-(5- | ylamino)-phenyl]-1-[4- | | | |
| d]pyrimidin-4-ylamine | trifluoromethyl- | (2-methoxy-ethoxy)- | | | |
| (A) | benzoxazol-2-yl)- | cyclohexyl]-1H- | | | |
| | amine (H, G, D) | pyrazolo[3,4- | | | |
| | | d]pyrimidin-4-ylamine | | | |
| trans-3-Iodo-1-[4-(2- | (5-Fluoro-benzoxazol- | trans-3-[3-Fluoro-4-(5- | 93 | 10.17 min | 534 (M − H)⁻ |
| methoxy-ethoxy)- | 2-yl)-[2-fluoro-4- | fluoro-benzoxazol-2- | | (a) | |
| cyclohexyl]-1H- | (4,4,5,5-tetramethyl- | ylamino)-phenyl]-1-[4- | | | |
| pyrazolo[3,4- | [1,3,2]dioxaborolan-2- | (2-methoxy-ethoxy)- | | | |
| d]pyrimidin-4-ylamine | yl)-phenyl]-amine | cyclohexyl]-1H- | | | |
| (A) | (G, D) | pyrazolo[3,4- | | | |
| | | d]pyrimidin-4-ylamine | | | |
| trans-3-Iodo-1-[4-(2- | (5-Chloro-7-methyl- | trans-3-[4-(5-Chloro-7- | 94 | 11.33 min | 548 (M + H)⁺ |
| methoxy-ethoxy)- | benzoxazol-2-yl)-[4- | methyl-benzoxazol-2- | | (a) | |
| cyclohexyl]-1H- | (4,4,5,5-tetramethyl- | ylamino)-phenyl]-1-[4- | | | |
| pyrazolo[3,4- | [1,3,2]dioxaborolan-2- | (2-methoxy-ethoxy)- | | | |
| d]pyrimidin-4-ylamine | yl)-phenyl]-amine | cyclohexyl]-1H- | | | |
| (A) | (F, H, G, D) | pyrazolo[3,4- | | | |
| | | d]pyrimidin-4-ylamine | | | |
| trans-5-Iodo-7-[4-(2- | (5-Chloro-7-methyl- | trans-5-[4-(5-Chloro-7- | 95 | 11.45 min | 547 (M + H)⁺ |
| methoxy-ethoxy)- | benzoxazol-2-yl)-[4- | methyl-benzoxazol-2- | | (a) | |
| cyclohexyl]-7H- | (4,4,5,5-tetramethyl- | ylamino)-phenyl]-7-[4- | | | |
| pyrrolo[2,3- | [1,3,2]dioxaborolan-2- | (2-methoxy-ethoxy)- | | | |
| d]pyrimidin-4-ylamine | yl)-phenyl]-amine | cyclohexyl]-7H- | | | |
| (A, B) | (F, H, G, D) | pyrrolo[2,3- | | | |
| | | d]pyrimidin-4-ylamine | | | |
| trans-5-Iodo-7-[4-(2- | (7-Chloro-5-methyl- | trans-5-[4-(7-Chloro-5- | 96 | 11.56 min | 547 (M + H)⁺ |
| methoxy-ethoxy)- | benzoxazol-2-yl)-[4- | methyl-benzoxazol-2- | | (a) | |
| cyclohexyl]-7H- | (4,4,5,5-tetramethyl- | ylamino)-phenyl]-7-[4- | | | |
| pyrrolo[2,3- | [1,3,2]dioxaborolan-2- | (2-methoxy-ethoxy)- | | | |
| d]pyrimidin-4-ylamine | yl)-phenyl]-amine | cyclohexyl]-7H- | | | |
| (A, B) | (F, H, G, D) | pyrrolo[2,3- | | | |
| | | d]pyrimidin-4-ylamine | | | |
| trans-4-[4-(4-Amino-5- | (5,7-Dimethyl- | trans-4-(4-{4-Amino-5- | 97 | 2.52 min (f) | 565 (M + H)⁺ |
| iodo-pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexyl]-1-methyl- | [1,3,2]dioxaborolan-2- | ylamino)-phenyl]- | | | |
| piperazin-2-one | yl)-phenyl]-amine (D) | pyrrolo[2,3- | | | |
| (B, A, T, J(M)) | | d]pyrimidin-7-yl}- | | | |
| | | cyclohexyl)-1-methyl- | | | |
| | | piperazin-2-one | | | |
| trans-2-[4-(4-Amino-5- | (5,7-Dimethyl- | trans-2-(4-{4-Amino-5- | 98 | 2.30 min (f) | 512 (m + H)⁺ |
| iodo-pyrrolo[2,3- | benzoxazol-2-yl)-[4- | [4-(5,7-dimethyl- | | | |
| d]pyrimidin-7-yl)- | (4,4,5,5-tetramethyl- | benzoxazol-2- | | | |
| cyclohexylamino]- | [1,3,2]dioxaborolan-2- | ylamino)-phenyl]- | | | |
| ethanol (B, A, T, J) | yl)-phenyl]-amine (D) | pyrrolo[2,3- | | | |
| | | d]pyrimidin-7-yl}- | | | |
| | | cyclohexylamino)- | | | |
| | | ethanol | | | |
| trans-4-[4-(4-Amino-3- | (5,7-Dimethyl- | trans-4-(4-{4-Amino-3- | 99 | 2.59 min (f) | 553 (M + H)⁺ |
| iodo-pyrazolo[3,4- | benzoxazol-2-yl)-[4- | [4-(5,7-dimethyl- | | | |
| d]pyrimidin-1-yl)- | (4,4,5,5-tetramethyl- | benzoxazol-2- | | | |
| cyclohexyl]-morpholin- | [1,3,2]dioxaborolan-2- | ylamino)-phenyl]- | | | |
| 3-one (A(J, (BB), T, U)) | yl)-phenyl]-amine (D) | pyrazolo[3,4- | | | |
| | | d]pyrimidin-1-yl}- | | | |
| | | cyclohexyl)-morpholin- | | | |
| | | 3-one | | | |
| trans-3-Iodo-1-[4-(2- | (5-Chloro-benzoxazol- | trans-3-[4-(5-Chloro- | 100 | 3.80 min (f) | 588 (M + H)⁺ |
| trifluoromethoxy- | 2-yl)-[4-(4,4,5,5- | benzoxazol-2- | | | |
| ethoxy)-cyclohexyl]- | tetramethyl- | ylamino)-phenyl]-1-[4- | | | |
| 1H-pyrazolo[3,4- | [1,3,2]dioxaborolan-2- | (2-trifluoromethoxy- | | | |
| d]pyrimidin-4-ylamine | yl)-phenyl]-amine (D) | ethoxy)-cyclohexyl]- | | | |
| (A(X, (CC), (DD), N)) | | 1H-pyrazolo[3,4- | | | |
| | | d]pyrimidin-4-ylamine | | | |
| trans-{5-Iodo-7-[4-(4- | (5,7-Dimethyl- | trans-5-[4-(5,7- | 101 | 8.55 min | 569 (M + H)⁺ |
| methyl-piperazin-1-yl)- | benzoxazol-2-yl)-[2- | Dimethyl-benzoxazol- | | (b) | |
| cyclohexyl]-7H- | fluoro-4-(4,4,5,5- | 2-yl | | | |
| pyrrolo[2,3-d]pyrimidin- | tetramethyl- | amino)-3-fluoro- | | | |
| 4-ylamine} | [1,3,2]dioxaborolan-2- | phenyl]-7-[4-(4-methyl- | | | |
| (A, B, T, J,) | yl)-phenyl]-amine | piperazin-1-yl)- | | | |
| | (G, D) | cyclohexyl]-7H- | | | |
| | | pyrrolo[2,3- | | | |
| | | d]pyrimidin-4-ylamine | | | |
| (S)-5-Iodo-7-(1- | (5,7-Dimethyl- | 5-[4-(5,7-Dimethyl- | 102 | 12.14 min | 472 (M + H)⁺ |
| methyl-pyrrolidin-3-yl)- | benzoxazol-2-yl)-[2- | benzoxazol-2-yl | | (e) | |
| 7H-pyrrolo[2,3- | fluoro-4-(4,4,5,5- | amino)-3-fluoro- | | | |
| d]pyrimidin-4-ylamine | tetramethyl- | phenyl]-7-(S)-(1- | | | |
| (A(R), L, B, J) | [1,3,2]dioxaborolan-2- | methyl- | | | |
| | yl)-phenyl]-amine | pyrrolidin-3-yl)-7H- | | | |
| | (G, D) | pyrrolo[2,3- | | | |
| | | d]pyrimidin-4-ylamine | | | |
| cis-{4-[4-(4-Amino-3- | (5,7-Dimethyl- | cis-{4-(4-{4-Amino-3- | 103 | 7.66 min | 570 (M + H)⁺ |
| iodo-pyrazolo[3,4- | benzoxazol-2-yl)-[2- | [4-(5,7-dimethyl- | | (b) | |
| d]pyrimidin-1-yl)- | fluoro-4-(4,4,5,5- | benzoxazol-2- | | | |
| cyclohexyl]-piperazin- | tetramethyl-[1,3, | ylamino)-3-fluoro- | | | |
| 2-one} (A, T, J) | 2]dioxaborolan-2-yl)- | phenyl]-pyrazolo[3,4- | | | |
| | phenyl]-amine (G, D) | d]pyrimidin-1-yl}- | | | |
| | | cyclohexyl)-piperazin- | | | |
| | | 2-one} | | | |
| trans-{4-[4-(4-Amino- | (5,7-Dimethyl- | trans-{4-(4-{4-Amino- | 104 | 14.91 min | 570 (M + H)⁺ |
| 3-iodo-pyrazolo[3,4- | benzoxazol-2-yl)-[4- | 3-[4-(5,7-dimethyl- | | (e) | |
| d]pyrimidin-1-yl)- | (4,4,5,5-tetramethyl- | benzoxazol-2- | | | |
| cyclohexyl]-piperazin- | [1,3,2]dioxaborolan-2- | ylamino)-3-fluoro- | | | |
| 2-one} (A, T, J) | yl)-phenyl]-amine (E, | phenyl]-pyrazolo[3,4- | | | |
| | D) | d]pyrimidin-1-yl}- | | | |
| | | cyclohexyl)-piperazin- | | | |
| | | 2-one} | | | |
| trans-{4-[4-(4-Amino- | (5-Chloro-7-methyl- | trans-{4-(4-{4-Amino- | 105 | 15.64 min | 552 (M − H)⁻ |
| 3-iodo-pyrazolo[3,4- | benzoxazol-2-yl)-[4- | 3-[4-(5-chloro-7- | | (e) | |
| d]pyrimidin-1-yl)- | (4,4,5,5-tetramethyl- | methyl-benzoxazol-2- | | | |
| cyclohexyl]-piperazin- | [1,3,2]dioxaborolan-2- | ylamino)-phenyl]- | | | |
| 2-one} (A, T, J) | yl)-phenyl]-amine | pyrazolo[3,4- | | | |
| | (E, D) | d]pyrimidin-1-yl}- | | | |
| | | cyclohexyl)-piperazin- | | | |
| | | 2-one} | | | |
| trans-{4-[4-(4-Amino- | Benzoxazol-2-yl-[4- | trans-{4-(4-{4-Amino- | 106 | 12.91 min | 522 (M − H)⁻ |
| 3-iodo-pyrazolo[3,4- | (4,4,5,5-tetramethyl- | 3-[4-(benzoxazol-2- | | (e) | |
| d]pyrimidin-1-yl)- | [1,3,2]dioxaborolan-2- | ylamino)-phenyl]- | | | |
| cyclohexyl]-piperazin- | yl)-phenyl]-amine | pyrazolo[3,4- | | | |
| 2-one} (A, T, J) | (E, D) | d]pyrimidin-1-yl}- | | | |
| | | cyclohexyl)-piperazin- | | | |
| | | 2-one} | | | |
| trans-{4-[4-(4-Amino- | (5,7-Dimethyl- | trans-{4-(4-{4-Amino- | 107 | 15.77 min | 564 (M − H)⁻ |
| 3-iodo-pyrazolo[3,4- | benzoxazol-2-yl)-[4- | 3-[4-(5,7-dimethyl- | | (e) | |
| d]pyrimidin-1-yl)- | (4,4,5,5-tetramethyl- | benzoxazol-2- | | | |
| cyclohexyl]-1-methyl- | [1,3,2]dioxaborolan-2- | ylamino)-phenyl]- | | | |
| piperazin-2-one} | yl)-phenyl]-amine | pyrazolo[3,4- | | | |
| (A, T, J(M)) | (E, D) | d]pyrimidin-1-yl}- | | | |
| | | cyclohexyl)-1-methyl- | | | |
| | | piperazin-2-one} | | | |
| cis-{4-[4-(4-Amino-3- | Benzoxazol-2-yl-[4- | cis-{4-(4-{4-Amino-3- | 108 | 14.39 min | δ (DMSO-d₆) |
| iodo-pyrazolo[3,4- | (4,4,5,5-tetramethyl- | [4-(benzoxazol-2- | | (e) | 10.86, 8.23, |
| d]pyrimidin-1-yl)- | [1,3,2]dioxaborolan-2- | ylamino)-phenyl]- | | | 7.94, 7.66, |
| cyclohexyl]-1-methyl- | yl)-phenyl]-amine | pyrazolo[3,4- | | | 7.51, 7.25, |
| piperazin-2-one} | (E, D) | d]pyrimidin-1-yl}- | | | 7.16, 4.81, |
| (A, T, J(M)) | | cyclohexyl)-1-methyl- | | | 3.07, 2.84, |
| | | piperazin-2-one} | | | 2.72, 2.32-2.23, |
| | | | | | 2.12-2.08, |
| | | | | | 1.72-1.60 |
| trans-{4-[4-(4-Amino- | (5-Chloro-7-methyl- | trans-{4-(4-{4-Amino- | 109 | 16.45 min | 584 (M − H)⁻ |
| 3-iodo-pyrazolo[3,4- | benzoxazol-2-yl)-[4- | 3-[4-(5-chloro-7- | | (e) | |
| d]pyrimidin-1-yl)- | (4,4,5,5-tetramethyl- | methyl-benzoxazol-2- | | | |
| cyclohexyl]-1-methyl- | [1,3,2]dioxaborolan-2- | ylamino)-phenyl]- | | | |
| piperazin-2-one} (A, T, | yl)-phenyl]-amine | pyrazolo[3,4- | | | |
| J(M)) | (E(F, H), D) | d]pyrimidin-1-yl}- | | | |
| | | cyclohexyl)-1-methyl- | | | |
| | | piperazin-2-one} | | | |
| trans-{4-[4-(4-Amino- | Benzoxazol-2-yl-[4- | trans-{4-(4-{4-Amino- | 110 | 13.57 min | 536 (M − H)⁻ |
| 3-iodo-pyrazolo[3,4- | (4,4,5,5-tetramethyl- | 3-[4-(benzoxazol-2- | | (e) | |
| d]pyrimidin-1-yl)- | [1,3,2]dioxaborolan-2- | ylamino)-phenyl]- | | | |
| cyclohexyl]-1-methyl- | yl)-phenyl]-amine | pyrazolo[3,4- | | | |
| piperazin-2-one} | (E, D) | d]pyrimidin-1-yl}- | | | |
| (A, T, J(M)) | | cyclohexyl)-1-methyl- | | | |
| | | piperazin-2-one} | | | |
| cis-{4-[4-(4-Amino-3- | (5-Chloro-7-methyl- | cis-{4-(4-{4-Amino-3- | 111 | 17.55 min | δ (CDCl₃) |
| iodo-pyrazolo[3,4- | benzoxazol-2-yl)-[4- | [4-(5-Chloro-7-methyl- | | (e) | 8.38, 7.79, |
| d]pyrimidin-1-yl)- | (4,4,5,5-tetramethyl- | benzoxazol-2- | | | 7.52, 6.97, |
| cyclohexyl]-1-methyl- | [1,3,2]dioxaborolan-2- | ylamino)-phenyl]- | | | 5.47, 4.90, |
| piperazin-2-one} (A, T, | yl)-phenyl]-amine | pyrazolo[3,4- | | | 3.36, 3.26, |
| J(M)) | (E (F, H), D) | d]pyrimidin-1-yl}- | | | 2.98, 2.77, |
| | | cyclohexyl)-1-methyl- | | | 2.45, 2.40, |
| | | piperazin-2-one} | | | 2.15, 1.83, |
| | | | | | 0.90-0.83 |
| trans-{2-{[4-(4-Amino- | (5,7-Dimethyl- | trans-{2-[(4-{4-Amino- | 112 | 9.16 min | 610 (M + H)⁺ |
| 3-iodo-pyrazolo[3,4- | benzoxazol-2-yl)-[4- | 3-[4-(5,7-dimethyl- | | (b) | |
| d]pyrimidin-1-yl)- | (4,4,5,5-tetramethyl- | benzoxazol-2- | | | |
| cyclohexyl]-methyl- | [1,3,2]dioxaborolan-2- | ylamino)-phenyl]- | | | |
| amino}-1-morpholin-4- | yl)-phenyl]-amine | pyrazolo[3,4- | | | |
| yl-ethanone} | (E, D) | d]pyrimidin-1-yl}- | | | |
| (A, T, J, P, Q) | | cyclohexyl)-methyl- | | | |
| | | amino]-1-morpholin-4- | | | |
| | | yl-ethanone} | | | |
| trans-{2-{[4-(4-Amino- | (5,7-Dimethyl- | trans-{2-[(4-{4-Amino- | 113 | 9.23 min | 568 (M + H)⁺ |
| 3-iodo-pyrazolo[3,4- | benzoxazol-2-yl)-[4- | 3-[4-(5,7-dimethyl- | | (b) | |
| d]pyrimidin-1-yl)- | (4,4,5,5-tetramethyl- | benzoxazol-2- | | | |
| cyclohexyl]-methyl- | [1,3,2]dioxaborolan- | ylamino)-phenyl]- | | | |
| amino}-N,N-dimethyl- | 2-yl)-phenyl]- | pyrazolo[3,4- | | | |
| acetamide} | amine (E, D) | d]pyrimidin-1-yl}- | | | |
| (A, T, J, P, Q) | | cyclohexyl)-methyl- | | | |
| | | amino]-N,N-dimethyl- | | | |
| | | acetamide} | | | |
| cis-{[4-(4-Amino-3- | (5,7-Dimethyl- | cis-[(4-{4-Amino-3-[4- | 114 | 10.74 min | 541 (M + H)⁺ |
| iodo-pyrazolo[3,4- | benzoxazol-2-yl)-[4- | (5,7-dimethyl- | | (b) | |
| d]pyrimidin-1-yl)- | (4,4,5,5-tetramethyl- | benzoxazol-2- | | | |
| cyclohexyl]-methyl- | [1,3,2]dioxaborolan-2- | ylamino)-phenyl]- | | | |
| amino}-acetic acid | yl)-phenyl]-amine | pyrazolo[3,4- | | | |
| (A, T, J, P) | (E, D) | d]pyrimidin-1-yl}- | | | |
| | | cyclohexyl)-methyl- | | | |
| | | amino]-acetic acid | | | |
| cis-{2-{[4-(4-Amino-3- | (5,7-Dimethyl- | cis-{2-[(4-{4-Amino-3- | 115 | 9.24 min | 610 (M + H)⁺ |
| iodo-pyrazolo[3,4- | benzoxazol-2-yl)-[4- | [4-(5,7-dimethyl- | | (b) | |
| d]pyrimidin-1-yl)- | (4,4,5,5-tetramethyl- | benzoxazol-2- | | | |
| cyclohexyl]-methyl- | [1,3,2]dioxaborolan- | ylamino)-phenyl]- | | | |
| amino}-1-morpholin- | 2-yl)-phenyl]- | pyrazolo[3,4- | | | |
| 4-yl-ethanone} | amine (E, D) | d]pyrimidin-1-yl}- | | | |
| (A, T, J, P, Q) | | cyclohexyl)-methyl- | | | |
| | | amino]-1-morpholin-4- | | | |
| | | yl-ethanone} acetate | | | |
| | | salt | | | |
| trans-{4-[4-(4-Amino- | (5-Chloro-benzoxazol- | trans-{4-(4-{4-Amino- | 116 | 10.26 min | 615 (M − H)⁻ |
| 5-iodo-pyrrolo[2,3- | 2-yl)-[2-fluoro-4- | 5-[4-(5-chloro- | | (a) | |
| d]pyrimidin-7-yl)- | (4,4,5,5-tetramethyl- | benzoxazol-2- | | | |
| cyclohexyl]-1-isopropyl- | [1,3,2]dioxaborolan-2- | ylamino)-3-fluoro- | | | |
| piperazin-2-one} | yl)-phenyl]-amine | phenyl]-pyrrolo[2,3- | | | |
| (A, B, T, J(M)) | (G, D) | d]pyrimidin-7-yl}- | | | |
| | | cyclohexyl)-1- | | | |
| | | isopropyl-piperazin-2- | | | |
| | | one} | | | |
| trans-{4-[4-(4-Amino- | (5,7-Dimethyl- | trans-{4-(4-{4-Amino- | 117 | 10.71 min | 593 (M + H)⁺ |
| 5-iodo-pyrrolo[2,3- | benzoxazol-2-yl)-[4- | 5-[4-(5,7-dimethyl- | | (a) | |
| d]pyrimidin-7-yl)- | (4,4,5,5-tetramethyl- | benzoxazol-2- | | | |
| cyclohexyl]-1- | [1,3,2]dioxaborolan- | ylamino)-phenyl]- | | | |
| isopropyl-piperazin-2- | 2-yl)-phenyl]- | pyrrolo[2,3- | | | |
| one} (A, B, T, J(M)) | amine (E, D) | d]pyrimidin-7-yl}- | | | |
| | | cyclohexyl)-1- | | | |
| | | isopropyl-piperazin-2- | | | |
| | | one} | | | |
| trans-{4-[4-(4-Amino- | [2-Fluoro-4-(4,4,5,5- | trans-{4-(4-{4-Amino- | 118 | 9.96 min | 597 (M + H)⁺ |
| 5-iodo-pyrrolo[2,3- | tetramethyl-[1, | 5-[3-fluoro-4-(5- | | (a) | |
| d]pyrimidin-7-yl)- | 3,2]dioxaborolan-2- | methyl-benzoxazol-2- | | | |
| cyclohexyl]-1- | yl)-phenyl]-(5- | ylamino)-phenyl]- | | | |
| isopropyl-piperazin-2- | methyl-benzoxazol-2- | pyrrolo[2,3- | | | |
| one} (A, B, T, J(M)) | yl)-amine (G, D) | d]pyrimidin-7-yl}- | | | |
| | | cyclohexyl)-1- | | | |
| | | isopropyl-piperazin-2- | | | |
| | | one} | | | |
| trans-{4-[4-(4-Amino- | (5,7-Dimethyl- | trans-{4-(4-{4-Amino- | 119 | 10.95 min | 609 (M − H)⁻ |
| 5-iodo-pyrrolo[2,3- | benzoxazol-2-yl)-[2- | 5-[4-(5,7-dimethyl- | | (a) | |
| d]pyrimidin-7-yl)- | fluoro-4-(4,4,5,5- | benzoxazol-2- | | | |
| cyclohexyl]-1- | tetramethyl- | ylamino)-3-fluoro- | | | |
| isopropyl-piperazin-2- | [1,3,2]dioxaborolan-2- | phenyl]-pyrrolo[2,3- | | | |
| one} (A, B, T, J(M)) | yl)-phenyl]-amine | d]pyrimidin-7-yl}- | | | |
| | (G, D) | cyclohexyl)-1- | | | |
| | | isopropyl-piperazin-2- | | | |
| | | one} | | | |
| trans-{4-[4-(4-Amino- | (5,7-Dimethyl- | trans-{4-(4-{4-Amino- | 120 | 10.98 min | 598 (M + H)⁺ |
| 3-iodo-pyrazolo[3,4- | benzoxazol-2-yl)-[2- | 3-[4-(5,7-dimethyl- | | (a) | |
| d]pyrimidin-1-yl)- | fluoro-4-(4,4,5,5- | benzoxazol-2- | | | |
| cyclohexyl]-6,6- | tetramethyl- | ylamino)-3-fluoro- | | | |
| dimethyl-piperazin-2- | [1,3,2]dioxaborolan-2- | phenyl]-pyrazolo[3,4- | | | |
| one} (A, T, J(U.S. Pat. No. | yl)-phenyl]-amine | d]pyrimidin-1-yl}- | | | |
| 4,251,438)) | (G, D) | cyclohexyl)-6,6- | | | |
| | | dimethyl-piperazin-2- | | | |
| | | one} | | | |
| trans-{4-[4-(4-Amino- | (5,7-Dimethyl- | 4-(4-{4-Amino-3-[4- | 121 | 10.60 min | 580 (M + H)⁺ |
| 3-iodo-pyrazolo[3,4- | benzoxazol-2-yl)-[4- | (5,7-dimethyl- | | (a) | |
| d]pyrimidin-1-yl)- | (4,4,5,5-tetramethyl- | benzoxazol-2- | | | |
| cyclohexyl]-6,6-di | [1,3,2]dioxaborolan-2- | ylamino)-phenyl]- | | | |
| methyl-piperazin-2- | yl)-phenyl]-amine | pyrazolo[3,4- | | | |
| one} (A, T, J(U.S. Pat. No. | (E, D) | d]pyrimidin-1-yl}- | | | |
| 4,251,438)) | | cyclohexyl)-6,6- | | | |
| | | dimethyl-piperazin-2- | | | |
| | | one | | | |
| 3-Iodo-1-isopropyl-1H- | (5,7-Dimethyl- | 3-[4-(5,7-Dimethyl- | 122 | 11.17 min | 414 (M + H)⁺ |
| pyrazolo[3,4- | benzoxazol-2-yl)-[4- | benzoxazol-2- | | (a) | |
| d]pyrimidin-4-ylamine | (4,4,5,5-tetramethyl- | ylamino)-phenyl]-1- | | | |
| (O) | [1,3,2]dioxaborolan-2- | isopropyl-1H- | | | |
| | yl)-phenyl]-amine | pyrazolo[3,4- | | | |
| | (E, D) | d]pyrimidin-4-ylamine | | | |
| 1-(1-Ethyl-propyl)-3- | (5,7-Dimethyl- | 3-[4-(5,7-Dimethyl- | 123 | 12.18 min | 442 (M + H)⁺ |
| iodo-1H-pyrazolo[3,4- | benzoxazol-2-yl)-[4- | benzoxazol-2-yl | | (a) | |
| d]pyrimidin-4-ylamine | (4,4,5,5-tetramethyl- | amino)-phenyl]-1-(1- | | | |
| (O) | [1,3,2]dioxaborolan- | ethyl-propyl)-1 | | | |
| | 2-yl)-phenyl]- | H-pyrazolo[3,4- | | | |
| | amine (E, D) | d]pyrimidin-4-ylamine | | | |
| 7-tert-Butyl-5-iodo-7H- | (5,7-Dimethyl- | 7-tert-Butyl-5-[4-(5,7- | 124 | 12.79 min | 445 (M + H)⁺ |
| pyrrolo[2,3- | benzoxazol-2-yl)-[2- | dimethyl-benzoxazol- | | (a) | |
| d]pyrimidin-4-ylamine | fluoro-4-(4,4,5,5- | 2-ylamino)-3-fluoro- | | | |
| (O, B) | tetramethyl- | phenyl]-7H- | | | |
| | [1,3,2]dioxaborolan-2- | pyrrolo[2,3- | | | |
| | yl)-phenyl]-amine | d]pyrimidin-4-ylamine | | | |
| | (G, D) | | | | |
| 7-tert-Butyl-5-iodo-7H- | (5,7-Dimethyl- | 7-tert-Butyl-5-[4-(5,7- | 125 | 12.49 min | 427 (M + H)⁺ |
| pyrrolo[2,3- | benzoxazol-2-yl)-[4- | dimethyl-benzoxazol- | | (a) | |
| d]pyrimidin-4-ylamine | (4,4,5,5-tetramethyl- | 2-ylamino)-phenyl]- | | | |
| (O, B) | [1,3,2]dioxaborolan-2- | 7H-pyrrolo[2,3- | | | |
| | yl)-phenyl]-amine | d]pyrimidin-4-ylamine | | | |
| | (E, D) | | | | |
| 7-tert-Butyl-5-iodo-7H- | [2-Fluoro-4-(4,4,5,5- | 7-tert-Butyl-5-[3-fluoro- | 126 | 12.11 min | 431 (M + H)⁺ |
| pyrrolo[2,3- | tetramethyl-[1, | 4-(5-methyl- | | (a) | |
| d]pyrimidin-4-ylamine | 3,2]dioxaborolan-2- | benzoxazol-2- | | | |
| (O, B) | yl)-phenyl]-(5- | ylamino)-phenyl]-7H- | | | |
| | methyl-benzoxazol-2- | pyrrolo[2,3- | | | |
| | yl)-amine (G, D) | d]pyrimidin-4-ylamine | | | |
| 7-tert-Butyl-5-iodo-7H- | [2-Fluoro-4-(4,4,5,5- | 7-tert-Butyl-5-[3-fluoro- | 127 | 12.43 min | 485 (M + H)⁺ |
| pyrrolo[2,3- | tetramethyl- | 4-(5-trifluoromethyl- | | (a) | |
| d]pyrimidin-4-ylamine | [1,3,2]dioxaborolan-2- | benzoxazol-2- | | | |
| (O, B) | yl)-phenyl]-(5 | ylamino)-phenyl]-7H- | | | |
| | trifluoromethyl- | pyrrolo[2,3- | | | |
| | benzoxazol-2-yl)- | d]pyrimidin-4-ylamine | | | |
| | amine (G(F, H), D) | | | | |
| 7-tert-Butyl-5-iodo-7H- | (5-Fluoro-benzoxazol- | 7-tert-Butyl-5-[3-fluoro- | 128 | 11.66 min | 435 (M + H)⁺ |
| pyrrolo[2,3- | 2-yl)-[2-fluoro-4- | 4-(5-fluoro- | | (a) | |
| d]pyrimidin-4-ylamine | (4,4,5,5-tetramethyl- | benzoxazol-2- | | | |
| (O, B) | [1,3,2]dioxaborolan-2- | ylamino)-phenyl]-7H- | | | |
| | yl)-phenyl]-amine | pyrrolo[2,3- | | | |
| | (G(F, H), D) | d]pyrimidin-4-ylamine | | | |
| trans-{5-Iodo-7-[4-(2- | (5-Chloro-benzoxazol- | trans-{5-[4-(5-Chloro- | 129 | 10.92 min | 551 (M + H)⁺ |
| methoxy-ethoxy)- | 2-yl)-[2-fluoro-4- | benzoxazol-2- | | (a) | |
| cyclohexyl]-7H- | (4,4,5,5-tetramethyl- | ylamino)-3-fluoro- | | | |
| pyrrolo[2,3- | [1,3,2]dioxaborolan-2- | phenyl]-7-[4-(2- | | | |
| d]pyrimidin-4-ylamine} | yl)-phenyl]-amine | methoxy-ethoxy)- | | | |
| (A, B) | (G, D) | cyclohexyl]-7H- | | | |
| | | pyrrolo[2,3- | | | |
| | | d]pyrimidin-4-ylamine} | | | |
| trans-{5-Iodo-7-[4-(2- | (5-Chloro-benzoxazol- | trans-{5-[4-(5-Chloro- | 130 | 10.83 min | 533 (M + H)⁺ |
| methoxy-ethoxy)- | 2-yl)-[4-(4,4,5,5- | benzoxazol-2- | | (a) | |
| cyclohexyl]-7H- | tetramethyl- | ylamino)-phenyl]-7-[4- | | | |
| pyrrolo[2,3- | [1,3,2]dioxaborolan-2- | (2-methoxy-ethoxy)- | | | |
| d]pyrimidin-4-ylamine} | yl)-phenyl]-amine | cyclohexyl]-7H- | | | |
| (A, B) | (E, D) | pyrrolo[2,3- | | | |
| | | d]pyrimidin-4-ylamine} | | | |
| trans-{4-[4-(4-Amino- | (5-Chloro-benzoxazol- | trans-{4-(4-{4-Amino- | 131 | 10.01 min | 604 (M + H)⁺ |
| 3-iodo-pyrazolo[3,4- | 2-yl)-[2-fluoro-4- | 3-[4-(5-chloro- | | (a) | |
| d]pyrimidin-1-yl)- | (4,4,5,5-tetramethyl- | benzoxazol-2- | | | |
| cyclohexyl]-6,6- | [1,3,2]dioxaborolan-2- | ylamino)-3-fluoro- | | | |
| dimethyl-piperazin-2- | yl)-phenyl]-amine | phenyl]-pyrazolo[3,4- | | | |
| one} (A, T, J(U.S. Pat. No. | (G, D) | d]pyrimidin-1-yl}- | | | |
| 4,251,438)) | | cyclohexyl)-6,6- | | | |
| | | dimethyl-piperazin-2- | | | |
| | | one} | | | |
| trans-{4-[4-(4-Amino- | (5-Chloro-benzoxazol- | trans-{4-(4-{4-Amino- | 132 | 9.93 min | 586 (M + H)⁺ |
| 3-iodo-pyrazolo[3,4- | 2-yl)-[4-(4,4,5,5- | 3-[4-(5-chloro- | | (a) | |
| d]pyrimidin-1-yl)- | tetramethyl- | benzoxazol-2- | | | |
| cyclohexyl]-6,6- | [1,3,2]dioxaborolan-2- | ylamino)-phenyl]- | | | |
| dimethyl-piperazin-2- | yl)-phenyl]-amine | pyrazolo[3,4- | | | |
| one} (A, T, J(U.S. Pat. No. | (E, D) | d]pyrimidin-1-yl}- | | | |
| 4,251,438)) | | cyclohexyl)-6,6- | | | |
| | | dimethyl-piperazin-2- | | | |
| | | one} | | | |
| trans-{3-Iodo-1-(4- | (7-Chloro-5-methyl- | trans-{3-[4-(7-Chloro- | 133 | 12.41 min | 559 (M + H)⁺ |
| morpholin-4-yl- | benzoxazol-2-yl)-[4- | 5-methyl-benzoxazol | | (h) | |
| cyclohexyl)-1H- | (4,4,5,5-tetramethyl- | 2-ylamino)-phenyl]-1- | | | |
| pyrazolo[3,4- | [1,3,2]dioxaborolan-2- | (4-morpholin-4-yl- | | | |
| d]pyrimidin-4-ylamine} | yl)-phenyl]-amine | cyclohexyl)-1H- | | | |
| (A, T, J) | (G(F, H)) | pyrazolo[3,4- | | | |
| | | d]pyrimidin-4-ylamine} | | | |
| trans-{3-Iodo-1-(4- | (5-Chloro-7-methyl- | trans-{3-[4-(5-Chloro- | 134 | 12.22 min | 559 (M + H)⁺ |
| morpholin-4-yl- | benzoxazol-2-yl)-[4- | 7-methyl-benzoxazol | | (e) | |
| cyclohexyl)-1H- | (4,4,5,5-tetramethyl- | 2-ylamino)-phenyl]-1- | | | |
| pyrazolo[3,4- | [1,3,2]dioxaborolan-2- | (4-morpholin-4-yl- | | | |
| d]pyrimidin-4-ylamine} | yl)-phenyl]-amine | cyclohexyl)-1H- | | | |
| (A, T, J) | (G(F, H)) | pyrazolo[3,4- | | | |
| | | d]pyrimidin-4-ylamine} | | | |
| 3-Iodo-1-[1-(2- | (5-Methyl-benzoxazol- | 1-[1-(2-Methoxy- | 135 | 7.09 min | 499 (M + H)⁺ |
| methoxy-ethyl)- | 2-yl)-[4-(4,4,5,5- | ethyl)-piperidin-3-yl]-3- | | (b) | |
| piperidin-3-yl]-1H- | tetramethyl- | [4-(5-methyl- | | | |
| pyrazolo[3,4- | [1,3,2]dioxaborolan-2- | benzoxazol-2- | | | |
| d]pyrimidin-4-ylamine | yl)-phenyl]-amine | ylamino)-phenyl]-1H- | | | |
| (A, L, I) | (G, D) | pyrazolo[3,4- | | | |
| | | d]pyrimidin-4-ylamine | | | |
| | | acetate salt | | | |
| 3-Iodo-1-[1-(2- | (5-Ethyl-benzoxazol-2- | 1-[1-(2-Methoxy- | 136 | 7.56 min | 513 (M + H)⁺ |
| methoxy-ethyl)- | yl)-[4-(4,4,5,5- | ethyl)-piperidin-3-yl]-3- | | (b) | |
| piperidin-3-yl]-1H- | tetramethyl- | [4-(5-ethyl- | | | |
| pyrazolo[3,4- | [1,3,2]dioxaborolan-2- | benzoxazol-2- | | | |
| d]pyrimidin-4-ylamine | yl)-phenyl]-amine | ylamino)-phenyl]-1H- | | | |
| (A, L, I) | (G, D) | pyrazolo[3,4- | | | |
| | | d]pyrimidin-4-ylamine | | | |
| | | acetate salt | | | |
| 3-Iodo-1-[1-(2- | (5-Chloro-benzoxazol- | 1-[1-(2-Methoxy- | 137 | 7.34 min | 519 (M + H)⁺ |
| methoxy-ethyl)- | 2-yl)-[4-(4,4,5,5- | ethyl)-piperidin-3-yl]-3- | | (b) | |
| piperidin-3-yl]-1H- | tetramethyl- | [4-(5-chloro- | | | |
| pyrazolo[3,4- | [1,3,2]dioxaborolan-2- | benzoxazol-2- | | | |
| d]pyrimidin-4-ylamine | yl)-phenyl]-amine | ylamino)-phenyl]-1H- | | | |
| (A, L, I) | (G, D) | pyrazolo[3,4- | | | |
| | | d]pyrimidin-4-ylamine | | | |
| | | acetate salt | | | |
| cis-3-Iodo-1-(4- | (5,7-Dimethyl- | cis-3-[4-(5,7-Dimethyl- | 138 | 11.9 min | 539 (M + H)⁺ |
| morpholin-4-yl- | benzoxazol-2-yl)-[4- | benzoxazol-2- | | (b) | |
| cyclohexyl)-1H- | (4,4,5,5-tetramethyl- | ylamino)-phenyl]-1-(4- | | | |
| pyrazolo[3,4- | [1,3,2]dioxaborolan-2- | morpholin-4-yl- | | | |
| d]pyrimidin-4-ylamine | yl)-phenyl]-amine | cyclohexyl)-1H- | | | |
| (A, T, H) | (G, D) | pyrazolo[3,4- | | | |
| | | d]pyrimidin-4-ylamine | | | |
| trans-3-Iodo-1-(4- | (5,7-Dimethyl- | trans-3-[4-(5,7- | 139 | 11.9 min | 557 (M + H)⁺ |
| morpholin-4-yl- | benzoxazol-2-yl)-[2- | Dimethyl-benzoxazol- | | (b) | |
| cyclohexyl)-1H- | fluoro-4-(4,4,5,5- | 2-ylamino)-3-fluoro- | | | |
| pyrazolo[3,4- | tetramethyl- | phenyl]-1-(4- | | | |
| d]pyrimidin-4-ylamine | [1,3,2]dioxaborolan-2- | morpholin-4-yl- | | | |
| (A, T, H) | yl)-phenyl]-amine | cyclohexyl)-1H- | | | |
| | (E, D) | pyrazolo[3,4- | | | |
| | | d]pyrimidin-4-ylamine | | | |
| 2-[3-(4-Amino-3-iodo- | (5,7-Dimethyl- | 2-(3-{4-Amino-3-[4- | 140 | 7.4 min (b) | 512 (M + H)⁺ |
| pyrazolo[3,4- | benzoxazol-2-yl)-[4- | (5,7-dimethyl- | | | |
| d]pyrimidin-1-yl)- | (4,4,5,5-tetramethyl- | benzoxazol-2- | | | |
| piperidin-1-yl]- | [1,3,2]dioxaborolan-2- | ylamino)-phenyl]- | | | |
| acetamide (A, L, I) | yl)-phenyl]-amine | pyrazolo[3,4- | | | |
| | (G, D) | d]pyrimidin-1-yl}- | | | |
| | | piperidin-1-yl)- | | | |
| | | acetamide | | | |
| 4-(4-Amino-3-iodo- | (5,7-Dimethyl- | 4-{4-Amino-3-[4-(5,7- | 141 | 10.9 min | 466 (M − H)⁻ |
| pyrazolo[3,4- | benzoxazol-2-yl)-[4- | dimethyl-benzoxazol- | | (b) | |
| d]pyrimidin-1-yl)- | (4,4,5,5-tetramethyl- | 2-ylamino)-phenyl]- | | | |
| cyclohexanone (A, T) | [1,3,2]dioxaborolan-2- | pyrazolo[3,4- | | | |
| | yl)-phenyl]-amine | d]pyrimidin-1-yl}- | | | |
| | (G, D) | cyclohexanone | | | |
| 1-(1,4-Dioxa- | (5,7-Dimethyl- | 3-[4-(5,7-Dimethyl- | 142 | 11.8 min | 512 (M + H)⁺ |
| spiro[4.5]dec-8-yl)-3- | benzoxazol-2-yl)-[4- | benzoxazol-2- | | (b) | |
| iodo-1H-pyrazolo[3,4- | (4,4,5,5-tetramethyl- | ylamino)-phenyl]-1- | | | |
| d]pyrimidin-4-ylamine | [1,3,2]dioxaborolan-2- | (1,4-dioxa- | | | |
| (A) | yl)-phenyl]-amine | spiro[4.5]dec-8-yl)-1H- | | | |
| | (G, D) | pyrazolo[3,4- | | | |
| | | d]pyrimidin-4-ylamine | | | |
| 1-Cyclopentyl-3-iodo- | (5,7-Dimethyl- | 1-Cyclopentyl-3-[4- | 143 | 13.0 min | 440 (M + H)⁺ |
| 1H-pyrazolo[3,4- | benzoxazol-2-yl)-[4- | (5,7-dimethyl- | | (b) | |
| d]pyrimidin-4-ylamine | (4,4,5,5-tetramethyl- | benzoxazol-2- | | | |
| (A) | [1,3,2]dioxaborolan-2- | ylamino)-phenyl]-1H- | | | |
| | yl)-phenyl]-amine | pyrazolo[3,4- | | | |
| | (G, D) | d]pyrimidin-4-ylamine | | | |
| 1-Cyclopentyl-3-iodo- | Benzoxazol-2-yl-[4- | 3-[4-(Benzoxazol-2- | 144 | 11.9 min | 412 (M + H)⁺ |
| 1H-pyrazolo[3,4- | (4,4,5,5-tetramethyl- | ylamino)-phenyl]-1- | | (b) | |
| d]pyrimidin-4-ylamine | [1,3,2]dioxaborolan-2- | cyclopentyl-1H- | | | |
| (A) | yl)-phenyl]-amine | pyrazolo[3,4- | | | |
| | (G, D) | d]pyrimidin-4-ylamine | | | |
| 1-(1,4-Dioxa- | Benzoxazol-2-yl-[4- | 3-[4-(Benzoxazol-2- | 145 | 10.7 min | 484 (M + H)⁺ |
| spiro[4.5]dec-8-yl)-3- | (4,4,5,5-tetramethyl- | ylamino)-phenyl]-1- | | (b) | |
| iodo-1H-pyrazolo[3,4- | [1,3,2]dioxaborolan-2- | (1,4-dioxa- | | | |
| d]pyrimidin-4-ylamine | yl)-phenyl]-amine | spiro[4.5]dec-8-yl)-1H- | | | |
| (A) | (G, D) | pyrazolo[3,4- | | | |
| | | d]pyrimidin-4-ylamine | | | |
| trans-4-(4-Amino-3- | (5-Chloro-7-methyl- | trans-4-{4-Amino-3-[4- | 146 | 10.5 min | 488 (M − H)⁻ |
| iodo-pyrazolo[3,4- | benzoxazol-2-yl)-[4- | (5-chloro-7-methyl- | | (b) | |
| d]pyrimidin-1-yl)- | (4,4,5,5-tetramethyl- | benzoxazol-2- | | | |
| cyclohexanol (A, T, U) | [1,3,2]dioxaborolan-2- | ylamino)-phenyl]- | | | |
| | yl)-phenyl]-amine | pyrazolo[3,4- | | | |
| | (F, H, G, D) | d]pyrimidin-1-yl}- | | | |
| | | cyclohexanol | | | |
| cis-4-(4-Amino-3-iodo- | (5-Chloro-7-methyl- | cis-4-{4-Amino-3-[4-(5- | 147 | 10.7 min | 488 (M − H)⁻ |
| pyrazolo[3,4- | benzoxazol-2-yl)-[4- | chloro-7-methyl- | | (b) | |
| d]pyrimidin-1-yl)- | (4,4,5,5-tetramethyl- | benzoxazol-2- | | | |
| cyclohexanol (A, T, U) | [1,3,2]dioxaborolan-2- | ylamino)-phenyl]- | | | |
| | yl)-phenyl]-amine | pyrazolo[3,4- | | | |
| | (F, H, G, D) | d]pyrimidin-1-yl}- | | | |
| | | cyclohexanol | | | |
| 1-(1,4-Dioxa- | (5-Chloro-7-methyl- | 3-[4-(5-Chloro-7- | 148 | 3.47 min (f) | 532 (M + H)⁺ |
| spiro[4.5]dec-8-yl)-3- | benzoxazol-2-yl)-[4- | methyl-benzoxazol-2- | | | |
| iodo-1H-pyrazolo[3,4- | (4,4,5,5-tetramethyl- | ylamino)-phenyl]-1- | | | |
| d]pyrimidin-4-ylamine | [1,3,2]dioxaborolan-2- | (1,4-dioxa- | | | |
| (A) | yl)-phenyl]-amine | spiro[4.5]dec-8-yl)-1H- | | | |
| | (F, H, G, D) | pyrazolo[3,4- | | | |
| | | d]pyrimidin-4-ylamine | | | |
| 3-Iodo-1-(tetrahydro- | (5,7-Dimethyl- | 3-[4-(5,7-Dimethyl- | 149 | 23.2 min | 470 (M − H)⁻ |
| thiopyran-4-yl)-1H- | benzoxazol-2-yl)-[4- | benzoxazol-2- | | (c) | |
| pyrazolo[3,4- | (4,4,5,5-tetramethyl- | ylamino)-phenyl]-1- | | | |
| d]pyrimidin-4-ylamine | [1,3,2]dioxaborolan-2- | (tetrahydro-thiopyran- | | | |
| (U, A) | yl)-phenyl]-amine | 4-yl)-1H-pyrazolo[3,4- | | | |
| | (G, D) | d]pyrimidin-4-ylamine | | | |
| 1-Cyclopentyl-3-iodo- | (5-Chloro-7-methyl- | 3-[4-(5-Chloro-7- | 150 | 23.8 min | 460 (M + H)⁺ |
| 1H-pyrazolo[3,4- | benzoxazol-2-yl)-[4- | methyl-benzoxazol-2- | | (d) | |
| d]pyrimidin-4-ylamine | (4,4,5,5-tetramethyl- | ylamino)-phenyl]-1- | | | |
| (A) | [1,3,2]dioxaborolan-2- | cyclopentyl-1H- | | | |
| | yl)-phenyl]-amine | pyrazolo[3,4- | | | |
| | (F, H, G, D) | d]pyrimidin-4-ylamine | | | |
| trans-4-[4-(4-Amino-3- | (5-Methyl-benzoxazol- | trans-4-(4-{4-Amino-3- | 151 | 8.7 min (a) | 550 (M − H)⁻ |
| iodo-pyrazolo[3,4- | 2-yl)-[4-(4,4,5,5- | [4-(5-methyl- | | | |
| d]pyrimidin-1-yl)- | tetramethyl- | benzoxazol-2- | | | |
| cyclohexyl]-1-methyl- | [1,3,2]dioxaborolan-2- | ylamino)-phenyl]- | | | |
| piperazin-2-one | yl)-phenyl]-amine | pyrazolo[3,4- | | | |
| (A, T, J(M)) | (G, D) | d]pyrimidin-1-yl}- | | | |
| | | cyclohexyl)-1-methyl- | | | |
| | | piperazin-2-one | | | |
| trans-4-[4-(4-Amino-3- | (5-Ethyl-benzoxazol-2- | trans-4-(4-{4-Amino-3- | 152 | 9.2 min (a) | δ (DMSO-d₆) |
| iodo-pyrazolo[3,4- | yl)-[4-(4,4,5,5- | [4-(5-ethyl- | | | 10.79, 8.23, |
| d]pyrimidin-1-yl)- | tetramethyl- | benzoxazol-2- | | | 7.92, 7.65, |
| cyclohexyl]-1-methyl- | [1,3,2]dioxaborolan-2- | ylamino)-phenyl]- | | | 7.40, 7.33, |
| piperazin-2-one | yl)-phenyl]-amine | pyrazolo[3,4- | | | 6.99, 4.66, |
| (A, T, J(M)) | (G, D) | d]pyrimidin-1-yl}- | | | 3.24, 2.82, |
| | | cyclohexyl)-1-methyl- | | | 2.82, 2.76, |
| | | piperazin-2-one | | | 2.69, 2.00, |
| | | | | | 1.48, 1.22 |
| trans-4-[4-(4-Amino-3- | (5-Chloro-benzoxazol- | trans-4-(4-{4-Amino-3- | 153 | 9.0 min (a) | 570 (M − H)⁻ |
| iodo-pyrazolo[3,4- | 2-yl)-[4-(4,4,5,5- | [4-(5-chloro- | | | |
| d]pyrimidin-1-yl)- | tetramethyl- | benzoxazol-2- | | | |
| cyclohexyl]-1-methyl- | [1,3,2]dioxaborolan-2- | ylamino)-phenyl]- | | | |
| piperazin-2-one | yl)-phenyl]-amine | pyrazolo[3,4- | | | |
| (A, T, J(M)) | (G, D) | d]pyrimidin-1-yl}- | | | |
| | | cyclohexyl)-1-methyl- | | | |
| | | piperazin-2-one | | | |
| trans-4-[4-(4-Amino-3- | [4-(4,4,5,5- | trans-4-[4-{4-Amino-3- | 154 | 9.4 min (a) | 620 (M − H)⁻ |
| iodo-pyrazolo[3,4- | Tetramethyl- | [4-(5-trifluoromethoxy- | | | |
| d]pyrimidin-1-yl)- | [1,3,2]dioxaborolan-2- | benzoxazol-2- | | | |
| cyclohexyl]-1-methyl- | yl)-phenyl]-(5- | ylamino)phenyl]- | | | |
| piperazin-2-one | trifluoromethoxy- | pyrazolo[3,4- | | | |
| (A, T, J(M)) | benzoxazol-2-yl)- | d]pyrimidin-1-yl}- | | | |
| | amine (G, D) | cyclohexyl)-1-methyl- | | | |
| | | piperazin-2-one | | | |
| trans-1-[4-(4-Amino-3- | (5,7-Dimethyl- | trans-1-[4-{4-Amino-3- | 155 | 10.7 min | 542 (M + H)⁺ |
| iodo-pyrazolo[3,4- | benzoxazol-2-yl)-[4- | [4-(5,7-dimethyl- | | (a) | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol (X, | yl)-phenyl]-amine (G, | pyrazolo[3,4- | | | |
| A) | D) | d]pyrimidin-1-yl}- | | | |
| | | cyclohexyloxy)-2- | | | |
| | | methyl-propan-2-ol | | | |
| trans-1-[4-(4-Amino-3- | (5,7-Dimethyl- | trans-1-(4-{4-Amino-3- | 156 | 11.0 min | 560 (M + H)⁺ |
| iodo-pyrazolo[3,4- | benzoxazol-2-yl)-[2- | [4-(5,7-dimethyl- | | (a) | |
| d]pyrimidin-1-yl)- | fluoro-4-(4,4,5,5- | benzoxazol-2- | | | |
| cyclohexyloxy]-2- | tetramethyl- | ylamino)-3-fluoro- | | | |
| methyl-propan-2-ol | [1,3,2]dioxaborolan-2- | phenyl]-pyrazolo[3,4- | | | |
| (X, A) | yl)-phenyl]-amine | d]pyrimidin-1-yl}- | | | |
| | (E, D) | cyclohexyloxy)-2- | | | |
| | | methyl-propan-2-ol | | | |
| trans-1-[4-(4-Amino-3- | (5-Fluoro-benzoxazol- | trans-1-[4-{4-Amino-3- | 157 | 10.0 min | 550 (M + H)⁺ |
| iodo-pyrazolo[3,4- | 2-yl)-[2-fluoro-4- | [3-fluoro-4-(5-fluoro- | | (a) | |
| d]pyrimidin-1-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrazolo[3,4- | | | |
| (X, A) | (G, D) | d]pyrimidin-1-yl}- | | | |
| | | cyclohexyloxy]-2- | | | |
| | | methyl-propan-2-ol | | | |
| trans-1-[4-(4-Amino-3- | [2-Fluoro-4-(4,4,5,5- | trans-1-(4-{4-Amino-3- | 158 | 16.5 min | 598 (M − H)⁻ |
| iodo-pyrazolo[3,4- | tetramethyl- | [3-fluoro-4-(5- | | (d) | |
| d]pyrimidin-1-yl)- | [1,3,2]dioxaborolan-2- | trifluoromethyl- | | | |
| cyclohexyloxy]-2- | yl)-phenyl]-(5- | benzoxazol-2- | | | |
| methyl-propan-2-ol | trifluoromethyl- | ylamino)-phenyl]- | | | |
| (X, A) | benzoxazol-2-yl)- | pyrazolo[3,4- | | | |
| | amine (H, G, D) | d]pyrimidin-1-yl}- | | | |
| | | cyclohexyloxy)-2- | | | |
| | | methyl-propan-2-ol | | | |
| trans-3-Iodo-1-[4-(2- | (5-Fluoro-7-methyl- | trans-3-[4-(5-Fluoro-7- | 159 | 10.5 min | 532 (M + H)⁺ |
| methoxy-ethoxy)- | benzoxazol-2-yl)-[4- | methyl-benzoxazol-2- | | (a) | |
| cyclohexyl]-1H- | (4,4,5,5-tetramethyl- | ylamino)-phenyl]-1-[4- | | | |
| pyrazolo[3,4- | [1,3,2]dioxaborolan-2- | (2-methoxy-ethoxy)- | | | |
| d]pyrimidin-4-ylamine | yl)-phenyl]-amine | cyclohexyl]-1H- | | | |
| (A) | (F, H, G, D) | pyrazolo[3,4- | | | |
| | | d]pyrimidin-4-ylamine | | | |
| trans-5-Iodo-7-[4-(2- | [2-Fluoro-4-(4,4,5,5- | trans-5-[3-Fluoro-4-(5- | 160 | 11.0 min | δ (DMSO-d₆) |
| methoxy-ethoxy)- | tetramethyl- | trifluoromethyl- | | (a) | 10.79, 8.28, |
| cyclohexyl]-7H- | [1,3,2]dioxaborolan-2- | benzoxazol-2- | | | 8.15, 7.82, |
| pyrrolo[2,3- | yl)-phenyl]-(5- | ylamino)-phenyl]-7-[4- | | | 7.73, 7.52, |
| d]pyrimidin-4-ylamine | trifluoromethyl- | (2-methoxy-ethoxy)- | | | 7.37, 6.22, |
| (A, B) | benzoxazol-2-yl)- | cyclohexyl]-7H- | | | 4.60, 3.58, |
| | amine (H, G, D) | pyrrolo[2,3- | | | 3.45, 3.37, |
| | | d]pyrimidin-4-ylamine | | | 3.26, 2.13, |
| | | | | | 1.36 |
| cis-3-Iodo-1-(1-oxo- | (5,7-Dimethyl- | cis-3-[4-(5,7-Dimethyl- | 161 | 9.0 min (a) | 486 (M − H)⁻ |
| tetrahydro-thiopyran- | benzoxazol-2-yl)-[4- | benzoxazol-2- | | | |
| 4-yl)-1H-pyrazolo[3,4- | (4,4,5,5-tetramethyl- | ylamino)-phenyl]-1-(1- | | | |
| d]pyrimidin-4-ylamine | [1,2,3]dioxaborolan-2- | oxo-tetrahydro- | | | |
| (A, EE) | yl)-phenyl]amine | thiopyran-4-yl)-1H- | | | |
| | (G, D) | pyrazolo[3,4- | | | |
| | | d]pyrimidin-4-ylamine | | | |
| trans-3-Iodo-1-(1-oxo- | (5,7-Dimethyl- | trans-3-[4-(5,7- | 162 | 9.3 min (a) | 486 (M − H)⁻ |
| tetrahydro-thiopyran- | benzoxazol-2-yl)-[4- | Dimethyl-benzoxazol- | | | |
| 4-yl)-1H-pyrazolo[3,4- | (4,4,5,5-tetramethyl- | 2-ylamino)-phenyl]-1- | | | |
| d]pyrimidin-4-ylamine | [1,3,2]dioxaborolan-2- | (1-oxo-tetrahydro- | | | |
| (A, EE) | yl)-phenyl]amine | thiopyran-4-yl)-1H- | | | |
| | (G, D) | pyrazolo[3,4- | | | |
| | | d]pyrimidin-4-ylamine | | | |
| trans-1-[4-(4-Amino-5- | (5,7-Dimethyl- | trans-1-[4-{4-Amino-5- | 163 | 10.89 min | 541 (M + H)⁺ |
| iodo-pyrrolo[2,3- | benzoxazol-2-yl)-[4- | [4-(5,7-dimethyl- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine (G, | pyrrolo[2,3- | | | |
| (X, A) | D) | d]pyrimidin-7-yl}- | | | |
| | | cyclohexyloxy)-2- | | | |
| | | methyl-propan-2-ol | | | |
| trans-1-[4-(4-Amino-5- | (5-Chloro-7-methyl- | trans-1-(4-{4-Amino-5- | 164 | 11.24 min | 559 (M − H)⁻ |
| iodo-pyrrolo[2,3- | benzoxazol-2-yl)-[4- | [4-(5-chloro-7-methyl- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrrolo[2,3- | | | |
| (X, A) | (F, H, G, D) | d]pyrimidin-7-yl}- | | | |
| | | cyclohexyloxy)-2- | | | |
| | | methyl-propan-2-ol | | | |
| trans-1-[4-(4-Amino-5- | (5-Methyl-7-chloro- | trans-1-(4-{4-Amino-5- | 165 | 11.36 min | 559 (M − H)⁻ |
| iodo-pyrrolo[2,3- | benzoxazol-2-yl)-[4- | [4-(5-methyl-7-chloro- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrrolo[2,3- | | | |
| (X, A) | (F, H, G, D) | d]pyrimidin-7-yl}- | | | |
| | | cyclohexyloxy)-2- | | | |
| | | methyl-propan-2-ol | | | |
| trans-1-[4-(4-Amino-5- | (5-Fluoro-7-methyl- | trans-1-(4-{4-Amino-5- | 166 | 10.47 min | 543 (M − H)⁻ |
| iodo-pyrrolo[2,3- | benzoxazol-2-yl)-[4- | [4-(5-fluoro-7-methyl- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrrolo[2,3- | | | |
| (X, A) | (F, H, G, D) | d]pyrimidin-7-yl}- | | | |
| | | cyclohexyloxy)-2- | | | |
| | | methyl-propan-2-ol | | | |
| trans-1-[4-(4-Amino-5- | (5-Chloro-benzoxazol- | trans-1-(4-{4-Amino-5- | 167 | 10.64 min | 545 (M − H)⁻ |
| iodo-pyrrolo[2,3- | 2-yl)-[4-(4,4,5,5- | [4-(5-chloro- | | (b) | |
| d]pyrimidin-7-yl)- | tetramethyl- | benzoxazol-2- | | | |
| cyclohexyloxy]-2- | [1,3,2]dioxaborolan-2- | ylamino)-phenyl}- | | | |
| methyl-propan-2-ol | yl)-phenyl]-amine | pyrrolo[2,3- | | | |
| (X, A) | (G, D) | d]pyrimidin-7-yl}- | | | |
| | | cyclohexyloxy)-2- | | | |
| | | methyl-propan-2-ol | | | |
| trans-1-[4-(4-Amino-5- | (5,7-Dimethyl- | trans-1-(4-{4-Amino-5- | 168 | 11.13 min | 557 (M − H)⁻ |
| iodo-pyrrolo[2,3- | benzoxazol-2-yl)-[2- | [4-(5,7-dimethyl- | | (b) | |
| d]pyrimidin-7-yl)- | fluoro-4-(4,4,5,5- | benzoxazol-2- | | | |
| cyclohexyloxy]-2- | tetramethyl- | ylamino)-3-fluoro- | | | |
| methyl-propan-2-ol) | [1,3,2]dioxaborolan-2- | phenyl]-pyrrolo[2,3- | | | |
| (X, A) | yl)-phenyl]-amine | d]pyrimidin-7-yl}- | | | |
| | (E, D) | cyclohexyloxy)-2- | | | |
| | | methyl-propan-2-ol | | | |
| trans-1-[4-(4-Amino-5- | [2-Fluoro-4-(4,4,5,5- | trans-1-[4-{4-Amino-5- | 169 | 10.88 min | 597 (M − H)⁻ |
| iodo-pyrrolo[2,3- | tetramethyl- | [3-fluoro-4-(5- | | (b) | |
| d]pyrimidin-7-yl)- | [1,3,2]dioxaborolan-2- | trifluoromethyl- | | | |
| cyclohexyloxy]-2- | yl)-phenyl]-(5- | benzoxazol-2- | | | |
| methyl-propan-2-ol) | trifluoromethyl- | ylamino)-phenyl]- | | | |
| (X, A) | benzoxazol-2-yl)- | pyrrolo[2,3- | | | |
| | amine (F, H, G, D) | d]pyrimidin-7-yl}- | | | |
| | | cyclohexyloxy)-2- | | | |
| | | methyl-propan-2-ol | | | |
| trans-1-[4-(4-Amino-5- | (5-Chloro-benzoxazol- | trans-1-(4-{4-Amino-5- | 170 | 10.73 min | 563 (M − H)⁻ |
| iodo-pyrrolo[2,3- | 2-yl)-[2-fluoro-4- | [4-(5-chloro- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-3-fluoro- | | | |
| methyl-propan-2-ol | yl)-phenyl]-amine | phenyl]-pyrrolo[2,3- | | | |
| (X, A) | (E, D) | d]pyrimidin-7-yl}- | | | |
| | | cyclohexyloxy)-2- | | | |
| | | methyl-propan-2-ol | | | |
| trans-1-[4-(4-Amino-5- | (7-Chloro-5-fluoro- | trans-1-(4-{4-Amino-5- | 171 | 11.04 min | 563 (M − H)⁻ |
| iodo-pyrrolo[2,3- | benzoxazol-2-yl)-[4- | [4-(7-chloro-5-fluoro- | | (b) | |
| d]pyrimidin-7-yl)- | (4,4,5,5-tetramethyl- | benzoxazol-2- | | | |
| cyclohexyloxy]-2- | [1,3,2]dioxaborolan-2- | ylamino)-phenyl]- | | | |
| methyl-propan-2-ol | yl)-phenyl]-amine | pyrrolo[2,3- | | | |
| (X, A) | (F, H, G, D) | d]pyrimidin-7-yl}- | | | |
| | | cyclohexyloxy)-2- | | | |
| | | methyl-propan-2-ol | | | |
| (3-Bromo-1-methyl- | (5,7-Dimethyl- | {3-[4-(5,7-Dimethyl- | 172 | 11.59 min | 490 (M + H)⁺ |
| 1H-pyrazolo[3,4- | benzoxazol-2-yl)-[2- | benzoxazol-2- | | (a) | |
| d]pyrimidin-6-yl)- | fluoro-4-(4,4,5,5 | ylamino)-phenyl]-1- | | | |
| phenethyl-amine | tetramethyl- | methyl-1H- | | | |
| (preparation #29) | [1,3,2]dioxaborolan-2- | pyrazolo[3,4- | | | |
| | yl)-phenyl]-amine | d]pyrimidin-6-yl}- | | | |
| | (G, D) | phenethyl-amine | | | |
| trans-1-{2-[4-(4- | (5-Chloro-benzoxazol- | trans-1-[2-(4-{4- | 173 | 3.75 min (f) | 638 (M + H)⁺ |
| Amino-3-iodo- | 2-yl)-[2-fluoro-4 | Amino-3-[4-(5-chloro- | | | |
| pyrazolo[3,4- | (4,4,5,5-tetramethyl- | benzoxazol-2- | | | |
| d]pyrimidin-1-yl)- | [1,3,2]dioxaborolan-2- | ylamino)-3-fluoro- | | | |
| cyclohexyloxy]-1,1 | yl)-phenyl]-amine | phenyl]-pyrazolo[3,4- | | | |
| dimethyl-ethoxy}-2- | (G, D) | d]pyrimidin-1-yl]- | | | |
| methyl-propan-2-ol | | cyclohexyloxy)-1,1- | | | |
| (X, A) | | dimethyl-ethoxy]-2- | | | |
| | | methyl-propan-2-ol | | | |

### General Procedure D: Conversion of a Bromide to a Boronate

A mixture of bis(pinacolato)diboron (1-1.5 equivalents, preferably 1.3 equivalents), an aryl bromide (preferably 1 equivalent), dichloro[1,1′bis(diphenylphosphino)ferrocene]-palladium (II) dichloromethane adduct (0.03-0.15 equiv, preferably 0.10 equivalents) and a base (for example, sodium acetate or potassium acetate, preferably potassium acetate) (1.5-3.0 equivalents, preferably 2.5 equivalents) is heated in an organic solvent (for example, N,N-dimethylformamide, dioxane, or tetrahydrofuran, preferably N,N-dimethylformamide) at about 50-100° C. (preferably 80° C.) for about 1-24 hours (preferably 15 hours) under an inert atmosphere. The mixture is allowed to cool to ambient temperature, and the solvent is removed under reduced pressure. The solid residue can then be purified by flash column chromatography or crystallization.

### Illustration of General Procedure D

### Preparation #4: 2-Methyl-4-(4,4,5,5-tetramethyl-[1,3,2]dioxaborolan-2-yl)-phenylamine

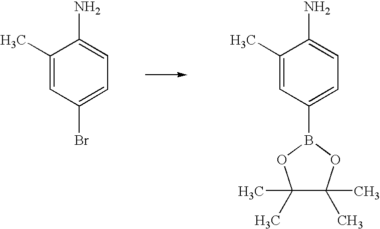

A mixture of 4-bromo-2-methylaniline (0.250 g, 1.34 mmol), bis(pinacolato)diboron (0.442 g, 1.747 mmol), dichloro[1,1′bis(diphenylphosphino)-ferrocene]palladium (II) dichloromethane adduct (0.110 g, 0.134 mmol), and potassium acetate (0.329 g, 3.357 mmol) was heated in N,N-dimethylformamide (5 mL) at about 80° C. for about 15 h under an atmosphere of nitrogen. The mixture was allowed to cool to ambient temperature and was purified by flash column chromatography on silica gel using ethyl acetate/heptane (3:7) as the mobile phase to give 2-methyl-4-(4,4,5,5-tetramethyl-[1,3,2]dioxaborolan-2-yl)-phenylamine as a yellow oil (0.213 g, 0.914 mmol); RP-HPLC (25% to 100% acetonitrile/0.1 M aqueous ammonium acetate, buffered to pH 4.5, over 10 min at 1.0 mL/min; λ=254 nm; Hypersil C18, 100 Å, 5 μm, 250×4.6 mm column) Rₜ 11.02 min.

### General Procedure E: Cyclization of an Aminoalcohol to a Benzoxazole

An aminophenol (1-2 equivalents, preferably 1 equivalent) is added to a solution of an aryl isothiocyanate (1-2 equivalents, preferably 1 equivalent) in an organic solvent (for example, tetrahydrofuran or pyridine) at about −40 to 50° C. The mixture is stirred at about 0 to 50° C. for about 1-24 hours. 1-(3-Dimethylaminopropyl)-3-ethylcarbodiimide hydrochloride (EDC) (1-2 equivalents, preferably 1 equivalent) is added to the reaction and the mixture is heated at about 40-80° C. for about 1-24 hours (preferably 15 hours). The mixture is allowed to cool to ambient temperature and the solvent is removed under reduced pressure to afford the product that can be further purified by crystallization or chromatography.

### Illustration of General Route E

### Preparation #5: (4-Bromo-phenyl)-(5-chloro-benzoxazol-2-yl)-amine

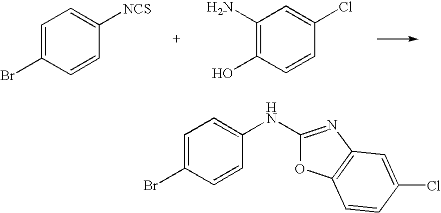

To a solution of 4-bromophenyl isothiocyanate (46.2 g, 0.216 mol) in tetrahydrofuran (750 mL) was added 2-amino-4-chlorophenol (31.0 g, 0.216 mol), at room temperature under a nitrogen atmosphere. The reaction mixture was stirred at room temperature for about 2 hours. 1-(3-Dimethylaminopropyl)-3-ethylcarbodiimide hydrochloride (49.7 g, 0.259 mol) was added to the reaction mixture and the mixture was stirred at about 50° C. for about 15 hours. The solvent was removed under reduced pressure and the residue was partitioned between 0.1 N aqueous hydrochloric acid (250 mL) and dichloromethane (300 mL). The resulting crude precipitate was collected by filtration and washed with water (200 mL). The remaining organic layer of the filtrate was separated and the aqueous layer was extracted with additional dichloromethane (2×200 mL). The combined organic layers were dried over magnesium sulfate and the solvent was removed under reduced pressure. The crude product (30 g) was then purified by flash column chromatography on silica using heptane/ethyl acetate (90:10 to 75:25) as a mobile phase to afford (4-bromo-phenyl)-(5-chloro-benzoxazol-2-yl)-amine as a pink solid (15.8 g, 0.048 mol); RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 12.984 min.

### General Procedure F: Nitration of Phenols

A substituted phenol (preferably 1.0 equiv.) is dissolved in an organic solvent (for example, diethyl ether or ethylene glycol dimethyl ether, preferably ethylene glycol dimethyl ether) and the resulting solution is cooled to about −60° C. (preferably, about −50° C). Nitronium tetrafluoroborate (1-2 equivalents, preferably 1.02 equiv.) is added and the reaction mixture is gradually warmed to ambient temperature while stirring under a nitrogen atmosphere for about 2-96 hours. The organic solvent is removed under reduced pressure and the residue can be further purified by chromatography or crystallization.

### Illustration of General Procedure F

### Preparation #6: 5-Bromo-2-hydroxy-3-nitrobenzonitrile

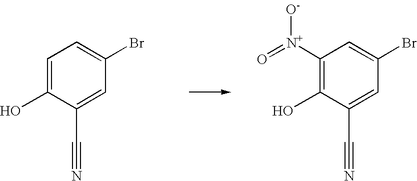

5-Bromo-2-hydroxybenzonitrile (5.04 g, 0.0254 mol) was dissolved in ethylene glycol dimethyl ether (60 mL) and the resulting solution was cooled to about −55° C. Nitronium tetrafluoroborate (3.58 g, 0.0259 mol) was added at once and the reaction mixture was gradually warmed to ambient temperature while stirring under continuous nitrogen flow for about 24 hours. Ethylene glycol dimethyl ether was removed under reduced pressure and the residue was passed through a silica gel pad (40 g) eluting with ethyl acetate (800 mL). Ethyl acetate was removed under reduced pressure and the residue was triturated with cold ether (15 mL). The precipitate was collected by filtration and dried to yield 5-bromo-2-hydroxy-3-nitrobenzonitrile (3.30 g, 0.0136 mol) as a yellow solid; m/z: (M−H)⁻ 241 and 243.

### General Procedure G: Transformation of an Aniline to an Aminobenzoxazole

To a solution of the aniline (preferably 1 equivalent) in an organic solvent (for example, dichloromethane, acetonitrile, or pyridine, preferably pyridine) at about 0-25° C. (preferably 25° C.) was added a thiocarbonyl (for example, 1,1′-thiocarbonyldi-2(1H)-pyridone or 1,1′-thiocarbonyl-diimidazole, preferably 1,1′-thiocarbonyldiimidazole) (1-5 equivalents, preferably 1.05 equivalents) for about 0.5-2 hours (preferably about 1 hour), and the mixture was stirred at about 0-50° C. (preferably about 25° C.) for about 1-5 hours (preferably about 2 hours). A 2-aminophenol (1-2 equivalents, preferably 1 equivalent) is added to the reaction mixture and stirred for about 1-12 hours (preferably 2 hours) at about 0-50° C. (preferably about 25° C.). A carbodiimide, (preferably 1-(3-dimethylaminopropyl)-3-ethylcarbodiimide) (1-5 equivalents, preferably 1.2 equivalents) is added to the reaction and the mixture is stirred at about 25-70° C. (preferably about 50° C.) for about 1-48 hours (preferably about 12 hours). The mixture is cooled to ambient temperature and the solvent is removed under reduced pressure. The residue is partitioned between an aqueous acidic solution and an organic solvent, the organic layer is separated and the aqueous layer is further extracted with an organic solvent. The combined organic extracts are dried over a desiccant. The solvents are evaporated under reduced pressure to afford the product that can be further purified by crystallization or chromatography.

### Illustration of General Procedure G

### Preparation #7: (4-Bromo-2-fluoro-phenyl)-(5,7-dimethyl-benzoxazol-2-yl)-amine

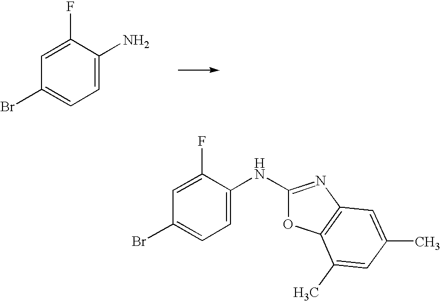

To a solution of 4-bromo-2-fluoroaniline (5 g, 26.3 mmol) in pyridine (150 mL) at ambient temperature was added 1,1′-thiocarbonyldiimidazole (4.92 g, 27.6 mmol). The reaction mixture was stirred, under an atmosphere of nitrogen, for about 1 hour. 2,4-Dimethyl-6-aminophenol (3.61 g, 26.3 mmol) was added to the reaction mixture and the mixture was stirred at room temperature, under nitrogen, for about 45 additional minutes. Pyridine was removed from the reaction mixture under reduced pressure. The residue was taken up in acetonitrile (200 mL), 1-(3-dimethylaminopropyl)-3-ethylcarbodiimide (6.05 g, 31.6 mmol) was added, and the mixture was stirred at ambient temperature for about 20 hours, before being heated at about 60° C. for about 5 additional hours. The acetonitrile was removed under reduced pressure and the residue was taken up in ethyl acetate (100 mL), then washed with 0.5 N hydrochloric acid (4×100 mL) and brine (100 mL). The organic layer was dried over magnesium sulfate and the organic solvent was removed under reduced pressure. The crude product was triturated in hot methanol to afford (4-bromo-2-fluoro-phenyl)-(5, 7-dimethyl-benzoxazol-2-yl)-amine as a pink solid (4.24 g, 12.6 mmol); ¹H NMR (DMSO-d₆, 400 MHz) δ 10.51, 8.28, 7.60, 7.47, 7.07, 6.79, 2.38, 2.33; RP-HPLC (5% to 95% acetonitrile/0.05M aqueous ammonium acetate, buffered to pH 4.5, over 10 min at 1 mL/min; λ=254 nm; Hypersil C18, 100 Å, 5 μm, 250×4.6 mm column) Rₜ 10.36 min.

Other products obtained using general procedure G are shown (Table 4). The method used to determine the HPLC retention time is given in a lower-case letter in parentheses (see Table 1).

TABLE 4
Examples Synthesized using general procedure G

| | | | | | m/z or ¹H |
|---|---|---|---|---|---|
| | | | | | NMR |
| | | | | HPLC RT | δ (DMSO- |
| Aniline precursor | Amino phenol | Product | Example # | (Method) | d₆) |
| trans-3-(4-Amino-3- | 2-Amino-4,6-dimethyl- | trans-3-[3-Chloro-4- | 174 | 3.13 min (f) | 586 (M + H)⁺ |
| chloro-phenyl)-1-[4-(4- | phenol | (5,7-dimethyl- | | | |
| methyl-piperazin-1-yl)- | | benzoxazol-2- | | | |
| cyclohexyl]-1H- | | ylamino)-phenyl]-1-[4- | | | |
| pyrazolo[3,4- | | (4-methyl-piperazin-1- | | | |
| d]pyrimidin-4-ylamine | | yl)-cyclohexyl]-1H- | | | |
| (A,T,J,C(D)) | | pyrazolo[3,4- | | | |
| | | d]pyrimidin-4-ylamine | | | |
| trans-3-(4-Amino- | 2-Amino-6-bromo-4- | trans-3-[4-(7-Bromo-5- | 175 | 3.48 min (f) | 592 (M + H)⁺ |
| phenyl)-1-[4-(2- | methyl-phenol (H) | methyl-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 2-Amino-6-bromo-4- | trans-3-[4-(7-Bromo-5- | 176 | 3.63 min (f) | 610 (M + H)⁺ |
| fluoro-phenyl)-1-[4-(2- | methyl-phenol (H) | methyl-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyrazolo[3,4- | | | |
| | | d]pyrimidin-4-ylamine | | | |
| trans-3-(4-Amino- | 2-Amino-6-bromo-4- | trans-3-[4-(7-Bromo-5- | 177 | 3.30 min (f) | 596 (M + H)⁺ |
| phenyl)-1-[4-(2- | fluoro-phenol (F,H) | fluoro-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 2-Amino-6-bromo-4- | trans-3-[4-(7-Bromo-5- | 178 | 3.22 min (f) | 614 (M + H)⁺ |
| fluoro-phenyl)-1-[4-(2- | fluoro-phenol (F,H) | fluoro-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyrazolo[3,4- | | | |
| | | d]pyrimidin-4-ylamine | | | |
| trans-5-(4-Amino- | 2-Amino-6-bromo-4- | trans-5-[4-(7-Bromo-5- | 179 | 3.40 min (f) | 595 (M + H)⁺ |
| phenyl)-7-[4-(2- | fluoro-phenol (F,H) | fluoro-benzoxazol-2- | | | |
| methoxy-ethoxy)- | | ylamino)-phenyl]-7-[4- | | | |
| cyclohexyl]-7H- | | (2-methoxy-ethoxy)- | | | |
| pyrrolo[2,3- | | cyclohexyl]-7H- | | | |
| d]pyrimidin-4-ylamine | | pyrrolo[2,3- | | | |
| (A,C) | | d]pyrimidin-4-ylamine | | | |
| trans-5-(4-Amino- | 2-Amino-6-bromo-4- | trans-5-[4-(7-Bromo-5- | 180 | 3.46 min (f) | 591 (M + H)⁺ |
| phenyl)-7-[4-(2- | methyl-phenol (H) | methyl-benzoxazol-2- | | | |
| methoxy-ethoxy)- | | ylamino)-phenyl]-7-[4- | | | |
| cyclohexyl]-7H- | | (2-methoxy-ethoxy)- | | | |
| pyrrolo[2,3- | | cyclohexyl]-7H- | | | |
| d]pyrimidin-4-ylamine | | pyrrolo[2,3- | | | |
| (A,C) | | d]pyrimidin-4-ylamine | | | |
| trans-4-{4-[4-Amino-3- | 2-Amino-4,6-dimethyl- | trans-4-(4-{4-Amino-3- | 181 | 2.60 min (f) | 596 (M + H)⁺ |
| (4-amino-3-methoxy- | phenol | [4-(5,7-dimethyl- | | | |
| phenyl)-pyrazolo[3,4- | | benzoxazol-2- | | | |
| d]pyrimidin-1-yl]- | | ylamino)-3-methoxy- | | | |
| cyclohexyl}-1-methyl- | | phenyl]-pyrazolo[3,4- | | | |
| piperazin-2-one (A,T,J | | d]pyrimidin-1-yl}- | | | |
| (M),C,L) | | cyclohexyl)-1-methyl- | | | |
| | | piperazin-2-one | | | |
| trans-4-{4-[4-Amino-3- | 2-Amino-4-methyl- | trans-4-(4-{4-Amino-3- | 182 | 2.52 min (f) | 582 (M + H)⁺ |
| (4-amino-3-methoxy- | phenol | [3-methoxy-4-(5- | | | |
| phenyl)-pyrazolo[3,4- | | methyl-benzoxazol-2- | | | |
| d]pyrimidin-1-yl]- | | ylamino)-phenyl]- | | | |
| cyclohexyl}-1-methyl- | | pyrazolo[3,4- | | | |
| piperazin-2-one | | d]pyrimidin-1-yl}- | | | |
| (A,T,J(M),C,L) | | cyclohexyl)-1-methyl- | | | |
| | | piperazin-2-one | | | |
| trans-4-{4-[4-Amino- | 2-Amino-4-chloro- | trans-4-(4-{4-Amino-3- | 183 | 2.48 min (f) | 588 (M + H)⁺ |
| 3-(4-amino-3-fluoro- | phenol | [4-(5-chloro- | | | |
| phenyl)-pyrazolo[3,4- | | benzoxazol-2- | | | |
| d]pyrimidin-1-yl]- | | ylamino)-3-fluoro- | | | |
| cyclohexyl}-1-methyl- | | phenyl]-pyrazolo[3,4- | | | |
| piperazin-2-one (A,T,J | | d]pyrimidin-1-yl}- | | | |
| (M),C,L) | | cyclohexyl)-1-methyl- | | | |
| | | piperazin-2-one | | | |
| trans-4-{4-[4-Amino-3- | 2-Amino-4-chloro- | trans-4-(4-{4-Amino-3- | 184 | 2.53 min (f) | 602 (M + H)⁺ |
| (4-amino-3-methoxy- | phenol | [4-(5-chloro- | | | |
| phenyl)-pyrazolo[3,4- | | benzoxazol-2- | | | |
| d]pyrimidin-1-yl]- | | ylamino)-3-methoxy- | | | |
| cyclohexyl}-1-methyl- | | phenyl]-pyrazolo[3,4- | | | |
| piperazin-2-one (A,T, | | d]pyrimidin-1-yl}- | | | |
| J(M),C,L) | | cyclohexyl)-1-methyl- | | | |
| | | piperazin-2-one | | | |
| trans-4-{4-[4-Amino-3- | 2-Amino-4-methyl- | trans-4-(4-{4-Amino-3- | 185 | 2.38 min (f) | 570 (M + H)⁺ |
| (4-amino-3-fluoro- | phenol | [3-fluoro-4-(5-methyl- | | | |
| phenyl)-pyrazolo[3,4- | | benzoxazol-2- | | | |
| d]pyrimidin-1-yl]- | | ylamino)-phenyl]- | | | |
| cyclohexyl}-1-methyl- | | pyrazolo[3,4- | | | |
| piperazin-2-one (A,T,J | | d]pyrimidin-1-yl}- | | | |
| (M),C,L) | | cyclohexyl)-1-methyl- | | | |
| | | piperazin-2-one | | | |
| trans-4-{4-[4-Amino-3- | 2-Amino-4,6-methyl- | trans-4-(4-{4-Amino-3- | 186 | 2.60 min (f) | 584 (M + H)⁺ |
| (4-amino-3-fluoro- | phenol | [4-(5,7-dimethyl- | | | |
| phenyl)-pyrazolo[3,4- | | benzoxazol-2- | | | |
| d]pyrimidin-1-yl]- | | ylamino)-3-fluoro- | | | |
| cyclohexyl}-1-methyl- | | phenyl]-pyrazolo[3,4- | | | |
| piperazin-2-one (A,T,J | | d]pyrimidin-1-yl}- | | | |
| (M),C,L) | | cyclohexyl)-1-methyl- | | | |
| | | piperazin-2-one | | | |
| trans-4-{4-[4-Amino-3- | 2-Amino-4-fluoro- | trans-4-(4-{4-Amino-3- | 187 | 2.37 min (f) | 586 (M + H)⁺ |
| (4-amino-3-methoxy- | phenol (F,H) | [4-(5-fluoro- | | | |
| phenyl)-pyrazolo[3,4- | | benzoxazol-2- | | | |
| d]pyrimidin-1-yl]- | | ylamino)-3-methoxy- | | | |
| cyclohexyl}-1-methyl- | | phenyl]-pyrazolo[3,4- | | | |
| piperazin-2-one (A,T,J | | d]pyrimidin-1-yl}- | | | |
| (M),C,L) | | cyclohexyl)-1-methyl- | | | |
| | | piperazin-2-one | | | |
| trans-3-(4-Amino- | 2-Amino-4-chloro-6- | trans-3-[4-(7-Chloro-5- | 188 | 3.28 min (f) | 552 (M + H)⁺ |
| phenyl)-1-[4-(2- | fluoro-phenol (F,H) | fluoro-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino- | 2-Amino-4,6-difluoro- | trans-3-[4-(5,7- | 189 | 3.07 min (f) | 536 (M + H)⁺ |
| phenyl)-1-[4-(2- | phenol (F,H) | Difluoro-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 2-Amino-4,6-difluoro- | trans-3-[4-(5,7- | 190 | 3.10 min (f) | 554 (M + H)⁺ |
| fluoro-phenyl)-1-[4-(2- | phenol (F,H) | Difluoro-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyrazolo[3,4- | | | |
| | | d]pyrimidin-4-ylamine | | | |
| trans-3-(4-Amino-3- | 2-Amino-4-chloro-6- | trans-3-[4-(7-Chloro-5- | 191 | 3.35 min (f) | 570 (M + H)⁺ |
| fluoro-phenyl)-1-[4-(2- | fluoro-phenol (F,H) | fluoro-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyazolo[3,4- | | | |
| | | d]pyrimidin-4-ylamine | | | |
| trans-5-(4-Amino- | 2-Amino-4,6-difluoro- | trans-5-[4-(5,7- | 192 | 3.10 min (f) | 533 (M − H)⁻ |
| phenyl)-7-[4-(2- | phenol (F,H) | Difluoro-benzoxazol-2- | | | |
| methoxy-ethoxy)- | | ylamino)-phenyl]-7-[4- | | | |
| cyclohexyl]-7H- | | (2-methoxy-ethoxy)- | | | |
| pyrrolo[2,3- | | cyclohexyl]-7H- | | | |
| d]pyrimidin-4-ylamine | | pyrrolo[2,3- | | | |
| (A,C) | | d]pyrimidin-4-ylamine | | | |
| trans-5-(4-Amino- | 2-Amino-4-chloro-6- | trans-5-[4-(7-Chloro-5- | 193 | 3.30 min (f) | 553 (M + H)⁺ |
| phenyl)-7-[4-(2- | fluoro-phenol (F,H) | fluoro-benzoxazol-2- | | | |
| methoxy-ethoxy)- | | ylamino)-phenyl]-7-[4- | | | |
| cyclohexyl]-7H- | | (2-methoxy-ethoxy)- | | | |
| pyrrolo[2,3- | | cyclohexyl]-7H- | | | |
| d]pyrimidin-4-ylamine | | pyrrolo[2,3- | | | |
| (A,C) | | d]pyrimidin-4-ylamine | | | |
| trans-5-(4-Amino-3- | 2-Amino-4,6-difluoro- | trans-5-[4-(5,7- | 194 | 3.12 min (f) | 551 (M − H)⁻ |
| fluoro-phenyl)-7-[4-(2- | phenol (F,H) | Difluoro-benzoxazol-2- | | | |
| methoxy-ethoxy)- | | ylamino)-3-fluoro- | | | |
| cyclohexyl]-7H- | | phenyl]-7-[4-(2- | | | |
| pyrrolo[2,3- | | methoxy-ethoxy)- | | | |
| d]pyrimidin-4-ylamine | | cyclohexyl]-7H- | | | |
| (A,C,L) | | pyrrolo[2,3- | | | |
| | | d]pyrimidin-4-ylamine | | | |
| trans-5-(4-Amino-3- | 2-Amino-4-chloro-6- | trans-5-[4-(7-Chloro-5- | 195 | 3.23 min (f) | 567 (M − H)⁻ |
| fluoro-phenyl)-7-[4-(2- | fluoro-phenol (F,H) | methyl-benzoxazol-2- | | | |
| methoxy-ethoxy)- | | ylamino)-3-fluoro- | | | |
| cyclohexyl]-7H- | | phenyl]-7-[4-(2- | | | |
| pyrrolo[2,3- | | methoxy-ethoxy)- | | | |
| d]pyrimidin-4-ylamine | | cyclohexyl]-7H- | | | |
| (A,C,L) | | pyrrolo[2,3- | | | |
| | | d]pyrimidin-4-ylamine | | | |
| trans-3-(4-Amino- | 2-Amino-4-bromo-6- | trans-3-[4-(5-Bromo-7- | 196 | 3.35 min (f) | 592 (M + H)⁺ |
| phenyl)-1-[4-(2- | methyl-phenol (F,H) | methyl-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 2-Amino-4-bromo-6- | trans-3-[4-(5-Bromo-7- | 197 | 3.48 min (f) | 611 (M + H)⁺ |
| fluoro-phenyl)-1-[4-(2- | methyl-phenol (F,H) | methyl-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyrazolo[3,4- | | | |
| | | d]pyrimidin-4-ylamine | | | |
| 5-(4-Amino-phenyl)-7- | 2-Amino-4-bromo-6- | trans-5-[4-(5-Bromo-7- | 198 | 3.40 min (f) | 592 (M + H)⁺ |
| [4-(2-methoxy-ethoxy)- | methyl-phenol (F,H) | methyl-benzoxazol-2- | | | |
| cyclohexyl]-7H- | | ylamino)-phenyl]-7-[4- | | | |
| pyrrolo[2,3- | | (2-methoxy-ethoxy)- | | | |
| d]pyrimidin-4-ylamine | | cyclohexyl]-7H- | | | |
| (B,A,C) | | pyrrolo[2,3- | | | |
| | | d]pyrimidin-4-ylamine | | | |
| cis-3-(4-Amino-3- | 2-Amino-4,6-dimethyl- | cis-3-[4-(5,7-Dimethyl- | 199 | 2.28 min (f) | 582 (M + H)⁺ |
| methoxy-phenyl)-1-[4- | phenol | benzoxazol-2-yl- | | | |
| (4-methyl-piperazin-1- | | amino)-3-methoxy- | | | |
| yl)-cyclohexyl]-1H- | | phenyl]-1-[4-(4-methyl- | | | |
| pyrazolo[3,4-d] | | piperazin-1-yl)- | | | |
| pyrimidin-4-ylamine | | cyclohexyl]-1H- | | | |
| (A,T,J,C,L) | | pyrazolo[3,4- | | | |
| | | d]pyrimidin-4-ylamine | | | |
| trans-3-(4-Amino- | 2-Amino-4-chloro-6- | trans-3-[4-(5-Chloro-7- | 200 | 7.60 min (b) | 561 (M − H)⁻ |
| phenyl)-1-(4- | fluoro-phenol (F,H) | fluoro-benzoxazol-2- | | | |
| morpholin-4-yl- | | ylamino)-phenyl]-1-(4- | | | |
| cyclohexyl)-1H- | | morpholin-4-yl- | | | |
| pyrazolo[3,4- | | cyclohexyl)-1H- | | | |
| d]pyrimidin-4-ylamine | | pyrazolo[3,4- | | | |
| (U,A,T,J) | | d]pyrimidin-4-ylamine | | | |
| trans-3-(4-Amino- | 2-Amino-6-ethyl-4- | trans-3-[4-(7-Ethyl-5- | 201 | 7.51 min (b) | 557 (M + H)⁺ |
| phenyl)-1-(4- | fluoro-phenol (F,H) | fluoro-benzoxazol-2- | | | |
| morpholin-4-yl- | | ylamino)-phenyl]-1-(4- | | | |
| cyclohexyl)-1H- | | morpholin-4-yl- | | | |
| pyrazolo[3,4- | | cyclohexyl)-1H- | | | |
| d]pyrimidin-4-ylamine | | pyrazolo[3,4- | | | |
| (U,A,T,J) | | d]pyrimidin-4-ylamine | | | |
| trans-3-(4-Amino- | 3-Amino-5-bromo-4- | trans-2-{4-[4-Amino-1- | 202 | 7.14 min (b) | 612 (M − H)⁻ |
| phenyl)-1-(4- | hydroxy-benzonitrile | (4-morpholin-4-yl- | | | |
| morpholin-4-yl- | (F,H) | cyclohexyl)-1H- | | | |
| cyclohexyl)-1H- | | pyrazolo[3,4- | | | |
| pyrazolo[3,4- | | d]pyrimidin-3-yl]- | | | |
| d]pyrimidin-4-ylamine | | phenylamino}-7- | | | |
| (U,A,T,J) | | bromo-benzoxazole-5- | | | |
| | | carbonitrile | | | |
| trans-3-(4-Amino- | 2-Amino-6-methoxy-4- | trans-3-[4-(7-Methoxy- | 203 | 8.57 min (b) | 553 (M − H)⁻ |
| phenyl)-1-(4- | methyl-phenol (F,H) | 5-methyl-benzoxazol- | | | |
| morpholin-4-yl- | | 2-ylamino)-phenyl]-1- | | | |
| cyclohexyl)-1H- | | (4-morpholin-4-yl- | | | |
| pyrazolo[3,4- | | cyclohexyl)-1H- | | | |
| d]pyrimidin-4-ylamine | | pyrazolo[3,4- | | | |
| (U,A,T,J) | | d]pyrimidin-4-ylamine | | | |
| | | bis acetic acid salt | | | |
| trans-3-(4-Amino- | 2-Amino-6-chloro-4- | trans-3-[4-(7-Chloro-5- | 204 | 11.43 min (a) | 548 (M + H)⁺ |
| phenyl)-1-[4-(2- | methyl-phenol (F,H) | methyl-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 2-Amino-6-chloro-4- | trans-3-[4-(7-Chloro-5- | 205 | 11.69 min (a) | 564 (M − H)⁻ |
| fluoro-phenyl)-1-[4-(2- | methyl-phenol (F,H) | methyl-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl)-1H- | | | |
| (A,C,L) | | pyrazolo[3,4- | | | |
| | | d]pyrimidin-4-ylamine | | | |
| trans-3-(4-Amino-3- | 2-Amino-4-chloro-6- | trans-3-[4-(5-Chloro-7- | 206 | 11.47 min (a) | 568 (M − H)⁻ |
| fluoro-phenyl)-1-[4-(2- | fluoro-phenol (F,H) | fluoro-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl]-1H- | | | |
| (A,C,L) | | pyrazolo[3,4- | | | |
| | | d]pyrimidin-4-ylamine | | | |
| trans-3-(4-Amino- | 2-Amino-4-chloro-6- | trans-3-[4-(5-Chloro-7- | 207 | 11.32 min (a) | 552 (M + H)⁺ |
| phenyl)-1-[4-(2- | fluoro-phenol (F,H) | fluoro-benzoxazol-2- | | | |
| methoxy-ethoxy)- | | ylamino)-phenyl]-1-[4- | | | |
| cyclohexyl]-1H- | | (2-methoxy-ethoxy)- | | | |
| pyrazolo[3,4- | | cyclohexyl]-1H- | | | |
| d]pyrimidin-4-ylamine | | pyrazolo[3,4- | | | |
| (A,C) | | d]pyrimidin-4-ylamine | | | |
| trans-5-(4-Amino-3- | 2-Amino-4-chloro-6- | trans-5-[4-(5-Chloro-7- | 208 | 11.62 min (a) | 565 (M + H)⁺ |
| fluoro-phenyl)-7-[4-(2- | methyl-phenol (F,H) | methyl-benzoxazol-2- | | | |
| methoxy-ethoxy)- | | ylamino)-3-fluoro- | | | |
| cyclohexyl]-7H- | | phenyl]-7-[4-(2- | | | |
| pyrrolo[2,3- | | methoxy-ethoxy)- | | | |
| d]pyrimidin-4-ylamine | | cyclohexyl]-7H- | | | |
| (A,B,C,L) | | pyrrolo[2,3- | | | |
| | | d]pyrimidin-4-ylamine | | | |
| trans-5-(4-Amino-3- | 2-Amino-4-chloro-6- | trans-5-[4-(5-Chloro-7- | 209 | 11.47 min (a) | 569 (M + H)⁺ |
| fluoro-phenyl)-7-[4-(2- | fluoro-phenol (F,H) | fluoro-benzoxazol-2- | | | |
| methoxy-ethoxy)- | | ylamino)-3-fluoro- | | | |
| cyclohexyl]-7H- | | phenyl]-7-[4-(2- | | | |
| pyrrolo[2,3- | | methoxy-ethoxy)- | | | |
| d]pyrimidin-4-ylamine | | cyclohexyl]-7H- | | | |
| (A,B,C,L) | | pyrrolo[2,3- | | | |
| | | d]pyrimidin-4-ylamine | | | |
| trans-5-(4-Amino-3- | 2-Amino-6-chloro-4- | trans-5-[4-(7-Chloro-5- | 210 | 11.71 min (a) | 565 (M + H)⁺ |
| fluoro-phenyl)-7-[4-(2- | methyl-phenol (F,H) | methyl-benzoxazol-2- | | | |
| methoxy-ethoxy)- | | ylamino)-3-fluoro- | | | |
| cyclohexyl]-7H- | | phenyl]-7-[4-(2- | | | |
| pyrrolo[2,3- | | methoxy-ethoxy)- | | | |
| d]pyrimidin-4-ylamine | | cyclohexyl]-7H- | | | |
| (A,B,C,L) | | pyrrolo[2,3- | | | |
| | | d]pyrimidin-4-ylamine | | | |
| trans-3-(4-Amino-3- | 2-Amino-4-chloro-6- | trans-3-[4-(5-Chloro-7- | 211 | 11.60 min (a) | 564 (M − H)⁻ |
| fluoro-phenyl)-1-[4-(2- | methyl-phenol | methyl-benzoxazol-2- | | | |
| methoxy-ethoxy)- | | ylamino)-3-fluoro- | | | |
| cyclohexyl]-1H- | | phenyl]-1-[4-(2- | | | |
| pyrazolo[3,4- | | methoxy-ethoxy)- | | | |
| d]pyrimidin-4-ylamine | | cyclohexyl)-1H- | | | |
| (A,C,L) | | pyrazolo[3,4- | | | |
| | | d]pyrimidin-4-ylamine | | | |
| trans-4-{4-[4-Amino-3- | 2-Amino-4-fluoro- | trans-4-(4-{4-Amino-3- | 212 | 6.47 min (g) | 574 (M + H)⁺ |
| (4-amino-3-fluoro- | phenol (H) | [3-fluoro-4-(5-fluoro- | | | |
| phenyl)-pyrazolo[3,4- | | benzoxazol-2- | | | |
| d]pyrimidin-1-yl]- | | ylamino)-phenyl]- | | | |
| cyclohexyl}-1-methyl- | | pyrazolo[3,4- | | | |
| piperazin-2-one | | d]pyrimidin-1-yl}- | | | |
| (A,T,J(M),C(D),L) | | cyclohexyl)-1-methyl- | | | |
| | | piperazin-2-one | | | |
| trans-4-{4-[4-Amino-3- | 2-Amino-4- | trans-4-(4-{4-Amino-3- | 213 | 2.50 min (f) | 624 (M + H)⁺ |
| (4-amino-3-fluoro- | trifluoromethyl-phenol | [3-fluoro-4-(5- | | | |
| phenyl)-pyrazolo[3,4- | (H) | trifluoromethyl- | | | |
| d]pyrimidin-1-yl]- | | benzoxazol-2- | | | |
| cyclohexyl}-1-methyl- | | ylamino)-phenyl]- | | | |
| piperazin-2-one | | pyrazolo[3,4- | | | |
| (A,T,J(M),C(D),L) | | d]pyrimidin-1-yl}- | | | |
| | | cyclohexyl)-1-methyl- | | | |
| | | piperazin-2-one | | | |
| trans-4-{4-[4-Amino-5- | 3-Amino-pyridin-4- | trans-4-(4-{4-Amino-5- | 214 | 1.09 min (f) | 538 (M + H)⁺ |
| (4-amino-phenyl)- | ol(H) | [4-(oxazolo[4,5- | | | |
| pyrrolo[2,3- | | c]pyridin-2-ylamino)- | | | |
| d]pyrimidin-7-yl]- | | phenyl]-pyrrolo[2,3- | | | |
| cyclohexyl}-1-methyl- | | d]pyrimidin-7-yl}- | | | |
| piperazin-2-one | | cyclohexyl)-1-methyl- | | | |
| (B,A,T,J(M),C(D)) | | piperazin-2-one | | | |
| trans-{4-{4-[4-Amino- | 2-Amino-6-chloro-4- | trans-{4-(4-{4-Amino- | 215 | 10.45 min (a) | 622 (M + H)⁺ |
| 3-(4-amino-3-fluoro-phenyl)- | fluoro-phenol (F,H) | 3-[4-(7-chloro-5-fluoro- | | | |
| pyrazolo[3,4- | | benzoxazol-2- | | | |
| d]pyrimidin-1-yl]- | | ylamino)-3-fluoro- | | | |
| cyclohexyl}-6,6- | | phenyl)-pyrazolo[3,4- | | | |
| dimethyl-piperazin- | | d]pyrimidin-1-yl}- | | | |
| 2-one} (A,T,J(US | | cyclohexyl)-6,6- | | | |
| Patent 4,251,438),C,L) | | dimethyl-piperazin-2- | | | |
| | | one} | | | |
| trans-{4-{4-[4-Amino- | 2-Amino-4-bromo-6- | trans-{4-(4-{4-Amino- | 216 | 11.46 min (a) | 622 (M + H)⁺ |
| 3-(4-amino-3-fluoro- | methyl-phenol (F,H) | 3-[4-(5-bromo-7- | | | |
| phenyl)-pyrazolo[3,4- | | methyl-benzoxazol-2- | | | |
| d]pyrimidin-1-yl]- | | ylamino)-3-fluoro- | | | |
| cyclohexyl}-6,6- | | phenyl]-pyrazolo[3,4- | | | |
| dimethyl-piperazin-2- | | d]pyrimidin-1-yl}- | | | |
| one} (A,T,J(US Patent | | cyclohexyl)-6,6- | | | |
| 4,251,438),C,L) | | dimethyl-piperazin-2- | | | |
| | | one} | | | |
| cis-{5-(4-Amino-3- | 2-Amino-6-chloro-3,4- | cis-{5-(4-(7-Chloro- | 217 | 14.54 min (e) | 603 (M + H)⁺ |
| fluoro-phenyl)-7-[4-(4- | dimethyl-phenol | 4,5-dimethyl- | | | |
| methyl-piperazin-1-yl)- | (H) | benzoxazol-2- | | | |
| cyclohexyl]-7H- | | ylamino)-3-fluoro- | | | |
| pyrrolo[2,3- | | phenyl]-7-[4-(4-methyl- | | | |
| d]pyrimidin-4-ylamine} | | piperazin-1-yl)- | | | |
| (A,B,T,J,C,L) | | cyclohexyl)-7H- | | | |
| | | pyrrolo[2,3- | | | |
| | | d]pyrimidin-4-ylamine} | | | |
| cis-{5-(4-Amino-3- | 2-Amino-4-chloro-6- | cs-{5-[4-(5-Chloro-7- | 218 | 13.36 min (e) | 589 (M + H)⁺ |
| fluoro-phenyl)-7-[4-(4- | methyl-phenol (F,H) | methyl-benzoxazol-2- | | | |
| methyl-piperazin-1-yl)- | | ylamino)-3-fluoro- | | | |
| cyclohexyl]-7H- | | phenyl)-7-[4-(4-methyl | | | |
| pyrrolo[2,3- | | piperazin-1-yl)- | | | |
| d]pyrimidin-4-ylamine} | | cyclohexyl]-7H- | | | |
| (A,B,T,J,C,L) | | pyrrolo[2,3- | | | |
| | | d]pyrimidin-4-ylamine} | | | |
| cis-{5-(4-Amino-3- | 2-Amino-6-chloro-4- | cis-{5-[4-(7-Chloro-5- | 219 | 13.34 min (e) | 589 (M + H)⁺ |
| fluoro-phenyl)-7-[4-(4- | methyl-phenol (F,H) | methyl-benzoxazol-2- | | | |
| methyl-piperazin-1-yl)- | | ylamino)-3-fluoro- | | | |
| cyclohexyl]-7H- | | phenyl]-7-[4-(4-methyl | | | |
| pyrrolo[2,3- | | piperazin-1-yl)- | | | |
| d]pyrimidin-4-ylamine} | | cyclohexyl)-7H- | | | |
| (A,B,T,J,C,L) | | pyrrolo[2,3- | | | |
| | | d]pyrimidin-4-ylamine} | | | |
| cis-{3-(4-Amino- | 2-Amino-4-chloro-6- | cis-{3-[4-(5-Chloro-7- | 220 | 12.73 min (e) | 572 (M + H)⁺ |
| phenyl)-1-[4-(4- | methyl-phenol (F,H) | methyl-benzoxazol-2- | | | |
| methyl-piperazin-1-yl)- | | ylamino)-phenyl]-1-[4- | | | |
| cyclohexyl]-1H- | | (4-methyl-piperazin-1- | | | |
| pyrazolo[3,4- | | yl)-cyclohexyl]-1H- | | | |
| d]pyrimidin-4-ylamine} | | pyrazolo[3,4- | | | |
| (A,T,J,C) | | d]pyrimidin-4-ylamine} | | | |
| cis-{3-(4-Amino- | 2-Amino-6-chloro-4- | cis-{3-[4-(7-Chloro-5- | 221 | 12.71 min (e) | 572 (M + H)⁺ |
| phenyl)-1-[4-(4- | methyl-phenol (F,H) | methyl-benzoxazol-2- | | | |
| methyl-piperazin-1-yl)- | | ylamino)-phenyl]-1-[4- | | | |
| cyclohexyl]-1H- | | (4-methyl-piperazin-1- | | | |
| pyrazolo[3,4- | | yl)-cyclohexyl]-1H- | | | |
| d]pyrimidin-4-ylamine} | | pyrazolo[3,4- | | | |
| (A,T,J,C) | | d]pyrimidin-4-ylamine) | | | |
| trans-{3-(4-Amino- | 2-Amino-6-chloro-4- | trans-{3-[4-(7-Chloro- | 222 | 11.94 min (e) | 563 (M + H)⁺ |
| phenyl)-1-(4- | fluoro-phenol (F,H) | 5-fluoro-benzoxazol-2- | | | |
| morpholin-4-yl- | | ylamino)-phenyl]-1-(4- | | | |
| cyclohexyl)-1H- | | morpholin-4-yl- | | | |
| pyrazolo[3,4- | | cyclohexyl)-1H- | | | |
| d]pyrimidin-4-ylamine} | | pyrazolo[3,4- | | | |
| (A,T,J,C) | | d]pyrimidin-4-ylamine} | | | |
| trans-{3-(4-Amino- | 2-Amino-4,6-difluoro- | trans-{3-[4-(7- | 223 | 11.22 min (e) | 547 (M + H)⁺ |
| phenyl)-1-(4- | phenol (F,H) | Difluoro-benzoxazol-2- | | | |
| morpholin-4-yl | | ylamino)-phenyl]-1-(4- | | | |
| cyclohexyl)-1H- | | morpholin-4-yl- | | | |
| pyrazolo[3,4- | | cyclohexyl)-1H- | | | |
| d]pyrimidin-4-ylamine} | | pyrazolo[3,4- | | | |
| (A,T,J,C) | | d]pyrimidin-4-ylamine} | | | |
| trans-{3-(4-Amino- | 2-Amino-6-bromo-4- | trans-{3-[4-(7-Bromo- | 224 | 12.07 min (e) | 609 (M + H)⁺ |
| phenyl)-1-(4- | fluoro-phenol (F,H) | 5-fluoro-benzoxazol-2- | | | |
| morpholin-4-yl- | | ylamino)-phenyl]-1-(4- | | | |
| cyclohexyl)-1H- | | morpholin-4-yl- | | | |
| pyrazolo[3,4- | | cyclohexyl)-1H- | | | |
| d]pyrimidin-4-ylamine} | | pyrazolo[3,4- | | | |
| (A,T,J,C) | | d]pyrimidin-4-ylamine} | | | |
| trans-{3-(4-Amino- | 2-Amino-4,-fluoro-6- | trans-{3-[4-(5-Fluoro- | 225 | 11.27 min (e) | 543 (M + H)⁺ |
| phenyl)-1-(4- | methyl-phenol (F,H) | 7-methyl-benzoxazol- | | | |
| morpholin-4-yl- | | 2-ylamino)-phenyl]-1- | | | |
| cyclohexyl)-1H- | | (4-morpholin-4-yl- | | | |
| pyrazolo[3,4- | | cyclohexyl)-1H- | | | |
| d]pyrimidin-4-ylamine} | | pyrazolo[3,4- | | | |
| (A,T,J,C) | | d]pyrimidin-4-ylamine} | | | |
| trans-{3-(4-Amino- | 2-Amino-6-chloro-4- | trans-{3-[4-(7-Chloro- | 226 | 13.59 min (e) | 613 (M + H)⁺ |
| phenyl)-1-(4- | trifluoromethyl- | 5-trifluoromethyl- | | | |
| morpholin-4-yl- | phenol (F,H) | benzoxazol-2- | | | |
| cyclohexyl)-1H- | | ylamino)-phenyl]-1-(4- | | | |
| pyrazolo[3,4- | | morpholin-4-yl- | | | |
| d]pyrimidin-4-ylamine} | | cyclohexyl)-1H- | | | |
| (A,T,J,C) | | pyrazolo[3,4- | | | |
| | | d]pyrimidin-4-ylamine} | | | |
| trans-{3-(4-Amino- | 2-Amino-4,bromo-6- | trans-{3-[4-(5-Bromo- | 227 | 12.62 min (e) | 605 (M + H)⁺ |
| phenyl)-1-(4- | methyl-phenol (F,H) | 7-methyl-benzoxazol- | | | |
| morpholin-4-yl- | | 2-ylamino)-phenyl]-1- | | | |
| cyclohexyl)-1H- | | (4-morpholin-4-yl- | | | |
| pyrazolo[3,4- | | cyclohexyl)-1H- | | | |
| d]pyrimidin-4-ylamine} | | pyrazolo[3,4- | | | |
| (A,T,J,C) | | d]pyrimidin-4-ylamine} | | | |
| trans-{3-(4-Amino- | 3-Amino-5-fluoro-4- | trans-{2-{4-[4-Amino- | 228 | 8.34 min (b) | 554 (M + H)⁺ |
| phenyl)-1-(4- | hydroxy- | 1-(4-morpholin-4-yl- | | | |
| morpholin-4- | benzonitrile(F,H) | cyclohexyl)-1H- | | | |
| -yl-cyclohexyl)-1H- | | pyrazolo[3,4- | | | |
| pyrazolo[3,4-d]pyrimidin- | | d]pyrimidin-3-yl]- | | | |
| 4-ylamine} | | phenylamino}-7-fluoro- | | | |
| (A,T,J,C) | | benzoxazole-5- | | | |
| | | carbonitrile} | | | |
| trans-{3-(4-Amino-3- | 2-Amino-4-fluoro-6- | trans-{3-[3-Fluoro-4-(5- | 229 | 10.7 min (a) | δ 10.79, |
| fluoro-phenyl)-1-[4-(2- | methyl-phenol (EP) | fluoro-7-methyl- | | | 8.5, 8.24, |
| methoxy-ethoxy)- | 409484) | benzoxazol-2- | | | 7.52, 7.1, |
| cyclohexyl]-1H- | | ylamino-phenyl]-1-[4- | | | 6.9, 4.7, |
| pyrazolo[3,4- | | (2-methoxy-ethoxy)- | | | 3.57, |
| d]pyrimidin-4-ylamine | | cyclohexyl]-1H- | | | 3.45, 3.4, |
| (A,C,L) | | pyrazolo[3,4- | | | 3.25, |
| | | d]pyrimidin-4-ylamine | | | 2.44, 2.0 |
| | | acetate salt | | | 1.4 |
| trans-3-(4-Amino- | 3-Amino-5-bromo-2- | trans-2-(4-{4-Amino-1- | 230 | 11.1 min (a) | 605 (M + H)⁺ |
| phenyl)-1-[4-(2- | hydroxy-benzonitrile | [4-(2-methoxy-ethoxy)- | | | |
| methoxy-ethoxy)- | (F,H) | cyclohexyl]-1H- | | | |
| cyclohexyl]-1H- | | pyrazolo[3,4- | | | |
| pyrazolo[3,4- | | d]pyrimidin-3-yl}- | | | |
| d]pyrimidin-4-ylamine | | phenylamino)-5- | | | |
| (A,C) | | bromo-benzoxazole-7- | | | |
| | | carbonitrile | | | |
| trans-3-(4-Amino- | 2-Amino-4- | trans-1-[4-(2-Methoxy- | 231 | 10.5 min (a) | 568 (M + H)⁺ |
| phenyl)-1-[4-(2- | trifluoromethyl-phenol | ethoxy)-cyclohexyl]-3- | | | |
| methoxy-ethoxy)- | (F,H) | [4-(5-trifluoromethyl- | | | |
| cyclohexyl]-1H- | | benzoxazol-2- | | | |
| pyrazolo[3,4- | | ylamino)-phenyl]-1H- | | | |
| d]pyrimidin-4-ylamine | | pyrazolo[3,4- | | | |
| (A,C) | | d]pyrimidin-4-ylamine | | | |
| trans-3-(4-Amino-3- | 3-Amino-5-bromo-2- | trans-3-[3-Fluoro-4-(5- | 232 | 11.2 min (a) | δ 11.3, |
| fluoro-phenyl)-1-[4-(2- | hydroxy-benzonitrile | bromo-7-cyano- | | | 8.4, 8.24, |
| methoxy-ethoxy)- | (F,H) | benzoxazol-2- | | | 8.05, |
| cyclohexyl]-1H- | | ylamino)-phenyl]-1-[4- | | | 7.89, |
| pyrazolo[3,4- | | (2-methoxy-ethoxy)- | | | 7.56, 4.7, |
| d]pyrimidin-4-ylamine | | cyclohexyl]-1H- | | | 3.57, |
| (A,C,L) | | pyrazolo[3,4- | | | 3.44, 3.4, |
| | | d]pyrimidin-4-ylamine | | | 3.25, 2.1, |
| | | acetate salt | | | 1.4 |
| trans-5-(4-Amino- | 2-Amino-4-fluoro-6- | trans-5-[4-(5-Fluoro-7- | 233 | 11.3 min (a) | δ 11.39, |
| phenyl)-7-[4-(2- | methyl-phenol (EP | methyl-benzoxazol-2- | | | 8.13, |
| methoxy-ethoxy)- | 409484) | ylamino)-phenyl]-7-[4- | | | 8.03, |
| cyclohexyl]-7H- | | (2-methoxy-ethoxy)- | | | 7.85, |
| pyrrolo[2,3- | | cyclohexyl]-7H- | | | 7.50, |
| d]pyrimidin-4-ylamine | | pyrrolo[2,3- | | | 7.40, 6.0, |
| (A,C) | | d]pyrimidin-4-ylamine | | | 4.59, |
| | | | | | 3.58, |
| | | | | | 3.43, 3.4, |
| | | | | | 3.2, 2.10, |
| | | | | | 1.94, |
| | | | | | 1.40 |
| trans-5-(4-Amino- | 2-Amino-4- | trans-7-[4-(2-Methoxy- | 234 | 10.9 min (a) | δ (DMSO) |
| phenyl)-7-[4-(2- | trifluoromethyl-phenol | ethoxy)-cyclohexyl]-5- | | | 11.03, |
| methoxy-ethoxy)- | (F,H) | [4-(5-trifluoromethyl- | | | 8.13, |
| cyclohexyl]-7H- | | benzoxazol-2- | | | 7.84, |
| pyrrolo[2,3- | | ylamino)-phenyl]-7H- | | | 7.73, |
| d]pyrimidin-4-ylamine | | pyrrolo[2,3- | | | 7.50, |
| (A,C) | | d]pyrimidin-4-ylamine | | | 7.40, 4.6, |
| | | | | | 3.58, |
| | | | | | 3.45, 3.4, |
| | | | | | 3.26, 2.1, |
| | | | | | 1.95, 1.4 |
| trans-5-(4-Amino- | 3-Amino-5-bromo-2- | trans-2-(4{4-Amino-7- | 235 | 10.6 min (a) | δ (DMSO) |
| phenyl)-7-[4-(2- | hydroxy-benzonitrile | [4-(2-methoxy-ethoxy)- | | | 10.91, |
| methoxy-ethoxy)- | (F,H) | cyclohexyl]-7H- | | | 8.13 |
| cyclohexyl]-7H- | | pyrrolo[2,3- | | | 7.82, |
| pyrrolo[2,3- | | d]pyrimidin-5-yl}- | | | 7.45, |
| d]pyrimidin-4-ylamine | | phenylamino)-5- | | | 7.38, 7.2, |
| (A,C) | | bromo-benzoxazole-7- | | | 6.8, 6.0, |
| | | carbonitrile | | | 4.6, 3.57, |
| | | | | | 3.44, 3.4, |
| | | | | | 2.4, 2.1 |
| | | | | | 1.94, 1.4 |
| trans-5-(4-Amino-3- | 2-Amino-4-fluoro-6- | trans-5-[3-Fluoro-4-(5- | 236 | 10.8 min (a) | δ (DMSO) |
| fluoro-phenyl)-7-[4-(2- | methyl-phenol (EP | fluoro-7-methyl- | | | 10.62, |
| methoxy-ethoxy)- | 409484) | benzoxazol-2- | | | 8.28, |
| cyclohexyl]-7H- | | ylamino)-phenyl]-7-[4- | | | 8.14, |
| pyrrolo[2,3- | | (2-methoxy-ethoxy)- | | | 7.50, |
| d]pyrimidin-4-ylamine | | cyclohexyl]-7H- | | | 7.35, 7.1, |
| (A,C,L) | | pyrrolo[2,3- | | | 6.8, 6.2, |
| | | d]pyrimidin-4-ylamine | | | 4.6, 3.57, |
| | | | | | 3.44, 3.4, |
| | | | | | 3.26, |
| | | | | | 2.43, |
| | | | | | 2.10, |
| | | | | | 1.96, 1.4 |

### General Procedure H: Reduction of a Nitroaromatic Compound to an Aniline

A mixture of a nitroaromatic compound (preferably 1 equivalent), sodium dithionite (1-10 equivalents, preferably 4 equivalents), ethyl viologen dibromide (0-1 equivalent, preferably 0.04 equivalent), and potassium carbonate (0-5 equivalents, preferably 5 equivalents) is heated in a mixture of organic solvent (preferably ethanol or dichloromethane) and water at about 20-80° C. (preferably about 60° C.) for about 1-120 hours (preferably about 2 hours) under an inert atmosphere. The mixture is allowed to cool to ambient temperature and the organic solvent is removed under reduced pressure. The resulting aqueous mixture is extracted with an organic solvent. The organic layer is separated and washed with a saturated brine solution. The retained organic layer is then dried over a desiccant. The solvent is evaporated under reduced pressure to afford the product that can be readily utilized or further purified by crystallization or chromatography.

### Illustration of General Procedure H

### Preparation #8: 3-Amino-5-bromo-2-hydroxy-benzonitrile

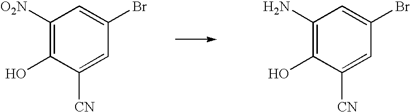

A mixture of 5-bromo-2-hydroxy-3-nitro-benzonitrile (prepared using general procedure F) (4.01 g, 16.5 mmol) and sodium dithionite (11.5 g, 66 mmol) was heated in a mixture of ethanol (200 mL) and water (100 mL) at about 60° C. for about 2 hours under an atmosphere of nitrogen. The mixture was allowed to cool to ambient temperature and organic solvent was removed under reduced pressure. The aqueous mixture was extracted with dichloromethane (200 mL). The organic extracts were combined and washed with saturated brine solution (100 mL). The organic layer was separated and dried over anhydrous magnesium sulfate. The solvent was evaporated under reduced pressure to give 3-amino-5-bromo-2-hydroxy-benzonitrile as a rusty pink solid (2.14 g, 10.0 mmol): RP-HPLC (5% to 95% acetonitrile/0.05M aqueous ammonium acetate, buffered to pH 4.5, over 10 min at 1.7 mL/min; λ=254 nm; Hypersil C18, 100Å, 5 μm, 250×4.6 mm column) Rₜ 8.5 min; ¹H NMR (400 MHz, DMSO-d₆) ε 6.99 (1H, s) and 6.92 (1H, s).

### General procedure I: Alkylation of Nitrogen-Based Nucleophile

A mixture of an alkylating agent, such as mesylate, tosylate, chloride, iodide, or bromide, preferably mesylate (1-1.5 equivalents, preferably 1 equivalent), a nitrogen based nucleophile (preferably a 1H-pyrazolo[3,4-d]pyrimidine or a 7H-pyrrolo[2,3-d]pyrimidine), and a base (for example, sodium carbonate, sodium hydride, potassium carbonate or cesium carbonate, preferably sodium carbonate) (1-10 equivalents, preferably 1.5 equivalents) is heated in an organic solvent (for example, ethylene glycol dimethyl ether, N,N-dimethylformamide, 1-methyl-2-pyrrolidinone, or dimethyl sulfoxide, preferably, N,N-dimethylformamide) at 20-130° C. (preferably about 100° C.) for 1-60 hours (preferably about 30 hours) under an inert atmosphere. The mixture is allowed to cool to ambient temperature and the contents are poured into ice water. The organic layer is separated and the aqueous layer is further extracted with an organic solvent. The combined organic extracts are dried over a desiccant. The solvents are evaporated under reduced pressure to afford the product that can be further purified by crystallization or chromatography.

### An Illustration of General Procedure I

### Preparation #9: 3-(4-Amino-3-iodo-pyrazolo[3,4-d]pyrimidin-1-yl)-azetidine-1-carboxylic acid tert-butyl ester

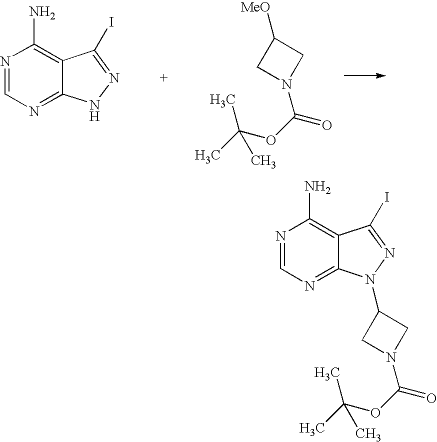

A mixture of 3-iodo-1H-pyrazolo[3,4-d]pyrimidin-4-ylamine (2.68 g, 0.0103 mol), 3-methanesulfonyloxy-azetidine-1-carboxylic acid tert-butyl ester (described in patent WO 02/080926 A1) (2.58 g, 0.0103 mol) and cesium carbonate (4.36 g, 0.0134 mol) was heated in N,N-dimethylformamide (40 mL) at about 90° C. for about 48 hours under an atmosphere of nitrogen. The mixture was allowed to cool to ambient temperature then it was poured into ice water (30 mL) and extracted with 5% methanol/dichloromethane (2×200 mL). The combined organic extracts were dried over magnesium sulfate. The solvents were evaporated under reduced pressure to leave a tan solid. The solids were dissolved in dichloromethane and the solution was cooled to about 0° C. After 16 hours the resulting precipitate was collected and dried to afford 3-(4-amino-3-iodo-pyrazolo[3,4-d]pyrimidin-1-yl)-azetidine-1-carboxylic acid tert-butyl ester as a white solid (2.022 g, 0.00486 mol); RP-HPLC (30% to 95% acetonitrile/0.01M aqueous ammonium acetate over 4.5 min at 0.8 mL/min; λ=190-700 nm; Genesis C18, 120Å, 3 μm, 30×4.6 mm column; electrospray ionization method observing both positive and negative ions) Rₜ 2.28 min; m/z: (M+H)⁺ 417.

Other products obtained using general procedure I are shown (Table 5). The method used to determine the HPLC retention time is given in a lower-case letter in parentheses (see Table 1).

TABLE 5
Examples synthesized using general procedure I

| | Alkylating | | | HPLC RT | |
|---|---|---|---|---|---|
| Substrate | Agent | Product | Example # | (Method) | m/z |
| 3-[4-(5,7-Dimethyl- | Bromo- | (3-{4-Amino-3-[4-(5,7-dimethyl- | 237 | 13.64 min | 480 (M + H)+ |
| benzoxazol-2- | acetonitrile | benzoxazol-2-ylamino)-phenyl]- | | (I) | |
| ylamino)-phenyl]-1- | | pyrazolo[3,4-d]pyrimidin-1-yl}- | | | |
| piperidin-3-yl-1H- | | piperidin-1-yl)-acetonitrile | | | |
| pyrazolo[3,4- | | | | | |
| d]pyrimidin-4- | | | | | |
| ylamine | | | | | |
| (A(R),L,C(E,D)) | | | | | |
| trans-{3-[4-(7- | Methyl iodide | trans-{3-{4-[(7-Chloro-5-methyl- | 238 | 15.64 min | 573 (M + H)+ |
| Chloro-5-methyl- | | benzoxazol-2-yl)-methyl-amino]- | | (I) | |
| benzoxazol-2- | | phenyl}-1-(4- | | | |
| ylamino)-phenyl]-1- | | morpholin-4-yl-cyclohexyl)-1H- | | | |
| (4-morpholin-4-yl- | | pyrazolo[3,4-d]pyrimidin-4- | | | |
| cyclohexyl)-1H- | | ylamine} | | | |
| pyrazolo[3,4- | | | | | |
| d]pyrimidin-4- | | | | | |
| ylamine} | | | | | |
| (A,T,J,C,G(F,H)) | | | | | |
| 3-(4-amino-3-{4- | Bromo-acetic | (3-{4-Amino-3-[4-(5,7-dimethyl- | 239 | 8.9 min (b) | 541 (M + H)+ |
| [(5,7-dimethyl-1,3- | acid ethyl ester | benzoxazol-2-ylamino)-phenyl]- | | | |
| benzoxazol-2- | | pyrazolo[3,4-d]pyrimidin-1-yl}- | | | |
| yl)amino]phenyl}- | | piperidin-1-yl)-acetic acid ethyl | | | |
| 1H-pyrazolo[3,4- | | ester | | | |
| d]pyrimidin-1- | | | | | |
| yl)hexahydropyridini | | | | | |
| um chloride (A,C,L) | | | | | |
| 3-(4-amino-3-{4- | Bromo- | (3-{4-Amino-3-[4-(5,7-dimethyl- | 240 | 8.5 min (b) | 494 (M + H)+ |
| [(5,7-dimethyl-1,3- | acetonitrile | benzoxazol-2-ylamino)-phenyl]- | | | |
| benzoxazol-2- | | pyrazolo[3,4-d]pyrimidin-1-yl}- | | | |
| yl)amino]phenyl}- | | piperidin-1-yl)-acetonitrile | | | |
| 1H-pyrazolo[3,4- | | | | | |
| d]pyrimidin-1- | | | | | |
| yl)hexahydropyridini | | | | | |
| um chloride (A,C,L) | | | | | |
| 3-(4-amino-3-{4- | 2-Bromo- | 2-(3-{4-Amino-3-[4-(5,7- | 241 | 7.0 min (b) | 499 (M + H)+ |
| [(5,7-dimethyl-1,3- | ethanol | dimethyl-benzoxazol-2- | | | |
| benzoxazol-2- | | ylamino)-phenyl]-1-methyl- | | | |
| yl)amino]phenyl}- | | pyrazolo[3,4-d]pyrimidin-1-yl}- | | | |
| 1H-pyrazolo[3,4- | | piperidin-1-yl)-ethanol acetic | | | |
| d]pyrimidin-1- | | acid salt | | | |
| yl)hexahydropyridinium | | | | | |
| chloride (A,C,L) | | | | | |
| 3-(4-amino-3-{4- | 1-Chloro-2- | 3-[4-(5,7-Dimethyl-benzoxazol- | 242 | 8.0 min (b) | 529 (M + H)+ |
| [(5,7-dimethyl-1,3- | methylsulfanyl- | 2-ylamino)-phenyl]-1-[1-(2- | | | |
| benzoxazol-2- | ethane | methylsulfanyl-ethyl)-piperidin- | | | |
| yl)amino]phenyl}- | | 3-yl]-1H-pyrazolo[3,4- | | | |
| 1H-pyrazolo[3,4- | | d]pyrimidin-4-ylamine | | | |
| d]pyrimidin-1- | | | | | |
| yl)hexahydropyridinium | | | | | |
| chloride (A,C,L) | | | | | |
| N2-(4-{4-amino-1- | Bromo- | ((3R)-3-{4-Amino-3-[4-(5,7- | 243 | 8.46 min | 494 (M + H)+ |
| [(3R)hexahydro-3- | acetonitrile | dimethyl-benzoxazol-2- | | (b) | |
| pyridinyl]-1H- | | ylamino)-phenyl]-pyrazolo[3,4- | | | |
| pyrazolo[3,4- | | d]pyrimidin-1-yl}-piperidin-1-yl)- | | | |
| d]pyrimidin-3- | | acetonitrile | | | |
| yl}phenyl)-5,7- | | | | | |
| dimethyl-1,3- | | | | | |
| benzoxazol-2-amine | | | | | |
| (A,L,C) | | | | | |
| N2-(4-{4-amino-1- | Bromo- | ((3S)3-{4-Amino-3-[4-(5,7- | 244 | 8.48 min | 494 (M + H)+ |
| [(3S)hexahydro-3- | acetonitrile | dimethyl-benzoxazol-2- | | (b) | |
| pyridinyl]-1H- | | ylamino)-phenyl]-1-methyl- | | | |
| pyrazolo[3,4- | | pyrazolo[3,4-d]pyrimidin-1-yl}- | | | |
| d]pyrimidin-3- | | piperidin-1-yl)-acetonitrile acetic | | | |
| yl}phenyl)-5,7- | | acid salt | | | |
| dimethyl-1,3- | | | | | |
| benzoxazol-2-amine | | | | | |
| (A,L,C) | | | | | |
| N2-(4-{4-amino-1- | Bromo- | [(3S)3-(4-Amino-3-{4- | 245 | 8.40 min | 533 (M + H)+ |
| [(3S)hexahydro-3- | acetonitrile | [cyanomethyl-(5,7-dimethyl- | | (b) | |
| pyridinyl]-1H- | | benzoxazol-2-yl)-amino]- | | | |
| pyrazolo[3,4- | | phenyl}-1-methyl-pyrazolo[3,4- | | | |
| d]pyrimidin-3- | | d]pyrimidin-1-yl)-piperidin-1-yl]- | | | |
| yl}phenyl)-5,7- | | acetonitrile acetic acid salt | | | |
| dimethyl-1,3- | | | | | |
| benzoxazol-2-amine | | | | | |
| (A,L,C) | | | | | |
| 3-[4-(5,7-Dimethyl- | 2-Chloro-N,N- | 2-(3-{4-Amino-3-[4-(5,7- | 246 | 11.6 min | 540 (M + H)+ |
| benzoxazol-2- | dimethyl- | dimethyl-benzoxazol-2- | | (b) | |
| ylamino)-phenyl]-1- | acetamide | ylamino)-phenyl]-pyrazolo[3,4- | | | |
| piperidin-3-yl-1H- | | d]pyrimidin-1-yl}-piperidin-1-yl)- | | | |
| pyrazolo[3,4- | | N,N-dimethyl-acetamide | | | |
| d]pyrimidin-4- | | | | | |
| ylamine (A,L,C) | | | | | |
| 3-[4-(5,7-Dimethyl- | 2-Chloro-1- | 2-(3-{4-Amino-3-[4-(5,7- | 247 | 12.1 min | 582 (M + H)+ |
| benzoxazol-2- | morpholin-4-yl- | dimethyl-benzoxazol-2- | | (b) | |
| ylamino)-phenyl]-1- | ethanone | ylamino)-phenyl]-1-methyl- | | | |
| piperidin-3-yl-1H- | | pyrazolo[3,4-d]pyrimidin-1-yl}- | | | |
| pyrazolo[3,4- | | piperidin-1-yl)-1-morpholin-4-yl- | | | |
| d]pyrimidin-4- | | ethanone acetic acid salt | | | |
| ylamine (A,L,C) | | | | | |

### General Procedure J: Reductive Coupling of an Amine with a Ketone

A mixture of a ketone (3-20 equivalents, preferably 1 equivalent), an amine (or an amine salt) (1-4 equivalents, preferably 3 equivalents) and acetic acid (preferably 4 equivalents) is stirred in a mixture of organic solvents (preferably 1,2-dichloroethane and 1-methyl-2-pyrrolidinone) at ambient temperature for about 2 hours under an atmosphere of nitrogen. Then a reducing reagent (preferably sodium triacetoxyborohydride) (1.5-14 equivalents, preferably 1.5 equivalents) is added and the mixture is stirred at ambient temperature for about 12 hours to 7 days (preferably about 12 hours). The mixture is quenched with an aqueous basic solution (for example, saturated aqueous sodium bicarbonate solution) and extracted with organic solvent. The combined organic extracts are dried over a desiccant. The solvents are evaporated under reduced pressure to afford the product that can be further purified by crystallization or chromatography.

### Illustration of General Procedure J

### Preparation #10: trans-{4-[4-(4-Amino-5-iodo-pyrrolo[2,3-d]pyrimidin-7-yl)-cyclohexyl]-1-methyl-piperazin-2-one}

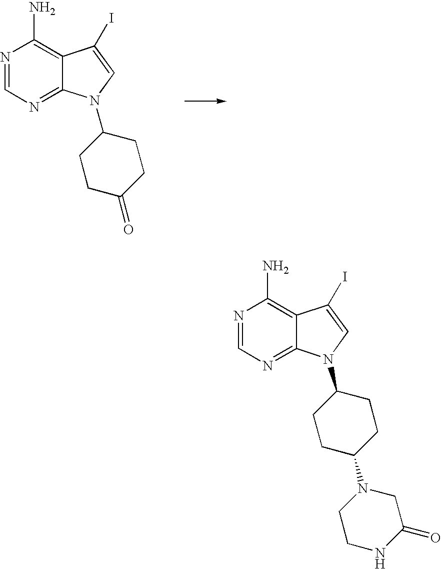

A mixture of 4-(4-amino-5-iodo-pyrrolo[2,3-d]pyrimidin-7-yl)-cyclohexanone (prepared by general procedures A and T) (17.6 g, 48.2 mmol), 4-methyl-3-oxo-piperazin-1-ium monotrifluoroacetate (44.03 g, 193 mmol), and acetic acid (11.05 mL, 193 mmol) was stirred in a mixture of 1,2-dichloroethane (1000 mL) and 1-methyl-2-pyrrolidinone (50 mL) at ambient temperature for about 2.5 hours under an atmosphere of nitrogen. Then sodium triacetoxyborohydride (15.337 g, 72.4 mmol) was added in one portion and the mixture was stirred at ambient temperature for about 12 hours. The mixture was quenched with saturated sodium carbonate aqueous solution until pH ≧7 and extracted with dichloromethane/methanol (95:5, 1000 mL). The combined organic extracts were dried over magnesium sulfate. Dichloromethane and methanol were evaporated under reduced pressure to afford a solid that was purified by flash column chromatography on silica using a gradient of methanol/ethyl acetate/triethylamine (1:98:1 to 7:92:1) as a mobile phase to give trans-{4-[4-(4-amino-5-iodo-pyrrolo[2,3-d]pyrimidin-7-yl)-cyclohexyl]-1-methyl-piperazin-2-one} as a white solid (5.07 g, 11 mmol). ¹H NMR (DMSO-d₆, 400 MHz) δ 8.08, 7.53, 6.51, 5.76, 4.49, 3.29, 3.12, 2.73, 2.45, 1.90, 1.18; RP-HPLC (30% to 95% acetonitrile/0.01M aqueous ammonium acetate over 4.5 min at 0.8 mL/min; λ=190-700 nm; Genesis C18, 120Å, 3 μm, 30×4.6 mm column; electrospray ionization method observing both positive and negative ions) Rₜ 1.37 min; m/z: (M+H)⁺ 455.

Other products obtained using general procedure J are shown (Table 6). The method used to determine the HPLC retention time is given in a lower-case letter in parentheses (see Table 1).

TABLE 6
Examples synthesized using general procedure J

| | | | | HPLC RT | m/z or 1H |
|---|---|---|---|---|---|
| Ketone | Amine | Product | Example # | (Method) | NMR |
| 4-{4-Amino-5-[3- | Dimethyl-amine | cis-{7-(4- | 248 | 11.25 min (e) | δ (DMSO) 8.30, |
| fluoro-4-(5- | | Dimethylamino- | | | 8.15, 7.44, 7.35, |
| methyl- | | cyclohexyl)-5-[3-fluoro- | | | 7.27, 6.95, |
| benzoxazol-2- | | 4-(5-methyl- | | | 6.18, 4.67, 2.37, |
| ylamino)-phenyl]- | | benzoxazol-2- | | | 2.21, 2.09, 2.06, |
| pyrrolo[2,3- | | ylamino)-phenyl]-7H- | | | 1.70, 1.58 |
| d]pyrimidin-7-yl}- | | pyrrolo[2,3-d]pyrimidin- | | | |
| cyclohexanone | | 4-ylamine}, bisacetic | | | |
| (A,B,T,C,L,G) | | acid salt | | | |
| 4-{4-Amino-5-[3- | Dimethyl-amine | trans-{7-(4- | 249 | 11.48 min (e) | δ (DMSO) 8.31, |
| fluoro-4-(5- | | Dimethylamino- | | | 8.14, 7.50, 7.38, |
| methyl- | | cyclohexyl)-5-[3-fluoro- | | | 7.32, 7.27, |
| benzoxazol-2- | | 4-(5-methyl- | | | 6.95, 6.17, 4.56, |
| ylamino)-phenyl]- | | benzoxazol-2- | | | 2.37, 2.31, 2.22, |
| pyrrolo[2,3- | | ylamino)-phenyl]-7H- | | | 1.90, 1.43 |
| d]pyrimidin-7-yl}- | | pyrrolo[2,3-d]pyrimidin- | | | |
| cyclohexanone | | 4-ylamine}, acetic acid | | | |
| (A,B,T,C,L,G) | | salt | | | |
| 4-{4-Amino-5-[4- | Piperazin-2-one | trans-{4-(4-{4-Amino-5- | 250 | 13.16 min (e) | 569 (M + H)⁺ |
| (5,7-dimethyl- | | [4-(5,7-dimethyl- | | | |
| benzoxazol-2- | | benzoxazol-2- | | | |
| ylamino)-3-fluoro- | | ylamino)-3-fluoro- | | | |
| phenyl]- | | phenyl]-pyrrolo[2,3- | | | |
| pyrrolo[2,3- | | d]pyrimidin-7-yl}- | | | |
| d]pyrimidin-7-yl}- | | cyclohexyl)-piperazin- | | | |
| cyclohexanone | | 2-one} | | | |
| (A,B,T,C,L,G) | | | | | |
| 4-{4-Amino-5-[4- | Piperazin-2-one | cis-{4-(4-{4-Amino-5- | 251 | 13.25 min (e) | 569 (M + H)⁺ |
| (5,7-dimethyl- | | [4-(5,7-dimethyl- | | | |
| benzoxazol-2- | | benzoxazol-2- | | | |
| ylamino)-3-fluoro- | | ylamino)-3-fluoro- | | | |
| phenyl]- | | phenyl]-pyrrolo[2,3- | | | |
| pyrrolo[2,3- | | d]pyrimidin-7-yl}- | | | |
| d]pyrimidin-7-yl}- | | cyclohexyl)-piperazin- | | | |
| cyclohexanone | | 2-one} | | | |
| (A,B,T,C,L,G) | | | | | |
| 4-{4-Amino-3-[4- | 1-Isopropyl- | trans-{4-(4-{4-Amino-3- | 252 | 9.86 min (a) | δ(DMSO) 10.85, |
| (5,7-dimethyl- | piperazin-2-one, | [4-(5,7-dimethyl- | | | 8.23, 7.92, |
| benzoxazol-2- | trifluoroacetic | benzoxazol-2- | | | 7.65, 7.12, 6.80 |
| ylamino)-phenyl]- | acid salt (M) | ylamino)-phenyl]- | | | 4.65, 3.15, 2.74 |
| pyrazolo[3,4- | | pyrazolo[3,4- | | | 2.40, 2.34, |
| d]pyrimidin-1-yl}- | | d]pyrimidin-1-yl}- | | | 2.01, 1.48, 1.05 |
| cyclohexanone | | cyclohexyl)-1- | | | |
| (A,T,C,G) | | isopropyl-piperazin-2- | | | |
| | | one} | | | |
| 4-{4-Amino-3-[4- | [1,4]Diazepan-5- | cis-1-(4-{4-Amino-3-[4- | 253 | 2.27 min (f) | 566 (M + H)⁺ |
| (5,7-dimethyl- | one | (5,7-dimethyl- | | | |
| benzoxazol-2- | | benzoxazol-2- | | | |
| ylamino)-phenyl]- | | ylamino)-phenyl]- | | | |
| pyrazolo[3,4- | | pyrazolo[3,4- | | | |
| d]pyrimidin-1-yl}- | | d]pyrimidin-1-yl}- | | | |
| cyclohexanone | | cyclohexyl)- | | | |
| (A,T,C) | | [1,4]diazepan-5-one | | | |
| 4-{4-Amino-3-[4- | [1,4]Diazepan-5- | trans-1-(4-{4-Amino-3- | 254 | 2.25 min (f) | 566 (M + H)⁺ |
| (5,7-dimethyl- | one | [4-(5,7-dimethyl- | | | |
| benzoxazol-2- | | benzoxazol-2- | | | |
| ylamino)-phenyl]- | | ylamino)-phenyl]- | | | |
| pyrazolo[3,4- | | pyrazolo[3,4- | | | |
| d]pyrimidin-1-yl}- | | d]pyrimidin-1-yl}- | | | |
| cyclohexanone | | cyclohexyl)- | | | |
| (A,T,C) | | [1,4]diazepan-5-one | | | |
| 4-{4-Amino-3-[4- | 4-Methyl- | cis-1-(4-{4-Amino-3-[4- | 255 | 2.47 min (f) | 580 (M + H)⁺ |
| (5,7-dimethyl- | [1,4]diazepan-5- | (5,7-dimethyl- | | | |
| benzoxazol-2- | one (M) | benzoxazol-2- | | | |
| ylamino)-phenyl]- | | ylamino)-phenyl]- | | | |
| pyrazolo[3,4- | | pyrazolo[3,4- | | | |
| d]pyrimidin-1-yl}- | | d]pyrimidin-1-yl}- | | | |
| cyclohexanone | | cyclohexyl)-4-methyl- | | | |
| (A,T,C) | | [1,4]diazepan-5-one | | | |
| 4-{4-Amino-3-[4- | 4-Methyl- | trans-1-(4-{4-Amino-3- | 256 | 2.35 min (f) | 580 (M + H)⁺ |
| (5,7-dimethyl- | [1,4]diazepan-5- | [4-(5,7-dimethyl- | | | |
| benzoxazol-2- | one (M) | benzoxazol-2- | | | |
| ylamino)-phenyl]- | | ylamino)-phenyl]- | | | |
| pyrazolo[3,4- | | pyrazolo[3,4- | | | |
| d]pyrimidin-1-yl}- | | d]pyrimidin-1-yl}- | | | |
| cyclohexanone | | cyclohexyl)-4-methyl- | | | |
| (A,T,C) | | [1,4]diazepan-5-one | | | |
| 4-{4-Amino-5-[3- | 4-Methyl- | trans-1-(4-{4-Amino-5- | 257 | 2.23 min (f) | 583 (M + H)⁺ |
| fluoro-4-(5- | [1,4]diazepan-5- | [3-fluoro-4-(5-methyl- | | | |
| methyl- | one (M) | benzoxazol-2- | | | |
| benzoxazol-2- | | ylamino)-phenyl]- | | | |
| ylamino)-phenyl]- | | pyrrolo[2,3-d]pyrimidin- | | | |
| pyrrolo[2,3- | | 7-yl}-cyclohexyl)-4- | | | |
| d]pyrimidin-7-yl}- | | methyl-[1,4]diazepan- | | | |
| cyclohexanone | | 5-one | | | |
| (B,A,T,C) | | | | | |
| 4-{4-Amino-5-[3- | 4-Methyl- | cis-1-(4-{4-Amino-5-[3- | 258 | 2.36 min (f) | 583 (M + H)⁺ |
| fluoro-4-(5- | [1,4]diazepan-5- | fluoro-4-(5-methyl- | | | |
| methyl- | one (M) | benzoxazol-2- | | | |
| benzoxazol-2- | | ylamino)-phenyl]- | | | |
| ylamino)-phenyl]- | | pyrrolo[2,3-d]pyrimidin- | | | |
| pyrrolo[2,3- | | 7-yl}-cyclohexyl)-4- | | | |
| d]pyrimidin-7-yl}- | | methyl-[1,4]diazepan- | | | |
| cyclohexanone | | 5-one acetic acid salt | | | |
| (B,A,T,C) | | | | | |
| 4-{4-Amino-3-[4- | 6,6-Dimethyl- | trans-4-(4-{4-Amino-3- | 259 | 21.20 min (C) | 599 (M − H)⁻ |
| (5-chloro-7- | piperazin-2-one | [4-(5-chloro-7-methyl- | | | |
| methyl- | | benzoxazol-2- | | | |
| benzoxazol-2- | | ylamino)-phenyl]- | | | |
| ylamino)-phenyl]- | | pyrazolo[3,4- | | | |
| pyrazolo | | d]pyrimidin-1-yl}- | | | |
| [3,4- | | cyclohexyl)-6,6- | | | |
| d]pyrimidin-1-yl}- | | dimethyl-piperazin-2- | | | |
| cyclohexanone | | one | | | |
| 4-{4-Amino-3-[4- | 6,6-Dimethyl- | cis-4-(4-{4-Amino-3-[4- | 260 | 21.63 min (c) | 599 (M − H)⁻ |
| (5-chloro-7- | piperazin-2-one | (5-chloro-7-methyl- | | | |
| methyl- | | benzoxazol-2- | | | |
| benzoxazol-2- | | ylamino)-phenyl]- | | | |
| ylamino)-phenyl]- | | pyrazolo[3,4- | | | |
| pyrazolo | | d]pyrimidin-1-yl}- | | | |
| [3,4- | | cyclohexyl)-6,6- | | | |
| d]pyrimidin-1-yl}- | | dimethyl-piperazin-2- | | | |
| cyclohexanone | | one | | | |
| 4-{4-Amino-5-[4- | Trifluoro- | trans-4-(4-{4-Amino-5- | 261 | 2.63 min (f) | 583 (M + H)⁺ |
| (5,7-dimethyl- | acetate4-methyl- | [4-(5,7-dimethyl- | | | |
| benzoxazol-2- | 3-oxo-piperazin- | benzoxazol-2- | | | |
| ylamino)-3-fluoro- | 1-ium (M,L) | ylamino)-3-fluoro- | | | |
| phenyl]- | | phenyl]-pyrrolo[2,3- | | | |
| pyrrolo[2,3- | | d]pyrimidin-7-yl}- | | | |
| d]pyrimidin-7-yl}- | | cyclohexyl)-1-methyl- | | | |
| cyclohexanone | | piperazin-2-one | | | |
| (B,A,T,C(D)) | | | | | |
| 4-{4-Amino-5-[3- | Trifluoro-acetate | trans-4-(4-{4-Amino-3- | 262 | 9.1 min (a) | 567 (M − H)⁻ |
| fluoro-4-(5- | 4-methyl-3-oxo- | [4-(5-methyl- | | | |
| methyl- | piperazin-1-ium | benzoxazol-2- | | | |
| benzoxazol-2- | (M) | ylamino)-phenyl]- | | | |
| ylamino)-phenyl]- | | pyrolo[3,4-d]pyrimidin- | | | |
| pyrrolo[2,3- | | 1-yl}-cyclohexyl)-1- | | | |
| d]pyrimidin-7-yl}- | | methyl-piperazin-2-one | | | |
| cyclohexanone | | | | | |
| (A,T,B,C) | | | | | |
| Tetrahydro-4H- | 3-[4-(5,7- | 3-[4-(5,7-Dimethyl- | 263 | 9.4 min (a) | 553 (M − H)⁻ |
| thiopyran-4-one | Dimethyl- | benzoxazol-2- | | | |
| | benzoxazol-2- | ylamino)-phenyl]-1-[1- | | | |
| | ylamino)-phenyl]- | (tetrahydro-thiopyran- | | | |
| | 1-piperidin-4-yl- | 4-yl)-piperidin-4-yl]-1H- | | | |
| | 1H-pyrazolo[3,4- | pyrazolo[3,4- | | | |
| | d]pyrimidin-4- | d]pyrimidin-4-ylamine | | | |
| | ylamine (A,L,C) | acetic acid salt | | | |
| 4H-Thiopyran-4- | 3-[4-(5,7- | 3-[4-(5,7-Dimethyl- | 264 | 9.0 min (a) | 587 (M + H)⁺ |
| one, tetrahydro-, | Dimethyl- | benzoxazol-2- | | | |
| 1,1-dioxide | benzoxazol-2- | ylamino)-phenyl]-1-[1- | | | |
| | ylamino)-phenyl]- | (1,1-dioxo-hexahydro- | | | |
| | 1-piperidin-4-yl- | 1l6-thiopyran-4-yl)- | | | |
| | 1H-pyrazolo[3,4- | piperidin-4-yl]-1H- | | | |
| | d]pyrimidin-4- | pyrazolo[3,4- | | | |
| | ylamine (A,L,C) | d]pyrimidin-4-ylamine | | | |
| | | acetic acid salt | | | |

### General Procedure K: Ketalization of a Ketone

A mixture of a ketone (preferably 1 equivalent), a butanediol (1-50 equivalents, preferably 20 equivalents), and p-toluenesulfonic acid (0.05-1 equivalents, preferably 0.2 equivalents) is heated in an organic solvent (preferably toluene) at about 50-120° C. (preferably at reflux temperature) over 1-10 days (preferably 5 days) under an inert atmosphere. The by-product water is removed (preferably in a Dean-Stark trap filled with activated molecular sieves (3 Å bead, 4-8 mesh)). The mixture is allowed to cool to ambient temperature. The solvent is removed under reduced pressure to yield the crude product, which can be further purified by distillation, chromatography or crystallization to afford the product.

### Illustration of General Procedure K

### Example #265

N2-(4-{4-amino-1-[(2R,3R)-2,3-dimethyl-1,4-dioxaspiro[4.5]dec-8-yl]-1H-pyrazolo[3,4-d]pyriridin-3-yl}phenyl)-5,7-dimethyl-1,3-benzoxazol-2-amine

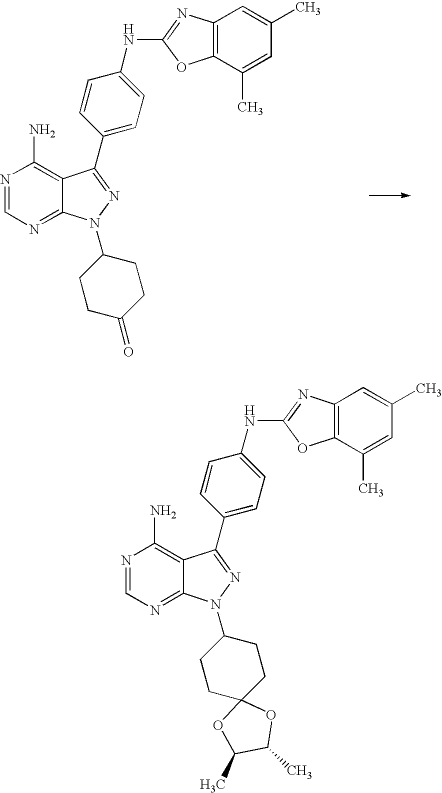

A mixture of 4-(4-amino-3-{4-[(5,7-dimethyl-1,3-benzoxazol-2-yl)amino]phenyl}-1H-pyrazolo[3,4-d]pyrimidin-1-yl)-1-cyclohexanone (prepared using general procedures A, T, and C) (0.40 g, 0.86 mmol), (R,R)-1,2-butanediol (0.29 g, 3.22 mmol), and p-toluenesulfonic acid monohydrate (0.03 g, 0.16 mmol) was heated in toluene (30 mL) at reflux over 5 days, during which time the water was collected in a Dean-Stark trap filled with molecular sieves (3 Å bead, 4-8 mesh). Additional (R,R)-1,2-butanediol (0.87 g, 9.66 mmol) was required for the reaction to reach completion. The reaction was allowed to cool to ambient temperature and the solvent was removed under reduced pressure. The resulting crude product was purified by flash chromatography on silica using a gradient of 0%-3% methanol (containing 2% of aqueous 28% ammonia) in dichloromethane as the mobile phase to afford N2-(4-{4-amino-1-[(2R,3R)-2,3-dimethyl-1,4-dioxaspiro[4.5]dec-8-yl]-1H-pyrazolo[3,4-d]pyrimidin-3-yl}phenyl)-5,7-dimethyl-1,3-benzoxazol-2-amine (0.276 g, 0.51 mmol) as a white solid: RP-HPLC (5% to 95% acetonitrile/0.05M aqueous ammonium acetate, buffered to pH 4.5, over 10 min at 1.7 mL/min; λ=254 nm; Hypersil C18, 100Å, 5 μm, 250×4.6 mm column); Rₜ 12.13 min; m/z (M+H)⁺ 540.

Other products obtained using general procedure K are shown (Table 7). The method used to determine the HPLC retention time is given in a lower-case letter in parentheses (see Table 1).

TABLE 7
Examples synthesized using general procedure K

| Butanediol | | | HPLC RT | |
|---|---|---|---|---|
| Precursor | Product | Example # | (Method) | M/z |
| meso-2,3- | N2-(4-{4-amino-1- | 266 | 12.1 | 540 (M + H)′ |
| butanediol | [(2R,3S)-2,3- | | min(a) | |
| | dimethyl-1,4- | | | |
| | dioxaspiro[4,5]dec-8- | | | |
| | yl]-1H-pyrazolo[3,4- | | | |
| | d]pyrimidin-3- | | | |
| | yl}phenyl)-5,7- | | | |
| | dimethyl-1,3- | | | |
| | benzoxazol-2-amine | | | |
| meso-2,3- | N2-(4-{4-amino-1- | 267 | 14.2 | 540 (M + H)′ |
| butanediol | [(2S,3S)-2,3- | | min (b) | |
| | dimethyl-1,4- | | | |
| | dioxaspiro[4,5}dec-8- | | | |
| | yl-1H-pyrazolo[3,4- | | | |
| | d]pyrimidin-3- | | | |
| | yl}phenyl)-5,7- | | | |
| | dimethyl-1,3- | | | |
| | benzoxazol-2-amine | | | |

### General Procedure L: Removal of a Boc-Protecting Group

A mixture of a tert-butyl carbamate (1-1.5 equivalents, preferably 1 equivalent), an organic solvent (for example 1,4 dioxane or dichloromethane, preferably dichloromethane) and an acid (540 equivalents, preferably 20 equivalents) (for example hydrochloric acid or trifluoroacetic acid, preferably trifluoroacetic acid) is mixed at about 0-60° C. (preferably about 25° C.) for about 1-24 hours (preferably about 14 hours) under an inert atmosphere. The mixture is neutralized with an aqueous base (such as sodium carbonate or potassium carbonate, preferably sodium carbonate). The organic layer is separated and the aqueous layer is further extracted with an organic solvent. The combined organic extracts are dried over a desiccant. The solvents are evaporated under reduced pressure to afford the product that can be further purified by crystallization or chromatography.

### Illustration of General Procedure L

### Example #268

7-Azetidin-3-yl-5-[4-(5,7-dimethyl-benzoxazol-2-ylamino)-phenyl]-7H-pyrrolo[2,3-d]pyrimidin-4-ylamine

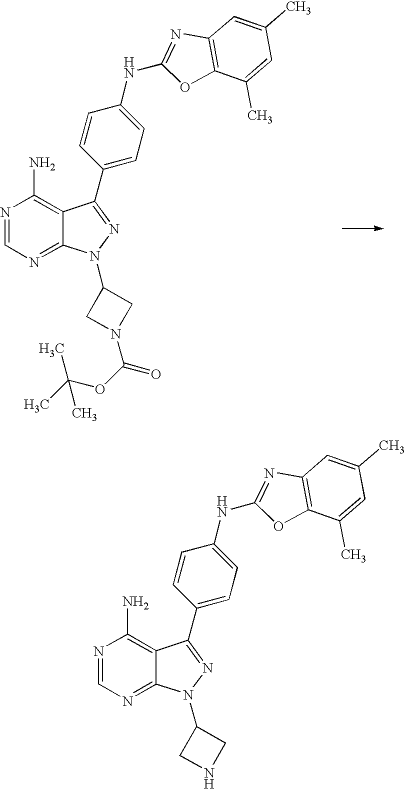

A mixture of 3-{4-amino-5-[4-(5,7-dimethyl-benzoxazol-2-ylamino)-phenyl]-pyrrolo[2,3-d]pyrimidin-7-yl}-azetidine-1-carboxylic acid tert-butyl ester (prepared by general procedures I and C) (1.23 g, 0.002336 mol) and trifluoroacetic acid (1.81 mL, 0.023 mol) was mixed in dichloromethane (18 mL) at ambient temperature for about 24 hours under an atmosphere of nitrogen. The mixture was diluted with aqueous sodium carbonate the organic layer was separated and dried over a desiccant. The precipitate in the aqueous layer was collected and combined with the organic phase. The solvent was removed under reduced pressure to afford 7-azetidin-3-yl-5-[4-(5, 7-dimethyl-benzoxazol-2-ylamino)-phenyl]-7H-pyrrolo[2,3-d]pyrimidin-4-ylamine as a tan solid (1.00 g, 0.00234 mol); RP-HPLC (30% to 95% acetonitrile/0.01M aqueous ammonium acetate over 4.5 min at 0.8 mLmin; λ=190-700 nm; Genesis C18, 120Å, 3 μm, 30×4.6 mm column; electrospray ionization method observing both positive and negative ions) Rₜ 2.13 min.; m/z: (M+H)⁺ 427.

Other products obtained using general procedure L are shown (Table 8). The method used to determine the HPLC retention time is given in a lower-case letter in parentheses (see Table 1).

TABLE 8
Examples synthesized using general procedure L

| | | | HPLC RT | | |
|---|---|---|---|---|---|
| Boc Protected amine | Product | Example # | (method) | | m/z |
| 3-{4-Amino-3-[4-(5,7- | 1-Azetidin-3-yl-3-[4- | 269 | 2.45 | min (f) | 443 (M − H)⁻ |
| dimethyl-benzoxazol- | (5,7-dimethyl- | | | | |
| 2-ylamino)-3-fluoro- | benzoxazol-2- | | | | |
| phenyl]-pyrazolo[3,4- | ylamino)-3-fluoro- | | | | |
| d]pyrimidin-1-yl}- | phenyl]-1H- | | | | |
| azetidine-1-carboxylic | pyrazolo[3,4- | | | | |
| acid tert-butyl ester (I | d]pyrimidin-4-ylamine | | | | |
| (WO 02/080926 A1),C) | | | | | |
| cis-{4-(4-{4-Amino-5- | cis-{5-[4-(5,7-Dimethyl- | 270 | 12.74 | min (e) | 555 (M + H)⁺ |
| [4-(5,7-dimethyl- | benzoxazol-2- | | | | |
| benzoxazol-2- | ylamino)-3-fluoro- | | | | |
| ylamino)-3-fluoro- | phenyl]-7-(4-piperazin- | | | | |
| phenyl]-pyrrolo[2,3- | 1-yl-cyclohexyl)-7H- | | | | |
| d]pyrimidin-7-yl}- | pyrrolo[2,3-d]pyrimidin- | | | | |
| cyclohexyl)-piperazine- | 4-ylamine} | | | | |
| 1-carboxylic acid tert- | | | | | |
| butyl ester} (A,B,T,J,C | | | | | |
| L,G) | | | | | |
| trans-{4-(4-{4-Amino-5- | trans-{5-[4-(5,7- | 271 | 12.26 | min (e) | 555 (M + H)⁺ |
| [4-(5,7-dimethyl- | Dimethyl-benzoxazol- | | | | |
| benzoxazol-2- | 2-ylamino)-3-fluoro- | | | | |
| ylamino)-3-fluoro- | phenyl]-7-(4-piperazin- | | | | |
| pheny]-pyrrolo[2,3- | 1-yl-cyclohexyl)-7H- | | | | |
| d]pyrimidin-7-yl}- | pyrrolo[2,3-d]pyrimidin- | | | | |
| cyclohexyl)-piperazine- | 4-ylamine} | | | | |
| 1-carboxylic acid tert- | | | | | |
| butyl ester} | | | | | |
| (A,B,T,J,C,L,G) | | | | | |

### General Procedure M: N-Alkylation of Lactam

The amine functionality of a lactam amine (1-2 equivalents, preferably 1 equivalent) is acylated with an appropriate protecting group (for example, di-tert-butyl dicarbonate) (1-2 equivalents, preferably 1.05 equivalents) in tetrahydrofuran at ambient temperature for about 1-24 hours (preferably about 15 hours). The solvent is removed under reduced pressure and the resulting residue is washed with an organic solvent (for example, heptane or ethyl acetate). The residue is dissolved in an organic solvent (for example, a mixture of tetrahydrofuran and N,N-dimethylformamide), and treated with a base (for example, sodium hydride) (1-2 equivalents, preferably 1.5 equivalents) at ambient temperature for about 0.5-12 h (preferably 1 hour), then an alkyl halide (1-4 equivalents, preferably 1.05 equivalents) (for example, iodomethane) is added. The reaction mixture is stirred at about 0-75° C. (preferably ambient temperature) for about 1-24 hours (preferably 15 hours). The solvent is removed and extractive work-up affords a product that can be further purified by chromatography. The protecting group on the amine functionality is removed (for example, removal of the Boc-group is detailed in general procedure L) to afford the product or the product salt that can be further purified by crystallization or chromatography.

### Illustration of General Procedure M

### Preparation #11: 4-Methyl-[1,4]diazepan-5-one monotrifluoroacetate

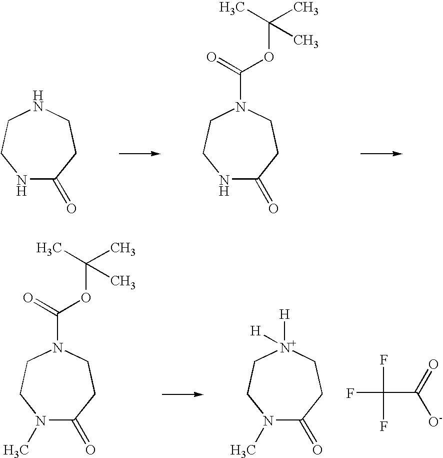

Di-tert-butyl dicarbonate (3.162 g, 0.01447 mol) was added to a suspension of [1,4]diazepan-2-one (1.562 g, 0.01338 mol) in tetrahydrofuran (90 mL), and the mixture was stirred at ambient temperature for about 15 hours. The solvent was removed under reduced pressure. The residue was washed with ethyl acetate to give 5-oxo-[1,4]diazepane-1-carboxylic acid tert-butyl ester as a white solid (2.866 g, 0.01338 mol). The solid was dissolved in a mixture of tetrahydrofuran (80 mL) and N,N-dimethylformamide (30 mL), and sodium hydride (60% dispersion in mineral oil, 0.849 g, 0.0212 mol) was added. After about 1 hour, iodomethane (0.93 ml, 0.01835 mol) was added slowly to the reaction mixture. The mixture was stirred at ambient temperature for about 15 h, then the solvents were removed under reduced pressure. The residue was partitioned between saturated aqueous ammonium chloride solution (100 mL) and dichloromethane. The organic layer was separated and the aqueous layer was further extracted with dichloromethane. The combined organic extracts were dried over magnesium sulfate. The solvent was evaporated under reduced pressure to leave a dark brown solid which was purified by flash column chromatography on silica gel using ethyl acetate as a mobile phase to give 4-methyl-5-oxo (1,4]diazepane-1-carboxylic acid tert-butyl ester as a white solid (2.790 g, 0.0122 mol). The solid was dissolved in dichloromethane (20 mL), the solution was cooled to 0° C., and trifluoroacetic acid (10 ml, 0.1298 mol) was added. The reaction mixture was allowed to warm to room temperature. The solvent was evaporated under reduced pressure to afford 4-methyl-[1,4]diazepan-5-one monotrifluoroacetate as a brown oil ( 2.962 g, 0.01223 mol); ¹H NMR (DMSO-d₆, 400 MHz) δ 3.64 (m, 2H), 3.23 (m, 4H), 2.89 (s, 2H), 2.74 (m, 2H).

### General Procedure N: Debenzylation of a Benzyl Ether Compound

A mixture of a benzyl ether (preferably 1 equivalent) and palladium on carbon (10% by weight) (0.01-0.50 equivalents, preferably 0.10 equivalents) in an organic solvent (for example, ethanol, ethyl acetate, ethylene glycol dimethyl ether, or toluene, preferably ethanol) is stirred under a hydrogen atmosphere at about 20-120° C. (preferably about 20° C.) for about 1-48 hours (preferably 12 hours). The mixture is filtered through a Celite column that is washed with additional organic solvent. The solvent is removed under reduced pressure to give the desired product that can be further purified by crystallization or chromatography.

### Illustration of General Procedure N

### Preparation #12: cis-4-(2-Cyclopropoxy-ethoxy)-cyclohexanol

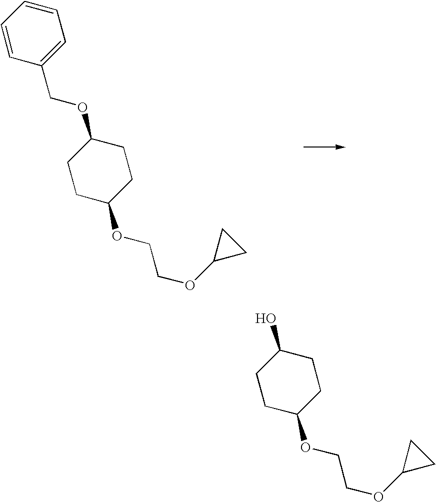

A mixture of cis-[4-(2-cyclopropoxy-ethoxy)-cyclohexyloxymethyl]-benzene (prepared by general procedure X) (0.580 g, 0.0020 mol) and palladium on carbon (10% by weight) (0.212 g, 0.10 equivalent) in ethanol (25 mL) was stirred under a hydrogen atmosphere at about 20° C. for about 12 hours, then filtered through a Celite column. The solvent was removed under reduced pressure to give cis-4-(2-cyclopropoxy-ethoxy)-cyclohexanol (0.441 g, 0.0020 mol); ¹H NMR (CDCl₃, 400 MHz) δ 3.74, 3.64-3.66, 3.55-3.57, 3.44, 3.35, 1.76-1.88, 1.63-1.68, 1.55-1.59, 0.58-0.59, 0.45-0.46; TLC (dichloromethane/ethyl acetate=4:1) R_{f} 0.10.

### General Procedure O: Mitsunobu Coupling of a pyrazolo[3,4-d]pyrimidine or a pyrrolo[2,3-d]pyrimidine with an Alcohol Using a Resin Bound Phosphine Source

A mixture of pyrazolo[3,4-d]pyrimidine or pyrrolo[2,3-d]pyrimidine (preferably 1 equivalent), an alcohol (1-5 equivalents, preferably 2 equivalents), a resin-bound phosphine (1-5 equivalents, preferably 2.2 equivalents), and an azodicarboxylate (for example, diisopropylazodicarboxylate) (1-5 equivalents, preferably 2.2 equivalents) is stirred in an anhydrous solvent (for example, tetrahydrofuran) at about 0-100° C. (preferably about 20° C.) for about 1-48 hours (preferably about 2 hours) under an inert atmosphere. The crude mixture is filtered though a pad of Celite to remove the resin-bound phosphine reagent. The filtrate is collected and the solvent is removed under reduced pressure to afford the crude product that can be further purified by crystallization or chromatography.

### Illustration of General Procedure O

### Preparation #13. 1-(2-Fluoro-1-fluoromethyl-ethyl)-3-iodo-1H-pyrazolo[3,4-d]pyrimidin-4-ylamine

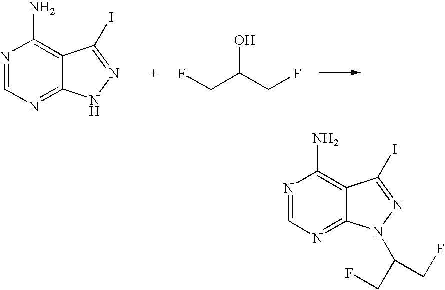

A mixture of 3-iodo-1H-pyrazolo[3,4-d]pyrimidin-4-ylamine (0.250 g, 0.958 mmol) and polystyrene-bound triphenylphosphine (0.7 g, 3 mmol phosphine/g resin, 2.10 mmol) was loaded into a reaction vessel equipped with a magnetic stirring bar. The flask was flushed with nitrogen, and anhydrous tetrahydrofuran (10 mL), and diisopropylazodicarboxylate (0.424 g, 2.10 mmol) were added. 1,3-Difluoro-2-propanol (0.182 g, 1.89 mmol) was added and the mixture was stirred for about 2 hours at ambient temperature. The crude mixture was then filtered though a pad of Celite and the solid was washed with tetrahydrofuran (3×3 mL). The filtrate was concentrated under reduced pressure to give 1-(2-fluoro-]-fluoromethyl-ethyl)-3-iodo-1H-pyrazolo[3,4-d]pyrimidin-4-ylamine; RP-HPLC (5%-95% acetonitrile/0.05 M ammonium acetate over 10 min at 1.7 mL/min; λ=254 nm; Hypersil C18, 5 μm, 100Å, 250×4.6 mm) Rₜ 7.92 min; m/z: (M+H)⁺ 340.

### General Procedure P: Ester Hydrolysis

An ester (preferably 1 equivalent) and a base (lithium hydroxide, sodium hydroxide, or potassium hydroxide, preferably lithium hydroxide) (1-3 equivalents, preferably 1.2 equivalents) are heated in a mixture of water and an organic solvent (for example, methanol or dimethyl sulfoxide, preferably methanol) at about 50-100° C. (preferably about 60° C.) for about 1-24 hours (preferably about 12 hours). After cooling to ambient temperature, the volatile solvents are removed under reduced pressure to afford the product that can be further purified by crystallization or chromatography.

### Illustration of General Procedure P

### Example #272

trans-4-{4-Amino-3-[4-(5,7-dimethyl-benzoxazol-2-ylamino)-phenyl]-pyrazolo[3,4-d]pyrimidin-1-yl}-1-ethyl-cyclohexanecarboxylic acid

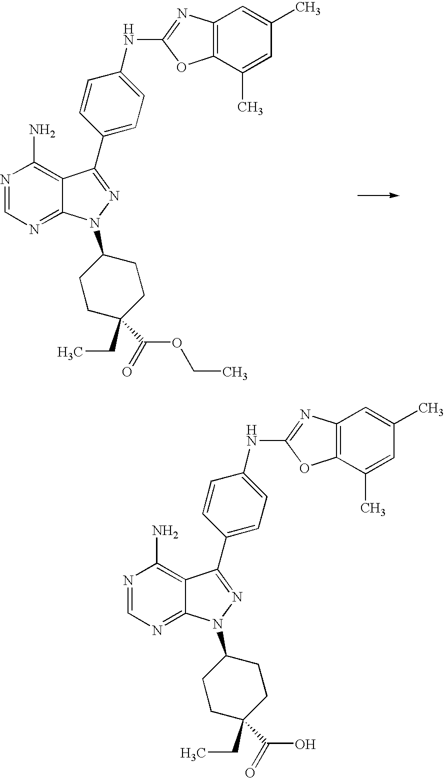

A solution of trans-4-{4-amino-3-[4-(5,7-dimethyl-benzoxazol-2-ylamino)-phenyl]-pyrazolo[3,4-d]pyrimidin-1-yl }-1-ethyl-cyclohexanecarboxylic acid ethyl ester (prepared by general procedures S, A, and C) (3.94 g, 7.13 mmol) in aqueous potassium hydroxide (1 N, 16.4 mL, 16.4 mmol) and dimethyl sulfoxide (20 mL) was heated at about 100° C. for about 15 h. The reaction mixture was cooled to ambient temperature, and aqueous hydrochloric acid (1 N, 20 mL, 20 mmol) was added, affording a precipitate. The solid was filtered, rinsed sequentially with water, and ether, and dried under vacuum to afford trans-4-{4-amino-3-[4-(5,7-dimethyl-benzoxazol-2-ylamino)-phenyl]-pyrazolo[3,4-d]pyrimidin-1-yl}-1-ethyl-cyclohexanecarboxylic acid as an orange solid; RP-HPLC (30% to 95% acetonitrile/0.01M aqueous ammonium acetate over 4.5 min at 0.8 muimin; λ=190-700 nm; Genesis C18, 120Å, 3 μm, 30×4.6 mm column; electrospray ionization method observing both positive and negative ions) Rₜ 2.28; m/z (M+H)⁺ 526.

Other products obtained using general procedure P are shown (Table 9). The method used to determine the HPLC retention time is given in a lower-case letter in parentheses (see Table 1).

TABLE 9
Examples synthesized using general procedure P

| | | | HPLC RT | | |
|---|---|---|---|---|---|
| Ester Precursor | Product | Example # | (method) | | m/z |
| trans-4-{4-Amino-3-[4- | trans-4-{4-Amino-3-[4- | 273 | 7.87 | min (l) | 470 (M + H)⁺ |
| (benzoxazol-2- | (benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}- | d]pyrimidin-1-yl}- | | | | |
| cyclohexanecarboxylic | cyclohexanecarboxylic | | | | |
| acid ethyl ester (A,C,P) | acid | | | | |
| trans-4-{4-Amino-3-[4- | trans-4-{4-Amino-3-[4- | 274 | 2.28 | min (f) | 511 (M)⁻ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}-1- | d]pyrimidin-1-yl}-1- | | | | |
| methyl-cyclohexanecarboxylic | methyl- | | | | |
| acid ethyl | cyclohexanecarboxylic | | | | |
| ester (Example 6) | acid | | | | |

### General Procedure Q: EDC-Coupling of an Acid with an Amine

A mixture of a carboxylic acid (preferably 1 equivalent), an amine (free base or salt) (preferably an amine) (1-5 equivalents, preferably 3 equivalents), 1-(3-dimethylaminopropyl)-3-ethylcarbodiimide hydrochloride (1-3 equivalents, preferably 1.3 equivalents), and a hydroxybenzotriazole (1-hydroxy-7-azabenzotriazole or 1-hydroxybenzotriazole, preferably 1-hydroxy-7-azabenzotriazole) (1-1.5 equivalents, preferably 1 equivalent) is stirred in an organic solvent (dichloromethane or N,N-dimethylformamide, preferably N,N-dimethylformamide) at about 20° C.-60° C. (preferably ambient temperature) for about 15-48 hours (preferably about 15 hours). The solvent is evaporated under reduced pressure, and the mixture is extracted from water with an organic solvent. The organic extracts are dried over a desiccant, evaporated, and the product can be further purified by crystallization or chromatography.

### Illustration of General Procedure Q

### Example #275

trans-(4-{4-Amino-3-[4-(5,7-dimethyl-benzoxazol-2-ylamino)-phenyl]-pyrazolo[3,4-d]pyrimidin-1-yl}-1-ethyl-cyclohexyl)-morpholin-4-yl-methanone

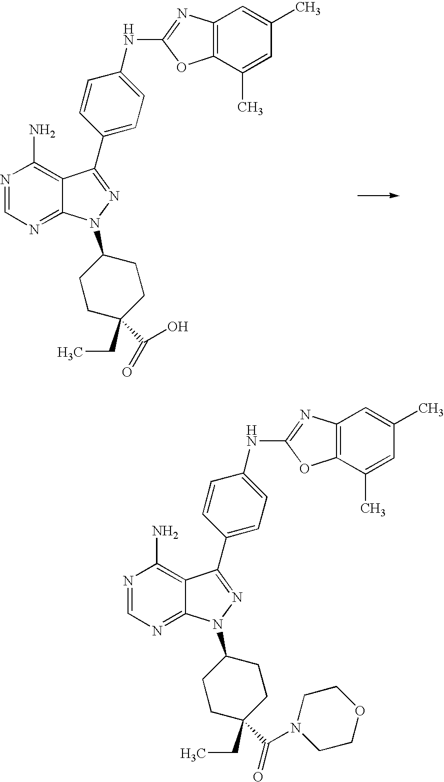

To a solution of trans-4-{4-amino-3-[4-(5,7-dimethyl-benzoxazol-2-ylamino)-phenyl]-pyrazolo[3,4-d]pyrimidin-1-yl}-1-ethyl-cyclohexanecarboxylic acid (example #272, 3.74 g, 7.13 mmol) in N,N-dimethylformamide (50 mL) was added 1-(3-dimethylaminopropyl)-3-ethylcarbodiimide hydrochloride (1.85 g, 9.65 mmol), morpholine (2.79 mL, 32.0 mmol), and 1-hydroxy-7-azabenzotriazole (0.97 g, 7.13 mmol). The mixture was stirred at ambient temperature for about 15 hours. The solvent was removed under reduced pressure, and the product was extracted from water with methanol/ethyl acetate (1:9). The organic fractions were dried over magnesium sulfate, filtered, and concentrated. The product was purified by flash column chromatography on silica gel pre-treated with triethylamine, using methanol/dichloromethane (1:24) as the mobile phase, to afford trans-(4-{4-amino-3-[4-(5,7-dimethyl-benzoxazol-2-ylamino)-phenyl]-pyrazolo[3,4-d]pyrimidin-1-yl}-1-ethyl-cyclohexyl)-morpholin-4-yl-methanone as a yellow solid (1.21 g, 2.04 mmol). RP-HPLC (30% to 95% acetonitrile/0.01M aqueous ammonium acetate over 4.5 min at 0.8 mL/min; λ=190-700 nm; Genesis C18, 120Å, 3 μm, 30×4.6 mm column; electrospray ionization method observing both positive and negative ions) Rₜ 3.03 min; m/z (M+H)⁺ 595.

Other products obtained using general procedure Q are shown (Table 10). The method used to determine the HPLC retention time is given in a lower-case letter in parentheses (see Table 1).

TABLE 10
Examples synthesized using general procedure Q

| | | | HPLC RT | | |
|---|---|---|---|---|---|
| Precursor | Product | Example # | (method) | | m/z |
| trans-4-{4-Amino-3-[4- | trans-(4-{4-Amino-3-[4- | 276 | 2.78 | min (f) | 567 (M + H)⁺ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}- | d]pyrimidin-1-yl}- | | | | |
| cyclohexane- | cyclohexyl)-morpholin- | | | | |
| carboxylic acid (A,C,P) | 4-yl-methanone | | | | |
| trans-4-{4-Amino-3-[4- | trans-4-{4-Amino-3-[4- | 277 | 2.85 | min (f) | 525 (M + H)⁺ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}- | d]pyrimidin-1-yl}- | | | | |
| cyclohexane- | cyclohexanecarboxylic | | | | |
| carboxylic acid (A,C,P) | acid dimethylamide | | | | |
| trans-4-{4-Amino-3-[4- | trans-(4-{4-Amino-3-[4- | 278 | 2.88 | min (f) | 551 (M + H)⁺ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}- | d]pyrimidin-1-yl}- | | | | |
| cyclohexane- | cyclohexyl)-pyrrolidin- | | | | |
| carboxylic acid (A,C,P) | 1-yl-methanone | | | | |
| trans-4-{4-Amino-3-[4- | trans-(4-{4-Amino-3-[4- | 279 | 2.85 | min (f) | 537 (M + H)⁺ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}- | d]pyrimidin-1-yl}- | | | | |
| cyclohexanecarboxylic | cyclohexyl)-azetidin-1- | | | | |
| acid (A,C,P) | yl-methanone | | | | |
| trans-4-{4-Amino-3-[4- | trans-4-{4-Amino-3-[4- | 280 | 3.52 | min (f) | 569 (M + H)⁺ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}-1- | d]pyrimidin-1-yl}-1- | | | | |
| ethyl-cyclohexane- | ethyl- | | | | |
| carboxylic acid | cyclohexanecarboxylic | | | | |
| (Example #273) | acid methoxy-methyl- | | | | |
| | amide | | | | |
| trans-4-{4-Amino-3-[4- | trans-(4-{4-Amino-3-[4- | 281 | 3.12 | min (f) | 565 (M − H)⁻ |
| (5,7-dimethyl- | (5,7-dimethyl- | | | | |
| benzoxazol-2- | benzoxazol-2- | | | | |
| ylamino)-phenyl]- | ylamino)-phenyl]- | | | | |
| pyrazolo[3,4- | pyrazolo[3,4- | | | | |
| d]pyrimidin-1-yl}-1- | d]pyrimidin-1-yl}-1- | | | | |
| ethyl-cyclohexanecarboxylic | ethyl-cyclohexyl)- | | | | |
| acid | azetidin-1-yl- | | | | |
| (Example #273) | methanone | | | | |
| 3-[4-(5,7-Dimethyl- | 1-(4-{4-Amino-3-[4- | 282 | 2.26 | min (k) | 571 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-3- | | | | |
| | hydroxy-2- | | | | |
| | hydroxymethyl-2- | | | | |
| | methyl-propan-1-one | | | | |
| 3-[4-(5,7-Dimethyl- | 1-(4-{4-Amino-3-[4- | 283 | 2.67 | min (k) | 563 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-2-(1H- | | | | |
| | imidazol-4-yl)- | | | | |
| | ethanone | | | | |
| 3-[4-(5,7-Dimethyl- | (4-{4-Amino-3-[4-(5,7- | 284 | 2.55 | min (k) | 553 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,C,L) | (tetrahydro-furan-2-yl)- | | | | |
| | methanone | | | | |
| 3-[4-(5,7-Dimethyl- | 5-(4-{4-Amino-3-[4- | 285 | 2.30 | min (k) | 590 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidine-1-carbonyl)- | | | | |
| | 1-methyl-1H-pyridin-2- | | | | |
| | one | | | | |
| 3-[4-(5,7-Dimethyl- | (4-{4-Amino-3-[4-(5,7- | 286 | 2.50 | min (k) | 567 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d)pyrimidin-1-yl}- | | | | |
| d]pyrimidin-4-ylamine | piperidin-1-yl)- | | | | |
| (A,C,L) | (tetrahydro-pyran-4- | | | | |
| | yl)-methanone | | | | |
| 3-[4-(5,7-Dimethyl- | 1-(4-{4-Amino-3-[4- | 287 | 2.51 | min (k) | 541 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-3- | | | | |
| | methoxy-propan-1-one | | | | |
| 3-[4-(5,7-Dimethyl- | 5-(4-{4-Amino-3-[4- | 288 | 2.22 | min (k) | 566 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidine-1-carbonyl)- | | | | |
| | pyrrolidin-2-one | | | | |
| 3-[4-(5,7-Dimethyl- | (4-{4-Amino-3-[4-(5,7- | 289 | 2.49 | min (k) | 553 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,C,L) | (tetrahydro-furan-3-yl)- | | | | |
| | methanone | | | | |
| 3-[4-(5,7-Dimethyl- | 4-(4-{4-Amino-3-[4- | 290 | 2.30 | min (k) | 594 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidine-1-carbonyl)- | | | | |
| | piperidine-2,6-dione | | | | |
| 3-[4-(5,7-Dimethyl- | 1-(4-{4-Amino-3-[4- | 291 | 2.49 | min (k) | 592 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-2- | | | | |
| | methyl-2-[1,2,4]triazol- | | | | |
| | 1-yl-propan-1-one | | | | |
| 3-[4-(5,7-Dimethyl- | 1-(4-{4-Amino-3-[4- | 292 | 2.72 | min (k) | 577 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-3-(1H- | | | | |
| | imidazol-4-yl)-propan- | | | | |
| | 1-one | | | | |
| 3-[4-(5,7-Dimethyl- | (4-{4-Amino-3-[4-(5,7- | 293 | 2.33 | min (k) | 549 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(1H- | | | | |
| (A,C,L) | imidazol-4-yl)- | | | | |
| | methanone | | | | |
| 3-[4-(5,7-Dimethyl- | 3-[2-(4-{4-Amino-3-[4- | 294 | 2.10 | min (k) | 609 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-2-oxo- | | | | |
| | ethyl]-piperazine-2,5- | | | | |
| | dione | | | | |
| 3-[4-(5,7-Dimethyl- | 4-(4-{4-Amino-3-[4- | 295 | 2.31 | min (k) | 604 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-4-oxo- | | | | |
| | butane-1-sulfonic acid | | | | |
| | amide | | | | |
| 3-[4-(5,7-Dimethyl- | (4-{4-Amino-3-[4-(5,7- | 296 | 2.86 | min (k) | 568 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,C,L) | morpholin-3-yl- | | | | |
| | methanone | | | | |
| 3-[4-(5,7-Dimethyl- | 1-(4-{4-Amino-3-[4- | 297 | 2.56 | min (k) | 581 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-2- | | | | |
| | (tetrahydro-pyran-4- | | | | |
| | yl)-ethanone | | | | |
| 3-[4-(5,7-Dimethyl- | (4-{4-Amino-3-[4-(5,7- | 298 | 2.50 | min (k) | 563 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(1- | | | | |
| (A,C,L) | methyl-1H-imidazol-2- | | | | |
| | yl)-methanone | | | | |
| 3-[4-(5,7-Dimethyl- | (4-{4-Amino-3-[4-(5,7- | 299 | 2.70 | min (k) | 590 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(2- | | | | |
| (A,C,L) | methoxy-pyridin-3-yl)- | | | | |
| | methanone | | | | |
| 3-[4-(5,7-Dimethyl- | trans-(4-{4-Amino-3-[4- | 300 | 2.71 | min (k) | 595 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-(4- | | | | |
| | methoxy-cyclohexyl)- | | | | |
| | methanone | | | | |
| 3-[4-(5,7-Dimethyl- | cis-(4-{4-Amino-3-[4- | 301 | 2.82 | min (k) | 595 (M + H)⁺ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | piperidin-1-yl)-(4- | | | | |
| | methoxy-cyclohexyl)- | | | | |
| | methanone | | | | |
| 3-[4-(5,7-Dimethyl- | 1-(3-{4-Amino-3-[4- | 302 | 2.46 | min (k) | 536 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | azetidin-1-yl)-2-(1H- | | | | |
| | imidazol-4-yl)- | | | | |
| | ethanone | | | | |
| 3-[4-(5,7-Dimethyl- | (3-{4-Amino-3-[4-(5,7- | 303 | 2.47 | min (k) | 526 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | azetidin-1-yl)- | | | | |
| (A,C,L) | (tetrahydro-furan-2-yl)- | | | | |
| | methanone | | | | |
| 3-[4-(5,7-Dimethyl- | 3-{4-Amino-3-[4-(5,7- | 304 | 2.49 | min (k) | 540 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | azetidin-1-yl)- | | | | |
| (A,C,L) | (tetrahydro-pyran-4- | | | | |
| | yl)-methanone | | | | |
| 3-[4-(5,7-Dimethyl- | 1-(3-{4-Amino-3-[4- | 305 | 2.42 | min (k) | 514 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | azetidin-1-yl)-3- | | | | |
| | methoxy-propan-1-one | | | | |
| 3-[4-(5,7-Dimethyl- | (3-{4-Amino-3-[4-(5,7- | 306 | 2.44 | min (k) | 533 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | azetidin-1-yl)-pyridin-3- | | | | |
| (A,C,L) | yl-methanone | | | | |
| 3-[4-(5,7-Dimethyl- | (3-{4-Amino-3-[4-(5,7- | 307 | 2.4 | min (k) | 526 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | azetidin-1-yl)- | | | | |
| (A,C,L) | (tetrahydro-furan-3-yl)- | | | | |
| | methanone | | | | |
| 3-[4-(5,7-Dimethyl- | (3-{4-Amino-3-[4-(5,7- | 308 | 2.63 | min (k) | 563 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | azetidin-1-yl)-(2- | | | | |
| (A,C,L) | methoxy-pyridin-3-yl)- | | | | |
| | methanone | | | | |
| 3-[4-(5,7-Dimethyl- | 1-(3-{4-Amino-3-[4- | 309 | 2.38 | min (k) | 565 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,C,L) | azetidin-1-yl)-2-methyl- | | | | |
| | 2-[1,2,4]triazol-1-yl- | | | | |
| | propan-1-one | | | | |

### General Procedure R: Boc-Protection of an Amine

To a solution of the amine (preferably 1 equivalent) in an organic solvent (for example, (dioxane/water or tetrahydrofuran) in the absence or presence of a base (for example, sodium carbonate, cesium carbonate, preferably sodium carbonate) (1-5 equivalents, preferably 2.4 equivalents) is added di-tert-butyldicarbonate (1-5 equivalents, preferably 1.2 equivalents). The reaction mixture is stirred at about 0-50° C. (preferably about 25° C.) for about 1-48 hours (preferably about 12 hours). The organic solvent is removed under reduced pressure. The residue is partitioned between water and an appropriate organic solvent, the organic layer is separated, and the aqueous layer is further extracted with an organic solvent. The combined organic extracts are dried over a desiccant. The solvents are evaporated under reduced pressure to afford the product that can be further purified by crystallization or chromatography.

### Illustration of General Procedure R

### Preparation #14. (R)-3-Hydroxy-piperidine-1-carboxylic acid tert-butyl ester

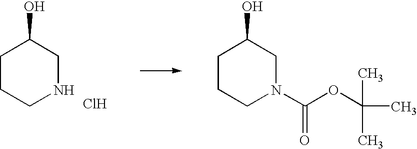

To a solution of (R)-3-hydroxypiperidine hydrochloride (10.3 g, 0.075 mol) in dioxane/water (80 mL each) was added di-tert-butyldicarbonate (20 g, 0.091 mol) and sodium carbonate (19 g, 0.182 mol). The mixture was stirred at room temperature for about 16 hours. The organic solvent was removed under reduced pressure and the aqueous layer was extracted with diethyl ether (3×100 mL). The combined organic extracts were washed with brine (100 mL), dried over magnesium sulfate, and the solvent was removed under reduced pressure to afford (R)-3-hydroxy-piperidine-1-carboxylic acid tert-butyl ester (15.1 g, 0.075 mol) as a colorless oil; m/z (M+H)⁺ 202.

### General Procedure S: α-Alkylation of a Hydroxy Alkyl Carboxylate

A mixture of a hydroxy alkyl carboxylate (preferably 1 equivalent), a silylating agent (tert-butyldimethylsilyl chloride or triethylsilyl chloride, preferably tert-butyldimethylsilyl chloride) (1-3 equivalents, preferably 1.15 equivalents), a base (imidazole or triethylamine, preferably imidazole), and a catalyst (pyridine or 4-dimethylaminopyridine, preferably 4-dimethylaminopyridine) (0.01 to 1.0 equivalents, preferably 0.04 equivalents) are stirred in an organic solvent (dichloromethane or N,N-dimethylformamide, preferably N,N-dimethylformamide) at ambient temperature for about 1-24 hours (preferably about 15 hours). The solvent is removed under reduced pressure, and the product is extracted from water with an organic solvent. The organic extracts are dried over a desiccant and concentrated to afford the silyl ether that can be further purified by chromatography. The resulting silyl ether (preferably 1 equivalent) is dissolved in an organic solvent (ether or tetrahydrofuran, preferably tetrahydrofuran), and enolized with a strong base (preferably lithium diisopropylamide) (2-4 equivalents, preferably 2.5 equivalents) at about −78 to 25° C. (preferably about 0° C.). An alkyl halide (preferably methyl iodide or ethyl iodide) (1-10 equivalents, preferably 3.5 equivalents) is added and the reaction is stirred at about −78 to 25° C. (preferably about 25° C.) for about 2-24 hours (preferably about 4 hours). The solvents are removed under reduced pressure, and the alkylated ester can be further purified by chromatography. The alkylated ester (preferably 1 equivalent) is mixed with a fluoride source (potassium fluoride or tetrabutylammonium fluoride, preferably tetrabutylammonium fluoride) (1-2 equivalents, preferably1.2 equivalents) in an organic solvent (preferably tetrahydrofuran) at about 0-50° C. (preferably about 25° C.) for about 1-24 hours (preferably about 15 hours). The solvent is removed under reduced pressure, and the product is extracted from water with an organic solvent, and can be further purified by chromatography or crystallization.

### Illustration of General Procedure S

### Preparation #15. trans-1-Ethyl-4-hydroxy-cyclohexanecarboxylic acid ethyl ester

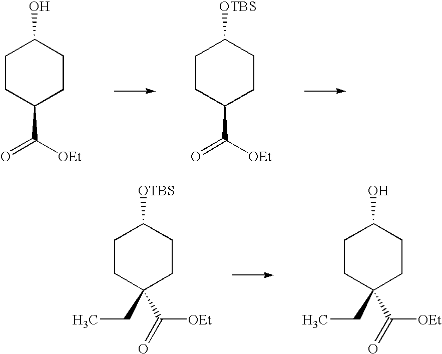

A mixture of ethyl-4-hydroxycyclohexanecarboxylate (16.0 g, 92.9 mmol), tert-butyldimethylsilylchloride (16.1 g, 106.8 mmol), imidazole (8.41 g, 123.5 mmol), and 4-dimethylaminopyridine (0.453 g, 3.71 mmol) in N,N-dimethylformamide (150 mL) was stirred at ambient temperature for about 15 hours. The solvent was evaporated under reduced pressure, and the product was extracted from aqueous ammonium chloride using ether/petroleum ether (1:1). The organic extracts were dried over magnesium sulfate and concentrated, and the residue was purified by flash column chromatography on silica gel using ether/petroleum ether (1:14) as the mobile phase to afford 4-(tert-butyl-dimethyl-silyloxy)cyclohexanecarboxylic acid ethyl ester (25.8 g, 90.2 mmol) as a colorless oil; m/z (M+H)⁺ 287. An ice-cooled solution of the above compound (25.8 g, 90.2 mmol) in tetrahydrofuran (50 mL) was added to a solution of lithium diisopropylamide (225 mmol) in tetrahydrofuran (200 mL) at about −78° C. The mixture was stirred at about −78° C. for about 1 hour, warmed to about 0° C. for about 5 minutes, and cooled again to about −78° C. Ethyl iodide (25.2 mL, 316 mmol) was added via a syringe, and the mixture was allowed to warm to ambient temperature. After 1 hour at ambient temperature, excess base was quenched with aqueous ammonium chloride, and volatile solvents were removed under reduced pressure. The product was extracted from water with ether and concentrated, and was purified by flash column chromatography on silica gel, using ether/petroleum ether (1:15) as the mobile phase to afford 4-(tert-butyl-dimethyl-silyloxy)-1-ethyl-cyclohexanecarboxylic acid ethyl ester (27.3 g, 86.9 mmol) as a colorless oil. The above product (27.3 g, 86.9 mmol) was mixed with a solution of tetrabutylammonium fluoride in tetrahydrofuran (1 M, 144 mL, 144 mmol) at about 0° C, and the reaction mixture was allowed to warm to ambient temperature for 15 hours. The tetrahydrofuran was removed under reduced pressure, and the residue was extracted from water (500 mL) with ether (5×500 mL). The combined organic extracts were dried over magnesium sulfate and concentrated. The product was purified by flash column chromatography on silica gel, using ether/petroleum ether (4:1) as the mobile phase to afford trans-1-ethyl-4-hydroxy-cyclohexanecarboxylic acid ethyl ester (17.39 g, 87.0 mmol) as a colorless oil; ¹H NMR (400 MHz, DMSO-d₆) δ: 4.45, 4.10, 2.05, 1.69, 1.41, 1.18, 1.11, 0.73.

### General Procedure T: Deketalization of a Protected Cyclohexanone

A mixture of a 8-substituted 1,4-dioxa-spiro[4.5]decane (preferably 1 equivalent) and an acid (for example, hydrochloric acid, sulfuric acid, oxalic acid, or trifluoroacetic acid, preferably oxalic acid) (1-10 equivalents, preferably 3 equivalents) is heated in a mixture of water and an organic solvent (for example, acetone, ethanol, ethyl acetate, ethylene glycol dimethyl ether, tetrahydrofuran, toluene, or a mixture of the listed solvents, preferably tetrahydrofuran/water 2:1) at about 0° C.-120° C. (preferably about 70° C.) for about 1-48 hours (preferably 6 hours). The solvent is removed under reduced pressure. The residue is partitioned between an aqueous solution and an organic solvent, the organic layer is separated and the aqueous layer is further extracted with organic solvent. The combined organic extracts are dried over a desiccant. The solvents are evaporated under reduced pressure to afford the desired product that can be further purified by crystallization or chromatography.

### Illustration of General Procedure T

### Preparation #16. 4-(4-Chloro-5-iodo-pyrrolo[2,3-d]pyrimidin-7-yl)-cyclohexanone

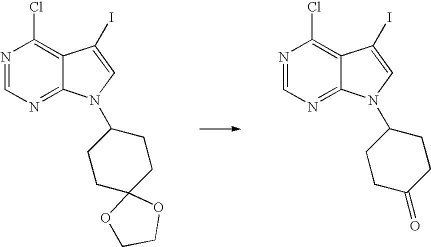

To a suspension of 4-chloro-7-(1,4-dioxa-spiro[4.5]dec-8-yl)-5-iodo-7H-pyrrolo[2,3-d]pyrimidine (prepared via general procedure A) (0.420 g, 0.0010 mol) in acetone (20 mL) at about 0° C., hydrochloric acid (6.0 M, 0.55 mL, 0.0033 mol) was added slowly through a dropping funnel. The reaction mixture was stirred at about 0° C. for about 1 hour, at ambient temperature for about 24 hours. Additional hydrochloric acid (6.0 N, 0.25 mL, 0.0015 mol) was added and the reaction mixture was stirred at ambient temperature for a further 3 days. The solvent was removed under reduced pressure, and the residue was washed with water. The resulting precipitate was filtered and washed with water (100 mL). Drying under reduced pressure afforded 4-(4-chloro-5-iodo-pyrrolo[2,3-d]pyrimidin-7-yl)-cyclohexanone (0.319 g, 0.0085 mol); ¹H NMR (DMSO-d₆, 400 MHz) δ 8.674, 8.167, 5.263, 2.710-2.777, 2.295-2.392, 2.086; RP-HPLC (30% to 95% acetonitrile/0.01M aqueous ammonium acetate over 4.5 min at 0.8 mL/min; λ=190-700 nm; Genesis C18, 120Å, 3 μm, 30×4.6 mm column) Rₜ 2.80 min.

### General procedure U: Reduction of Ketone or Ester to an Alcohol

A reducing agent (sodium borohydride, lithium tri-sec-butylborohydride, lithium aluminum hydride, or lithium triethylborohydride, preferably sodium borohydride for ketones and lithium aluminium hydride for esters) (1-10 equivalents, preferably 2 equivalents) is added portionwise to a solution of a ketone or an ester (preferably 1 equivalent) in an organic solvent (methanol or tetrahydrofuran, preferably tetrahydrofuran) at about −78° C. to ambient temperature (preferably at about −70° C.). The reaction is stirred at ambient temperature for about 1-72 hours (preferably about 2 hours) until it reaches completion. The excess reducing agent is quenched by addition of small amount of water. The resulting mixture is partitioned between an aqueous layer and an organic solvent. The organic phase is separated, washed with a saturated brine solution and dried over a desiccant. The solvent is then removed under reduced pressure to yield the crude product that can be further purified by crystallization or chromatography.

### Illustration of General Procedure U

### Example #310

trans-4-{4-Amino-3-[4-(5,7-dimethyl-benzoxazol-2-ylamino)-phenyl]-pyrazolo[3,4-d]pyrimidin-1-yl}-cyclohexanol

### Example #311

cis-4-{4-Amino-3-[4-(5,7-dimethyl-benzoxazol-2-ylamino)-phenyl]-pyrazolo[3,4-d]pyrimidin-1-yl}-cyclohexanol

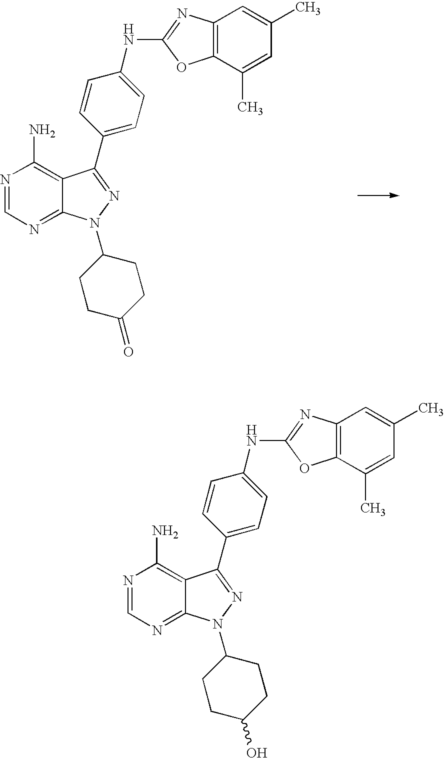

A mixture of 4-(4-amino-3-{4-[(5,7-dimethyl-1,3-benzoxazol-2-yl)amino]phenyl}-1H-pyrazolo[3,4-d]pyrimidin-1-yl)-1-cyclohexanone (0.15 g, 0.32 mmol) and sodium borohydride (0.015 g, 0.38 mmol) was stirred in methanol (10 mL) at ambient temperature for about 72 hours, during which additional sodium borohydride (0.055 g, 1.45 mmol) was added portionwise to drive the reaction to completion. The reaction was then quenched by the addition of water (0.1 mL), and the solvent was removed under reduced pressure. The crude solid was purified by flash chromatography on silica using a gradient of 0%-6% methanol (containing 2% of 28% aqueous ammonia) in dichloromethane as the mobile phase to afford a slower running fraction containing trans-4-{4-amino-3-[4-(5,7-dimethyl-benzoxazol-2-ylamino)-phenyl]-pyrazolo[3,4-d]pyrimidin-1-yl}-cyclohexanol (0.053 g, 0.11 mmol) as a white solid; RP-HPLC (5% to 95% acetonitrile/0.05M aqueous ammonium acetate, buffered to pH 4.5, over 10 min at 1 mL/min; λ=254 nm; Hypersil C18, 100Å, 5 μm, 250×4.6 mm column) Rₜ 10.05 min; m/z (M+H)⁺ 470.3; and a faster running fraction containing cis-4-{4-amino-3-[4-(5,7-dimethyl-benzoxazol-2-ylamino)-phenyl]-pyrazolo[3,4-d]pyrimidin-1-yl}-cyclohexanol (0.023 g, 0.05 mmol) as a white solid; RP-HPLC (5% to 95% acetonitrile/0.05M aqueous ammonium acetate, buffered to pH 4.5, over 10 min at 1 mL/min; λ=254 nm; Hypersil C18, 100Å, 5 μm, 250×4.6 mm column) Rₜ 10.29 min; m/z (M+H)⁺ 470.3.

Other products obtained using general procedure U are shown (Table 11).

The method used to determine the HPLC retention time is given in a lower-case letter in parentheses (see Table 1).

TABLE 11
Examples synthesized using general procedure U

| | | | HPLC RT | |
|---|---|---|---|---|
| Ester | Product | Example # | (Method) | m/z |
| trans-4-{4-Amino-3-[4- | trans-(4-{4-Amino-3-[4- | 312 | 3.22 min (f) | 512 (M + H)⁺ |
| (5,7-dimethyl- | (5,7-dimethyl- | | | |
| benzoxazol-2- | benzoxazol-2- | | | |
| ylamino)-phenyl]- | ylamino)-phenyl]- | | | |
| pyrazolo[3,4- | pyrazolo[3,4- | | | |
| d]pyrimidin-1-yl}-1- | d]pyrimidin-1-yl}-1- | | | |
| ethyl-cyclohexane- | ethyl-cyclohexyl)- | | | |
| carboxylic acid ethyl | methanol | | | |
| ester (Example #7) | | | | |

### General Procedure V: Mesylation of an Alcohol and Subsequent Displacement of the Mesylate Group

An alcohol (preferably 1 equivalent) is dissolved in a mixture of an organic solvent (preferably dichloromethane) and an organic base (sodium hydride, pyridine, preferably pyridine). Methanesulfonyl chloride (1-6 equivalents, preferably 1.6 equivalents) is added and the reaction mixture is stirred at about 10-60° C. (preferably about 25° C.) under continuous nitrogen flow for about 10-80 hours (preferably about 40 hours). The solvents are removed under reduced pressure and the residue is triturated with water. The precipitate is collected by filtration and washed with water. The precipitate is dried under reduced pressure and optionally purified by trituration, crystallization or chromatography.

A mesylate (preferably 1 equivalent) is dissolved in an organic solvent (N-methyl pyrrolidinone, dimethyl sulfoxide or N,N-dimethylformamide, preferably N,N-dimethylformamide) and an inorganic base (cesium carbonate, sodium carbonate or sodium hydride, preferably sodium hydride) (1-10 equivalents, preferably 5 equivalents) is added, followed by the addition of the nucleophile (1-10 equivalents, preferably 5 equivalents). The reaction mixture is heated at about 30-70° C. (preferably about 55° C.) for about 10-100 hours (preferably 24 hours) under continuous nitrogen flow. The reaction mixture is concentrated under reduced pressure and the residue is purified by crystallization or chromatography.

### Illustration of General Procedure V

### Preparation #17. trans-3-Iodo-1-(4-pyrazol-1-yl-cyclohexyl)-1H-pyrazolo[3,4-d]pyrimidin-4-ylamine

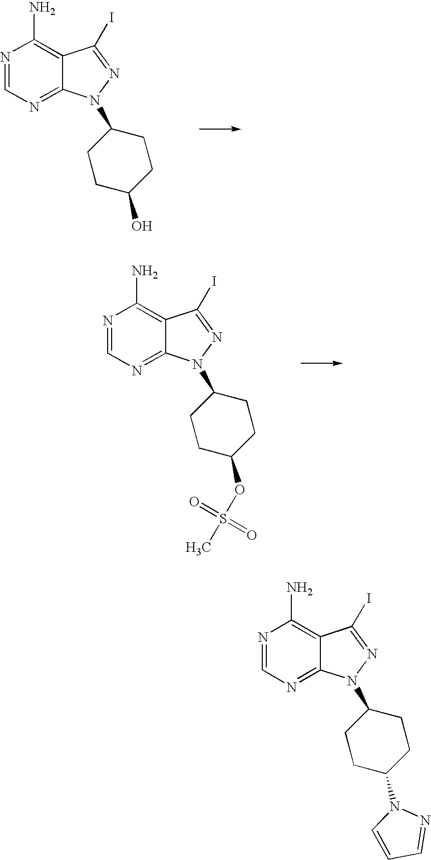

Methanesulfonyl chloride (0.72 mL, 0.00930 mol) was added to a mixture of cis-4-(4-amino-3-iodo-pyrazolo[3,4-d]pyrimidin-1-yl)-cyclohexanol (2.00 g, 0.00557 mol), dichloromethane (20 mL) and pyridine (20 mL). The reaction mixture was stirred at about 25° C. under continuous nitrogen flow for about 30 hours. The solvents were removed under reduced pressure and the residue was triturated with water (25 mL). The precipitate was collected by filtration, washed with water, dried under reduced pressure for 24 hours, placed on a glass filter, washed with ethyl acetate, and dried under reduced pressure for 24 hours to yield cis-4-(4-amino-3-iodo-pyrazolo[3,4-d]pyrimidin-1-yl)-cyclohexylmethanesulfonate (1.46 g, 0.003 mol) as an off-white solid; RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 12.14 min.

cis-4-(4-Amino-3-iodo-pyrazolo[3,4-d]pyrimidin-1-yl)-cyclohexyl methanesulfonate (0.68 g, 0.00156 mol) was dissolved in N,N-dimethylformamide (40 mL), and sodium hydride (60% dispersion in mineral oil, 0.31 g, 0.00764 mol) and pyrazole (0.53 g, 0.00778 mol) were added sequentially. The reaction mixture was heated at about 55° C. for about 24 hours under continuous nitrogen flow. The reaction mixture was concentrated under reduced pressure and the residue was purified by preparative HPLC to afford trans-3-iodo-1-(4-pyrazol-1-yl-cyclohexyl)-1H-pyrazolo[3,4-d]pyrimidin-4-ylamine (0.152 g, 0.000371 mol) as a white solid; m/z: (M+H)⁺ 410.

### General Procedure W: Acylation of an Amine with an Acid Chloride, Sulfonyl Chloride or an Anhydride

A mixture of an amine (1-1.25 equivalents, preferably 1 equivalent), a base (for example, pyridine, triethylamine or diisopropylethylamine, preferably triethylamine) (1-5 equivalents, preferably 4 equivalents) and either an acyl chloride, sulfonyl chloride or an acid anhydride (1-1.25 equivalents, preferably 1.04 equivalents) is stirred in an organic solvent (for example dichloromethane or tetrahydrofuran, preferably dichloromethane) at about −10° to 50° C. (preferably about 0° C.) for about 2-10 hours (preferably about 5 hours). The reaction is quenched with an alcohol (for example methanol or ethanol, preferably methanol) or water and the mixture is allowed to warm to ambient temperature. The solvents are removed under reduced pressure and the residue is optionally purified by chromatography or crystallization.

### Illustration of General Procedure W

### Example #313

1-(4-{4-Amino-3-[4-(5,7-dimethylbenzoxazol-2-ylamino)-phenyl]-pyrazolo[3,4 pyrimidin-1-yl}-piperidin-1-yl)-2-methylpropan-1-one

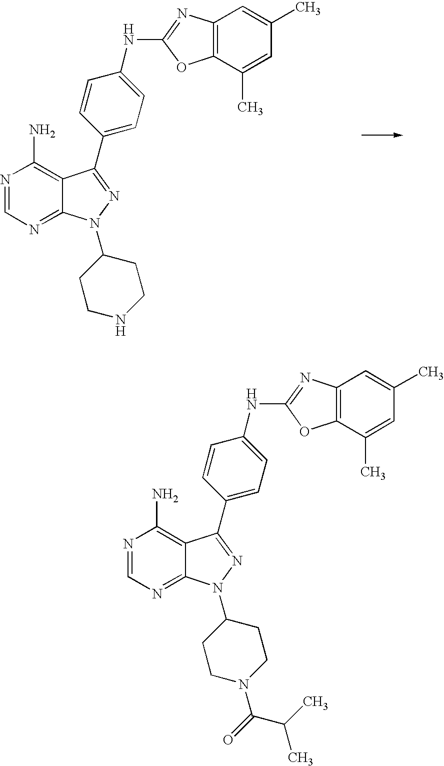

To a mixture of 3-[4-(5,7-dimethylbenzoxazol-2-ylamino)-phenyl]-1-piperidin-4-yl-1H-pyrazolo[3,4-d]pyrimidin-4-ylamine (0.0585, 0.13 mmol) and triethylamine (0.0547, 0.54 mmol) in anhydrous dichloromethane (2 ml) at about 0° C., was added a solution of isobutyryl chloride (0.0144 g, 0.135 mmol) in anhydrous dichloromethane (2 ml) and the resulting mixture was stirred for about 5 hours at about 0° C. Methanol (1 ml) was added and the resulting mixture was stirred for 1 hour. The solvents were removed under reduced pressure and the residue was purified by mass actuated preparative RP-HPLC (25% to 75% acetonitrile/0.05 M aqueous ammonium acetate, buffered to pH 4.5, over 7 min at 25 mL/min, 100% acetonitrile for 2 min, 100% to 25% acetonitrile/0.05 M mM ammonium acetate over 1.5 min; Hypersil BDS C18, 100 Å, 5 μm, 100×21.2 mm column) to afford 1-(4-{4-amino-3-[4-(5,7-dimethylbenzoxazol-2-ylamino)-phenyl]-pyrazolo[3,4-d]pyrimidin-1-yl}-piperidin-1-yl)-2-methylpropan-1-one (0.043 g, 0.08 mmol) as a white solid; RP-HPLC (5% to 95% acetonitrile/0.05 M ammonium acetate, buffered to pH 4.5, over 3.5 min at 2 mL/min, λ=250-470 nm; Pecosphere C18, 3 μm, 33×4.6 mm; electrospray ionization method observing both positive and negative ions) Rₜ 2.8 min; m/z: (M+H)⁺ 525.

Other products obtained using general procedure W are shown (Table 12). The method used to determine the HPLC retention time is given in a lower-case letter in parentheses (see Table 1).

TABLE 12
Examples synthesized using general procedure W

| | | | HPLC RT | | |
|---|---|---|---|---|---|
| Precursor | Product | Example # | (method) | | m/z |
| 3-[4-(5,7-Dimethyl- | 1-(4-{4-Amino-3-[4- | 314 | 3.11 | min (k) | 553 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-3,3- | | | | |
| | dimethylbutan-1-one | | | | |
| 3-[4-(5,7-Dimethyl- | 1-(4-{4-Amino-3-[4- | 315 | 2.47 | min (k) | 527 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-2- | | | | |
| | methoxyethanone | | | | |
| 3-[4-(5,7-Dimethyl- | 1-(4-{4-Amino-3-[4- | 316 | 2.95 | min (k) | 539 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-3- | | | | |
| | methylbutan-1-one | | | | |
| 3-[4-(5,7-Dimethyl- | 1-(4-{4-Amino-3-[4- | 317 | 2.66 | min (k) | 511 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-propan- | | | | |
| | 1-one | | | | |
| 3-[4-(5,7-Dimethyl- | 1-(4-{4-Amino-3-[4- | 318 | 2.81 | min (k) | 525 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-butan-1- | | | | |
| | one | | | | |
| 3-[4-(5,7-Dimethyl- | 1-(4-{4-Amino-3-[4- | 319 | 2.99 | min (k) | 539 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-pentan- | | | | |
| | 1-one | | | | |
| 3-[4-(5,7-Dimethyl- | 1-(4-{4-Amino-3-[4- | 320 | 3.18 | min (k) | 553 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-hexan- | | | | |
| | 1-one | | | | |
| 3-[4-(5,7-Dimethyl- | 1-(4-{4-Amino-3-[4- | 321 | 3.37 | min (k) | 567 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-heptan- | | | | |
| | 1-one | | | | |
| 3-[4-(5,7-Dimethyl- | (4-{4-Amino-3-[4-(5,7- | 322 | 2.73 | min (k) | 523 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,L,C) | cyclopropylmethanone | | | | |
| 3-[4-(5,7-Dimethyl- | (4-{4-Amino-3-[4-(5,7- | 323 | 2.91 | min (k) | 537 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,L,C) | cyclobutylmethanone | | | | |
| 3-[4-(5,7-Dimethyl- | (4-{4-Amino-3-[4-(5,7- | 324 | 3.07 | min (k) | 551 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,L,C) | cyclopentylmethanone | | | | |
| 3-[4-(5,7-Dimethyl- | (4-{4-Amino-3-[4-(5,7- | 325 | 3.19 | min (k) | 565 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,L,C) | cyclohexylmethanone | | | | |
| 3-[4-(5,7-Dimethyl- | (4-{4-Amino-3-[4-(5,7- | 326 | 2.79 | min (k) | 549 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-furan-2- | | | | |
| (A,L,C) | yl-methanone | | | | |
| 3-[4-(5,7-Dimethyl- | (4-{4-Amino-3-[4-(5,7- | 327 | 2.92 | min (k) | 565 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)- | | | | |
| (A,L,C) | thiophen-2-yl- | | | | |
| | methanone | | | | |
| 3-[4-(5,7-Dimethyl- | 1-(4-{4-Amino-3-[4- | 328 | 3.1 | min (k) | 553 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-2-ethyl- | | | | |
| | butan-1-one | | | | |
| 3-[4-(5,7-Dimethyl- | 1-(4-{4-Amino-3-[4- | 329 | 3.14 | min (k) | 553 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-4- | | | | |
| | methylpentan-1-one | | | | |
| 3-[4-(5,7-Dimethyl- | (4-{4-Amino-3-[4-(5,7- | 330 | 2.7 | min (k) | 578 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(3,5- | | | | |
| (A,L,C) | dimethyl-isoxazol-4-yl)- | | | | |
| | methanone | | | | |
| 3-[4-(5,7-Dimethyl- | 1-(4-{4-Amino-3-[4- | 331 | 2.8 | min (k) | 578 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-3- | | | | |
| | methylsulfanylpropan- | | | | |
| | 1-one | | | | |
| 3-[4-(5,7-Dimethyl- | (4-{4-Amino-3-[4-(5,7- | 332 | 2.83 | min (k) | 564 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(5- | | | | |
| (A,L,C) | methylisoxazol-3-yl)- | | | | |
| | methanone | | | | |
| 3-[4-(5,7-Dimethyl- | (4-{4-Amino-3-[4-(5,7- | 333 | 2.77 | min (k) | 581 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(4- | | | | |
| (A,L,C) | methyl- | | | | |
| | [1,2,3]thiadiazol-5-yl)- | | | | |
| | methanone | | | | |
| 3-[4-(5,7-Dimethyl- | (4-{4-Amino-3-[4-(5,7- | 334 | 3.02 | min (k) | 577 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(2,5- | | | | |
| (A,L,C) | dimethylfuran-3-yl)- | | | | |
| | methanone | | | | |
| 3-[4-(5,7-Dimethyl- | (4-{4-Amino-3-[4-(5,7- | 335 | 2.53 | min (k) | 561 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-pyrazin- | | | | |
| (A,L,C) | 2-yl-methanone | | | | |
| 3-[4-(5,7-Dimethyl- | (4-{4-Amino-3-[4-(5,7- | 336 | 2.78 | min (k) | 591 (M + H)⁺ |
| benzoxazol-2- | dimethylbenzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidin-1-yl)-(2-ethyl- | | | | |
| (A,L,C) | 5-methyl-2H-pyrazol-3- | | | | |
| | yl)-methanone | | | | |
| 3-[4-(5,7-Dimethyl- | 1-(4-{4-Amino-3-[4- | 337 | 2.49 | min (k) | 571 (M + H)⁺ |
| benzoxazol-2- | (5,7- | | | | |
| ylamino)-phenyl]-1- | dimethylbenzoxazol-2- | | | | |
| pyperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-2-(2- | | | | |
| | methoxyethoxy)- | | | | |
| | ethanone | | | | |
| 3-[4-(5,7-Dimethyl- | 3-[4-(5,7- | 338 | 3.34 | min (k) | 587 (M + H)⁺ |
| benzoxazol-2- | Dimethylbenzoxazol-2- | | | | |
| ylamino)-phenyl]-1- | ylamino)-phenyl]-1-(1- | | | | |
| piperidin-4-yl-1H- | trifluoromethanesulfon | | | | |
| pyrazolo[3,4- | yl-piperidin-4-yl)-1H- | | | | |
| d]pyrimidin-4-ylamine | pyrazolo[3,4- | | | | |
| (A,L,C) | d]pyrimidin-4-ylamine | | | | |
| 3-[4-(5,7-Dimethyl- | 1-(4-{4-Amino-3-[4- | 339 | 3.34 | min (k) | 567 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)- | | | | |
| pyrazolo[3,4- | phenyl]pyrazolo[3,4- | | | | |
| d]pyrimidin-4-ylamine | d]pyrimidin-1-yl}- | | | | |
| (A,L,C) | piperidin-1-yl)-5- | | | | |
| | methylhexan-1-one | | | | |
| 3-[4-(5,7-Dimethyl- | 3-[4-(5,7- | 340 | 2.96 | min (k) | 561 (M + H)⁺ |
| benzoxazol-2- | Dimethylbenzoxazol-2- | | | | |
| ylamino)-phenyl]-1- | ylamino)-phenyl]-1-[1- | | | | |
| piperidin-4-yl-1H- | (propane-2-sulfonyl)- | | | | |
| pyrazolo[3,4- | piperidin-4-yl]-1H- | | | | |
| d]pyrimidin-4-ylamine | pyrazolo[3,4d]pyrimidin- | | | | |
| (A,L,C) | 4-ylamine | | | | |
| 3-[4-(5,7-Dimethyl- | 3-[4-(5,7- | 341 | 2.82 | min (k) | 547 (M + H)⁺ |
| benzoxazol-2- | Dimethylbenzoxazol-2- | | | | |
| ylamino)-phenyl]-1- | ylamino)-phenyl]-1-(1- | | | | |
| piperidin-4-yl-1H- | ethanesulfonylpiperidin- | | | | |
| pyrazolo[3,4- | 4-yl)-1H-pyrazolo[3,4- | | | | |
| d]pyrimidin-4-ylamine | d]pyrimidin-4-ylamine | | | | |
| (A,L,C) | | | | | |
| 3-[4-(5,7-Dimethyl- | 3-[4-(5,7- | 342 | 2.98 | min (k) | 561 (M + H)⁺ |
| benzoxazol-2- | Dimethylbenzoxazol-2- | | | | |
| ylamino)-phenyl]-1- | ylamino)-phenyl]-1-[1- | | | | |
| piperidin-4-yl-1H- | (propane-1-sulfonyl)- | | | | |
| pyrazolo[3,4- | piperidin-4-yl]-1H- | | | | |
| d]pyrimidin-4-ylamine | pyrazolo[3,4d]pyrimidin- | | | | |
| (A,L,C) | 4-ylamine | | | | |
| 3-[4-(5,7-Dimethyl- | 1-[1-(Butane-1- | 343 | 3.14 | min (k) | 575 (M + H)⁺ |
| benzoxazol-2- | sulfonyl)-piperidin-4- | | | | |
| ylamino)-phenyl]-1- | yl)-3-[4-(5,7- | | | | |
| piperidin-4-yl-1H- | dimethylbenzoxazol-2- | | | | |
| pyrazolo[3,4- | ylamino)-phenyl]-1H- | | | | |
| d]pyrimidin-4-ylamine | pyrazolo[3,4- | | | | |
| (A,L,C) | d]pyrimidin-4-ylamine | | | | |
| 1-Azetidin-3-yl-3-[4- | 1-(3-{4-Amino-3-[4- | 344 | 2.38 | min (k) | 500 (M + H)⁺ |
| (5,7-dimethyl- | (5,7- | | | | |
| benzoxazol-2- | dimethylbenzoxazol-2- | | | | |
| ylamino)-phenyl]-1H- | ylamino)-phenyl]- | | | | |
| pyrazolo[3,4- | pyrazolo[3,4d]pyrimidin- | | | | |
| d]pyrimidin-4-ylamine | 1-yl}-azetidin-1-yl)-2- | | | | |
| (WO 01/19829) | methoxyethanone | | | | |
| 1-Azetidin-3-yl-3-[4- | 1-(3-{4-Amino-3-[4- | 345 | 2.48 | min (k) | 484 (M + H)⁺ |
| (5,7-dimethyl- | (5,7- | | | | |
| benzoxazol-2- | dimethylbenzoxazol-2- | | | | |
| ylamino)-phenyl]-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (WO 01/19829) | azetidin-1-yl)-propan- | | | | |
| | 1-one | | | | |
| 1-Azetidin-3-yl-3-[4- | (3-{4-Amino-3-[4-(5,7- | 346 | 2.58 | min (k) | 496 (M + H)⁺ |
| (5,7-dimethyl- | dimethylbenzoxazol-2- | | | | |
| benzoxazol-2- | ylamino)-phenyl]- | | | | |
| ylamino)-phenyl]-1H- | pyrazolo[3,4- | | | | |
| pyrazolo[3,4- | d]pyrimidin-1-yl}- | | | | |
| d]pyrimidin-4-ylamine | azetidin-1-yl)- | | | | |
| (WO 01/19829) | cyclopropylmethanone | | | | |
| 1-Azetidin-3-yl-3-[4- | (3-{4-Amino-3-[4-(5,7- | 347 | 2.72 | min (k) | 510 (M + H)⁺ |
| (5,7-dimethyl- | dimethylbenzoxazol-2- | | | | |
| benzoxazol-2- | ylamino)-phenyl]- | | | | |
| ylamino)-phenyl]-1H- | pyrazolo[3,4- | | | | |
| pyrazolo[3,4- | d]pyrimidin-1-yl}- | | | | |
| d]pyrimidin-4-ylamine | azetidin-1-yl)- | | | | |
| (WO 01/19829) | cyclobutylmethanone | | | | |
| 1-Azetidin-3-yl-3-[4- | 3-[4-(5,7- | 348 | 2.74 | min (k) | 520 (M + H)⁺ |
| (5,7-dimethyl- | Dimethylbenzoxazol-2- | | | | |
| benzoxazol-2- | ylamino)-phenyl]-1-(1- | | | | |
| ylamino)-phenyl]-1H- | ethanesulfonyl- | | | | |
| pyrazolo[3,4- | azetidin-3-yl)-1H- | | | | |
| d]pyrimidin-4-ylamine | pyrazolo[3,4- | | | | |
| (WO 01/19829) | d]pyrimidin-4-ylamine | | | | |
| 1-Azetidin-3-yl-3-[4- | 1-(3-{4-Amino-3-[4- | 349 | 2.98 | min (k) | 544 (M + H)⁺ |
| (5,7-dimethyl- | (5,7- | | | | |
| benzoxazol-2- | dimethylbenzoxazol-2- | | | | |
| ylamino)-phenyl]-1H- | ylamino)-3-fluoro- | | | | |
| pyrazolo[3,4- | phenyl]-pyrazolo[3,4- | | | | |
| d]pyrimidin-4-ylamine | d]pyrimidin-1-yl}- | | | | |
| (WO 01/19829) | azetidin-1-yl)-2- | | | | |
| | ethylbutan-1-one | | | | |
| 1-Azetidin-3-yl-3-[4- | (3-{4-Amino-3-[4-(5,7- | 350 | 2.68 | min (k) | 569 (M + H)⁺ |
| (5,7-dimethyl- | dimethylbenzoxazol-2- | | | | |
| benzoxazol-2- | ylamino)-3-fluoro- | | | | |
| ylamino)-phenyl]-1H- | phenyl]-pyrazolo[3,4- | | | | |
| pyrazolo[3,4- | d]pyrimidin-l-yl}- | | | | |
| d]pyrimidin-4-ylamine | azetidin-1-yl)-(3,5- | | | | |
| (WO 01/19829) | dimethyl-isoxazol-4-yl)- | | | | |
| | methanone | | | | |
| 1-Azetidin-3-yl-3-[4- | (3-{4-Amino-3-[4-(5,7- | 351 | 2.83 | min (k) | 555 (M + H)⁺ |
| (5,7-dimethyl- | dimethylbenzoxazol-2- | | | | |
| benzoxazol-2- | ylamino)-3-fluoro- | | | | |
| ylamino)-phenyl]-1H- | phenyl]-pyrazolo[3,4- | | | | |
| pyrazolo[3,4- | d]pyrimidin-1-yl}- | | | | |
| d]pyrimidin-4-ylamine | azetidin-1-yl)-(5- | | | | |
| (WO 01/19829) | methyl-isoxazol-3-yl)- | | | | |
| | methanone | | | | |
| 1-Azetidin-3-yl-3-[4- | 1-(3-{4-Amino-3-[4- | 352 | 2.39 | min (k) | 488 (M + H)⁺ |
| (5,7-dimethyl- | (5,7- | | | | |
| benzoxazol-2- | dimethylbenzoxazol-2- | | | | |
| ylamino)-phenyl]-1H- | ylamino)-3-fluoro- | | | | |
| pyrazolo[3,4- | phenyl]-pyrazolo[3,4- | | | | |
| d]pyrimidin-4-ylamine | d]pyrimidin-1-yl}- | | | | |
| (WO 01/19829) | azetidin-1-yl)-ethanone | | | | |
| 1-Azetidin-3-yl-3-[4- | 1-(3-{4-Amino-3-[4- | 353 | 2.88 | min (k) | 530 (M + H)⁺ |
| (5,7-dimethyl- | (5,7- | | | | |
| benzoxazol-2- | dimethylbenzoxazol-2- | | | | |
| ylamino)-phenyl]-1H- | ylamino)-3-fluoro- | | | | |
| pyrazolo[3,4- | phenyl]-pyrazolo[3,4- | | | | |
| d]pyrimidin-4-ylamine | d]pyrimidin-1-yl}- | | | | |
| (WO 01/19829) | azetidin-1-yl)-2,2- | | | | |
| | dimethylpropan-1-one | | | | |
| 3-[4-(5,7-Dimethyl- | 1-(4-{4-Amino-3-[4- | 354 | 3.13 | min (k) | 583 (M + H)⁺ |
| benzoxazol-2- | (5,7- | | | | |
| ylamino)-phenyl]-1- | dimethylbenzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-2,2,3,3- | | | | |
| | tetrafluoropropan-1- | | | | |
| | one | | | | |
| 3-[4-(5,7-Dimethyl- | 3-[4-(5,7-Dimethyl- | 355 | 12.4 | min (a) | δ (CDCl₃, |
| benzoxazol-2- | benzoxazol-2- | | | | 400 MHz) |
| ylamino)-phenyl]-1- | ylamino)-phenyl]-1-(1- | | | | 10.85, 8.25, |
| piperidin-4-yl-1H- | methanesulfonyl- | | | | 7.91, 7.68, |
| pyrazolo[3,4- | piperidin-4-yl)-1H- | | | | 7.11, 6.79, |
| d]pyrimidin-4-ylamine | pyrazolo[3,4- | | | | 4.87, 3.71, |
| (A,L,C) | d]pyrimidin-4-ylamine | | | | 3.03, 2.94, |
| | | | | | 2.40, 2.34, |
| | | | | | 2.22, and |
| | | | | | 2.06 |
| 3-[4-(5,7-Dimethyl- | 4-{4-Amino-3-[4-(5,7- | 356 | 13.0 | min (a) | 562 (M + H)⁺ |
| benzoxazol-2- | dimethyl-benzoxazol-2- | | | | |
| ylamino)-phenyl]-1- | ylamino)-phenyl]- | | | | |
| piperidin-4-yl-1H- | pyrazolo[3,4- | | | | |
| pyrazolo[3,4- | d]pyrimidin-1-yl}- | | | | |
| d]pyrimidin-4-ylamine | piperidine-1-sulfonic | | | | |
| (A,L,C) | acid dimethylamide | | | | |
| 3-[4-(5,7-Dimethyl- | 1-(4-{4-Amino-3-[4- | 357 | 11.9 | min (a) | 495 (M − H)⁻ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)- | | | | |
| | ethanone | | | | |
| 3-[4-(5,7-Dimethyl- | 1-(4-{4-Amino-3-[4- | 358 | 13.3 | min (a) | 537 (M − H)⁻ |
| benzoxazol-2- | (5,7-dimethyl- | | | | |
| ylamino)-phenyl]-1- | benzoxazol-2- | | | | |
| piperidin-4-yl-1H- | ylamino)-phenyl]- | | | | |
| pyrazolo[3,4- | pyrazolo[3,4- | | | | |
| d]pyrimidin-4-ylamine | d]pyrimidin-1-yl}- | | | | |
| (A,L,C) | piperidin-1-yl)-2,2- | | | | |
| | dimethyl-propan-1-one | | | | |
| 3-[4-(5,7-Dimethyl- | N-{3-[4-(5,7-Dimethyl- | 359 | 14.2 | min (a) | 621 (M − H)⁻ |
| benzoxazol-2- | benzoxazol-2- | | | | |
| ylamino)-phenyl]-1- | ylamino)-phenyl]-1-[1- | | | | |
| piperidin-4-yl-1H- | (2,2-dimethyl- | | | | |
| pyrazolo[3,4- | propionyl)-piperidin-4- | | | | |
| d]pyrimidin-4-ylamine | yl]-1H-pyrazolo[3,4- | | | | |
| (A,L,C) | d]pyrimidin-4-yl}-2,2- | | | | |
| | dimethyl-propionamide | | | | |

### General Procedure X: 0-Alkylation of an Alcohol

A mixture of an alcohol (preferably 1 equivalent) and a base (for example, sodium hydride, sodium hydroxide, potassium hydroxide, or sodium, preferably potassium hydroxide) (1-10 equivalents, preferably 4 equivalents) in an organic solvent (for example, acetone, ethanol, ethyl acetate, ethylene glycol dimethyl ether, tetrahydrofuran, 1,4-dioxane, or dimethyl sulfoxide, preferably dimethyl sulfoxide) is treated with an electrophilic compound (for example, an alkyl bromide, alkyl iodide, alkyl tosylate, or an epoxide, preferably an alkyl bromide) (1-10 equivalents, preferably 3 equivalents) at about 0-120° C. (preferably about 20° C.) for about 1-48 hours (preferably 18 hours). The reaction mixture is partitioned between an aqueous solution and an organic solvent, the organic layer is separated, and the aqueous layer is further extracted with an organic solvent. The combined organic extracts are dried over a desiccant. The solvents are evaporated under reduced pressure to afford the desired product that can be further purified by crystallization or chromatography.

### Illustration of General Procedure X

### Preparation #18. cis-4-(2-Hydroxy-2-methy-propoxy)-cyclohexanol

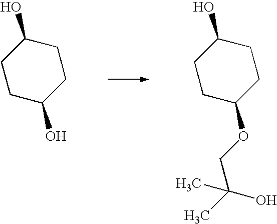

2,2-Dimethyl-oxirane (4.69 mL, 0.0526 mol) was added slowly to a mixture of cis-cyclohexane-1,4-diol (J. Org. Chem. 1962, 27, 4708-4709) (5.55 g, 0.0478 mol) and potassium hydroxide (3.22 g, 0.0573 mol) in dimethyl sulfoxide (50 mL). The reaction mixture was heated at about 50° C. for about 18 hours. The solvent was removed under reduced pressure. Water was added (100 mL), and the aqueous layer was extracted with diethyl ether (6×75 mL), then dichloromethane (3×100 mL). The combined organic layers were dried over magnesium sulfate and concentrated under reduced pressure. The residue was purified by flash chromatography on silica gel using ethyl acetate/dichloromethane (1:1) as the mobile phase to afford cis-4-(2-hydroxy-2-methy-propoxy)-cyclohexanol (3.55 g, 0.0189 mol); m/z 189 (M+H)⁺.

### General Procedure Y: 2,5-Diketopiperazine Synthesis

A mixture of a 2-halo-acetylaminoacetate (preferably 1 equivalent) and a primary amine (for example, methylamine, ethylamine, 2-propylamine) (1-10 equivalents, preferably 4 equivalents) is stirred in an organic solvent (for example, acetone, ethanol, ethyl acetate, ethylene glycol dimethyl ether, tetrahydrofuran, 1,4-dioxane, dimethyl sulfoxide, preferably tetrahydrofuran) at about 0-120° C. (preferably about 20° C.) for about 1-48 hours (preferably 18 hours). The precipitate from the reaction mixture is filtered, and washed with water. The solid is dried under reduced pressure to afford the desired product that can be further purified by crystallization or chromatography.

### Illustration of General Procedure Y

### Preparation #19. cis-1-[4-(4-Chloro-5-iodo-pyrrolo[2,3-d]pyrimidin-7-yl)-cyclohexyl]-4-methyl-piperazine-2,5-dione

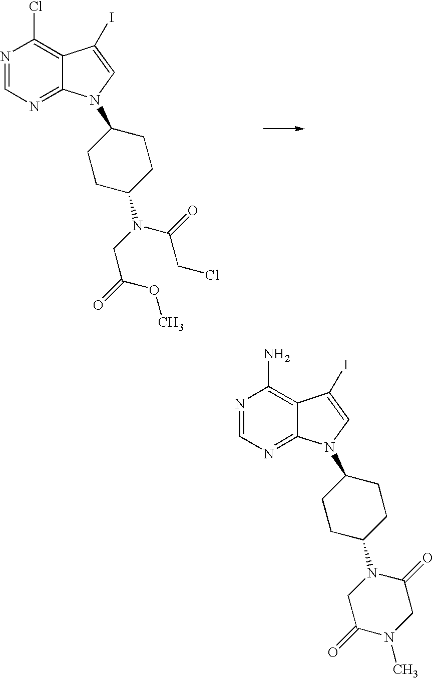

A mixture of methyl cis-{(2-chloro-acetyl)-[4-(4-chloro-5-iodo-pyrrolo[2,3-d]pyrimidin-7-yl)cyclohexyl]-amino}acetate (0.200 g, 0.00038 mol) and methylamine (2.0 M in tetrahydrofuran, 0.76 mL, 0.0015 mol) was stirred in tetrahydrofuran (8 mL) at about 20° C. for about 18 hours. The precipitate was filtered and washed with water. The solid was dried under reduced pressure to afford cis-1-[4-(4-chloro-5-iodo-pyrrolo[2,3-d]pyrimidin-7-yl)-cyclohexyl]-4-methyl-piperazine-2,5-dione (0.152 g, 0.00031 mol); RP-HPLC (30% to 95% acetonitrile/0.01M aqueous ammonium acetate over 4.5 min at 0.8 mL/min; λ=190-700 nm; Genesis C18, 120Å, 3 μm, 30×4.6 mm column; electrospray ionization method observing both positive and negative ions) Rₜ 2.13 min; m/z 488 (M+H)⁺.

### General Procedure Z: Homoketopiperazine Synthesis

A mixture of a diamine (2 equivalents) and a haloacetate (1 equivalent) in ethanol is left stirring overnight at ambient temperature and the resulting precipitate is removed by filtration. Sodium ethoxide (1 equivalent) is added to the filtrate and heated at reflux for 1-48 hours (preferably 16 hours). The mixture is allowed to cool to ambient temperature and the solvent is removed under reduced pressure to afford the product that can be further purified by crystallization or chromatography.

### Illustration of General Procedure Z

### Preparation #20. [1,4]-Diazepan-2-one

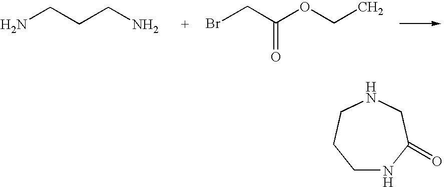

A mixture of propane-1,3-diamine (10.00 g, 0.135 mol) and bromoacetic acid ethyl ester (11.260 g, 0.067 mol) in ethanol (100 mL) was left stirring at room temperature under an atmosphere of nitrogen overnight. The precipitate was removed by filtration, and sodium ethoxide (5.200 g, 0.076 mol) was added to the resulting filtrate. The mixture was heated at reflux for about 16 hours. The mixture was allowed to cool to ambient temperature and the solvent was removed under reduced pressure to leave a black oil which was purified by flash column chromatography on silica gel using ethyl acetate, followed by ethyl acetate/methanol (70:30) as the mobile phase to give [1,4]-diazepan-2-one as a white solid (2.051 g, 0.0180 mol); ¹H NMR (DMSO-d₆, 400 MHz) δ 7.37 (s, 1H), 3.20 (s, 2H), 3.09 (t, 2H), 2.83 (t, 2H), 1.53 (m, 2H); m/z (M+H)⁺ 115.

### General procedure AA: Carbonylative Cyclization of Diamines and Aminoalcohols

Di-imidazol-1-yl-methanone (1-2 equivalents, preferably 1.5 equivalents) is added to a solution of an amino-alcohol or diamine (preferably 1 equivalent) in an organic solvent, such as tetrahydrofuran or N,N-dimethylformamide. The mixture is stirred for about 1-24 hours at about 0-50° C. The solvent is removed under reduced pressure to furnish the product which can be further purified by chromatography or crystallization.

### Illustration of General Procedure AA

### Preparation #21. 3-[4-(4-Amino-3-iodo-pyrazolo[3,4-d]pyrimidin-1-yl)-cyclohexyl]-oxazolidin-2-one

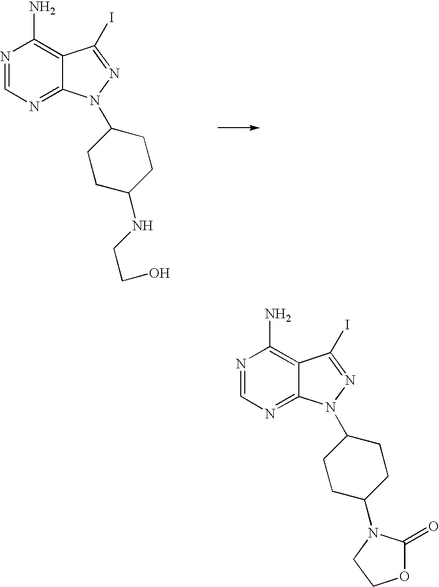

Carbonyldiimidazole (1.326 g, 8.16 mmol) was added to a solution of 2-[4-(4-amino-3-iodo-pyrazolo[3,4-d]pyrimidin-1-yl)-cyclohexylamino]ethanol (2.188 g, 5.44 mmol) in tetrahydrofuran (150 mL). The mixture was stirred for about 15 hours at ambient temperature. The solvent was removed under reduced pressure to afford the product which was purified by flash column chromatography on silica gel using dichloromethane/methanol/28% aqueous ammonia (98:1.9:0.1) as the mobile phase to afford 3-[4-(4-amino-3-iodo-pyrazolo[3,4-d]pyrimidin-1-yl)-cyclohexyl]-oxazolidin-2-one as a white solid (0.800 g, 1.87 mmol); RP-HPLC (10% to 80% acetonitrile/0.01M aqueous ammonium acetate, buffered to pH 4.5, over 6 min at 0.8 mL/min; λ=190-700 nm; Genesis C18, 120Å, 3 μm, 30×4.6 mm column, electrospray ionization method observing both positive and negative ions) Rₜ 4.42 min; m/z: (M+H)⁺ 429.

### General Procedure BB: Ketomorpholine Synthesis

To a solution of a substituted amino ethanol (1-2 equivalents, preferably 1 equivalent) in an organic solvent at ambient temperature (preferably toluene), was added chloroacetic acid methyl ester (1-2 equivalents, preferably 1 equivalent) and sodium hydride (1-2 equivalents, preferably 1.1 equivalents). The reaction mixture is stirred for about 10-30 minutes (preferably about 15 minutes) at ambient temperature, then refluxed for about 8-16 hours (preferably about 8 hours). After cooling to ambient temperature, the solvent is removed under reduced pressure. The residue is partitioned between an aqueous basic solution (for example, saturated potassium carbonate solution) and an organic solvent. The organic layer is separated and the aqueous layer is further extracted with organic solvent. The combined organic extracts are dried over desiccant and the solvent is removed under reduced pressure to afford the product that can be further purified by chromatography or crystallization.

### Illustration of General Procedure BB

### Preparation #22. 4-(1,4-Dioxa-spiro[4.5]dec-8-yl)-morpholin-3-one

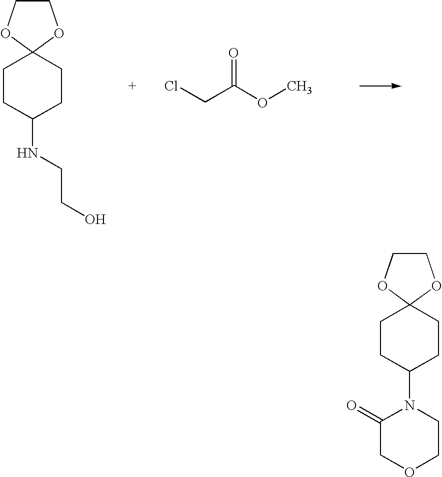

Chloroacetic acid methyl ester ( 2.8 mL, 32 mmol) and sodium hydride (60% oily dispersion, 1.4 g, 35 mmol) was added to a solution of 2-(1,4-dioxa-spiro[4.5]dec-8-ylamino)ethanol (6.42 g, 32 mmol) in toluene (100 mL), at room temperature under stirring. The reaction mixture was stirred for about 15 minutes at ambient temperature, then refluxed for about 8 hours. After cooling to ambient temperature, the toluene was removed under reduced pressure. The residue was partitioned between saturated aqueous potassium carbonate solution (40 mL) and ethyl acetate (50 mL). The organic layer was separated and the aqueous layer was further extracted with ethyl acetate (3×50 mL). The combined organic extracts were dried over magnesium sulfate and the solvent was removed under reduced pressure to yield 4-(1,4-dioxa-spiro[4.5]dec-8-yl)-morpholin-3-one (6.7 g, 27.8 mmol) as a yellow oil; ¹H NMR (CDCl₃, 400 MHz) δ 4.18, 3.95, 3.85, 3.30, 1.72.

### General Procedure CC: Deprotection of a Silyl-Protected Alcohol

A mixture of a silyl-protected alcohol and a fluoride source (for example, tetrabutyl ammonium fluoride) (10-20 equivalents, preferably about 16 equivalents) is stirred for about 24-72 hours (preferably about 48 hours) at about 25-60° C. (preferably about 40° C.). The solvent is removed under reduced pressure and the residue is partitioned between aqueous basic solution (for example, saturated sodium carbonate solution) and an organic solvent. The organic layer is separated and the aqueous layer further extracted with organic solvent. The combined organic extracts are dried over a desiccant and the solvent removed under reduced pressure. The compound can be further purified by chromatography or crystallization.

### Illustration of General Procedure CC

### Preparation #23. cis-{2-(4-Benzyloxy-cyclohexyloxy)-ethanol}

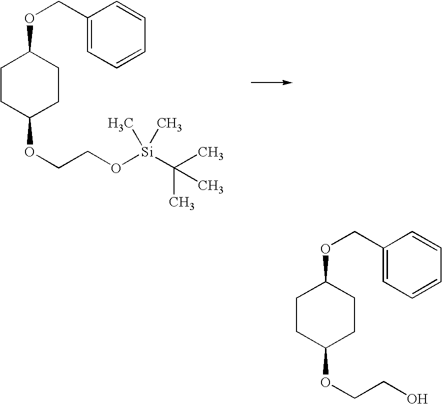

A mixture of cis-{[2-(4-benzyloxy-cyclohexyloxy)-ethoxy]-tert-butyl-dimethyl-silane} (3.49 g, 9.58 mmol), and tetrabutylammonium fluoride (1 M solution in tetrahydrofuran, 153 mL, 153 mmol) was stirred for about 48 hours at about 40° C. The solvent was removed under reduced pressure and the residue was partitioned between saturated aqueous sodium carbonate solution (40 mL) and dichloromethane (50 mL). The organic layer was separated and the aqueous layer further extracted with dichloromethane (3×50 mL). The combined organic extracts were dried over magnesium sulfate and the solvent was removed under reduced pressure to yield a yellow oil. The compound was further purified by flash chromatography on silica gel using ethyl acetatetheptane (1:1) as the mobile phase to yield cis-[2-(4-benzyloxy-cyclohexyloxy)ethanol (2.3 g, 9.2 mmol); ¹H NMR (chloroform-d₁, 400 MHz) δ 7.34, 4.52, 3.70, 3.54, 3.51, 3.40, 2.48, 1.85, 1.60; TLC (ethyl acetate/heptane 1:1) R_{f} 0.30

### General Procedure DD: Synthesis of a Trifluoromethoxy Ether

A mixture of a primary alcohol (preferably 1 equivalent), sodium hydride (1-10 equivalents, preferably 1.3 equivalents), imidazole (0.02-0.04 equivalent, preferably 0.03 equivalent) and a dry organic solvent (for example, dimethyl sulfoxide or tetrahydrofuran, preferably tetrahydrofuran) is refluxed under an atmosphere of nitrogen for about 3-5 hours (preferably about 3 hours). After cooling to ambient temperature, carbon disulfide (4-10 equivalents, preferably 5 equivalents) is added and the reaction is heated to reflux for about 30 minutes. The reaction mixture is cooled back to ambient temperature and iodomethane (2-7 equivalents, preferably 4.8 equivalents) is added. The resulting mixture is refluxed for about 30 minutes then neutralized with an acid (preferably acetic acid), washed with water, and extracted with an organic solvent. The combined organic extracts are dried over a desiccant and the solvent is removed under reduced pressure. The compound is further purified by flash chromatography to yield an alkyl dithiocarbonic acid S-methyl ester.

A polypropylene vessel was charged with 1,3-dibromo-5,5-dimethyl hydantoin (2-5 equivalents, preferably 3 equivalents) and dichloromethane. The suspension is cooled to about −78° C. and hydrogen fluoride (70% hydrogen fluoride in pyridine, 50-100 equivalents, preferably 80 equivalents) is added. The resulting suspension is stirred at about −78° C. A solution of dithiocarbonic acid S-methyl ester (1 equivalent) in dichloromethane at −78° C. is added. After the addition is complete, the acetone-dry ice bath is replaced by an ice-salt bath. The resulting red-brown reaction mixture is stirred at that temperature for about 30 minutes, then is diluted with ether (30 mL) at about 0° C., and is quenched by careful addition of an ice-cold solution of aqueous sodium hydrosulfite/sodium bicarbonate/sodium hydroxide (pH 10) until the red-brownish color disappears. The pH value is readjusted to 10 at about 0° C. by slow addition of ice-cooled sodium hydroxide (30% aqueous solution) and the resulting mixture is diluted with diethyl ether. The organic layer is separated, and the aqueous layer is extracted with diethyl ether. The combined organic phase is washed with brine, dried over desiccant, and the solvent removed under reduced pressure and further purified by chromatography or crystallization.

### Illustration of General Procedure DD

### Preparation #24. cis-{[4-(2-Trifluoromethoxy-ethoxy)-cyclohexyloxymethyl]-benzene}

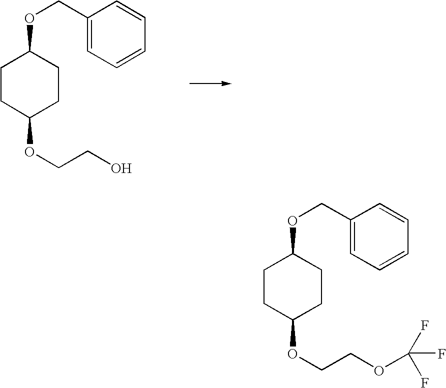

A mixture of cis-{2-{(4-benzyloxy)cyclohexyloxy)}-ethanol] (1.14 g, 4.56 mmol), sodium hydride (60% dispersion in mineral oil, 237 mg, 5.92 mmol) and imidazole (8.9 mg, 0.136 mmol) in dry tetrahydrofuran (19 mL) was heated at reflux under an atmosphere of nitrogen, for about 3 hours. After cooling to ambient temperature, carbon disulfide (1.37 mL, 22.79 mmol) was added and the mixture was heated at reflux for about 30 minutes. The reaction mixture was re-cooled to ambient temperature and iodomethane ( 1.36 mL, 21.88 mmol) was added dropwise. The resulting mixture was heated at reflux for about an additional 30 minutes. The reaction mixture was then neutralized with acetic acid, washed with water (10 mL), and extracted with dichloromethane (4×20 mL). The combined organic extracts were dried over magnesium sulfate and the solvent was removed under reduced pressure to yield an orange oil. The compound was purified by flash chromatography on silica gel using ethyl acetate/heptane (9:91) as a mobile phase to yield cis-{dithiocarbonic acid O-[2-(4-benzyloxy-cyclohexyloxy)-ethyl] ester S-methyl ester } (1.0 g, 2.94 mmol); ¹H NMR (CDCl₃, 400 MHz) δ 7.34, 4.76, 4.52, 3.80, 3.42, 2.56, 1.84, 1.59; TLC (ethyl acetate/heptane 3:7) R_{f} 0.57

A dry polypropylene round bottom tube was flushed with nitrogen and charged with 1,3-dibromo-5,5-dimethyl hydantoin ( 2.76 g, 9.46 mmol) and dichloromethane (70 mL). The suspension was cooled to −78° C. and stirred for about 10 minutes. To the mixture was slowly added hydrogen fluoride (70% hydrogen fluoride in pyridine, 6.31 mL, 252.4 mmol). The resulting suspension was stirred at about −78° C. and added dropwise to a solution of cis-{dithiocarbonic acid O-[2-(4-benzyloxy-cyclohexyloxy)-ethyl]ester S-methyl ester} ( 1.08 g, 3.16 mmol) in dichloromethane ( 10 mL) at about −78° C. via a cannula. After the addition was complete, the reaction was warmed to about −10° C. for 30 minutes, diluted with ether (30 mL) at about 0° C., then quenched by the addition of an ice-cold solution of sodium hydrosulfite/sodium bicarbonate/sodium hydroxide (pH 10), until the red-brownish color of the mixture disappeared at about 0° C. The pH value was readjusted to 10 at about 0° C. by the addition of ice-cooled sodium hydroxide (30% aqueous solution), and the mixture was diluted with ether (100 mL). The organic layer was separated, and the aqueous layer was extracted with diethyl ether (4×30 mL). The combined organic phase was washed with brine (50 mL), dried over magnesium sulfate, and the solvent removed under reduced pressure to yield a yellow oil. The compound was purified by flash chromatography on silica gel using ethyl acetate/heptane (1:20) as a mobile phase to yield cis-f[4-(2-trifluoromethoxy-ethoxy)-cyclohexyloxymethyl]-benzene} (571 mg, 1.80 mmol); ¹H NMR (CDCl₃, 400 MHz) δ 7.47, 7.24, 4.46, 4.07, 3.65, 3.45, 1.85, 1.58; TLC (ethyl acetate/heptane 1:10) R_{f} 0.21.

### General Procedure EE: Oxidation of a Sulfide to a Sulfoxide or a Sulfone

A mixture of a sulfide compound (preferably 1 equivalent), 3-chloroperoxybenzoic acid (1-5 equivalents, preferably 1 equivalent for oxidation to sulfoxide or 2 equivalents for oxidation to sulfone) and calcium carbonate (1-10 equivalents, preferably 4 equivalents) is stirred in an organic solvent (preferably dichloromethane) at ambient temperature for about 1-24 hours (preferably about 6 hours) until the reaction reaches completion. The solvent is removed under reduced pressure and the crude product can be further purified by crystallization or chromatography.

### Illustration of General Procedure EE

### Example #360

3-[4-(5,7-Dimethyl-benzoxazol-2-ylamino)-phenyl]-1-(1,1-dioxo-hexahydro-1-thiopyran-4-yl)-1H-pyrazolo[3,4-d]pyrimidin-4-ylamine

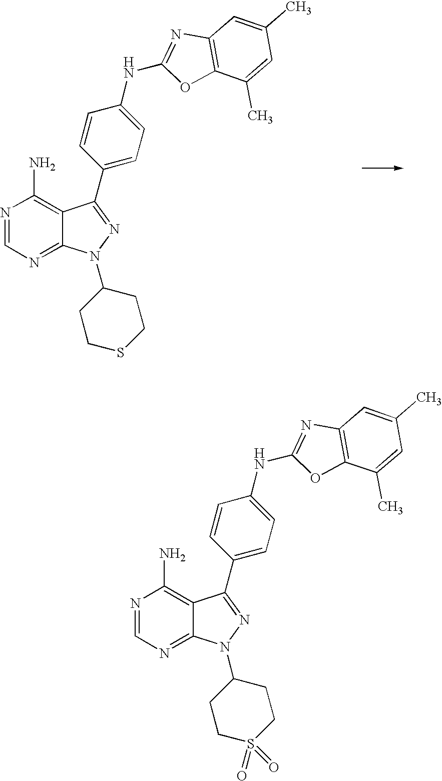

A mixture of 3-[4-(5,7-dimethyl-benzoxazol-2-ylamino)-phenyl]-1-(tetrahydro-thiopyran-4-yl)-1H-pyrazolo[3,4-d]pyrimidin-4-ylamine (0.2 g, 0.42 mmol), 3-chloroperoxybenzoic acid (0.183 g, 1.06 mmol) and calcium carbonate (0.17 g, 1.70 mmol) was stirred in dichloromethane (15 mL) at ambient temperature under an inert atmosphere for about 2 hours. Additional 3-chloroperoxybenzoic acid (0.135 g, 0.78 mmol) was added and reaction was stirred at ambient temperature for about 24 hours. The solvent was removed under reduced pressure and the crude product was purified by preparative RP-HPLC (10% to 60% acetonitrile/0.05M aqueous ammonium acetate, buffered to pH 4.5, over 25 min, then 60% to 100% acetonitrile/0.05M aqueous ammonium acetate, buffered to pH 4.5, over 5 min, at 21 mL/min; λ=254 nm; Hyperprep® HS C18, 8 μm, 250×21.2 mm column) to afford 3-[4-(5, 7-dimethyl-benzoxazol-2-ylamino)-phenyl]-1-(1,1-dioxo-hexahydro-1-thiopyran-4-yl)-1H-pyrazolo[3,4-d]pyrimidin-4-ylamine (0.080 g, 0.16 mmol) as an off-white solid; RP-HPLC (5% to 85% acetonitrile/0.05M aqueous ammonium acetate, buffered to pH 4.5, over 20 min at 1.7 mL/min; λ=254 nm; Hypersil C18, 100Å, 5 μm, 250×4.6 mm column) Rₜ 19.00 min; m/z: (M+H)⁺ 504.4.

### Example #361

trans-5-[4-(5,7-Dimethyl-benzoxazol-2-ylamino)-3-fluoro-phenyl]-7-[4-(2H-tetrazol-5-yl)-cyclohexyl]-7H-pyrrolo[2,3-d]pyrimidin-4-ylamine

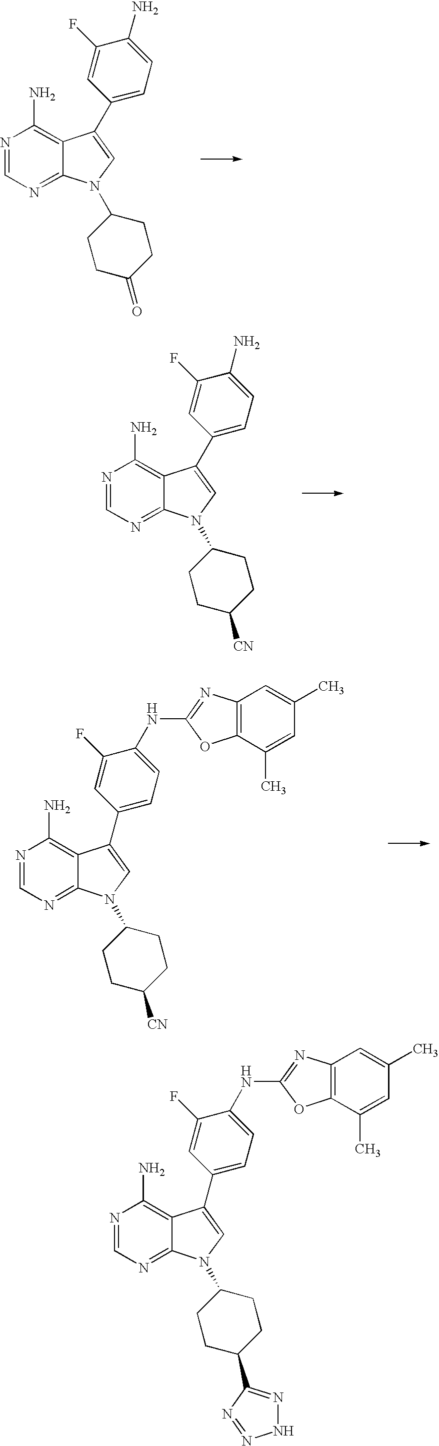

A mixture of 4-[4-amino-5-(4-amino-3-fluoro-phenyl)-pyrrolo[2,3-d]pyrimidin-7-yl]-cyclohexanone (prepared by general procedures A, B, T, C, and L) (0.200 g, 0.590 mmol) and tosylmethyl isocyanide (0.126 g, 0.645 mmol) in ethylene glycol dimethyl ether (6 mL) and tert-butyl alcohol (3 mL), cooled to about 0° C. Potassium tert-butoxide (0.148 g, 13.2 mmol) was added, and the mixture was warmed to ambient temperature and allowed to stir at that temperature for about 15 h. The mixture was diluted with water (10 mL) and the product was extracted with methanol/dichloromethane (1:9, 3×15 mL). The organic fractions were dried over magnesium sulfate and concentrated, and the product was purified by flash column chromatography on triethylamine-treated silica, using methanol/ethyl acetate (1:28) as the mobile phase to afford trans-4-[4-amino-5-(4-amino-3-fluoro-phenyl)-pyrrolo[2,3-d]pyrimidin-7-yl]-cyclohexanecarbonitrile (55 mg, 0.157 mmol) as an orange solid; RP-HP (25 to 100% acetonitrile in 0.1 M aqueous ammonium acetate over 10 min at 1 mL/min using a Hypersil HS C18, 250×4.6 mm column, λ=254 nm) Rₜ 7.67 min. Using general procedure G, the above compound was then used to form trans-4-{4-amino-5-[4-(5,7-dimethyl-benzoxazol-2-ylamino)-3-fluoro-phenyl]-pyrrolo[2,3-d]pyrimidin-7-yl}-cyclohexanecarbonitrile (0.038 g, 0.076 mmol); RP-HPLC (25% to 100% acetonitrile/0.1 M aqueous ammonium acetate, buffered to pH 4.5, over 10 min at 1.0 mL/min; λ=254 nm; Hypersil C18, 100Å, 5 μm, 250×4.6 mm column) Rₜ 11.43 min. The above compound (0.038 g, 0.076 mmol), sodium azide (30.0 mg, 0.46 mmol), and ammonium chloride (0.45 mmol) were stirred in N,N-dimethylformamide (2 mL) in a sealed tube at 115° C. for 4 days. The mixture was cooled to ambient temperature, filtered, and the product was purified by preparative HPLC (25 to 100% acetonitrile/0.1 M aqueous ammonium acetate, buffered to pH 4.5, over 20 min at 21 mL/min; λ=254 nm; Hypersil HS C18, 5 μm, 100Å, 250×21 mm column) to afford trans-5-[4-(5, 7-dimethyl-benzoxazol-2-ylamino)-3-fluoro-phenyl]-7-[4-(2H-tetrazol-5-yl)-cyclohexyl]-7H-pyrrolo[2,3-d]pyrimidin-4-ylamine (0.012 g, 0.022 mmol) as a yellow powder; RP-HPLC (25% to 100% acetonitrile/0.1 M aqueous ammonium acetate, buffered to pH 4.5, over 10 min at 1.0 mL/min; λ=254 nm; Hypersil C18, 100Å, 5 μm, 250×4.6 mm column) Rₜ 8.82 min; m/z (M+H)⁺ 539.

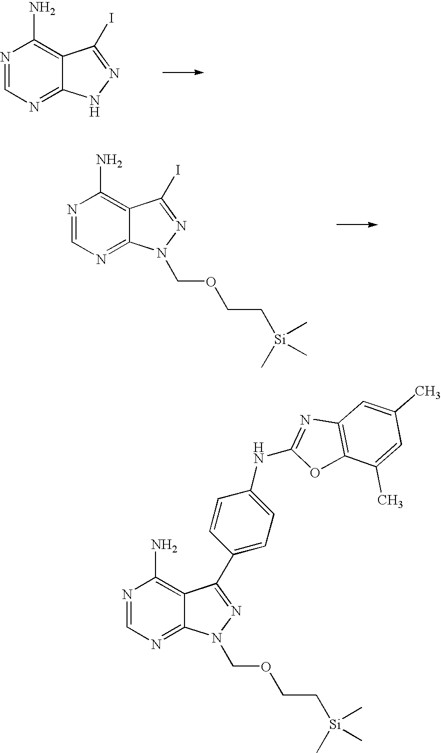

### Example #362

3-[4-(5,7-Dimethyl-benzoxazol-2-ylamino)-phenyl]-1-(2-trimethylsilanyl-ethoxymethyl)-1H-pyrazolo[3,4-d]pyrimidin-4-ylamine

A solution of 3-iodo-1H-pyrazolo[3,4-d]pyrimidin-4-ylamine (10 g, 38.31 mmol) in N,N-dimethylformamide (200 mL) and dimethyl sulfoxide (29 mL) was treated with sodium hydride (60% dispersion in mineral oil, 2.45 g, 61.29 mmol) under an inert atmosphere. After the hydrogen evolution had ceased, the reaction mixture was cooled to about 0° C. in an ice bath and {2-(chloromethoxy)ethyl}trimethylsilane (7.66 g, 45.97 mmol) was added slowly over about 30 min. The ice bath was removed and the reaction was stirred at ambient temperature for about 20 hours. The resulting mixture was poured into ice water (400 mL) and the precipitate was filtered and dried under reduced pressure to afford 3-iodo-1-(2-trimethylsilanyl-ethoxymethyl)-1H-pyrazolo[3,4-d]pyrimidin-4-ylamine (14 g, 35.78 mmol) as a white solid; m/z 392.1 (M+H)⁺.

Using general procedure C, 3-iodo-1-(2-trimethylsilanyl-ethoxymethyl)-1H-pyrazolo[3,4-d]pyrimidin-4-ylamine (1 g, 2.56 mmol) was reacted to afford 3-[4-(5,7-dimethyl-benzoxazol-2-ylamino)-phenyl]-1-(2-trimethylsilanyl-ethoxymethyl)-1H-pyrazolo[3,4-d]pyrimidin-4-ylamine (0.60 g, 1.2 mmol) as a light brown solid; RP-HPLC (5% to 95% acetonitrile/0.05M aqueous ammonium acetate, buffered to pH 4.5, over 10 min at 1.7 mumin; λ=254 mn; Hypersil C18, 100Å, 5 μm, 250×4.6 mm column) Rₜ 12.6 min; m/z (M+H)⁺ 502.

### Example #363

3-[4-(5,7-Dimethyl-benzoxazol-2-ylamino)-phenyl]-1H-pyrazolo[3,4-d]pyrimidin-4-ylamine

### Example #364

3-[4-(5,7-Dimethyl-benzoxazol-2-ylamino)-phenyl]-1-ethoxymethyl-1H-pyrazolo[3,4-d]pyrimidin-4-ylamine

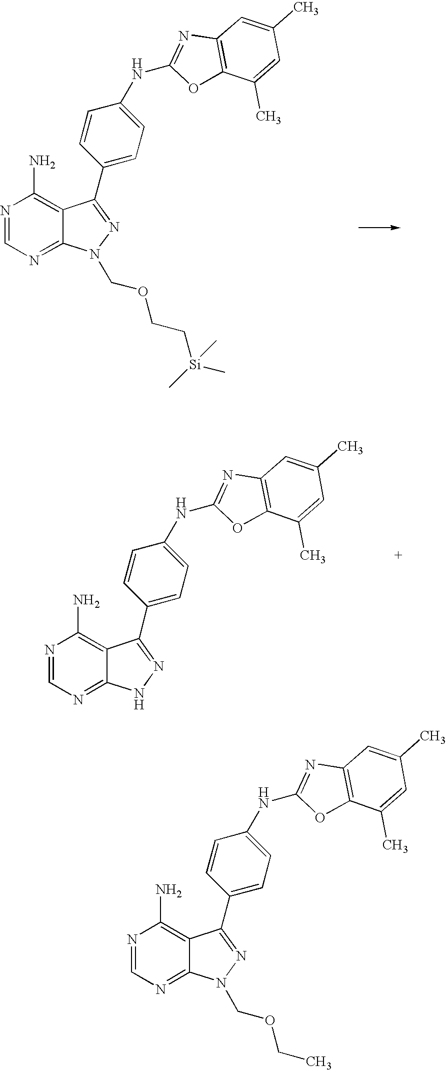

A suspension of 3-[4-(5,7-dimethyl-benzoxazol-2-ylamino)-phenyl]-1-(2-trimethylsilanyl-ethoxymethyl)-1H-pyrazolo[3,4-d]pyrimidin-4-yl amine (0.55 g, 1.1 mmol) in a mixture of aqueous hydrochloric acid (6 N, 12.5 mL) and ethanol (5 mL) was heated at about 50° C. for about 24 hours. The reaction mixture was then chilled in an ice bath and aqueous sodium hydroxide (50% w/w solution) was added dropwise to adjust the pH to 14. The resulting mixture was extracted with dichloromethane (2×20 mL). The combined organic layers were washed with a saturated brine solution (20 mL), dried over anhydrous magnesium sulfate, and concentrated under reduced pressure. The crude solid was purified by RP chromatography (10% to 60% acetonitrile/0.05M aqueous ammonium acetate, buffered to pH 4.5, over 25 min, then 60% to 100% acetonitrile/0.05M aqueous ammonium acetate, buffered to pH 4.5, over 5 min, at 21 mLjmin; λ=254 nm; Hyperprep(® HS C18, 8 μm, 250×21.2 mm column) to afford 3-[4-(5,7-dimethyl-benzoxazol-2-ylamino)-phenyl]-1H-pyrazolo[3,4-d]pyrimidin-4-ylamine (0.22 g, 0.59 mmol) as a white solid: RP-HPLC (5% to 95% acetonitrile/0.05M aqueous ammonium acetate, buffered to pH 4.5, over 10 min at 1.7 mL/min; λ=254 nm; Hypersil C18, 100 Å, 5 μm, 250×4.6 mm column) Rₜ 9.2 min; m/z (M−H)⁻370; and 3-[4-(5, 7-dimethyl-benzoxazol-2-ylamino)-phenyl]-1-ethoxymethyl-1H-pyrazolo[3,4-d]pyrimidin-4-ylamine (0.022 g, 0.05 mmol) as an off-white solid; RP-HPLC (5% to 95% acetonitrile/0.05M aqueous ammonium acetate, buffered to pH 4.5, over 10 min at 1.7 mL/min; λ=254 nm; Hypersil C18, 100Å, 5 μm, 250×4.6 mm column) Rₜ 10.9 min; m/z (M+H)⁺ 430.

### Example #365

cis-3-{4-Amino-5-[4-(5,7-dimethyl-benzoxazol-2-ylamino)-phenyl]-pyrrolo[2,3 pyrimidin-7-yl}-cyclopentanol

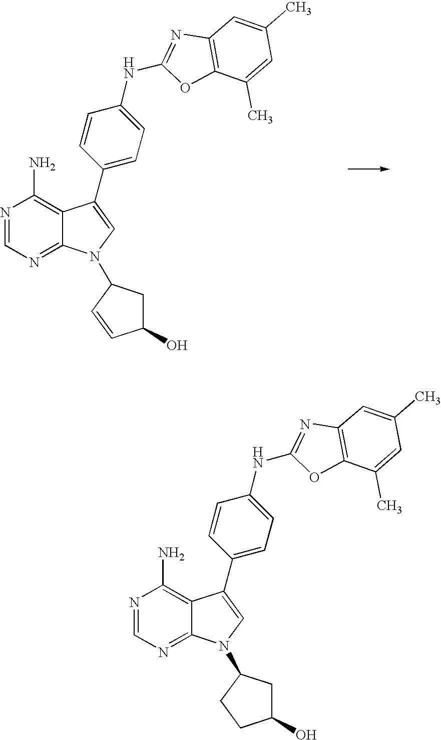

Palladium hydroxide (20 wt. % Pd) on carbon (0.080 g) was added slowly to a cold suspension of cis-4-{4-amino-5-[4-(5,7-dimethyl-benzoxazol-2-ylamino)-phenyl]-pyrrolo[2,3-d]pyrimidin-7-yl}-cyclopent-2-enol (0.1 g, 0.22 mmol) in methanol (100 mL). The mixture was stirred in a sealed tube under hydrogen pressure (50 psi) at ambient temperature for about 18 hours. The resulting mixture was filtered through Celite. The filtrate was concentrated and dried under reduced pressure to yield cis-3-{4-amino-5-[4-(5,7-dimethyl-benzoxazol-2-ylamino)-phenyl]-pyrrolo[2,3-d]pyrimidin-7-yl}-cyclopentanol (0.101 g, 0.22 mmol) as a white solid; RP-HPLC (5% to 95% acetonitrile/O.05M aqueous ammonium acetate, buffered to pH 4.5, over 10 min at 1 mumin; λ=254 nm; Hypersil C18, 100Å, 5 μm, 250×4.6 mm column) Rₜ 10.8 min; m/z (M+H)⁺ 455.

### Example #366

cis-3-{4-Amino-5-[4-(5-chloro-7-methyl-benzoxazol-2-ylamino)-phenyl]-pyrrolo[2,3-d]pyrimidin-7-yl}-cyclopentanol acetic acid salt

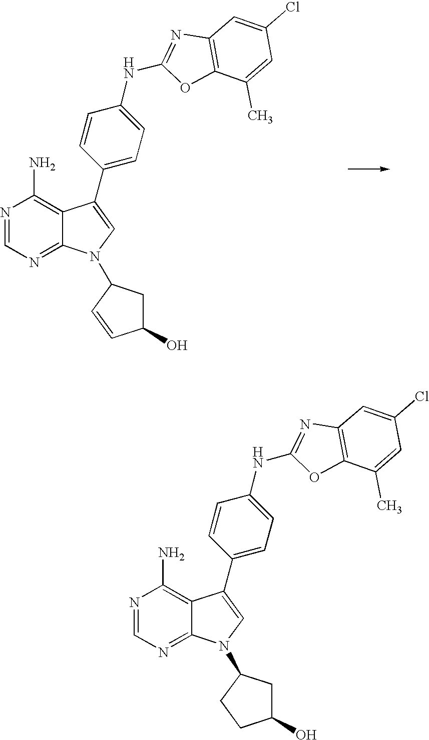

Palladium hydroxide (20 wt. % Pd) on carbon (0.080 g) was added slowly to a cold suspension of cis-4-{4-amino-5-[4-(5-chloro-7-methyl-benzoxazol-2-ylamino)-phenyl]-pyrrolo[2,3-d]pyrimidin-7-yl}-cyclopent-2-enol (0.1 g, 0.21 mmol) in methanol (100 mL). The mixture was stirred in a sealed tube under hydrogen pressure (50 psi) at ambient temperature for about 2 hours. The resulting mixture was filtered through Celite and the filtrate was concentrated under reduced pressure. The crude product was then purified via RP chromatography (10% to 60% acetonitrile/0.05M aqueous ammonium acetate, buffered to pH 4.5, over 25 min, then 60% to 100% acetonitrile/0.05M aqueous ammonium acetate, buffered to pH 4.5, over 5 min, at 21 mL/min; λ=254 nm; Hyperprep® HS C18, 100Å, 8 μm, 250×21.2 mm column) to afford cis-3-{4-amino-5-[4-(5-chloro-7-methyl-benzoxazol-2-ylamino)-phenyl]-pyrrolo[2, 3-d]pyrimidin-7-yl}-cyclopentanol monoacetate (0.049 g, 0.10 mmol) as a white solid; RP-HPLC (5% to 95% acetonitrile/0.05M aqueous ammonium acetate, buffered to pH 4.5, over 10 min at 1 mL/min; λ=254 nm; Hypersil C18, 100 Å, 5 μm, 250×4.6 mm column) Rₜ 11.2 min; m/z (M+H)⁺ 475.

### Example #367

trans-{4-{4-Amino-1-(4-morpholin-4-yl-cyclohexyl)-1H-pyrazolo[3,4-d]pyrimidin-3-yl]-phenyl}-benzothiazol-2-yl-methanol

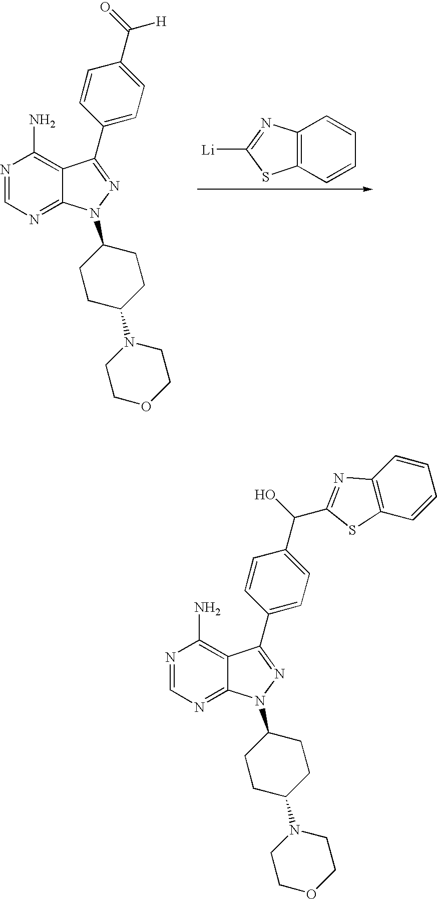

Benzothiazole (0.416 g, 3.08 mmol) and anhydrous tetrahydrofuran (15 mL) were loaded into a reaction vessel equipped with a magnetic stirring bar. The flask was flushed with nitrogen and the mixture was cooled to about −78° C. prior to the addition of n-butyl lithium (1.95 M in hexanes, 1.58 ml, 3.09 mmol). The reaction was stirred at about −78° C. for about 3 hours. Next, 4-[4-amino-1-(4-morpholin-4-yl-cyclohexyl)-1H-pyrazolo[3,4-d]pyrimidin-3-yl]-benzaldehyde (prepared from 3-iodo-1H-pyrazolo[3,4-d]pyrimidin-4-ylamine using general procedures A, T, J, and C) (0.50 g, 1.23 mmol) was added. The reaction was warmed to ambient temperature and stirred for about 16 hours. The reaction mixture was then quenched by addition of saturated aqueous ammonium chloride (40 mL). The tetrahydrofuran was removed under reduced pressure and the aqueous mixture was extracted with ethyl acetate (3×15 mL). The combined fractions were dried over anhydrous magnesium sulfate and concentrated under reduced pressure. The crude product was purified via RP-HPLC (10% to 80% acetonitrile/0.05 M aqueous ammonium acetate, buffered to pH 4.5, over 25 min at 21 mllmin; λ=254 nm; Hyperprep® C18, 100 Å, 8 μm, 250×21.2 mm) to give trans-{4-[4-amino-1-(4-morpholin-4-yl-cyclohexyl)-1H-pyrazolo[3,4-d]pyrimidin-3-yl]-phenyl}-benzothiazol-2-yl-methanol as a white solid (0.057 g, 0.105 mmol); RP-HPLC (5% to 85% acetonitrile/0.05 M ammonium acetate, buffered to pH 4.5, over 20 min at 1.0 mL/min, λ=254 nm; Hypersil C18, 5 μm, 100Å, 250×4.6 mm) Rₜ 14.06 min; m/z: (M+H)⁺ 542.

### Example #368

trans-{4-[4-Amino-1-(4-morpholin-4-yl-cyclohexyl)-1H-pyrazolo[3,4-d]pyrimidin-3-yl]-phenyl}-benzothiazol-2-yl-methanone

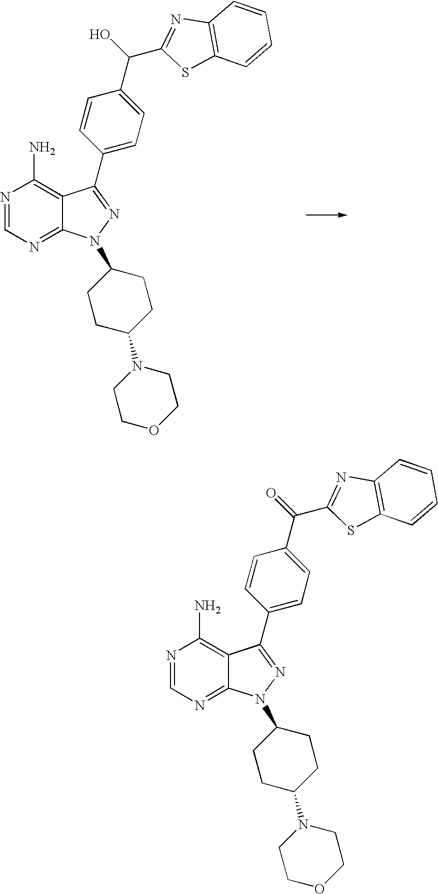

trans-{4-[4-Amino-1-(4-morpholin-4-yl-cyclohexyl)-1H-pyrazolo[3,4-d]pyrimidin-3-yl]-phenyl}-benzothiazol-2-yl-methanol (example #367) (0.050 g, 0.090 mmol), manganese dioxide (0.040 g, 0.460 mmol), and methanol (15 mL) were loaded into a reaction flask. The mixture was stirred under a nitrogen atmosphere at ambient temperature for about 48 hours. The crude mixture was filtered though a pad of Celite and washed with methanol (3×5 mL). The filtrate was concentrated under reduced pressure and the crude product purified via RP-HPLC (10%-80% acetonitrile/0.05 M ammonium acetate over 25 min at 21 mL/min; λ=254 nm; Hyperprep(® C18, 100Å, 8 μm, 250×21.2 mm) to give trans-(4-[4-amino-1-(4-morpholin-4-yl-cyclohexyl)-1H-pyrazolo[3,4-d]pyrimidin-3-yl]-phenyl}-benzothiazol-2-yl-methanone as a white solid (0.037 g, 0.0686 mmol); RP-HPLC (5% to 95% acetonitrile/0.05 M aqueous ammonium acetate, buffered to pH 4.5, over 10 min at 1.0 mL/min; λ=254 nm; Hypersil C18, 5 μm, 100Å, 250×4.6 mm column) Rₜ 9.66 min; m/z: (M+H)⁺ 540.

### Example #369

[4-(4-Amino-1-cyclopentyl-1H-pyrazolo[3,4-d]pyrimidin-3-yl)-phenyl]-benzothiazol-2-yl-methanol

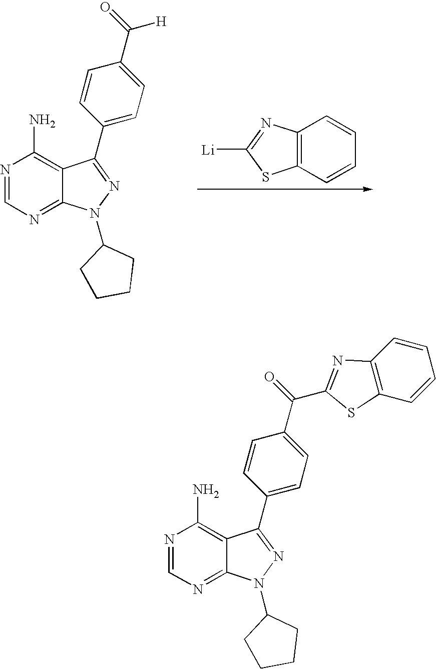

Benzothiazole (0.358 g, 2.65 mmol) and anhydrous tetrahydrofuran (10 mL) were loaded into a reaction vessel equipped with a magnetic stirring bar. The flask was flushed with nitrogen and the mixture was cooled to about −78° C. prior to the addition of n-butyl lithium (1.95 M in hexanes, 1.36 ml, 2.66 mmol). The reaction was stirred at about −78° C. for about 3 hours. Next, 4-(4-amino-1-cyclopentyl-1H-pyrazolo[3,4-d]pyrimidin-3-yl)-benzaldehyde (prepared from 3-iodo-1H-pyrazolo[3,4-d]pyrimidin-4-ylamine via general procedures A and C) (0.325 g, 1.06 mmol) was added. The reaction was allowed to warm to ambient temperature and stirred for about 16 hours. The reaction mixture was then quenched by addition of saturated aqueous ammonium chloride (40 mL). The tetrahydrofuran was removed under reduced pressure and the aqueous mixture was extracted with ethyl acetate (3×10 mL). The combined organic fractions were dried over anhydrous magnesium sulfate. The ethyl acetate was removed under reduced pressure and the crude product was purified via RP-HPLC (10% to 80% acetonitrile/0.05 M aqueous ammonium acetate, buffered to pH 4.5, over 25 min at 21 mL/min; λ=254 nm; Hyperprep® C18, 100Å, 8 μm, 250×21.2 mm column) to give [4-(4-amino-1-cyclopentyl-1H-pyrazolo[3,4-d]pyrimidin-3-yl)-phenyl]-benzothiazol-2-yl-methanol as a white solid (0.058 g, 0.131 mmol); RP-HPLC (5% to 95% acetonitrile/0.05 M aqueous ammonium acetate, buffered to pH 4.5, over 10 min at 1.0 mL/min; 15 min total run time; λ=254 nm; Hypersil C18, 5 μm, 100Å, 250×4.6 mm column) Rₜ 12.84 min; m/z: (M+H)⁺ 443.

### Example #370

[4-(4-Amino-1-cyclopentyl-1H-pyrazolo[3,4-d]pyrimidin-3-yl)-phenyl]-benzothiazol-2-yl-methanone

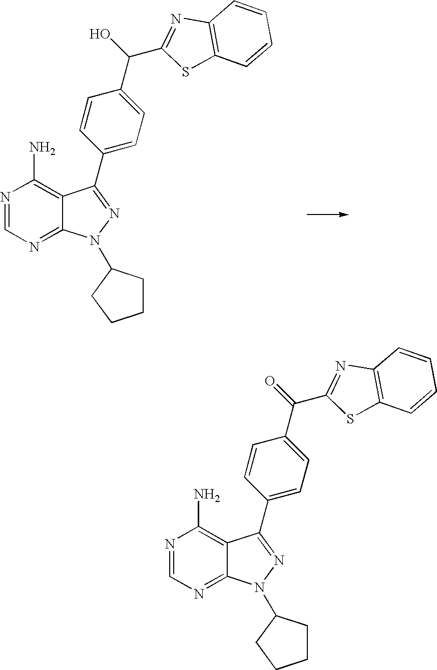

{4-[4-Amino-1-cyclopentyl-1H-pyrazolo[3,4-d]pyrimidin-3-yl]-phenyl}-benzothiazol-2-ylmethanol (example #369) (0.040 g, 0.090 mmol), manganese dioxide (0.0393 g, 0.452 mmol), and methanol (10 mL) were loaded into a reaction flask. The mixture was stirred under a nitrogen atmosphere at ambient temperature for about 48 hours. The crude mixture was filtered though a pad of Celite and washed with methanol (3×10 mL). The filtrate was concentrated under reduced pressure and the crude product purified via RP-HPLC (10% to 80% acetonitrile/0.05 M aqueous ammonium acetate, buffered to pH 4.5, over 25 min at 21 mL/min; λ=254 nm; Hyperprep® C18, 100Å, 8 μm, 250×21.2 mm column) to give [4-(4-amino-1-cyclopentyl-1H-pyrazolo[3,4-d]pyrimidin-3-yl)-phenyl]-benzothiazol-2-yl-methanone as a white solid (0.0086 g, 0.0195 mmol); RP-HPLC (5% to 85% acetonitfile/0.05 M ammonium acetate, buffered to pH 4.5, over 20 min at 1.0 mL/min; 30min total run time; λ=254 nm; Hypersil C18, 5 μm, 100Å, 250×4.6 mm column) Rₜ 27.96 min; m/z: (M+H)⁺ 441.

### Preparation #25. Toluene-4-sulfonic acid 2-cyclopropoxy-ethyl ester.

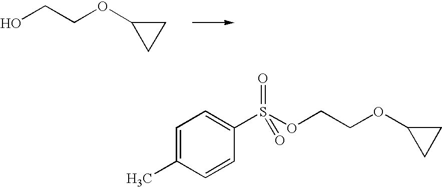

A solution of 2-cyclopropoxy-ethanol (0.102 g, 0.0010 mol) and triethylamine (0.153 mL, 0.0011 mol) in dichloromethane (2 mL) was cooled to about 0° C., and was treated slowly with a solution of 4-methyl-benzenesulfonyl chloride (0.228 g, 0.0012 mol) in dichloromethane (3 mL). The reaction mixture was stirred at about 0° C. for 1 hour, then at ambient temperature for about 18 hours. The reaction mixture was quenched with saturated sodium bicarbonate solution (10 mL), and was extracted with dichloromethane (3×30 mL). The combined organic layers were dried over magnesium sulfate, and the solvent was removed under reduced pressure. The residue was purified by flash column chromatography on silica gel using ethyl acetate/heptane (1:7) to afford toluene-4-sulfonic acid 2-cyclopropoxy-ethyl ester (0.129 g, 0.00050 mol) as a light yellow oil; RP-HPLC (30% to 95% acetonitrile/0.01M aqueous ammonium acetate, buffered to pH 4.5, over 4.5 min at 0.8 mL/min; λ=190-700 nm; Genesis C18, 120 A, 3 pm, 30 x 4.6 mm column; electrospray ionization method observing both positive and negative ions) Rₜ 3.02 min.

### Preparation #26. 3-Bromo-1-tert-butyl-1H-pyrazolo[3,4-d]pyrimidin-4-ylamine

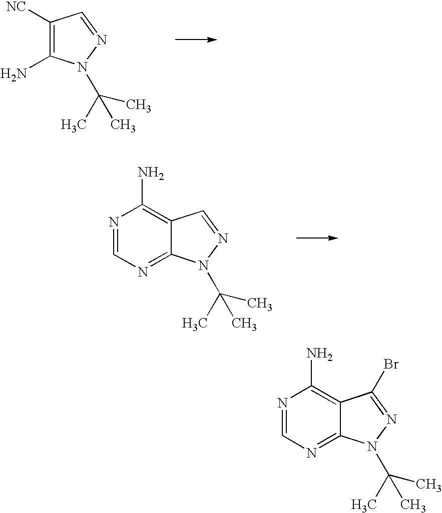

A suspension of 5-amino-1-tert-butyl-1H-pyrazole-4-carbonitrile (8.67 g, 0.0528 mol) in formamide (100 mL) was heated at 180° C. for about 4 hours. The reaction mixture was cooled, poured into ice water (200 mL), and the product was extracted with ethyl acetate (4×80 mL). The combined organic layers were dried over magnesium sulfate, and the solvent was removed under reduced pressure. The residue was taken up in ether (35 mL) and the precipitate was filtered, washed with ether (50 mL), and dried under reduced pressure to afford 1-tert-butyl-1H-pyrazolo[3,4-d]pyrimidin-4-ylamine (3.22 g, 0.0169 mol) as a white solid; RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 3 μm, 30×4.6 mm column) Rₜ 1.38 min.

A mixture of 1-tert-butyl-1H-pyrazolo[3,4-d]pyrimidin-4-ylamine (0.200 g, 0.0010 mol) and bromine (0.134 mL, 0.0026 mol) in H₂O (10 mL) was heated at about 90° C. for about 24 hours. It was neutralized with aqueous sodium hydroxide (2.0 N). The resulting white precipitate was filtered, washed with water, and dried to afford 3-bromo-1-tert-butyl-1H-pyrazolo[3,4-d]pyrimidin-4-ylamine (0.177 g, 0.66 mmol) as a white solid; RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 3 μm, 30×4.6 mm column) Rₜ 2.05 min.

### Example #371

trans-3-[3-[4-(5-Chloro-7-methyl-benzoxazol-2-ylamino)-phenyl]-1-(4-morpholin-4-yl-cyclohexyl)-1H-pyrazolo[3,4-d]pyrimidin-4-ylamino]-propionamide acetic acid salt

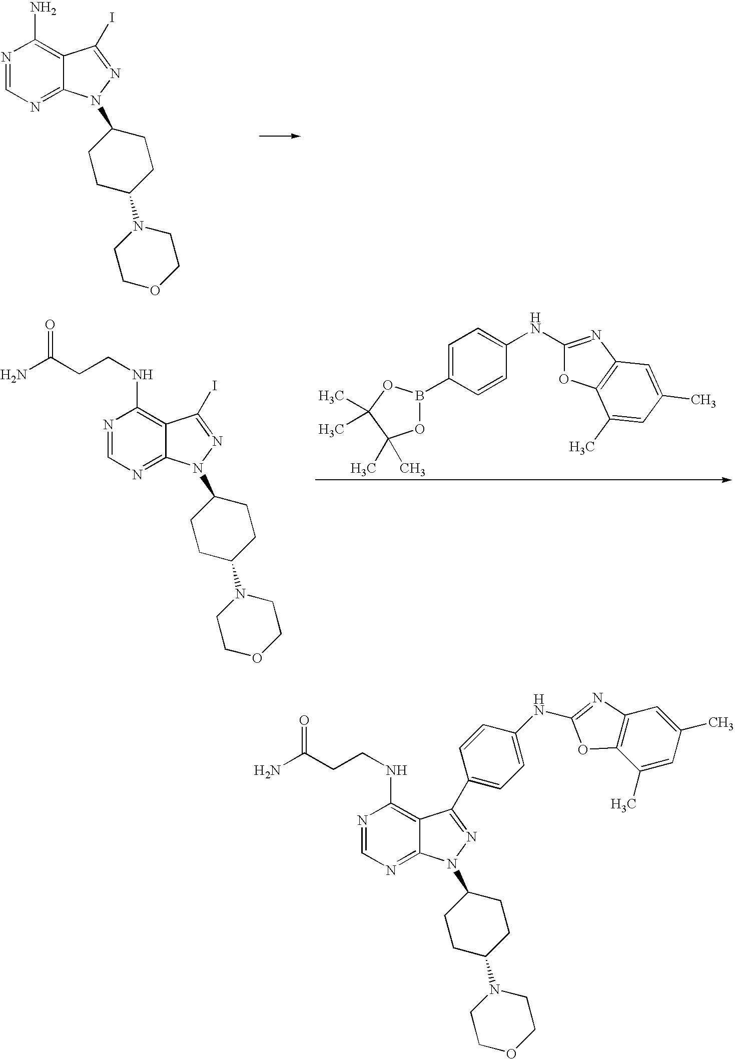

trans-3-Iodo-1-(4-morpholin-4-yl-cyclohexyl)-1H-pyrazolo[3,4-d]pyrimidin-4-ylamine (prepared using general procedures A, T and J) (0.1 g, 0.000234 mol), cesium carbonate (0.299 g, 0.000702 mol) and 3-chloropropionamide (0.025 g, 0.000234 mol)were dissolved in N,N-dimethylformamide (5 mL). The reaction mixture was stirred for about 40 hours, the insoluble residue was removed by filtration, and the filtrate was concentrated, then was purified by preparative RP-HPLC (10% to 60% acetonitrile/0.05M aqueous ammonium acetate, buffered to pH 4.5, over 25 min at 21 mL/min; λ=254 nm; Hypersil C18, 100 Å, 8 μm, 250×21.2 mm column) to yield trans-3-[3-iodo-1-(4-morpholin-4-yl-cyclohexyl)-1H-pyrazolo[3,4-d]pyrimidin-4-ylamino]-propionamide (0.075 g, 0.000151 mol) as a white solid; m/z (M+H)⁺ 500.

trans-3-[3-Iodo-1-(4-morpholin-4-yl-cyclohexyl)-1H-pyrazolo[3,4-d]pyrimidin-4-ylamino]-propionamide (0.075 g, 0.00015 mol) was coupled with (5-chloro-7-methyl-benzoxazol-2-yl)-[4-(4,4,5,5-tetramethyl-[1,3,2]dioxaborolan-2-yl)-phenyl]-amine (0.075 g, 0.000195 mol) (prepared using general procedures G and D) using general procedure C to yield trans-3-[3-[4-(5-chloro-7-methyl-benzoxazol-2-ylamino)-phenyl]-1-(4-morpholin-4-yl-cyclohexyl)-1H-pyrazolo[3,4-d]pyrimidin-4-ylamino]-propionamide monoacetate (0.020 g, 0.0000289 mol) as a white solid: ¹H NMR (DMSO-d₆, 400 MHz); δ 7.91, 7.60, 7.08, 6.87, 6.50, 4.64, 3.68, 3.54, 2.40, 2.08, 1.88, 1.44;

m/z (M−H)⁻629.

### Example #372

trans-4-{4-Amino-3-[4-(5,7-dimethyl-benzoxazol-2-ylamino)-phenyl]-pyrazolo[3,4-d]pyrimidin-1-yl}-1-methyl-cyclohexanol

### Example #373

cis-4-{4-Amino-3-[4-(5,7-dimethyl-benzoxazol-2-ylamino)-phenyl]-pyrazolo[3,4-d]pyrimidin-1-yl}-1-methyl-cyclohexanol

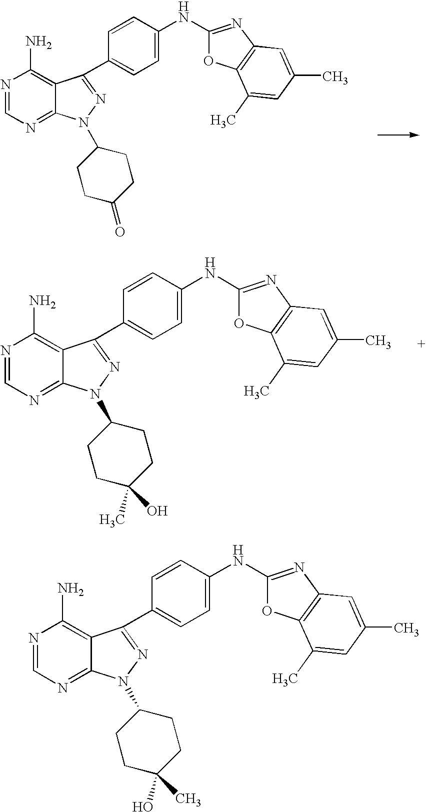

4-{4-Amino-3-[4-(5,7-dimethyl-benzoxazol-2-ylamino)-phenyl]-pyrazolo[3,4-d]pyrimidin-1-yl}-cyclohexanone (prepared using general procedures A, T, and C (G, D)) (5.00 g, 0.0107 mol) and zinc bromide (0.50 g, 0.0022 mol) were suspended in toluene (100 mL) and stirred at ambient temperature for about 10 minutes under continuous nitrogen flow. A solution of trimethylaluminum in toluene (2 M, 13.38 mL, 0.0267 mol) was added and the stirring was continued for about 2 hours. The addition of trimethylaluminum solution (2 M, 13.38 mL) was repeated four more times and the reaction mixture was quenched by a dropwise addition of saturated aqueous solution of ammonium chloride (100 mL). The resulting mixture was evaporated to dryness under reduced pressure; the residue was suspended in N,N-dimethylformamide (200 mL) and filtered through a Celite pad. The filtrate was concentrated and the residue was purified by preparative RP-HPLC (20% to 80% acetonitrile/0.05M aqueous ammonium acetate, buffered to pH 4.5, over 30 min at 21 mL/min; λ=254 nm; Hypersil C18, 100 Å, 8 μm, 250×21.2 mm column) to yield trans-4-{4-amino-3-[4-(5,7-dimethyl-benzoxazol-2-ylamino)-phenyl]-pyrazolo[3,4-d]pyrimidin-1-yl]-1-methyl-cyclohexanol (0.052 g, 0.107 mmol) as a white solid: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 19.62 min; ¹H NMR (DMSO-d₆, 400 MHz); δ 10.85, 8.23, 7.93, 7.67, 7.11, 6.79, 4.64, 4.46, 2.89, 2.73, 2.08, 1.91, 1.44; and cis-4-{4-amino-3-[4-(5,7-dimethyl-benzoxazol-2-ylamino)-phenyl]-pyrazolo[3,4-d]pyrimidin-1-yl}-1-methyl-cyclohexanol (0.104 g, 0.206 mmol) as a white solid; RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 20.33 min; ¹H NMR (DMSO-d₆, 400 MHz); δ 10.86, 8.23, 7.93, 7.67, 7.11, 6.79, 4.68, 4.20, 2.89, 2.73, 2.14, 1.83.

### Example #374

4-{4-Amino-3-[4-(5,7-dimethyl-benzoxazol-2-ylamino)-phenyl]-pyrazolo[3,4-d]pyrimidin-1-yl}-piperidine-1-carboxylic acid isopropyl ester

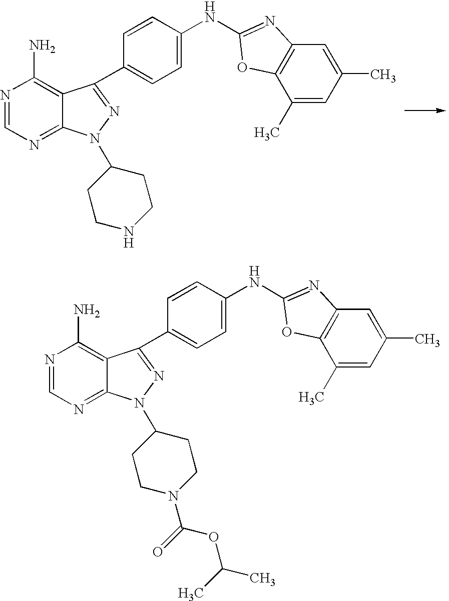

Triethylamine (0.1 mL, 0.72 mmol) was added to a suspension of 3-[4-(5,7-dimethyl-benzoxazol-2-ylamino)-phenyl]-1-piperidin-4-yl-1H-pyrazolo[3,4-d]pyrimidin-4-ylamine (prepared using general procedures A and C (G, D)) (0.109 g, 0.24 mmol) in dichloromethane (5 mL) and the resulting mixture was cooled to 0° C. while stirring under continuous nitrogen flow. A solution of isopropyl chloroformate in toluene (1 M, 0.24 mL, 0.00024 mol) was added dropwise and the reaction mixture was stirred at about 0° C. for about 1 hour. The solvents were removed under reduced pressure and the residue was purified by preparative RP-HPLC (20% to 90% acetonitrile/0.05M aqueous ammonium acetate, buffered to pH 4.5, over 30 min at 21 mL/min; λ=254 nm; Hypersil C18, 100 Å, 8 μm, 250×21.2 mm column) to yield 4-{4-amino-3-[4-(5,7-dimethyl-benzoxazol-2-ylamino)-phenyl]-pyrazolo[3,4-d]pyrimidin-1-yl}-piperidine-1-carboxylic acid isopropyl ester (0.080 g, 0.148 mmol) as a white solid: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 15.55 min; ¹H NMR (DMSO-d₆, 400 MHz); δ 10.86, 8.24, 7.93, 7.67, 7.11, 6.79, 4.94, 4.78, 4.12, 3.00, 2.41, 2.34, 2.01, 1.17.

### Example #375

4-{4-Amino-3-[4-(5,7-dimethyl-benzoxazol-2-ylamino)-phenyl]-pyrazolo[3,4-d]pyrimidin-1-yl}-piperidine-1-carboxylic acid methyl ester

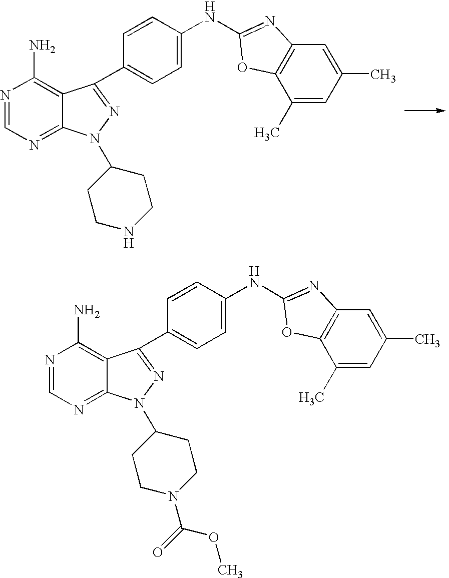

Triethylamine (0.1 mL, 0.72 mmol) was added to a suspension of 3-[4-(5,7-dimethyl-benzoxazol-2-ylamino)-phenyl]-1-piperidin-4-yl-1H-pyrazolo[3,4-d]pyrimidin-4-ylamine (prepared using general procedures A and C (G, D)) (0.110 g, 0.24 mmol) in dichloromethane (5 mL) and the resulting mixture was cooled to about 0° C. while stirring under continuous nitrogen flow. Methyl chloroformate (0.020 mL, 0.254 mmol) was added dropwise and the reaction mixture was stirred at about 0° C. for about 1 hour. The solvents were removed under reduced pressure and the residue was purified by preparative RP-HPLC (20% to 90% acetonitrile/0.05M aqueous ammonium acetate, buffered to pH 4.5, over 30 min at 21 ml/min; λ=254 nm; Hypersil C18, 100 Å, 8 μm, 250×21.2 mm column) to yield 4-{4-amino-3-[4-(5,7-dimethyl-benzoxazol-2-ylamino)-phenyl]-pyrazolo[3,4-d]pyrimidin-1-yl}-piperidine-1-carboxylic acid methyl ester (0.077 g, 0.15 mmol) as a white solid: RP-HPLC (30% to 95% acetonitrile/0.01M aqueous ammonium acetate, buffered to pH 4.5, over 4.5 min at 0.8 mL/min; λ=190-700 nm; Genesis C18, 120 Å, 3 μm, 30×4.6 mm column) Rₜ 2.77 min; m/z (M+H)⁺ 513.

### Example #376

trans-4-{4-Amino-3-[4-(5,7-dimethyl-benzoxazol-2-ylamino)-3-fluoro-phenyl]-pyrazolo[3,4-d]pyrimidin-1-yl}-cyclohexyl N,N-dimethyl carbamate

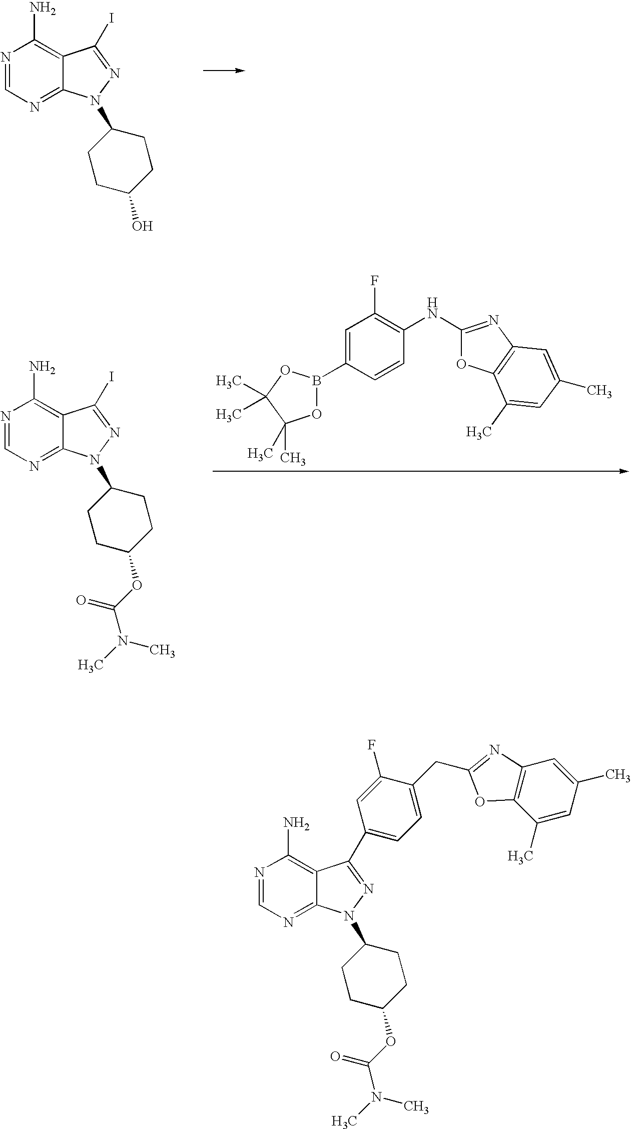

N,N-dimethylcarbamoyl chloride (0.63 g, 0.00585 mol) was added to a mixture of trans-4-(4-Amino-3-iodo-pyrazolo[3,4-d]pyrimidin-1-yl)-cyclohexanol (prepared using general procedures A, T and U) (0.20 g, 0.000557 mol) in N-methylpyrrolidinone (0.9 mL) and pyridine (0.1 mL). The reaction mixture was heated at about 75° C. for about 24 hours under a continuous flow of nitrogen. Additional N,N-dimethylcarbamoyl chloride (0.63 g, 0.00585 mol) was added and the reaction was stirred at about 75° C. for about an additional 24 hours. The reaction mixture was cooled to ambient temperature and purified by preparative RP-HPLC (10% to 60% acetonitrile/0.05M aqueous ammonium acetate, buffered to pH 4.5, over 25 min at 21 mL/min; ) λ=254 nm; Hypersil C18, 100 Å, 8 μm, 250×21.2 mm column) to yield trans-4-(4-amino-3-iodo-pyrazolo[3,4-d]pyrimidin-1-yl)-cyclohexyl N,N-dimethyl carbamate (0.019 g, 0.0445 mmol) as an off-white solid:

m/z(M+H)⁺ 431.

trans-4-(4-Amino-3-iodo-pyrazolo[3,4-d]pyrimidin-1-yl)-cyclohexyl N,N-dimethyl carbamate (0.06 g, 0.00014 mol) was reacted with (5,7-dimethyl-benzoxazol-2-yl)-[2-fluoro-4-(4,4,5,5-tetramethyl-[1,3,2]dioxaborolan-2-yl)-phenyl]-amine (0.07 g, 0.000182 mol) (prepared using general procedures G and D) using general procedure C to afford trans-4-{4-amino-3-[4-(5,7-dimethyl-benzoxazol-2-ylamino)-3-fluoro-phenyl]-pyrazolo[3,4-d]pyrimidin-1-yl}-cyclohexyl N,N-dimethyl carbamate (0.034 g, 0.000061 mol) as a white solid: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 16.44 min; ¹H NMR (DMSO-d₆, 400 MHz); δ 10.58, 8.50, 8.25, 7.52, 7.10, 6.80, 4.75, 4.58, 2.85, 2.41, 2.31, 2.01, 1.61.

### Example #377

trans-3-(4-{4-Amino-3-[4-(5,7-dimethyl-benzoxazol-2-ylamino)-phenyl]-pyrazolo[3,4-d]pyrimidin-1-yl}-cyclohexyl)-4H-[1,2,4]oxadiazol-5-one

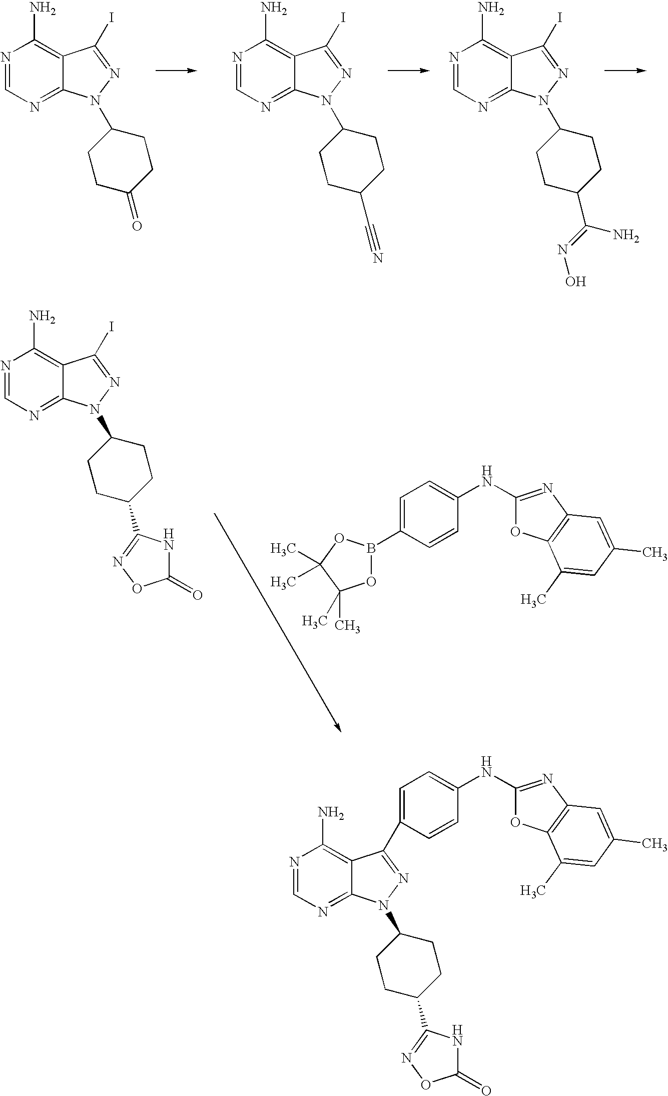

4-(4-Amino-3-iodo-pyrazolo[3,4-d]pyrimidin-1-yl)-cyclohexanone (prepared using general procedures A and T) (4.00 g, 0.0112 mol) was added to a mixture of ethylene glycol dimethyl ether (60 mL) and ethanol (2 mL). 1-Isocyano-methanesulfonyl-4-methyl-benzene (2.19 g, 0.0112 mol) was added and the resulting mixture was cooled to about 0° C. while stirring under continuous nitrogen flow. Potassium tert-butoxide (2.51 g, 0.0224) was added and the reaction mixture was stirred for about 16 hours while slowly warming to ambient temperature. The precipitate was filtered and the filtrate was concentrated under reduced pressure. The residue was subjected to flash chromatography on silica gel using dichloromethane/methanol/triethylamine (98:1:1) as the mobile phase to yield 4-(4-amino-3-iodo-pyrazolo[3,4-d]pyrimidin-1-yl)-cyclohexanecarbonitrile (1.9 g, 0.00516 mol) as an off-white solid as a mixture of cis- and trans-isomers: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 12.16 min. and 12.53 min.

The mixture of cis- and trans-4-(4-amino-3-iodo-pyrazolo[3,4-d]pyrimidin-1-yl)-cyclohexanecarbonitrile (1.5 g, 0.00408 mol), hydroxylamine hydrochloride (1.42 g, 0.0204 mol) and triethylamine (3.6 mL, 0.0204 mol) was heated in dimethyl sulfoxide (10 mL) at about 75° C. under a continuous flow of nitrogen for about 16 hours. The reaction mixture was poured into ice-cold water (120 mL) and the precipitate was collected by filtration, washed with water and dried to yield 4-(4-amino-3-iodo-pyrazolo[3,4-d]pyrimidin-1-yl)-N-hydroxy-cyclohexanecarboxamidine (1.20 g, 0.003 mol) as a yellow solid as a mixture of cis- and trans-isomers: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 9.00 min and 9.09 min.

The mixture of cis- and trans-4-(4-amino-3-iodo-pyrazolo[3,4-d]pyrimidin-1-yl)-N-hydroxy-cyclohexanecarboxamidine (0.215 g, 0.000536 mol) and pyridine (0.048 mL, 0.00059 mol) in N,N-dimethylformamide (5 mL) was cooled to about 0° C. while stirring under a continuous flow of nitrogen and 2-ethylhexyl chloroformate (0.105 mL, 0.000536 mol) was added dropwise. The stirring at about 0° C. was continued for about an additional 40 minutes and the reaction mixture was poured into ice-cold water (20 mL). The precipitate was collected by filtration and dried. It was triturated in xylenes and the suspension was heated at reflux for about 2 hours under a continuous flow of nitrogen. The solvent was removed under reduced pressure and the yellow residue was purified by preparative RP-HPLC (10% to 50% acetonitrile/0.05M aqueous ammonium acetate, buffered to pH 4.5, over 20 min at 21 mL/min; λ=254 nm; Hypersil C18, 100 Å, 8 μm, 250×21.2 mm column) to yield trans-3-[4-(4-amino-3-iodo-pyrazolo[3,4-d]pyrimidin-1-yl)-cyclohexyl]-4H-[1,2,4]oxadiazol-5-one (0.018 g, 0.0423 mmol) as a white solid: RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 11.27 min.

trans-3-[4-(4-Amino-3-iodo-pyrazolo[3,4-d]pyrimidin-1-yl)-cyclohexyl]-4H-[1,2,4]oxadiazol-5-one (0.032 g, 0.000075 mol) was reacted with (5,7-dimethyl-benzoxazol-2-yl)-[4-(4,4,5,5-tetramethyl-[1,3,2]dioxaborolan-2-yl)-phenyl]-amine (0.032 g, 0.00009 mol) (prepared using general procedures G and D) using the general procedure C to afford trans-3-(4-{4-amino-3-[4-(5,7-dimethyl-benzoxazol-2-ylamino)-phenyl]-pyrazolo[3,4-d]pyrimidin-1-yl}-cyclohexyl)-4H-[1,2,4]oxadiazol-5-one (0.021 g, 0.0000384 mol) as an off-white solid; RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 16.63 min; ¹H NMR (DMSO-d₆, 400 MHz); δ 10.86, 8.24, 7.95, 7.67, 7.11, 6.80, 4.71, 2.89, 2.73, 2.41, 2.31, 2.01.

### Example #378

trans-(4-{4-Amino-3-[4-(5,7-dimethyl-benzoxazol-2-ylamino)-phenyl]-pyrazolo[3,4-d]pyrimidin-1-yl}-cyclohexyloxy)-acetic acid

### Example #379

trans-2-(4-{4-Amino-3-[4-(5,7-dimethyl-benzoxazol-2-ylamino)-phenyl]-pyrazolo[3,4-d]pyrimidin-1-yl}-cyclohexyloxy)-ethanol

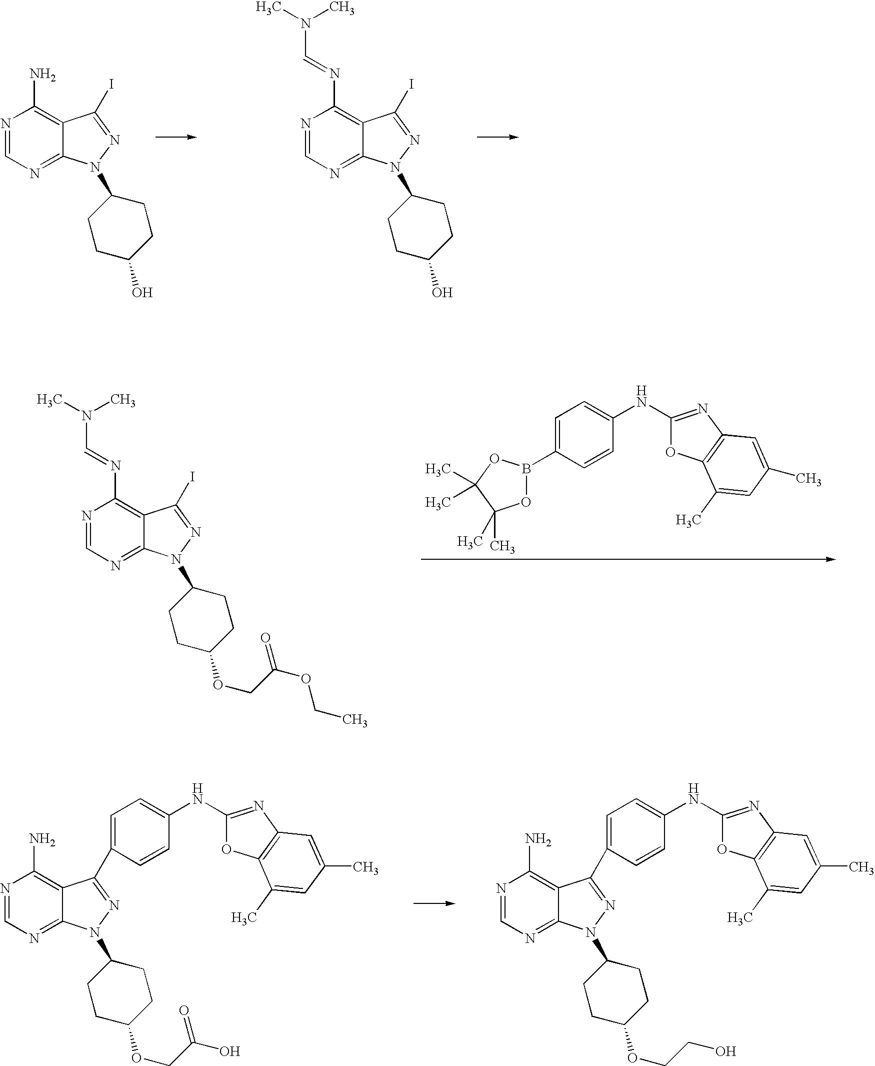

A mixture of trans-4-(4-amino-3-iodo-pyrazolo[3,4-d]pyrimidin-1-yl)-cyclohexanol (0.50 g, 0.00139 mol) (prepared using general procedures A, T and U) and dimethylformamide dimethyl acetal (0.24 mL, 0.00181 mol) in N,N-dimethylformamide (10 mL) was heated at about 85° C. under continuous nitrogen flow for about 16 hours. The solvent was removed under reduced pressure and the residue was triturated with ethyl acetate (20 mL). The precipitate was collected by filtration, washed with ethyl acetate and dried to yield trans-N′-[1-(4-hydroxy-cyclohexyl)-3-iodo-1H-pyrazolo[3,4-d]pyrimidin-4-yl]-N,N-dimethyl-formamidine (0.30 g, 0.00075 mol) as a yellow solid; RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) Rₜ 11.97 min.

Ethyl diazoacetate (0.047 mL, 0.046 mol) was added to a mixture of trans-N′-[1-(4-hydroxycyclohexyl)-3-iodo-1H-pyrazolo[3,4-d]pyrimidin-4-yl]-N,N-dimethyl-formamidine (0.20 g, 0.483 mmol) and rhodium acetate dimer (0.01 g, 0.023 mmol) in dichloromethane (5 mL) and the reaction mixture was stirred at ambient temperature under continuous nitrogen flow for about 78 hours. Additional ethyl diazoacetate (0.047 mL, 0.046 mol) was added after 4, 8, 72, 74 and 76 hours. The solvent was removed under reduced pressure and the residue was purified by preparative RP-HPLC (10% to 60% acetonitrile/0.05M aqueous ammonium acetate, buffered to pH 4.5, over 25 min at 21 mL/min; λ=308 nm; Hypersil C18, 100 Å, 8 μm, 250×21.2 mm column) to yield trans-{4-[4-(dimethylaminomethyleneamino)-3-iodo-pyrazolo[3,4-d]pyrimidin-1-yl]-cyclohexyloxy}-acetic acid ethyl ester (0.044 g, 0.0883 mmol) as an off-white solid; RP-HPLC (5% to 95% acetonitrile/0.05M aqueous ammonium acetate, buffered to pH 4.5, over 10 min at 1.7 mL/min; λ=308 nm; Hypersil C18, 100 Å, 5 μm, 250×4.6 mm column) Rₜ 10.68 min.

trans-{4-[4-(Dimethylaminomethyleneamino)-3-iodo-pyrazolo[3,4-d]pyrimidin-1-yl]-cyclohexyloxy}-acetic acid ethyl ester (0.27 g, 0.00054 mol) was coupled with (5,7-dimethyl-benzoxazol-2-yl)-[2-fluoro-4-(4,4,5,5-tetramethyl-[1,3,2]dioxaborolan-2-yl)-phenyl]-amine (0.236 g, 0.648 mmol) (prepared using general procedures G and D) using the general procedure C to afford trans-(4-{4-amino-3-[4-(5,7-dimethyl-benzoxazol-2-ylmethyl)-phenyl]-pyrazolo[3,4-d]pyrimidin-1-yl}-cyclohexyloxy)-acetic acid (0.209 g, 0.397 mmol) as a white solid; RP-HPLC (10% to 80% acetonitrile/0.01M aqueous ammonium acetate over 6 min at 0.8 mL/min; λ=190-700 nm; Genesis C18, 120 Å, 3 μm, 30×4.6 mm column); Rₜ 5.27 min; m/z (M+H)⁺ 528.

A solution of lithium aluminum hydride (1 M in tetrahydrofuran, 0.266 mL, 0.266 mmol) was added dropwise to a suspension of trans-(4-{4-amino-3-[4-(5,7-dimethyl-benzoxazol-2-ylmethyl)-phenyl]-pyrazolo[3,4-d]pyrimidin-1-yl}-cyclohexyloxy)-acetic acid (0.035 g, 0.064 mmol) in tetrahydrofuran (5 mL) and the reaction mixture was stirred at ambient temperature under continuous nitrogen flow for about 24 hours. The reaction was quenched by a dropwise addition of ice-cold water (1 mL) and the solvents were removed under reduced pressure and the residue was purified by preparative RP-HPLC (10% to 60% acetonitrile/0.05M aqueous ammonium acetate, buffered to pH 4.5, over 25 min at 21 mL/min; λ=308 nm; Hypersil C18, 100 Å, 8 μm, 250×21.2 mm column) to yield trans-2-(4-{4-amino-3-[4-(5,7-dimethyl-benzoxazol-2-ylmethyl)-phenyl]-pyrazolo[3,4-d]pyrimidin-1-yl}-cyclohexyloxy)-ethanol (0.026 g, 0.051 mmol) as a white solid: RP-HPLC (30% to 95% acetonitrile/0.01M aqueous ammonium acetate over 4.5 min at 0.8 mL/min; λ=190-700 nm; Genesis C18, 120 Å, 3 μm, 30×4.6 mm column); Rₜ 1.99 min; m/z (M+H)⁺ 514.

### Preparation #27. cis-{5-(4-Amino-3-fluoro-phenyl)-6-bromo-7-[4-(4-methyl-piperazin-1-yl)-cyclohexyl]-7H-pyrrolo[2,3-d]pyrimidin-4-ylamine}

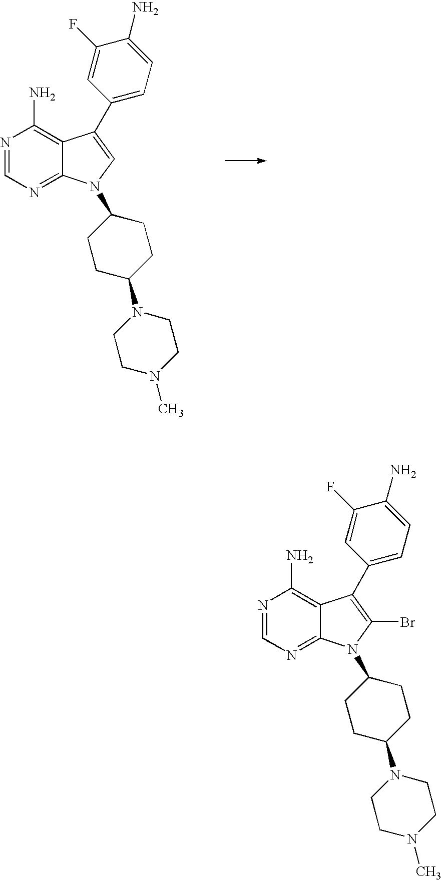

A 25-mL round bottom flask, equipped with a nitrogen inlet was charged with cis-{5-(4-amino-3-fluoro-phenyl)-7-[4-(4-methyl-piperazin-1-yl)-cyclohexyl]-7H-pyrrolo[2,3-d]pyrimidin-4-ylamine} (prepared using general procedures A, B, T, J, C, and L) (600 mg, 1.42 mmol) and N,N-dimethylformamide (10 mL). N-Bromosuccinimide (264 mg, 1.48 mmol) was added portionwise to the reaction mixture, over about 45 minutes. The reaction mixture was stirred at room temperature for about 29 hours. Additional N-bromosuccinimide (240 mg, 1.35 mmol) was added to the reaction mixture and the mixture stirred at room temperature for about 3 days. The solvent was removed under reduced pressure and the residue was taken up in N,N-dimethylformamide. The precipitate was filtered and washed with additional N,N-dimethylformamide. The combined organic washes and filtrate were concentrated to afford a thick orange-brown oil which was purified by mass actuated preparative RPLC (25% to 85% acetonitrile/0.05M aqueous ammonium acetate, buffered to pH 4.5, over 6.5 min at 24 mL/min; λ=254 nm; Hypersil C18, 130 Å, 5 μm, 100×21.2 mm column) to afford cis-{5-(4-amino-3-fluoro-phenyl)-6-bromo-7-[4-(4-methyl-piperazin-1-yl)-cyclohexyl]-7H-pyrrolo[2,3-d]pyrimidin-4-ylamine} as a pale brown solid (25 mg, 0.0498 mmol); m/z (M+H)⁺ 502.

### Example #380

cis-{5-[4-(7-Ethyl-5-methyl-benzoxazol-2-ylamino)-3-fluoro-phenyl]-7-[4-(4-methyl-piperazin-1-yl)-cyclohexyl]-7H-pyrrolo[2,3-d]pyrimidin-4-ylamine}

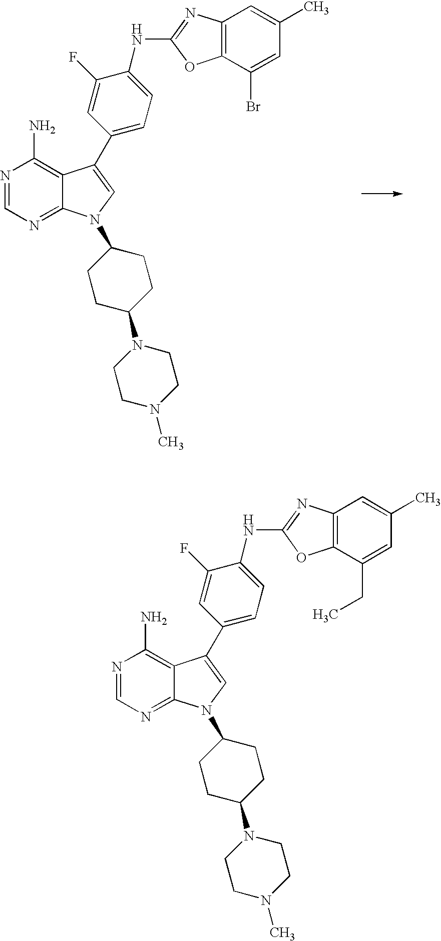

A 25-mL round bottom flask equipped with a reflux condenser fitted with a nitrogen inlet was charged with cis-{5-[4-(7-bromo-5-methyl-benzoxazol-2-ylamino)-3-fluoro-phenyl]-7- [4-(4-methyl-piperazin-1-yl)-cyclohexyl]-7H-pyrrolo[2,3-d]pyrimidin-4-ylamine} (prepared using general procedures A, B, T, J, C, L, and G (F, H)) (188 mg, 0.297 mmol), palladium (II) acetate (3 mg, 0.015 mmol), 2-(dicyclohexylphosphino)biphenyl (11 mg, 0.030 mmol), sodium carbonate (79 mg, 0.743 mmol), ethylene glycol dimethyl ether (2 mL) and water (1 mL). Triethylborane (1.0 M solution in tetrahydrofuran, 0.60 mL, 0.594 mmol) was added and the mixture was heated at about 80° C. for about 2 hours. Additional palladium (II) acetate (3 mg, 0.015 mmol), 2-(dicyclohexylphosphino)biphenyl (11 mg, 0.030 mmol) and triethylborane (0.25 mL, 0.25 mmol) were added to the reaction mixture and the mixture was stirred at room temperature for about 17 hours. Additional palladium (II) acetate (3 mg, 0.015 mmol), 2-(dicyclohexylphosphino)biphenyl (11 mg, 0.030 mmol) and triethylborane (0.25 mL, 0.25 mmol) were added and the mixture was stirred at about 80° C. for an additional hour. The reaction mixture was then cooled to room temperature and was partitioned between ethyl acetate (10 mL) and water (10 mL). The aqueous phase was separated and further extracted with ethyl acetate (2×10 mL). The combined organic phases were washed with brine (20 mL), dried over magnesium sulfate and the organic solvent was removed under reduced pressure to afford a red-brown oil. The product was purified by preparative reverse-phase HPLC (15% to 75% acetonitrile/0.05M aqueous ammonium acetate, buffered to pH 4.5, over 30 min at 21 mL/min; x =254 nm; Hypersil C18, 100 Å, 8 μm, 250×21.2 mm column) to afford cis-{5-[4-(7-ethyl-5-methyl-benzoxazol-2-ylamino)-3-fluoro-phenyl]-7-[4-(4-methyl-piperazin-1-yl)-cyclohexyl]-7H-pyrrolo[2,3-d]pyrimidin-4-ylamine] (85 mg, 0.146 mmol) as an off-white solid; RP-HPLC (Delta Pak C18, 5 μm, 300 Å, 15 cm; 5% to 85% acetonitrile/50 mM aqueous ammonium acetate, buffered to pH 4.5, over 20 min, 1 mL/min) Rₜ 13.80 min, m/z (M+H)⁺ 583.

### Example #381

cis-{7-[4-(4-Cyclopropyl-piperazin-1-yl)-cyclohexyl]-5-[4-(5,7-dimethyl-benzoxazol-2-ylamino)-3-fluoro-phenyl]-7H-pyrrolo[2,3-d]pyrimidin-4-ylamine}

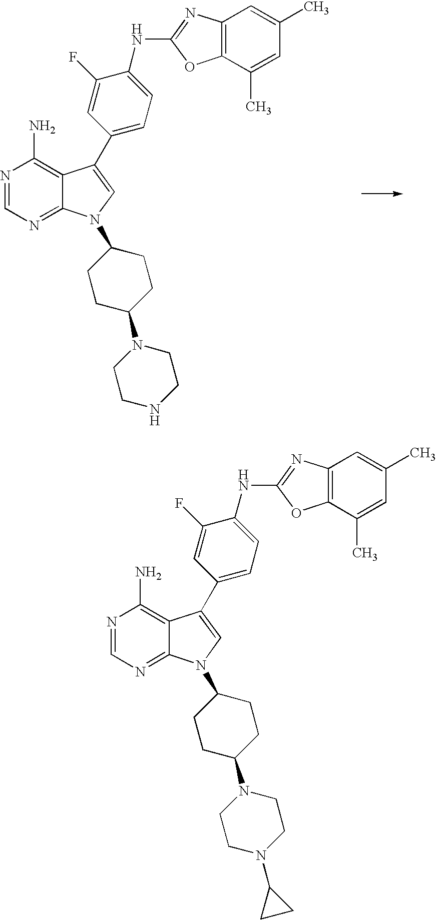

A 25-mL round bottom flask equipped with a reflux condenser, fitted with a nitrogen inlet was charged with cis-{5-[4-(5,7-dimethyl-benzoxazol-2-ylamino)-3-fluoro-phenyl]-7-(4-piperazin-1-yl-cyclohexyl)-7H-pyrrolo[2,3-d]pyrimidin-4-ylamine} (prepared by general procedures A, B, T, J, C, L, and G) (100 mg, 0.180 mmol), methanol (3 mL), acetic acid (108 mg, 1.80 mmol) and sodium cyanoborohydride (45 mg, 0.72 mmol) in methanol (3 mL). [(1-Ethoxycyclopropyl)oxy]-trimethylsilane (157 mg, 0.901 mmol) was added and the mixture was stirred at about 64° C. for about 21 hours and was then cooled to room temperature. The reaction mixture was filtered and the solids were washed with ethyl acetate. The combined organic washes and filtrate were concentrated to afford a thick yellow oil. The product was purified by preparative reverse-phase HPLC (15% to 75% acetonitrile/0.05M aqueous ammonium acetate, buffered to pH 4.5, over 30 min at 21 mL/min; λ=254 nm; Hypersil C18, 100 Å, 8 μm, 250×21.2 mm column) to afford cis-{7-[4-(4-cyclopropyl-piperazin-1-yl)-cyclohexyl]-5-[4-(5,7-dimethyl-benzoxazol-2-ylamino)-3-fluoro-phenyl]-7H-pyrrolo[2,3-d]pyrimidin-4-ylamine} as an off-white solid (15 mg, 0.025 mmol); RP-HPLC (Delta Pak C18, 5 μm, 300 Å, 15 cm; 5% to 85% acetonitrile/50 mM ammonium acetate over 20 min, 1 mL/min) Rₜ 13.495 min, m/z (M+H)⁺ 595.

### Example #382

cis-{6-Bromo-5-[4-(5,7-dimethyl-benzoxazol-2-ylamino)-3-fluoro-phenyl]-7-[4-(4-methyl-piperazin-1-yl)-cyclohexyl]-7H-pyrrolo[2,3-d]pyrimidin-4-ylamine}

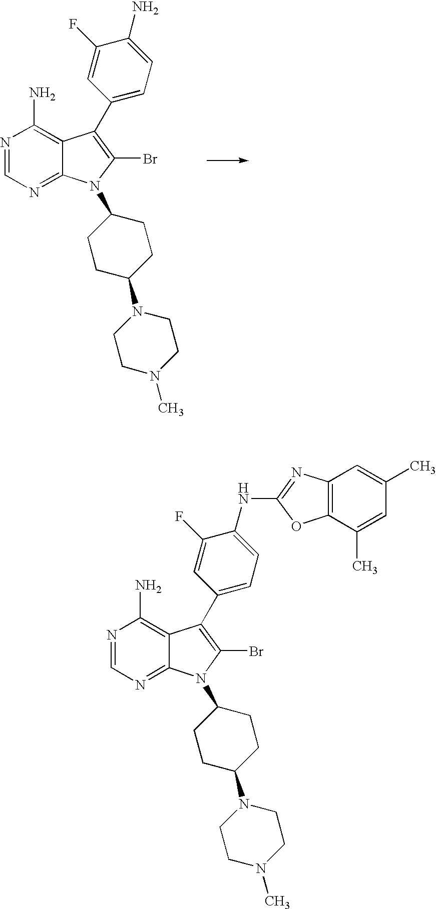

This compound was prepared from cis-{5-(4-amino-3-fluoro-phenyl)-6-bromo-7-[4-(4-methyl-piperazin-1-yl)-cyclohexyl]-7H-pyrrolo[2,3-d]pyrimidin-4-ylamine } (preparation #28) and 2-amino-4,6-dimethyl-phenol, using general procedure G, to afford cis-{6-bromo-5-[4-(5,7-dimethyl-benzoxazol-2-ylamino)-3-fluoro-phenyl]-7-[4-(4-methyl-piperazin-1-yl)-cyclohexyl]-7H-pyrrolo[2,3-d]pyrimidin-4-ylamine} as a beige solid (4 mg, 0.007 mmol); RP-HPLC (Delta Pak C18, 5 μm, 300 Å, 15 cm; 5% to 85% acetonitrile/50 mM aqueous ammonium acetate over 20 min, 1 mL/min) Rₜ 15.45 min, m/z (M+H)⁺ 647.

### Example #383

cis-{5-[4-(5,7-Dimethyl-benzoxazol-2-ylamino)-3-fluoro-phenyl]-7-[4-(4-methane-sulfonyl-piperazin-1-yl)-cyclohexyl]-7H-pyrrolo[2,3-d]pyrimidin-4-ylamine}

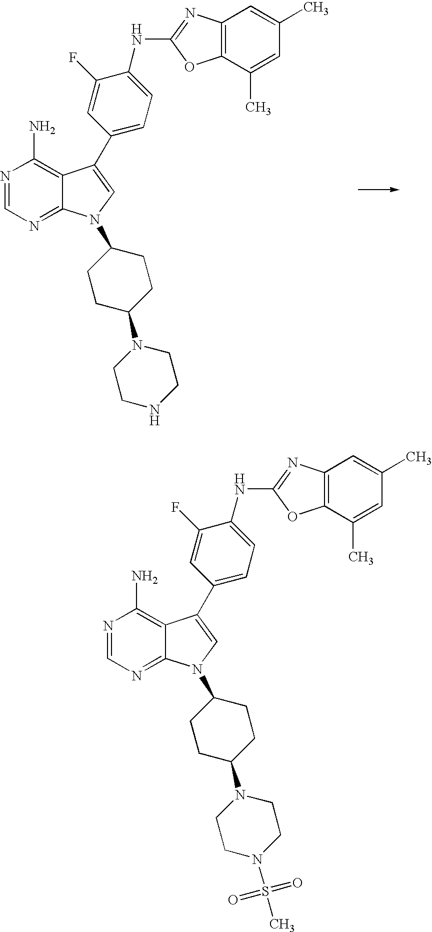

Methanesulfonyl chloride (7 μL, 0.093 mmol) was added to a solution of cis-{5-[4-(5,7-dimethyl-benzoxazol-2-ylamino)-3-fluoro-phenyl]-7-(4-piperazin-1-yl-cyclohexyl)-6,7-dihydro-5H-pyrrolo[2,3-d]pyrimidin-4-ylamine} (prepared using general procedures A, B, T, J, C, L, and G) (51 mg, 0.093 mmol) and triethylamine (13 μL, 0.093 mmol) in dichloromethane (7 mL) at about 0° C., under an inert atmosphere. The solution was warmed slowly to room temperature and the reaction mixture was stirred for about 3 weeks. Additional methanesulfonyl chloride (14 μL, 0.186 mmol) and triethylamine (26 μL, 0.186 mmol) were added to the reaction mixture during this time. The reaction mixture was quenched with saturated aqueous sodium bicarbonate (15 mL) and the crude product was extracted with dichloromethane (3×30 mL). The combined organic extracts were washed with brine (50 mL), dried over magnesium sulfate and the solvent was removed under reduced pressure. The crude mixture was purified by preparative reverse-phase HPLC (Delta Pak C18, 5 μm, 300 Å, 15 cm; 10% to 60% acetonitrile/50 mM aqueous ammonium acetate over 25 min, 20 mL/min) to afford cis-{5-[4-(5,7-dimethyl-benzoxazol-2-ylamino)-3-fluoro-phenyl]-7-[4-(4-methane-sulfonyl-piperazin-1-yl)-cyclohexyl]-7H-pyrrolo[2,3-d]pyrimidin-4-ylamine} as an off-white solid (12 mg, 0.019 mmol); RP-HPLC (Delta Pak C18, 5 μm, 300 Å, 15 cm; 5% to 95% acetonitrile/50 mM aqueous ammonium acetate over 10 min, 1 mL/min) Rₜ 9.06 min, m/z (M+H)⁺ 633.

### Example #384

3-[4-(5,7-Dimethyl-benzoxazol-2-ylamino)-phenyl]-1-(4-methylene-cyclohexyl)-1H-pyrazolo[3,4-d]pyrimidin-4-ylamine

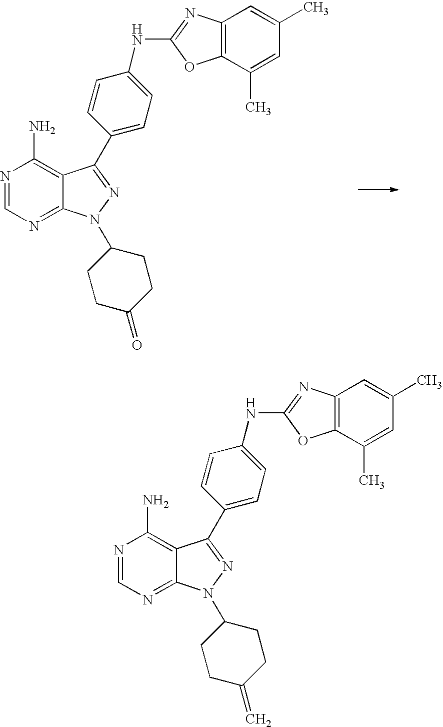

To a suspension of methyltriphenylphosphonium bromide (7.64 g, 21.4 mmol) in tetrahydrofuran (200 mL) at about −78° C. was added n-butyllithium (0.69 M in tetrahydrofuran, 31 mL, 21.4 mmol), such that the temperature of the reaction did not exceed about −50° C. The reaction mixture was slowly warmed to room temperature and stirred for about 2 hours. The mixture was then cooled back down to about −20° C. and a solution of 4-{4-amino-3-[4-(5,7-dimethyl-benzoxazol-2-ylamino)-phenyl]-pyrazolo[3,4-d]pyrimidin-1-yl}-cyclohexanone (prepared by general procedures A, T, and C) (5.0 g, 10.7 mmol) in tetrahydrofuran (80 mL) was added. The reaction mixture was then stirred at about 50° C. for about 16 hours. The solvent was removed under reduced pressure and the residue was partitioned between water (100 mL) and dichloromethane (150 mL). The organic layer was extracted and the aqueous layer was extracted with additional dichloromethane (2×150 mL). The combined organic fractions were washed with brine (150 mL), dried over magnesium sulfate and the solvent was removed under reduced pressure to afford an orange syrup. The crude product was purified by flash column chromatography on silica gel using dichloromethane/acetone (75:25) as the mobile phase to give 3-[4-(5,7-dimethyl-benzoxazol-2-ylamino)-phenyl]-1-(4-methylene-cyclohexyl)-1H-pyrazolo[3,4-d]pyrimidin-4-ylamine as a white solid (4.0 g, 8.59 mmol); RP-HPLC (Delta Pak C18, 5 μm, 300 Å, 15 cm; 5% to 95% acetonitrile/50 mM aqueous ammonium acetate over 10 min, 1.7 mL/min) Rₜ 12.82 min, m/z (M+H)⁺ 466.

### Example #385

cis-{3-[4-(5,7-Dimethyl-benzoxazol-2-ylamino)-phenyl]-1-(3-methyl-1-oxa-2-aza-spiro[4.5]dec-2-en-8-yl)-1H-pyrazolo[3,4-d]pyrimidin-4-ylamine}

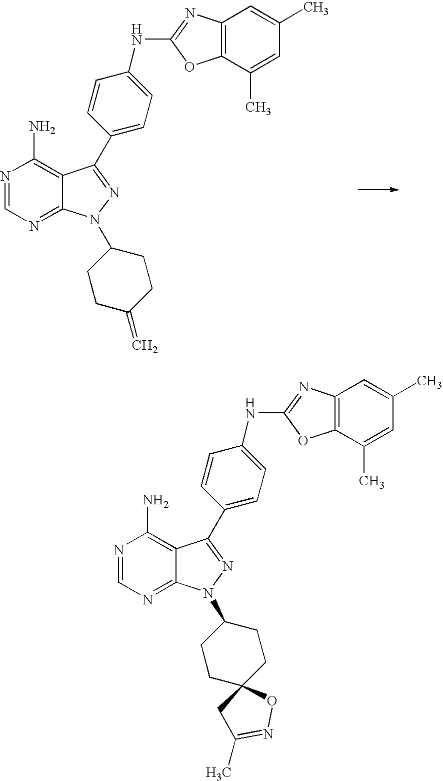

To a solution of acetaldoxime (102 mg, 1.72 mmol) in N,N-dimethylformamide (2.5 mL) at about 0° C. was added N-chlorosuccinimide (230 mg, 1.72 mmol). The reaction mixture was allowed to warm to room temperature and was stirred at this temperature for 1 hour under an atmosphere of nitrogen. A solution of 3-[4-(5,7-dimethyl-benzoxazol-2-ylamino)-phenyl]-1-(4-methylene-cyclohexyl)-1H-pyrazolo[3,4-d]pyrimidin-4-ylamine (Example #384) (1.00 g, 2.15 mmol) in N,N-dimethylformamide (12 mL) was added to the reaction mixture in one portion at room temperature, followed by a solution of triethylamine (250 μL, 1.80 mmol) in N,N-dimethylformamide (2 mL), which was added slowly over about 2 hours. The reaction mixture was stirred for about 15 hours at room temperature. The crude reaction mixture was partitioned between water (25 mL) and dichloromethane (25 mL), the organic layer was separated and the aqueous layer was extracted with additional dichloromethane (2×25 mL). The combined organic layers were dried over magnesium sulfate and the solvent was removed under reduced pressure. The crude mixture was purified by preparative reverse-phase HPLC (C18, 8 μm, 250×21.2 mm; 70% to 80% acetonitrile/50 mM aqueous ammonium acetate over 20 min, 21 mL/min) to afford cis-{3-[4-(5,7-dimethyl-benzoxazol-2-ylamino)-phenyl]-1-(3-methyl-1-oxa-2-aza-spiro[4.5]dec-2-en-8-yl)-1H-pyrazolo[3,4-d]pyrimidin-4-ylamine} as a light yellow solid (31 mg, 0.059 mmol); RP-HPLC (Delta Pak C18, 5 μm, 300 Å, 15 cm; 5% to 95% acetonitrile/50 mM aqueous ammonium acetate over 10 min, 1.7 mL/min) Rₜ 12.72 min, m/z (M+H)⁺ 523.

### Example #386

trans-3-(4-Benzoxazol-2-ylmethyl-phenyl)-1-[4-(4-methyl-piperazin-1-yl)-cyclohexyl]-1H-pyrazolo[3,4-d]pyrimidin-4-ylamine

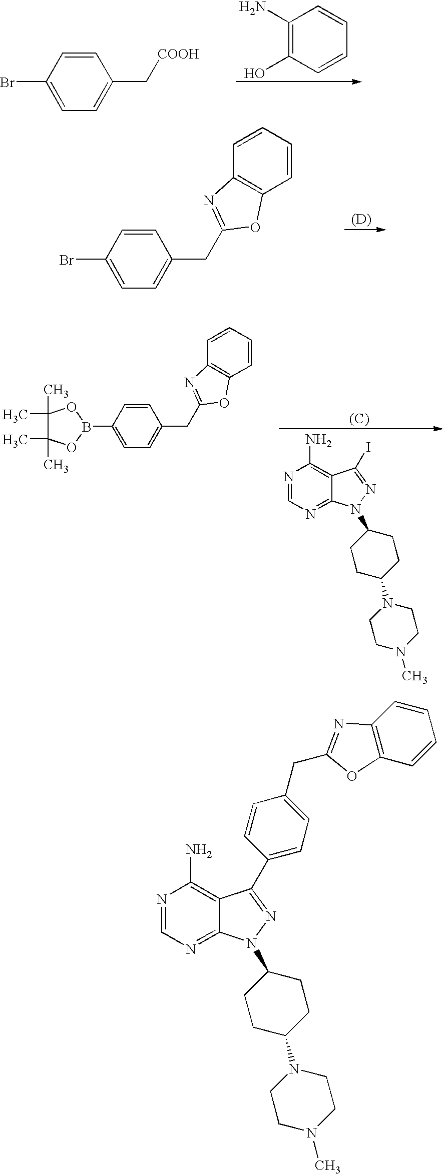

2-Aminophenol (0.257 g, 2.36 mmol) and 4-bromophenylacetic acid (0.500 g, 2.36 mmol) were heated together at about 200° C. in an open test tube for 1 hour. The reaction mixture was cooled to ambient temperature, dissolved in methanol-dichloromethane (1:20, 50 mL), and extracted with dilute aqueous sodium carbonate (10 mL). The organic layer was dried over magnesium sulfate, filtered, and concentrated. The residue was purified by flash column chromatography on silica gel, using ethyl acetate-heptane (15:85) as the mobile phase, to afford 2-(4-bromobenzyl)-benzoxazole (0.347 g, 1.20 mmol) as yellow flakes: m/z (M+H)⁺ 288, 290. 2-(4-Bromo-benzyl)-benzoxazole (0.100 g, 0.347 mmol) was converted to 2-[4-(4,4,5,5-tetramethyl-[1,3,2]dioxaborolan-2-yl)-benzyl]-benzoxazole using general procedure D, and the crude product was then reacted, using general procedure C, with trans-3-iodo-1-[4-(4-methyl-piperazin-1-yl)-cyclohexyl]-1H-pyrazolo[3,4-d]pyrimidin-4-ylamine (prepared using general procedures A, T, and J) to afford trans-3-(4-benzoxazol-2-ylmethyl-phenyl)-1-[4-(4-methyl-piperazin-1-yl)-cyclohexyl]-1H-pyrazolo[3,4-d]pyrimidin-4-ylamine as a white powder (0.102 g, 0.195 mmol); RP-HPLC (25% to 100% acetonitrile/0.1 M aqueous ammonium acetate, buffered to pH 4.5, over 10 min at 1.0 mL/min; λ=254 nm; Hypersil C18, 100 Å, 5 μm, 250×4.6 mm column) Rₜ 6.83 min; m/z (M+H)⁺ 523.

### Preparation #28. N-(3-Bromo-1-methyl-1H-pyrazolo[3,4-d]pyrimidin-6-yl)-phenethylamine

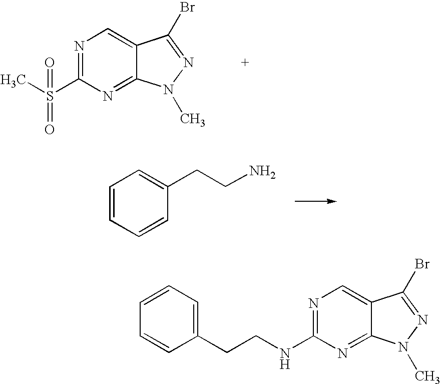

To a solution of 3-bromo-6-methanesulfonyl-1-methyl-1H-pyrazolo[3,4-d]pyrimidine (WO 03029209) (0.282 g, 0.969 mmol) in 1-methyl-2-pyrrolidinone (10 mL) was added phenethylamine (0.587 g, 4.85 mmol) and the reaction mixture was heated to about 50° C. After about an hour, water (40 mL) was added, followed by ethyl acetate (40 mL). The layers were separated. The aqueous portion was re-extracted with ethyl acetate (2×20 mL). The combined organic fractions were washed with brine, dried over magnesium sulfate, filtered, and evaporated. The crude yellow residue was purified by flash column chromatography on silica gel, using ethyl acetate/heptane (1:1) as the mobile phase, to give pure (3-bromo-1-methyl-1H-pyrazolo[3,4-d]pyrimidin-6-yl)-phenethyl-amine as a white solid (0.205 g, 0.636 mmol); RP-HPLC (5% to 95% acetonitrile/0.05 M ammonium acetate over 10 min at 1.7 mL/min; 15 min total run time; λ=254 nm; Hypersil C18, 5 μm, 100 Å, 250×4.6 mm) Rₜ 11.78 min; m/z: (M+H)⁺ 332 and 334.

### Preparation #29. N-(4-Bromo-phenyl)-[5-methyl-7-(3-morpholin-4-yl-propoxy)-benzoxazol-2-yl]-amine

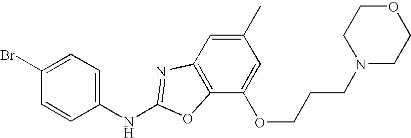

### Preparation #29.1. 2-(3-Bromo-propoxy)-1-methoxy-4-methyl-benzene

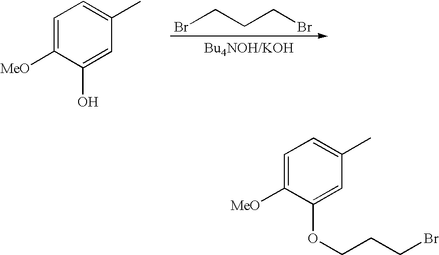

A solution of 2-methoxy-5-methyl-phenol (5.0 g, 36.2 mmol), 1,3-dibromopropane (40 mL, 360 mmol), tetrabutylammonium hydroxide (16 mL, 24.25 mmol) and 40% wt/wt aqueous potassium hydroxide (180 mmol) were stirred at 55° C. for 1 hour. The solution was diluted with ether, washed with water and brine, dried over magnesium sulfate, filtered and concentrated. The resulting oil was subjected to flash chromatography eluting with hexanes and gradually increasing the polarity to 10% ethyl acetate to give 2-(3-bromo-propoxy)-1-methoxy-4-methyl-benzene (6.5 g, 70 % yield).

### Preparation #29.2. 4-[3-(2-Methoxy-5-methyl-phenoxy)-propyl]-morpholine

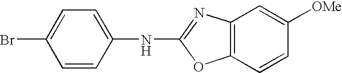

A solution of 2-(3-bromo-propoxy)-1-methoxy-4-methyl-benzene (3.0 g, 11.6 mmol) and morpholine (5.05 mL, 57.9 mmol) in THF (60 mL) was heated at 65° C. for 2 hours. The solution was cooled to room temperature, diluted with ether, washed with water and brine, dried over magnesium sulfate, filtered and concentrated to give 4-[3-(2-methoxy-5-methyl-phenoxy)-propyl]-morpholine (3.01 g, 97% yield); m/z: (M+H)⁺ 319, 321.

### Preparation #29.3. 4-Methyl-2-(3-morpholin-4-yl-propoxy)-phenol

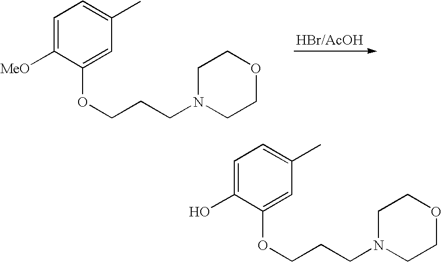

A solution of 4-[3-(2-methoxy-5-methyl-phenoxy)-propyl]-morpholine (3.0 g, 11.31 mmol) in acetic acid (25 mL) and HBr (25 mL) was stirred at 90° C. for 4 hours. The solution was cooled to room temperature and condensed. The resulting residue was taken up in ethyl acetate and washed with a saturated sodium bicarbonate solution. The aqueous layer was then saturated with sodium chloride and extracted with ethyl acetate. Organics were combined and the solvent was removed under reduced pressure to give 4-methyl-2-(3-morpholin-4-yl-propoxy)-phenol (2.36 g); m/z: (M+H)⁺ 251.

### Preparation #29.4. 4-Methyl-2-(3-morpholin-4-yl-propoxy)-6-nitro-phenol

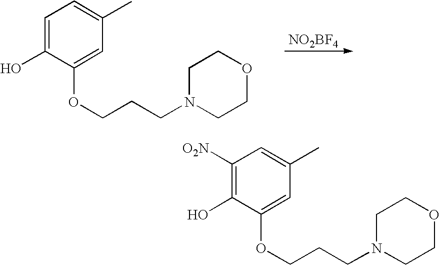

A −78° C. solution of 4-methyl-2-(3-morpholin-4-yl-propoxy)-phenol (2.36 g, 9.4 mol) in DME (66 mL) was added to a solution of NO₂BF₄ (11.27 mmol) in DME (44 mL) at −78° C. The solution was stirred and allowed to warm to −10° C. Ice was added to quench the solution. The solution was condensed to remove DME. The residue was cooled in an ice bath, neutralized with aqueous sodium bicarbonate, and extracted with methylene chloride. The organics were combined, condensed and the residue was subjected to flash chromatography on silica gel eluting with 1% TEA/1% MeOH/methylene chloride. The first fraction contained the desired product, an orange semi-solid. This material was subjected to a second flash column, eluting with 5:5:1 ethyl acetate:hexanes:methanol (to remove triethylamine salt) to give 4-methyl-2-(3-morpholin-4-yl-propoxy)-6-nitro-phenol (0.687 g, 25% yield); m/z: (M+H)⁺ 297.

### Preparation #29.5. 2-Amino-4-methyl-6-(3-morpholin-4-yl-propoxy)-phenol

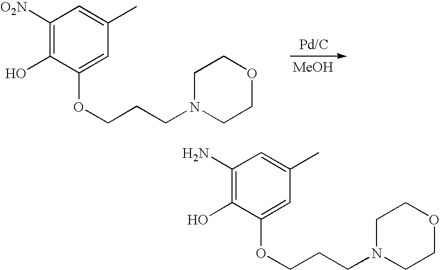

A solution of 4-methyl-2-(3-morpholin-4-yl-propoxy)-6-nitro-phenol (0.687 g, 2.32 mmol) and Pd/C (0.116 mmol) in methanol (65 mL) was stirred under an atmosphere of hydrogen for 4 hours. The system was purged with nitrogen, filtered through celite and condensed. The residue was triturated with methanol to give 2-amino-4-methyl-6-(3-morpholin-4-yl-propoxy)-phenol (0.315 g, 51% yield); m/z: (M+H)⁺ 267.

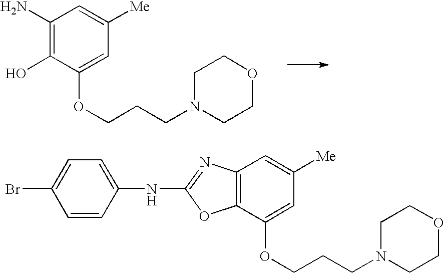

A solution of 2-amino-4-methyl-6-(3-morpholin-4-yl-propoxy)-phenol (0.315 g, 1.18 mmol) and 1-bromo-4-isothiocyanato-benzene (0.245 g, 1.14 mmol) in THF (6 mL) was stirred at room temperature under an atmosphere of nitrogen for 3.5 hours. EDCI (0.262 g, 1.36 mmol) was added and the solution was stirred at 50° C. overnight, then cooled to room temperature resulting in the oiling out of the product. The solution was concentrated and the residue was taken up in acetonitrile, heated to obtain a homogeneous solution and cooled to room temperature. The resulting precipitate was removed via vacuum filtration. The filtrate was condensed and the residue was partitioned between ethyl acetate and water. The organic layer was condensed and the residue was triturated from acetonitrile to give (4-bromo-phenyl)-[5-methoxy-7-(3-morpholin-4-yl-propoxy)-benzoxazol-2-yl]-amine (0.335 g, 61% yield); m/z (M⁺) 445, 447.

### Preparation #30. (4-Bromo-phenyl)-[5-methyl-7-(1-methyl-piperidin-4-ylmethoxy)-benzoxazol-2-yl]-amine

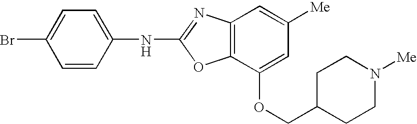

### Preparation #30.1. 4-(2-Methoxy-5-methyl-phenoxymethyl)-1-methyl-piperidine

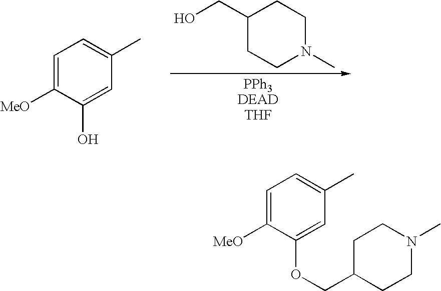

A 0° C. solution of 2-methoxy-5-methyl-phenol (3.45 g 25 mmol) in THF (110 mL) was treated with triphenylphosphine (7.87 g, 30.0 mmol), followed by DEAD (4.72 mL, 30.0 mmol), stirred for 5 minutes then treated with a solution of (1-methyl-piperidin-4-yl)-methanol (3.88 g, 30.0 mmol) in THF (140 mL). The ice bath was removed and the solution was stirred at room temperature overnight. The solution was condensed and subjected to flash chromatography on silica gel eluting with 5% MeOH/methylene chloride to give 4-(2-methoxy-5-methyl-phenoxymethyl)-1-methyl-piperidine (3.58 g, 57% yield); m/z: (M+H)⁺ 250.

The remainder of the synthesis was completed, using the route detailed for preparation #29, by substituting prep #30.1 for prep #29.2 to afford (4-bromo-phenyl)-[5-methyl-7-(1-methyl-piperidin-4-ylmethoxy)-benzoxazol-2-yl]-amine; m/z (M⁺) 429, 431.

### Preparation #31. (7-Allyl-5-methyl-benzoxazol-2-yl)-(4-bromo-phenyl)-amine

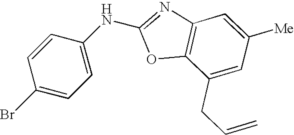

### Preparation #31.1. 2-Allyl-4-methyl-phenol

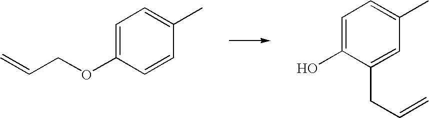

A solution of allyl para-tolyl ether (1 g) in N,N-diethylaniline (5 mL) was heated at 180° C. for 18 h, allowed to cool to r.t., then partitioned between 1N HCl and ether (2×). The combined ether extracts were dried (Na₂SO₄), concentrated and the residue was purified via silica gel chromatography eluting with 15:1 hexanes:EtOAc to give 2-allyl-4-methyl-phenol (0.67 g).

The remainder of the synthesis was completed, using the route detailed for preparation #29, by substituting prep #31.1 for prep #29.3 to afford (7-allyl-5-methyl-benzoxazol-2-yl)-(4-bromo-phenyl)-amine; m/z: (M+H)⁺ 342.9, 344.9.

### Preparation #32. (4-Bromo-phenyl)-[5-methyl-7-(3-morpholin-4-yl-propyl)-benzoxazol-2-yl]-amine

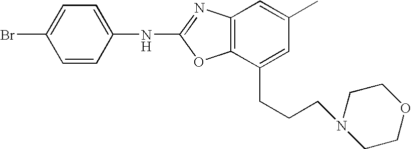

### Preparation #32.1. 3-[2-(4-Bromo-phenylamino)-5-methyl-benzoxazol-7-yl]-propan-1-ol

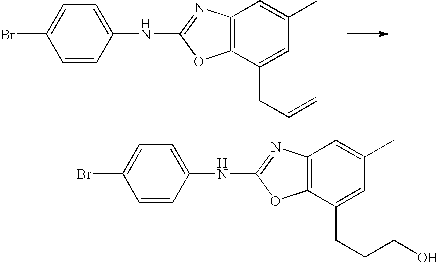

Borane-THF (98 mL, 1 M in THF, 98 mmol) was added dropwise to a 0° C. solution of preparation #31 (6.74 g, 19.6 mmol) in THF (300 mL). The resulting mixture was stirred at 0° C. for 3 h, carefully treated with 6N NaOH (13.5 mL), then with 30% H₂O₂ (27 mL), heated to 60° C. for 1.2 h, then quenched with saturated aqueous NaHSO₃ (added dropwise). The acidic mixture (pH 1-2) was neutralized with saturated aqueous NaHCO₃ and extracted with ether (2×). The combined ether extracts were dried (Na₂SO₄), concentrated and the residue was purified via silica gel chromatography eluting with 2:1 hexanes:EtOAc and subsequent trituration with CH₂Cl₂ afforded 3- [2-(4-bromo-phenylamino)-5-methyl-benzoxazol-7-yl]-propan-1-ol as a solid (3.98 g, 56% yield); m/z: (M+H)⁺ 360.9, 362.9.

### Preparation #32.2. Methanesulfonic acid 3-[2-(4-bromo-phenylamino)-5-methyl-benzoxazol-7-yl]-propyl ester

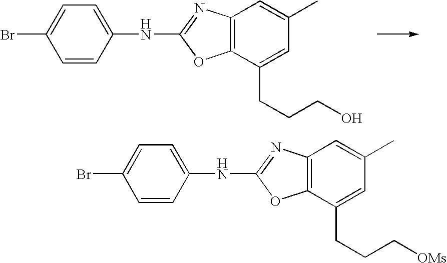

A 0° C. solution of 3-[2-(4-bromo-phenylamino)-5-methyl-benzoxazol-7-yl]-propan-1-ol (0.34 g, 0.94 mmol) in pyridine (10 mL) was treated with methanesulfonyl chloride (0.08 mL), stirred at 0° C. for 15 min, then r.t. for a further 2 h, then treated with additional methanesulfonyl chloride (0.1 mL) and stirred for 3 h. The mixture was partitioned between ether and water, and the organic extract was washed with brine, dried (MgSO₄), filtered and concentrated to give crude methanesulfonic acid 3-[2-(4-bromo-phenylamino)-5-methyl-benzoxazol-7-yl]-propyl ester (0.36 g); m/z: (M+H)⁺ 438.9, 440.7.

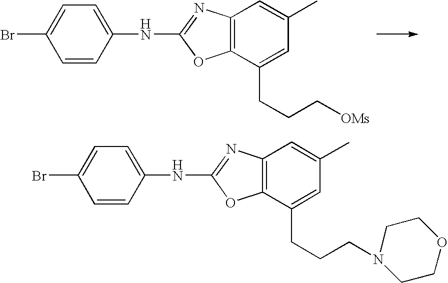

A solution of methanesulfonic acid 3-[2-(4-bromo-phenylamino)-5-methyl-benzoxazol-7-yl]-propyl ester (0.047 g) and morpholine (1 mL) in DMF (2 mL) was heated at 80° C. for 3 h, allowed to cool to r.t., then partitioned between water and ether. The organic extract was washed with brine, dried (MgSO₄), filtered, concentrated and the residue was triturated with ether to give (4-bromo-phenyl)-[5-methyl-7-(3-morpholin-4-yl-propyl)-benzoxazol-2-yl]-amine (0.03 g); m/z: (M+H)⁺ 429.8, 431.8.

### Preparation #33. 2-(4-Bromo-phenylamino)-5-methyl-benzoxazol-7-ol

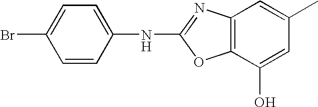

### Preparation #33.1. (4-Bromo-phenyl)-(7-methoxy-5-methyl-benzoxazol-2-yl)-amine

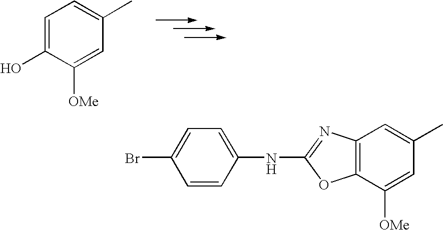

Substituting 2-methoxy-4-methyl phenol for preparation #29.3 and following the steps detailed to complete the synthesis of preparation #29 afforded (4-bromo-phenyl)-(7-methoxy-5-methyl-benzoxazol-2-yl)-amine.

A solution of (4-bromo-phenyl)-(7-methoxy-5-methyl-benzoxazol-2-yl)-amine (4.46 g) in acetic acid (60 mL) and 48% HBr (60 mL) was heated at reflux for 6 h, then stirred at r.t. for 10 h. The resulting precipitate was collected via filtration to give 2-(4-bromo-phenylamino)-5-methyl-benzoxazol-7-ol (3.66 g); m/z: (M+H)⁺ 318.9, 320.8.

### Preparation #34. (4-Bromo-phenyl)-(5-methoxy-benzoxazol-2-yl)-amine

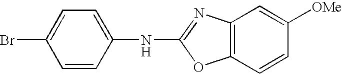

Substituting 2-nitro-4-methoxy phenol for preparation #29.4 and following the steps detailed to complete the synthesis of preparation #29 afforded (4-bromo-phenyl)-(5-methoxy-benzoxazol-2-yl)-amine; m/z: (M+H)⁺ 319, 321.

### Preparation #35. (4-Bromo-phenyl)-[5-methyl-7-(2-pyrrolidin-1-yl-ethoxy)-benzoxazol-2-yl]-amine

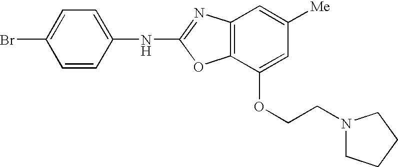

Using the route described from preparation #29.1 through preparation #29, by substituting 1,2-dibromoethane for 1,3-dibromopropane and pyrrolidine for morpholine afforded (4-bromo-phenyl)-[5-methyl-7-(2-pyrrolidin-1-yl-ethoxy)-benzoxazol-2-yl]-amine; m/z: (M+H)⁺ 416, 418.

### Preparation #36. (4-Bromo-phenyl)-[7-(2-dimethylamino-ethoxy)-5-methyl-benzoxazol-2-yl]-amine

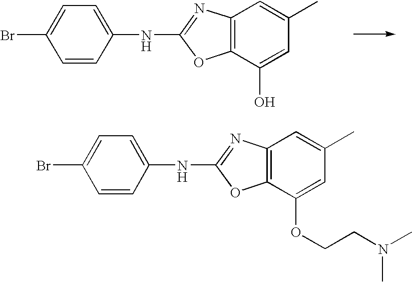

A mixture of preparation #33 (0.09 g, 0.28 mmol), Cs₂CO₃ (0.37 g, 1.13 mmol) and (2-chloro-ethyl)dimethylamine hydrochloride (0.044 g, 0.3 mmol) in DMF (1.5 mL) was heated at 80° C. for 8 h, cooled to r.t. then partitioned between water and ether (2×). The combined extracts were dried (MgSO₄), concentrated and the residue was purified via silica gel chromatography eluting with 5:4:1 hexanes:EtOAc:methanol to afford (4-bromo-phenyl)-[7-(2-dimethylamino-ethoxy)-5-methyl-benzoxazol-2-yl]-amine (0.103 g, 94% yield); m/z: (M+H)⁺ 389.9, 391.8.

### Preparation #37. 2-(4-Bromo-phenylamino)-5-chloro-benzoxazol-7-ol

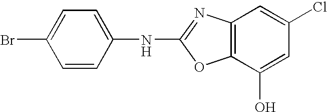

### Preparation #37.1. (4-Bromo-phenyl)-(5-chloro-7-methoxy-benzoxazol-2-yl)-amine

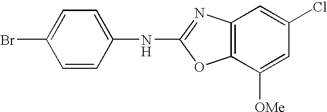

Substituting 4-chloro-2-methoxyphenol for preparation #29.3 and completing the synthetic route, for preparation #29, afforded (4-bromo-phenyl)-(5-chloro-7-methoxy-benzoxazol-2-yl)-amine.

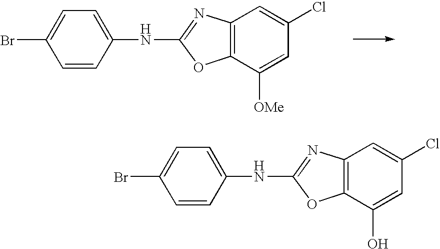

A mixture of (4-bromo-phenyl)-(5-chloro-7-methoxy-benzoxazol-2-yl)amine (0.687 g, 1.9 mmol), 2,4,6-collidine (10 mL) and LiI (1.04 g, 7.7 mmol) was heated to reflux overnight, cooled to r.t., diluted with 1N HCl and extracted with ether (4×). The combined ethereal extracts were dried (Na₂SO₄), filtered and concentrated to give 2-(4-bromo-phenylamino)-5-chloro-benzoxazol-7-ol (0.58 g, 88% yield); m/z: (M−H)⁻ 336.8, 338.8.

### Preparation #38. 2-(4-Bromo-2-fluoro-phenylamino)-5-chloro-benzoxazol-7-ol

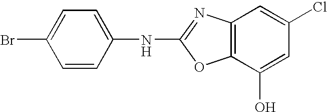

Following steps preparation #29.4 through preparation #29, substituting 4-chloro-2-methoxyphenol for preparation #29.3 and 4-bromo-2-fluoro-1-isothiocyanato-benzene for 4-bromo-1-isothiocyanato-benzene afforded 2-(4-bromo-2-fluoro-phenylamino)-5-chloro-benzoxazol-7-ol; m/z: (M+H)⁺ 356.8, 358.8.

### Preparation #39. 5-Iodo-7-(4-nitro-benzyl)-7H-pyrrolo[2,3-d]pyrimidin-4-ylamine

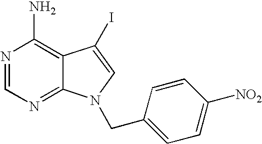

### Preparation #39.1. 4-Chloro-5-iodo-7-(4-nitro-benzyl)-7H-pyrrolo[2,3-d]pyrimidine

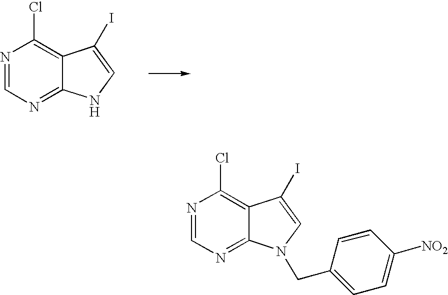

Sodium hydride (0.47 g, 60% oil dispersion, 11.8 mmol) was added in portions to a solution of 4-chloro-5-iodo-7H-pyrrolo[2,3-d]pyrimidine (3.0 g, 10.7 mmol) in DMF (50 mL) and the resulting mixture was stirred at r.t. for 40 min, then treated with 4-nitrobenzyl bromide (2.58 g, 11.8 mmol) and stirred for an additional 3 h. The mixture was diluted with water and extracted with THF-ether (2×). The extract was cooled to −20° C. for 4 h, and the resulting precipitate was collected via filtration to give 4-chloro-5-iodo-7-(4-nitro-benzyl)-7H-pyrrolo[2,3-d]pyrimidine (3.8 g); m/z: (M+H)⁺ 414.8.

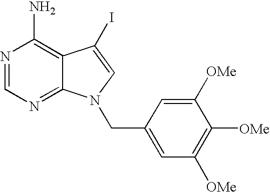

A mixture of 4-chloro-5-iodo-7-(4-nitro-benzyl)-7H-pyrrolo[2,3-d]pyrimidine (1 g) and conc. NH₄OH (15 mL) in dioxane (15 mL) was heated at 120° C. in a sealed tube for 4 h, allowed to cool to r.t. then treated with water (30 mL) and stirred for 1 h. The resulting precipitate was collected via filtration to give 5-iodo-7-(4-nitro-benzyl)-7H-pyrrolo[2,3-d]pyrimidin-4-ylamine (0.83 g, 87% yield); m/z: (M+H)⁺ 395.9.

### Preparation #40. 5-Iodo-7-(3,4,5-trimethoxybenzyl)-7H-pyrrolo[2,3-d]pyrimidin-4-ylamine

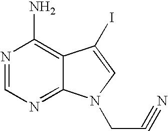

### Preparation #40.1. 4-Chloro-5-iodo-7-(3,4,5-trimethoxy-benzyl)-7H-pyrrolo[2,3-d]pyrimidine

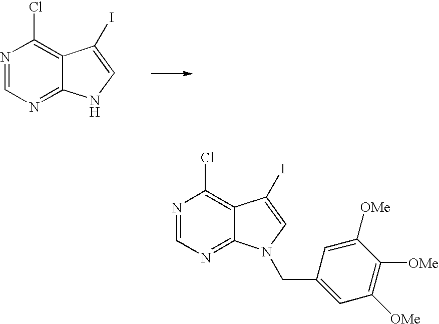

A solution of 4-chloro-5-iodo-7H-pyrrolo[2,3-d]pyrimidine (2.0 g, 7.16 mmol) in THF (75 mL) was sequentially treated with 3,4,5-trimethoxybenzyl alcohol (1.3 mL, 7.87 mmol), Ph₃P (3.8 g, 14.3 mmol), DIAD (2.91 mL, 14.3 mmol), stirred at r.t. for 18 h, then diluted with water and extracted with ether then CH₂Cl₂. The combined extracts were dried (MgSO₄), filtered and concentrated. The residue was triturated from ether then CH₂Cl₂ to give 4-chloro-5-iodo-7-(3,4,5-trimethoxy-benzyl)-7H-pyrrolo[2,3-d]pyrimidine (1.32 g); m/z: (M+H)⁺ 459.9.

4-Chloro-5-iodo-7-(3,4,5-trimethoxy-benzyl)-7H-pyrrolo[2,3-d]pyrimidine was reacted using the protocol detailed in the synthesis of preparation #39.2 to afford 5-iodo-7-(3,4,5-trimethoxy-benzyl)-7H-pyrrolo[2,3-d]pyrimidin-4-ylamine; m/z: (M+H)+ 441.2.

### Preparation #41. 3-Iodo-1-methyl-1H-pyrazolo[3,4-d]pyrimidin-4-ylamine

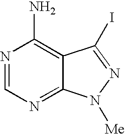

Substituting methyl iodide and 3-iodo-1H-pyrazolo[3,4-d]pyrimidin-4-ylamine for 4-nitrobenzyl bromide and 4-chloro-5-iodo-7H-pyrrolo[2,3-d]pyrimidine, respectively, in the synthesis of preparation #39.1 afforded 3-iodo-1-methyl-1H-pyrazolo[3,4-d]pyrimidin-4-ylamine; m/z: (M+H)⁺ 275.9.

### Preparation #42. 5-Iodo-7-methyl-7H-pyrrolo[2,3-d]pyrimidin-4-ylamine

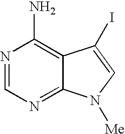

Substituting methyl iodide for 4-nitrobenzyl bromide in the synthesis of preparation #39 afforded 3-iodo-1-methyl-1H-pyrrolo[3,4-d]pyrimidin-4-ylamine; m/z: (M+H)⁺ 274.8.

### Preparation #43. N-[4-(4-Amino-5-iodo-pyrrolo[2,3-d]pyrimidin-7-ylmethyl)-phenyl]-methanesulfonamide

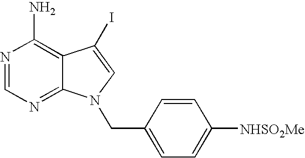

### Preparation 43.1. 7-(4-Amino-benzyl)-5-iodo-7H-pyrrolo[2,3-d]pyrimidin-4-ylamine

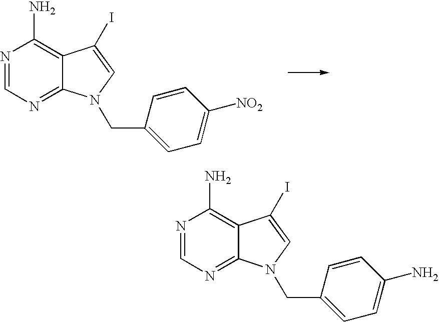

A mixture of 5-iodo-7-(4-nitrobenzyl)-7H-pyrrolo[2,3-d]pyrimidin-4-ylamine (preparation #39, 0.66 g, 1.67 mmol) and iron powder (0.28 g) in ethanol (10 mL) and water (5 mL) was stirred at 80° C. for 1 h, treated with an additional 0.1 g of iron powder, water (1 mL), THF (0.5 mL) and NH₄Cl (0.094 g) and stirred for another 4 h. The resulting suspension was filtered through celite washing with CH₂Cl₂ and methanol. The filtrate was washed with water, dried (MgSO₄), concentrated and the residue was purified via silica gel chromatography eluting with 3% MeOH:CH₂Cl₂ to give 7-(4-amino-benzyl)-5-iodo-7H-pyrrolo[2,3-d]pyrimidin-4-ylamine (0.33 g); m/z: (M+H)⁺ 365.9.

Methanesulfonyl chloride (0.033 mL) was added dropwise to a 0° C. solution of 7-(4-amino-benzyl)-5-iodo-7H-pyrrolo[2,3-d]pyrimidin-4-ylamine (0.15 g) in CH₂Cl₂ (10 mL) and pyridine (6 mL) and the resulting suspension was stirred at r.t for 19 h, then diluted with water. The precipitate was collected via filtration then dissolved in THF, dried (MgSO₄), filtered and concentrated to give N-[4-(4-amino-5-iodo-pyrrolo[2,3-d]pyrimidin-7-ylmethyl)-phenyl]-methanesulfonamide (0.13 g); m/z: (M+H)⁺ 443.8.

### Preparation #44. 1-[4-(4-Amino-5-iodo-pyrrolo[2,3-d]pyrimidin-7-ylmethyl)-phenyl]-3-(2-hydroxyethyl)urea

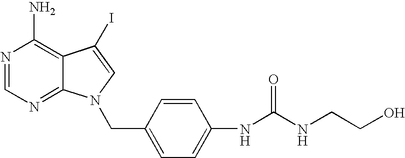

A cloudy solution of 7-(4-amino-benzyl)-5-iodo-7H-pyrrolo[2,3-d]pyrimidin-4-ylamine (0.154 g) and triethylamine (0.065 mL) in THF (8 mL) was treated with para-nitrophenylchloroformate (0.097 g) and stirred at 0° C. for 1.5 h. The reaction was then treated with ethanolamine (0.051 mL) and triethylamine (0.065 mL) and stirred at r.t. for 4 h. The mixture was diluted with water (20 mL) stirred at r.t. for 16 h, and the resulting precipitate was collected via filtration, washed with water, dried at 50° C. in a vacuum oven to give 1-[4-(4-amino-5-iodo-pyrrolo[2,3-d]pyrimidin-7-ylmethyl)-phenyl]-3-(2-hydroxy-ethyl)-urea (0.17 g); m/z: (M+H)⁺ 452.9.

### Preparation #45. (4-Amino-5-iodo-pyrrolo[2,3-d]pyrimnidin-7-yl)acetonitrile

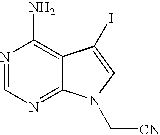

Substituting bromoacetonitrile for 4-nitrobenzyl bromide in the synthesis of preparation #39 afforded (4-amino-5-iodo-pyrrolo[2,3-d]pyrimidin-7-yl)-acetonitrile;); m/z: (M+H)⁺ 299.07.

### Preparation #46. 3-Iodo-1-pyridin-3-ylmethyl-1H-pyrazolo[3,4-d]pyrimidin-4-ylamine

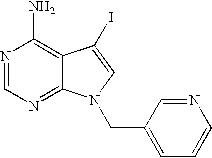

Substituting 3-bromomethylpyridine monohydrobromide for 4-nitrobenzyl bromide in the synthesis of preparation #39 afforded 3-iodo-1-pyridin-3-ylmethyl-1H-pyrazolo[3,4-d]pyrimidin-4-ylamine; m/z: (M+H)⁺ 351.9.

The following examples (#387 through #405) were synthesized by converting the bromides (for example, preparations #29 through #38) into the corresponding boronates using general procedure D and reacting the boronates with the iodides (for example, preparations #39 through #46) using general procedure C.

### Example #387

1-Cyclopentyl-3-[4-(5-methoxy-benzoxazol-2-ylamino)-phenyl]-1H-pyrazolo[3,4-d]pyrimidin-4-ylamine

### Example #388

3-[4-(5-Methoxy-benzoxazol-2-ylamino)-phenyl]-1-methyl-1H-pyrazolo[3,4-d]pyrimidin-4-ylamine

### Example #389

7-Methyl-5-{4-[5-methyl-7-(3-morpholin-4-yl-propoxy)-benzoxazol-2-ylamino]-phenyl}-7H-pyrrolo[2,3-d]pyrimidin-4-ylamine

### Example #390

Methyl-3-{4-[5-methyl-7-(3-morpholin-4-yl-propoxy)-benzoxazol-2-ylamino]-phenyl}-1H-pyrazolo[3,4-d]pyrimidin-4-ylamine

### Example #391

1-Methyl-3-{4-[5-methyl-7-(1-methyl-piperidin-4-ylmethoxy)-benzoxazol-2-ylamino]-phenyl}-1H-pyrazolo[3,4-d]pyrimidin-4-ylamine

### Example #392

1-Cyclopentyl-3-{4-[5-methyl-7-(2-pyrrolidin-1-yl-ethoxy)-benzoxazol-2-ylamino]-phenyl}-1H-pyrazolo[3,4-d]pyrimidin-4-ylamine

### Example #393

1-Methyl-3-{4-[5-methyl-7-(2-pyrrolidin-1-yl-ethoxy)-benzoxazol-2-ylamino]-phenyl}-1H-pyrazolo[3,4-d]pyrimidin-4-ylamine

### Example #394

7-Cyclopentyl-5-{4-[5-methyl-7-(3-morpholin-4-yl-propyl)-benzoxazol-2-ylamino]-phenyl}-7H-pyrrolo[2,3-d]pyrimidin-4-ylamine

### Example #395

7-Methyl-5-{4-[5-methyl-7-(3-morpholin-4-yl-propyl)-benzoxazol-2-ylamino]-phenyl}-7H-pyrrolo[2,3-d]pyrimidin-4-ylamine

### Example #396

3-[4-(7-Allyl-5-methyl-benzoxazol-2-ylamino)-phenyl]-1-cyclopentyl-1H-pyrazolo[3,4-d]pyrimidin-4-ylamine

### Example #397

1-Cyclopentyl-3-{4-[7-(2-dimethylamino-ethoxy)-5-methyl-benzoxazol-2-ylamino]-phenyl}-1H-pyrazolo[3,4-d]pyrimidin-4-ylamine

### Example #398

2-[4-(4-Amino-1-cyclopentyl-1H-pyrazolo[3,4-d]pyrimidin-3-yl)-phenylamino]-5-methyl-benzoxazol-7-ol

### Example #399

N-(4-{4-Amino-5-[4-(5,7-dimethyl-benzoxazol-2-ylamino)-phenyl]-pyrrolo[2,3-d]pyrimidin-7-ylmethyl}-phenyl)-methanesulfonamide

### Example #400

1-(4-{4-Amino-5-[4-(5,7-dimethyl-benzoxazol-2-ylamino)-phenyl]-pyrrolo[2,3-d]pyrimidin-7-ylmethyl}-phenyl)-3-(2-hydroxy-ethyl)-urea

### Example #401

5-[4-(5,7-Dimethyl-benzoxazol-2-ylamino)-phenyl]-7-(3,4,5-trimethoxy-benzyl)-7H-pyrrolo[2,3-d]pyrimidin-4-ylamine

### Example #402

5-[4-(5,7-Dimethyl-benzoxazol-2-ylamino)-phenyl]-7-(4-nitro-benzyl)-7H-pyrrolo[2,3-d]pyrimidin-4-ylamine

### Example #403

{4-Amino-5-[4-(5,7-dimethyl-benzoxazol-2-ylamino)-phenyl]-pyrrolo[2,3-d]pyrimidin-7-yl}-acetonitrile

### Example #404

5-[4-(5,7-Dimethyl-benzoxazol-2-ylamino)-3-fluoro-phenyl]-7-methyl-7H-pyrrolo[2,3-d]pyrimidin-4-ylamine

### Example #405

5-[4-(5,7-Dimethyl-benzoxazol-2-ylamino)-3-fluoro-phenyl]-7-pyridin-3-ylmethyl-7H-pyrrolo[2,3-d]pyrimidin-4-ylamine

The method used to determine the HPLC retention time is given in a lower-case letter in parentheses (see Table 1).

TABLE 13
Summary of the synthesis and analytical data for
examples 387 to 405

| | | | NMR 300 MHz(d₆- | m/z |
|---|---|---|---|---|
| Iodide | Bromide | Example # | DMSO)/LC-RT (method) | (M + H)⁺ |
| 4-Cyclopentyl-3-iodo- | Preparation | 387 | 0.59 min (h) | 442 |
| 1H-pyrazolo[3,4- | #34 | | | |
| d]pyrimidin-4-ylamine | | | | |
| (WO 01019829) | | | | |
| Preparation #41 | Preparation | 388 | 0.26 min (h) | 388 |
| | #34 | | | |
| Preparation #42 | Preparation | 389 | 1.97 min (h) | 514 |
| | #29 | | | |
| Preparation #41 | Preparation | 390 | 1.91 min (h) | 515 |
| | #29 | | | |
| 4-Cyclopentyl-3-iodo- | Preparation | 391 | 0.71 min (h) | 499 |
| 1H-pyrazolo[3,4- | #30 | | | |
| d]pyrimidin-4-ylamine | | | | |
| (WO 01019829) | | | | |
| 4-Cyclopentyl-3-iodo- | Preparation | 392 | 1.70(m, 4H) 1.90(m, 2H) | 539 |
| 1H-pyrazolo[3,4- | #35 | | 2.08(m, 4H) 2.36(m, | |
| d]pyrimidin-4-ylamine | | | 3H) 2.56(m, 4H) 2.84(t, | |
| (WO 01019829) | | | J=5.93Hz, 2H) 4.25(t, | |
| | | | J=5.76Hz, 2H) 5.23(m, | |
| | | | 1H) 6.69(s, 1H) 6.90(s, | |
| | | | 1H) 7.65(d, J=8.82Hz, 2H) | |
| | | | 7.89(d, J=8.48Hz, 2H) | |
| | | | 8.23(s, 1H) 10.89(s, | |
| | | | 1H) | |
| Preparation #41 | Preparation | 393 | 1.70(m, J=6.87, 3.31, | 485 |
| | #35 | | 3.05Hz, 4H) 2.36(s, 3H) | |
| | | | 2.55(m, 46H) 2.84(t, | |
| | | | J=5.76Hz, 2H) 3.95(s, 3H) | |
| | | | 4.25(t, J=5.93Hz, 2H) | |
| | | | 6.69(s, 1H) 6.90(s, 1H) | |
| | | | 7.66(d, J=8.81Hz, 2H) | |
| | | | 7.89(d, J=8.48Hz, 2H) | |
| | | | 8.25(s, 1H) 10.90(s, 1H) | |
| 7-Cyclopentyl-5-iodo- | Preparation | 394 | 1.79(m, 8H), 2.11(m, 2H), | 552.2 |
| 7H-pyrrolo[2,3- | #32 | | 2.35(s, 9H), 2.76(m, | |
| d]pyrimidin-4-ylamine | | | 2H), 3.57(m, 4H), 5.08(m, | |
| (WO 01019829) | | | 1H), 6.03(br. s., 2H), | |
| | | | 6.80(s, 1H), 7.10(s, 1H), | |
| | | | 7.36(s, 1H), 7.47(d, | |
| | | | J=8.82Hz, 2H), 7.85(d, | |
| | | | J=8.48Hz, 2H), 8.13(s, 1H), | |
| | | | 10.67(s, 1H). | |
| Preparation #42 | Preparation | 395 | 1.42 min (h) | 498.1 |
| | #32 | | | |
| 4-Cyclopentyl-3-iodo- | Preparation | 396 | 2.18 min (h) | 466.1 |
| 1H-pyrazolo[3,4- | #31 | | | |
| d]pyrimidin-4-ylamine | | | | |
| (WO 01019829) | | | | |
| 4-Cyclopentyl-3-iodo- | Preparation | 397 | 1.74 min (h) | 513.2 |
| 1H-pyrazolo[3,4- | #36 | | | |
| d]pyrimidin-4-ylamine | | | | |
| (WO 01019829) | | | | |
| 4-Cyclopentyl-3-iodo- | Preparation | 398 | 1.92 min (h) | 442.1 |
| 1H-pyrazolo[3,4- | #33 | | | |
| d]pyrimidin-4-ylamine | | | | |
| (WO 01019829) | | | | |
| Preparation #43 | N-(4- | 399 | 1.96 min (h) | 554.2 |
| | Bromophenyl)- | | | |
| | (5,7-dimethyl- | | | |
| | benzoxazol-2- | | | |
| | yl)amine(G) | | | |
| Preparation #44 | N-(4- | 400 | 1.82 min (h) | 563.2 |
| | Bromophenyl)- | | | |
| | (5,7-dimethyl- | | | |
| | benzoxazol-2- | | | |
| | yl)amine(G) | | | |
| Preparation #40 | N-(4- | 401 | 2.34(s, 3H), 2.39(s, 3H), | 551.2 |
| | Bromophenyl)- | | 3.62(s, 3H), 3.72(s, | |
| | (5,7-dimethyl- | | 6H), 5.29(s, 2H), | |
| | benzoxazol-2- | | 6.08(br. s., 2H), 6.73(s, 2H), | |
| | yl)amine(G) | | 6.78(s, 1H), 7.09(s, 1H), | |
| | | | 7.38(s, 1H), 7.45(d, | |
| | | | J=8.48Hz, 2H), 7.84(d, | |
| | | | J=8.81Hz, 2H), 8.20(s, 1H), | |
| | | | 10.71(s, 1H). | |
| Preparation #39 | N-(4- | 402 | 2.34(s, 3H), 2.40(s, 3H), | 506.1 |
| | Bromophenyl)- | | 5.55(s, 2H), 6.16(br. | |
| | (5,7-dimethyl- | | s., 2H), 6.78(s, 1H), | |
| | benzoxazol-2- | | 7.09(s, 1H), 7.43(s, 1H), | |
| | yl)amine(G) | | 7.48(m, 4H), 7.85(d, | |
| | | | J=8.82Hz, 2H), 8.16(s, 1H), | |
| | | | 8.21(m, 2H), 10.73(s, 1H). | |
| Preparation #45 | N-(4- | 403 | 2.34(s, 3H), 2.40(s, 3H), | 410.1 |
| | Bromophenyl)- | | 5.41(s, 2H), 6.23(br. | |
| | (5,7-dimethyl- | | s., 2H), 6.78(s, 1H), | |
| | benzoxazol-2- | | 7.10(s, 1H), 7.37(s, 1H), | |
| | yl)amine(G) | | 7.47(d, J=8.82Hz, 2H), | |
| | | | 7.87(d, J=8.48Hz, 2H), | |
| | | | 8.23(s, 1H), 10.76(s, | |
| | | | 1H). | |
| Preparation #42 | N-(4-Bromo-2- | 404 | 10.42(s, 1H); 8.34(t, 1H, | 403.1 |
| | fluoro-phenyl)- | | J=7.5); 8.16(s, 1H); 7.30-7.39(m, | |
| | (5,7-dimethyl- | | 3H); 7.21(s, 1H); | |
| | benzoxazol-2- | | 6.79(s, 1H); 6.18(s, 1H); | |
| | yl)amine(G) | | 3.75(s, 3H); 2.40(s, 3H); | |
| | | | 2.33(s, 3H). | |
| Preparation #46 | N-(4-Bromo-2- | 405 | 10.44(bs, 1H); 8.61(d, | 480.1 |
| | fluoro-phenyl)- | | 1H, J=4); 8.49(dd, 1H, | |
| | (5,7-dimethyl- | | J=4.8); 8.34(t, 1H, J=8); | |
| | benzoxazol-2- | | 8.19(s, 1H); 7.71(dt, 1H, | |
| | yl)amine(G) | | J=2.8); 7.52(s, 1H); 7.30-7.38(m, | |
| | | | 3H); 7.06(s, 1H); | |
| | | | 6.77(s, 1H); 6.24(bs, 2H); | |
| | | | 5.44(s, 2H); 2.39(s, 3H); | |
| | | | 2.33(s, 3H). | |

### Example #406

7-(4-Aminobenzyl)-5-[4-(5,7-dimethyl-benzoxazol-2-ylamino)-phenyl]-7H-pyrrolo[2,3-d]pyrimidin-4-ylamine

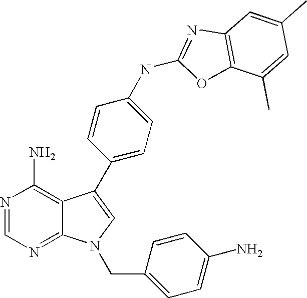

Reduction of example #402, using the general procedure H, afforded 7-(4-amino-benzyl)-5-[4-(5,7-dimethyl-benzoxazol-2-ylamino)-phenyl]-7H-pyrrolo[2,3-d]pyrimidin-4-ylamine; ¹H NMR (300 MHz, DMSO-d₆) δ 2.34 (s, 3 H), 2.39 (s, 3 H), 5.04 (s, 2 H), 5.16 (s, 2 H), 6.04 (br s, 2 H), 6.49 (d, J=8.48 Hz, 2 H), 6.77 (s, 1 H), 7.04 (d, J=8.48 Hz, 2 H), 7.09 (s, 1 H), 7.26 (s, 1 H), 7.42 (d, J=8.48 Hz, 2 H), 7.83 (d, J=8.81 Hz, 2 H), 8.17 (s, 1 H), 10.71 (s, 1 H); ); m/z: (M+H)⁺ 476.2

### Preparations #47-54. Aminobenzoxazole phenolic analogs

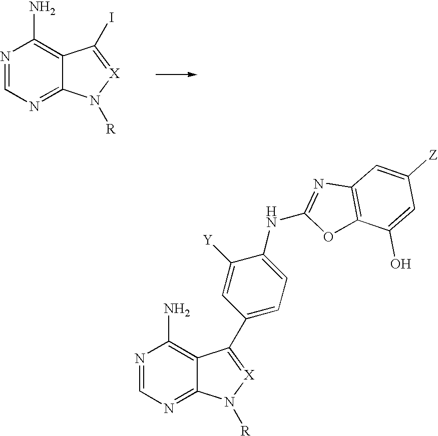

List of aminobenzoxazole phenol analogs (preparations # 47-54) were synthesized using the procedure detailed to prepare example #387 using reactants detailed in Table 14. The method used to determine the HPLC retention time is given in a lower-case letter in parentheses (see Table 1).

TABLE 14
Preparations #47 through #54

| | | | | | | | m/z |
|---|---|---|---|---|---|---|---|
| Iodide | Bromide | Phenol | R | X | Y | Z | (M + H)⁺ |
| Preparation # 42 | Preparation #33 | Preparation #47 | Me | CH | H | Me | 387.1 |
| 4-Cyclopentyl-3- | Preparation #37 | Preparation #48 | Cyclopentyl | N | H | Cl | 462 |
| iodo-1H- | | | | | | | |
| pyrazolo[3,4- | | | | | | | |
| d]pyrimidin-4- | | | | | | | |
| ylamine (WO | | | | | | | |
| 01019829) | | | | | | | |
| Preparation #42 | Preparation #37 | Preparation #49 | Me | CH | H | Cl | 405 (−) |
| Preparation #41 | Preparation #37 | Preparation #50 | Me | N | H | Cl | 406 (−) |
| 7-Cyclopentyl-5- | Preparation #37 | Preparation #51 | Cyclopentyl | CH | H | Cl | 461 |
| iodo-7H-pyrrolo[2,3- | | | | | | | |
| d]pyrimidin-4- | | | | | | | |
| ylamine (WO | | | | | | | |
| 01019829) | | | | | | | |
| Preparation #42 | Preparation #38 | Preparation #52 | Me | CH | F | Cl | 425 |
| 7-Cyclopentyl-5- | Preparation #38 | Preparation #53 | Cyclopentyl | CH | F | Cl | 479.2 |
| iodo-7H-pyrrolo[2,3- | | | | | | | |
| d]pyrimidin-4- | | | | | | | |
| ylamine (WO | | | | | | | |
| 01019829) | | | | | | | |
| trans-3-Iodo-1-(4- | Preparation #37 | Preparation #54 | trans- | N | H | Cl | 559.1 (−) |
| morpholin-4-yl- | | | cyclohexyl- | | | | |
| cyclohexyl)-1H- | | | morpholine | | | | |
| pyrazolo[3,4- | | | | | | | |
| d]pyrimidin-4-yl | | | | | | | |
| amine (A,T,J) | | | | | | | |

### Example #407

1-Cyclopentyl-3-{4-[5-methyl-7-(2-morpholin-4-yl-ethoxy)-benzoxazol-2-ylamino]-phenyl}-1H-pyrazolo[3,4-d]pyrimidin-4-ylamine

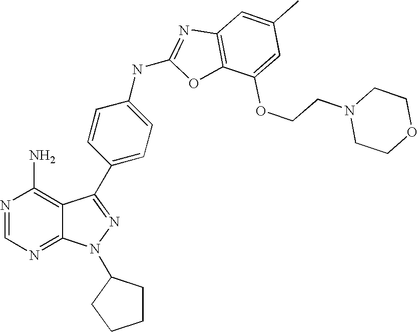

A solution of example #398 (0.16 g, 0.36 mmol), 4-(2-chloroethyl)morpholine (0.075 g, 0.4 mmol) and triethylamine (0.106 mL, 0.76 mmol) in DMF (15 mL) was treated with Cs₂CO₃ (130 mg), stirred at 50° C. for 3 h. An additional amount of Cs₂CO₃ (260 mg) was added and stirred at 50° C. for a further 2.5 h. The reaction mixture was cooled to r.t., diluted with water and extracted with ether 3 times. The combined extracts were dried (MgSO₄), concentrated and the residue was purified via silica gel chromatography eluting with hexanes: EtOAc: methanol: CH₂Cl₂ (5:4:1:1) to give 1-cyclopentyl-3-{4-[5-methyl-7-(2-morpholin-4-yl-ethoxy)-benzoxazol-2-ylamino]-phenyl}-1H-pyrazolo[3,4-d]pyrimidin-4-ylamine (45 mg, 22% yield); RP-HPLC (5% to 95% acetonitrile/0.1% H₃PO₄ (aq), over 7 minutes at 1.5 mL/min; λ=190-700 nm; Zorbax SB-C8 rapid resolution, 4.6 mm×75 mm, 3.5 μm column) Rₜ 1.76 min; m/z: (M+H)⁺ 555.2

Examples #408-428 were prepared via an alkylation of the corresponding phenol with an alkylating agent as described for preparation #29. 1.

### Example #408

1-Cyclopentyl-3-{4-[5-methyl-7-(2-morpholin-4-yl-ethoxy)-benzoxazol-2-ylamino]-phenyl}-1H-pyrazolo[3,4-d]pyrimidin-4-ylamine

### Example #409

4-{2-[4-(4-Amino-1-cyclopentyl-1H-pyrazolo[3,4-d]pyrimidin-3-yl)-phenylamino]-5-methyl-benzoxazol-7-yloxymethyl}-piperidine-1-carboxylic acid tert-butyl ester monotrifluoroacetate

### Example #410

7-Methyl-5-{4-[5-methyl-7-(2-morpholin-4-yl-ethoxy)-benzoxazol-2-ylamino]-phenyl}-7H-pyrrolo[2,3-d]pyrimidin-4-ylamine

### Example #411

5-{4-[7-(2-Dimethylamino-ethoxy)-5-methyl-benzoxazol-2-ylamino]-phenyl}-7-methyl-7H-pyrrolo[2,3-d]pyrimidin-4-ylamine

### Example #412

3-{4-[5-Chloro-7-(2-dimethylamino-ethoxy)-benzoxazol-2-ylamino]-phenyl}-1-cyclopentyl-1H-pyrazolo[3,4-d]pyrimidin-4-ylamine

### Example #413

3-{4-[5-Chloro-7-(2-methoxy-ethoxy)-benzoxazol-2-ylamino]-phenyl}-1-cyclopentyl-1H-pyrazolo[3,4-d]pyrimidin-4-ylamine

### Example #414

3-{4-[5-Chloro-7-(2-morpholin-4-yl-ethoxy)-benzoxazol-2-ylamino]-phenyl}-1-cyclopentyl-1H-pyrazolo[3,4-d]pyrimidin-4-ylamine

### Example #415

2-{2-[4-(4-Amino-1-cyclopentyl-1H-pyrazolo[3,4-d]pyrimidin-3-yl)-phenylamino]-5-chloro-benzoxazol-7-yloxy}-N,N-diethyl-acetamide

### Example #416

3-{4-[5-Chloro-7-(2-pyrrolidin-1-yl-ethoxy)-benzoxazol-2-ylamino]-phenyl}-1-cyclopentyl-1H-pyrazolo[3,4-d]pyrimidin-4-ylamine

### Example #417

5-{4-[5-Chloro-7-(2-morpholin-4-yl-ethoxy)-benzoxazol-2-ylamino]-phenyl}-7-methyl-7H-pyrrolo[2,3-d]pyrimidin-4-ylamine

### Example #418

3-{4-[5-Chloro-7-(2-morpholin-4-yl-ethoxy)-benzoxazol-2-ylamino]-phenyl}-1-methyl-1H-pyrazolo[3,4-d]pyrimidin-4-ylamine

### Example #419

5-{4-[5-Chloro-7-(5-chloro-thiophen-2-ylmethoxy)-benzoxazol-2-ylamino]-phenyl}-7-methyl-7H-pyrrolo[2,3-d]pyrimidin-4-ylamine

### Example #420

3-{4-[5-Chloro-7-(2-phenylsulfanyl-ethoxy)-benzoxazol-2-ylamino]-phenyl}-1-methyl-1H-pyrazolo[3,4-d]pyrimidin-4-ylamine

### Example #421

3-{4-[5-Chloro-7-(6-chloro-pyridin-3-ylmethoxy)-benzoxazol-2-ylamino]-phenyl}-1-methyl-1H-pyrazolo[3,4-d]pyrimidin-4-ylamine

### Example #422

5-{4-[5-Chloro-7-(3-morpholin-4-yl-propoxy)-benzoxazol-2-ylamino]-phenyl}-7-cyclopentyl-7H-pyrrolo[2,3-d]pyrimidin-4-ylamine

### Example #423

3-{4-[5-Chloro-7-(3-morpholin-4-yl-propoxy)-benzoxazol-2-ylamino]-phenyl}-1-cyclopentyl-1H-pyrazolo[3,4-d]pyrimidin-4-ylamine

### Example #424

5-{4-[5-Chloro-7-(3-morpholin-4-yl-propoxy)-benzoxazol-2-ylamino]-phenyl}-7-methyl-7H-pyrrolo[2,3-d]pyrimidin-4-ylamine

### Example #425

5-{4-[5-Chloro-7-(3-morpholin-4-yl-propoxy)-benzoxazol-2-ylamino]-3-fluoro-phenyl}-7-methyl-7H-pyrrolo[2,3-d]pyrimidin-4-ylamine

### Example #426

5-{4-[5-Chloro-7-(3-morpholin-4-yl-propoxy)-benzoxazol-2-ylamino]-3-fluoro-phenyl}-7-cyclopentyl-7H-pyrrolo[2,3-d]pyrimidin-4-ylamine

### Example #427

3-{4-[5-Chloro-7-(2-morpholin-4-yl-ethoxy)-benzoxazol-2-ylamino]-phenyl}-1-(4-morpholin-4-yl-cyclohexyl)-1H-pyrazolo[3,4-d]pyrimidin-4-ylamine

### Example #428

3-{4-[5-Chloro-7-(3-morpholin-4-yl-propoxy)-benzoxazol-2-ylamino]-phenyl}-1-(4-morpholin-4-yl-cyclohexyl)-1H-pyrazolo[3,4-d]pyrimidin-4-ylamine

The method used to determine the HPLC retention time is given in a lower-case letter in parentheses (see Table 1).

| | | | NMR 300 MHz (d₆-DMSO)/LC-RT | m/z |
|---|---|---|---|---|
| Phenol | Alkyl-X | Example # | (method) | (M + H)⁺ |
| Example | Cl(CH₂)₂morpholine | 408 | 1.76 (h) | 555.2 |
| #398 | | | | |
| Example | 1-Boc-4- | 409 | 2.47 (h) | 639.3 |
| #398 | (TsOCH₂)piperidine | | | |
| Preparation | Cl(CH₂)₂morpholine | 410 | 2.36(s, 3H), 2.49(m, 4H), 2.74(t, | 500.1 |
| #47 | | | J=5.59Hz, 2H), 3.59(t, J=4.59Hz, 4H), | |
| | | | 3.74(s, 3H), 4.27(t, J=5.59Hz, 2H), | |
| | | | 6.06(br. s., 2H), 6.69(s, 1H), 6.89(s, | |
| | | | 1H), 7.26(s, 1H), 7.45(d, J=8.82Hz, | |
| | | | 2H), 7.82(d, J=8.48Hz, 2H), 8.15(s, | |
| | | | 1H), 10.78(s, 1H). | |
| Preparation | Cl(CH₂)₂NMe₂ | 411 | 1.42 (h) | 458.1 |
| #47 | | | | |
| Preparation | Cl(CH₂)₂NMe₂ | 412 | 11.11(s, 1H); 8.23(s, 1H); 7.89(s, 1H); | 531.1 |
| #48 | | | 7.88(s, 1H); 7.68(s, 1H); 7.66(s, 1H); | |
| | | | 5.23(m, 1H, J=7.5); 4.27(t, 2H, J=5); | |
| | | | 2.67(t, 2H, J=5); 2.24(s, 6H); 2.01-2.13(m, | |
| | | | 4H); 1.87-1.96(m, 2H); 1.65-1.74(m, 2H). | |
| Preparation | Br(CH₂)₂OMe | 413 | 11.11(s, 1H,); 8.23(s, 1H); 7.89(s, 1H); | 518.1 |
| #48 | | | 7.86(s, 1H); 7.68(s, 1H); 7.65(s, 1H); | |
| | | | 7.19(d, 1H, J=3); 6.95(d, 1H, J=3); | |
| | | | 5.23(m, 1H, J=6); 4.30-4.36(m, 2H); | |
| | | | 3.68-3.75(m, 2H); 2.00-2.15(m, 4H); | |
| | | | 1.83-1.98(m, 2H); 1.63-1.78(m, 2H). | |
| Preparation | Cl(CH₂)₂morpholine | 414 | 11.11(s, 1H); 8.23(s, 1H); 7.90(s, 1H); | 575.1 |
| #48 | | | 7.87(s, 1H); 7.68(s, 1H); 7.65(s, 1H); | |
| | | | 7.17(d, 1H, J=3); 6.97(d, 1H, J=3); | |
| | | | 5.23(m, 1H, J=7.5); 4.23(t, 2H, J=4.5); | |
| | | | 3.56-3.62(m, 4H); 2.72-2.78(m, 2H); | |
| | | | 2.54-2.58(m, 1H); 2.40-2.46(m, 1H); | |
| | | | 2.26-2.30(m, 1H); 2.00-2.13(m, 4H); | |
| | | | 1.85-1.95(m, 2H); 1.63-1.76(m, 2H). | |
| Preparation | ClCH₂CONEt₂ | 415 | 11.09(s, 1H); 8.23(s, 1H); 7.90(s, 1H); | 575.1 |
| #48 | | | 7.87(s, 1H); 7.69(s, 1H); 7.66(s, 1H); | |
| | | | 7.18(d, 1H, J=1.5); 6.89(d, 1H, J=1.5); | |
| | | | 5.23(m, 1H, J=6); 5.03(s, 2H); 2.00-2.17(m, | |
| | | | 4H); 1.83-1.98(m, 2H); 1.62-1.77(m, | |
| | | | 2H); 1.18(t, 3H, J=6); 1.05(t, | |
| | | | 3H, J=6) | |
| Preparation | Cl(CH₂)₂pyrrolidine | 416 | 11.10(s, 1H); 8.23(s, 1H); 7.90(s, 1H); | 557.1 |
| #48 | | | 7.87(s, 1H); 7.68(s, 1H); 7.65(s, 1H); | |
| | | | 7.18(d, 1H, J=1.5); 6.97(d, 1H, J=1.5); | |
| | | | 5.23(m, 1H, J=6); 4.27-4.36(m, 2H); | |
| | | | 2.80-2.96(m, 2H); 2.57-2.65(m, 2H); | |
| | | | 2.00-2.17(m, 4H); 1.84-1.98(m, 2H); | |
| | | | 1.64-1.79(m, 6H) | |
| Preparation | Cl(CH₂)₂morpholine | 417 | 2.18 (h) | 520.1 |
| #49 | | | | |
| Preparation | Cl(CH₂)₂morpholine | 418 | 11.12(s, 1H); 8.25(s, 1H); 7.90(s, 1H); | 521.0 |
| #50 | | | 7.87(s, 1H); 7.69(s, 1H); 7.66(s, 1H); | |
| | | | 7.18(d, 1H, J=1.5); 6.98(d, 1H, J=1.5); | |
| | | | 4.28-4.36(m, 2H); 3.95(s, 3H); 3.56-3.64(m, | |
| | | | 4H); 2.71-2.79(m, 2H). | |
| Preparation | 4-(ClCH₂)-2-chloro- | 418 | 10.97(s, 1H); 8.15(s, 1H); 7.82(s, 1H); | 536.9 |
| #49 | thiophene | | 7.79(s, 1H); 7.53(s, 1H); 7.48(s, 1H); | |
| | | | 7.27(s, 1H); 7.20(d, 1H, J=1.5); 7.16(d, | |
| | | | 1H, J=1.5); 7.07-7.11(m, 2H); 6.04(bs, | |
| | | | 2H); 5.47(s, 2H); 3.74(s, 3H). | |
| Preparation | Br(CH₂)₂SPh | 420 | 11.13(s, 1H); 8.26(s, 1H); 7.90(s, 1H); | 544.0 |
| #50 | | | 7.87(s, 1H); 7.69(s, 1H); 7.66(s, 1H); | |
| | | | 7.42-7.45(m, 1H); 7.40-7.42(m, 1H); | |
| | | | 7.30-7.37(m, 2H); 7.17-7.26(m, 2H); | |
| | | | 6.91-6.95(m, 1H); 4.39(t, 2H, J=6); | |
| | | | 3.95(s, 3H); 3.43(t, 2H, J=6). | |
| Preparation | 2-chloro-5-(ClCH₂)- | 421 | 11.12(s, 1H); 8.57(d, 1H, J=3); 8.25(s, | 532.9 |
| #50 | pyridyl | | 1H); 7.99(dd, 1H, J=3, 7.5); 7.89(s, 1H); | |
| | | | 7.86(s, 1H); 7.69(s, 1H); 7.60-7.67(m, | |
| | | | 2H); 7.23(m, 2H); 7.10(d, 1H, J=1.5); | |
| | | | 5.38(s, 2H); 3.95(s, 3H). | |
| Preparation | Cl(CH₂)₃morpholine | 422 | 10.97(s, 1H); 8.12(s, 1H); 7.82(s, 1H); | 588.2 |
| #51 | | | 7.79(s, 1H); 7.49(s, 1H); 7.47(s, 1H); | |
| | | | 7.36(s, 1H); 7.16(d, 1H, J=1.5); 6.92(d, | |
| | | | 1H, J=1.5); 6.03(bs, 2H); 5.04-5.14(m, | |
| | | | 1H); 4.24(t, 2H, J=6); 3.58(4H, J=4.5); | |
| | | | 2.39-2.56(m, 6H); 2.04-2.18(m, 2H); | |
| | | | 1.82-2.00(m, 6H); 1.64-1.77(m, 2H). | |
| Preparation | Cl(CH₂)₃morpholine | 423 | 11.10(s, 1H); 8.23(s, 1H); 7.89(s, 1H); | 589.2 |
| #48 | | | 7.87(s, 1H); 7.68(s, 1H); 7.65(s, 1H); | |
| | | | 7.17(d, 1H, J=1.5); 6.94(d, 1H, J=1.5); | |
| | | | 5.23(m, 1H, J=7.5); 4.24(t, 2H, J=6); | |
| | | | 3.58(t, 2H, J=4.5); 3.23-3.40(m, 2H); | |
| | | | 2.36-2.59(m, 4H); 2.02-2.15(m, 4H); | |
| | | | 1.85-1.99(m, 4H); 1.65-1.77(m, 2H). | |
| Preparation | Cl(CH₂)₃morpholine | 424 | 11.02(s, 1H); 8.39(s, 1H); 7.87(s, 1H); | 534.1 |
| #49 | | | 7.84(s, 1H); 7.48-7.55(m, 2H); 7.47(s, | |
| | | | 1H); 7.20(d, 1H, J=1.5); 6.97(d, 1H, | |
| | | | J=1.5); 4.32(t, 2H, J=6); 3.95-4.09(m, | |
| | | | 2H); 3.83(s, 3H); 3.60-3.70(m, 2H); 3.24-3.45(m, | |
| | | | 2H); 3.07-3.21(m, 4H); 1.51-1.64(m, 2H). | |
| Preparation | Cl(CH₂)₃morpholine | 425 | 10.70(s, 1H); 8.28(t, 1H, J=7.5); 8.16(s, | 552.1 |
| #52 | | | 1H); 7.33-7.40(m, 2H); 7.32(s, 1H); | |
| | | | 7.14(d, 1H, J=1.5); 6.92(d, 1H, J=1.5); | |
| | | | 6.17(bs, 2H); 4.24(t, 2H, J=6); 3.74(s, | |
| | | | 3H); 3.58(t, 2H, J=6); 2.41-2.47(m, 2H); | |
| | | | 2.32-2.40(m, 4H); 1.87-1.97(m, 2H). | |
| Preparation | Cl(CH₂)₃morpholine | 426 | 8.22-8.32(m, 1H); 8.14(s, 1H); 7.48(s, | 606.1 |
| #53 | | | 1H); 7.31-7.44(m, 2H); 7.13(s, 1H); | |
| | | | 6.93(s, 1H); 6.16(bs, 2H); 5.02-5.15(m, | |
| | | | 1H); 4.24(t, 2H, J=6); 3.58(t, 2H, J=4.5); | |
| | | | 3.20-3.37(m, 4H); 2.32-2.54(m, 4H); | |
| | | | 2.04-2.19(m, 2H); 1.82-2.00(m, 4H); | |
| | | | 1.64-1.77(m, 2H). | |
| Preparation | Cl(CH₂)₂morpholine | 427 | 11.11(s, 1H); 8.23(s, 1H); 7.89(s, 1H); | 672.2 |
| #54 | | | 7.87(s, 1H); 7.67(s, 1H); 7.64(s, 1H); | |
| | | | 7.18(d, 1H, J=1.5); 6.98(d, 1H, J=1.5); | |
| | | | 4.65(bs, 1H); 4.32(t, 2H, J=6); 3.56-3.64(m, | |
| | | | 8H); 2.72-2.79(m, 2H); 1.94-2.13(m, | |
| | | | 6H); 1.40-1.58(m, 2H). | |
| Preparation | Cl(CH₂)₃morpholine | 428 | 11.11(bs, 1H); 8.22(s, 1H); 7.88(s, 1H); | 688.2 |
| #54 | | | 7.85(s, 1H); 7.66(s, 1H); 7.63(s, 1H); | |
| | | | 7.15(s, 1H); 6.91(s, 1H); 4.69-4.72(m, | |
| | | | 1H); 4.24(t, 2H, J=6); 3.75-3.83(m, 9H); | |
| | | | 2.30-2.46(m, 8H); 1.88-2.10(m, 10H); | |
| | | | 1.38-1.56(m, 2H). | |

### Example #429

1-Cyclopentyl-3-{4-[5-methyl-7-(piperidin-4-ylmethoxy)-benzoxazol-2-ylamino]-phenyl}-1H-pyrazolo[3,4-d]pyrimidin-4-ylamine

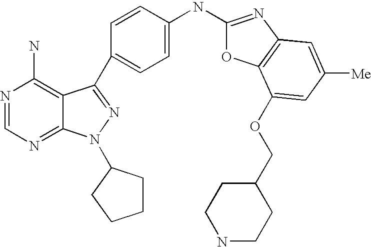

A 0° C. solution of example #409 (0.08 g) in CH₂Cl₂ (4 mL) and TFA (1 mL) was stirred at 0° C. for 1 h, then at r.t. for 4 h, before concentrating. The residue was triturated with ether and the precipitate was collected, dried under vacuum for 24 h to give 1-cyclopentyl-3-{4-[5-methyl-7-(piperidin-4-ylmethoxy)-benzoxazol-2-ylamino]-phenyl}-1H-pyrazolo[3,4-d]pyrimidin-4-ylamine bis-trifluoroacetate (52.7 mg); RP-HPLC (5% to 95% acetonitrile/0.1% H₃PO₄ (aq), over 7 minutes at 1.5 mL/min; λ=190-700 nm; Zorbax SB-C8 rapid resolution,4.6 mm×75 mm, 3.5 μm column) Rt 1.74 min; m/z: (M+H)⁺ 539.1

### Example #430

5-[4-(5,7-Dimethyl-benzoxazol-2-ylamino)-phenyl]-7H-pyrrolo[2,3-d]pyrimidin-4-ylamine

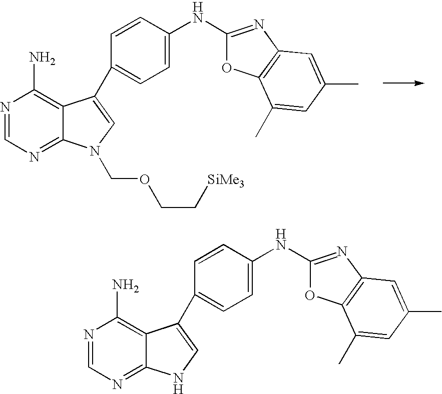

3-Iodo-1-(2-trimethylsilanyl-ethoxymethyl)-1H-pyrazolo[3,4-d]pyrimidin-4-ylamine (as detailed in the synthesis of Example #362) was reacted with (5,7-dimethyl-benzoxazol-2-yl)-[2-ethyl-4-(4,4,5,5-tetramethyl-[1,3,2]dioxaborolan-2-yl)-phenyl]-amine (G,D) using general procedure C to afford 5-[4-(5,7-dimethyl-benzoxazol-2-ylamino)-phenyl]-7-(2-trimethylsilanyl-ethoxymethyl)-7H-pyrrolo[2,3-d]pyrimidin-4-ylamine. A solution of this compound (0.128 g) in THF (20 mL) was treated with TBAF (5 mL, 1M in THF) stirred at reflux for 15 h, cooled to r.t. and partitioned between saturated aqueous NH₄Cl and ether (2×). The combined organic extracts were dried (MgSO₄), concentrated and the residue was purified via silica gel chromatography eluting with 5:4:1 hexanes:EtOAc:methanol to give 5-[4-(5,7-dimethyl-benzoxazol-2-ylamino)-phenyl]-7H-pyrrolo[2,3-d]pyrimidin-4-ylamine (9.3 mg); m/z: (M+H)⁺ 371.1; RP-HPLC (5% to 95% acetonitrile/0.1% H₃PO₄ (aq), over 7 minutes at 1.5 mL/min; λ=190-700 nm; Zorbax SB-C8 rapid resolution, 4.6 mm×75 mm, 3.5 μm column) Rt 1.74 min.

### Example #431

5-[4-(5,7-Dimethyl-benzoxazol-2-ylamino)-3-fluoro-phenyl]-7-methyl-7H-pyrrolo[2,3-d]pyrimidine-2,4-diamine

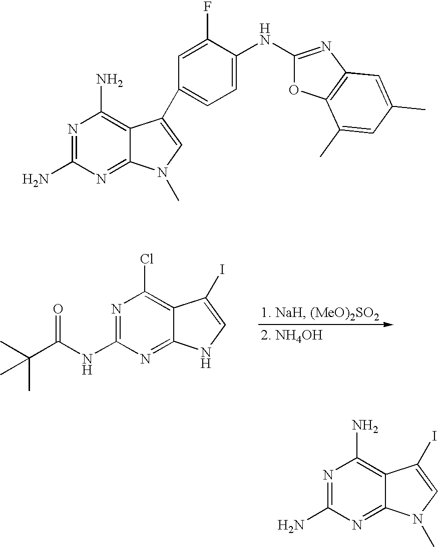

Substituting N-(4-chloro-5-iodo-7H-pyrrolo[2,3-d]pyrimidin-2-yl)-2,2-dimethyl-propionamide (Nucleic Acids Res., 26, 3353, 1998) and dimethylsulfate for 4-chloro-5-iodo-7H-pyrrolo[2,3-d]pyrimidine and 4-nitrobenzyl bromide, respectively in preparation #39.1, gave the methylated product that was reacted with ammonium hydroxide, as detailed in general procedure B, to give 5-iodo-7-methyl-7H-pyrrolo[2,3-d]pyrimidine-2,4-diamine.

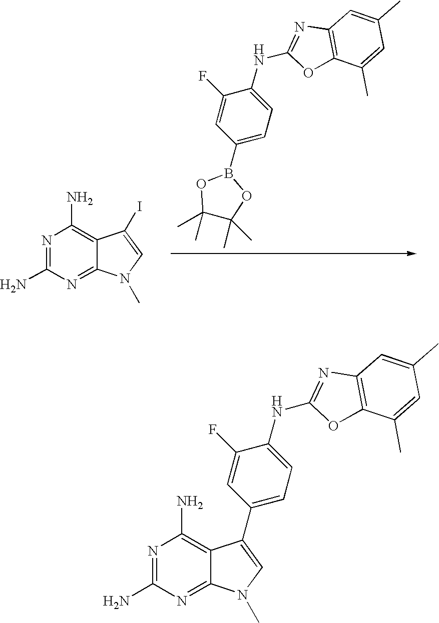

5-Iodo-7-methyl-7H-pyrrolo[2,3-d]pyrimidine-2,4-diamine was reacted with (5,7-dimethyl-benzoxazol-2-yl)- [2-fluoro-4-(4,4,5,5-tetramethyl-[1,3,2]dioxaborolan-2-yl)-phenyl]-amine (G,D), using general procedure C, to afford 5-[4-(5,7-dimethyl-benzoxazol-2-ylamino)-3-fluoro-phenyl]-7-methyl-7H-pyrrolo[2,3-d]pyrimidine-2,4-diamine; m/z: (M+H)⁺ 418.2; ¹H NMR (300 MHz, DMSO-d₆) δ 2.33 (s, 3 H), 2.39 (s, 3 H), 3.56 (s, 3 H), 5.72 (s, 4 H), 6.78 (s, 1 H), 6.91 (s, 1 H), 7.06 (s, 1 H), 7.27 (m, 2 H), 8.28 (t, J=8.31 Hz, 1 H), 10.36 (s, 1 H).

### Example #432

cis-5-[4-(5,7-Dimethyl-benzoxazol-2-ylamino)-3-fluoro-phenyl]-7-(4-morpholin-4-yl-cyclohexyl)-7H-pyrrolo[2,3-d]pyrimidine-2,4-diamine

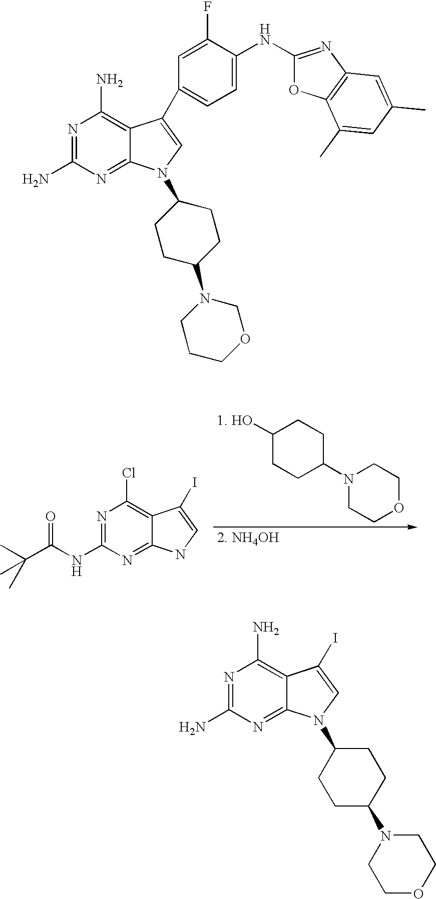

Reaction of N-(4-chloro-5-iodo-7H-pyrrolo[2,3-d]pyrimidin-2-yl)-2,2-dimethyl-propionamide (Nucleic Acids Res., 26, 3353, 1998) with 4-morpholin-4-yl cyclohexanol under conditions detailed in general procedure A gave the alkylated product that under went aminolysis, via general procedure B, to afford 5-iodo-7-(4-morpholin-4-yl-cyclohexyl)-7H-pyrrolo[2,3-d]pyrimidine-2,4-diamine. This product was subsequently reacted with (5,7-dimethyl-benzoxazol-2-yl)-[2-fluoro-4-(4,4,5,5-tetramethyl-[1,3,2]dioxaborolan-2-yl)-phenyl]-amine (G,D), using general procedure C, to afford a mixture of diastereoisomers that were separated by chromatography to yield cis-5-[4-(5,7-dimethyl-benzoxazol-2-ylamino)-3-fluoro-phenyl]-7-(4-morpholin-4-yl-cyclohexyl)-7H-pyrrolo[2, 3-d]pyrimidine-2,4-diamine; m/z: (M+H)⁺ 571.3; ¹H NMR (300 MHz, DMSO-d₆) δ 1.50 (m, 2 H), 1.65 (m, 2 H), 2.01 (m, 4 H), 2.17 (m, 1 H), 2.33 (s, 3 H), 2.39 (m, 7 H), 3.64 (m, 4 H), 4.49 (m, 1 H), 5.70 (s, 4 H), 6.78 (s, 1 H), 6.94 (s, 1 H), 7.06 (s, 1 H), 7.32 (m, 2 H), 8.26 (t, J=8.81 Hz, 1 H), 10.36 (s, 1 H).

Examples #433-446 were made synthesized by reacting trans-3-iodo-1-(4-morpholin-4-yl-cyclohexyl)-1H-pyrazolo[3,4-d]pyrimidin-4-yl amine (A, T, J, C) with the appropriately substituted 2-aminophenol, using general procedure G. 2-Aminophenols, that are not commercially available, were synthesized either from the corresponding 2-nitrophenol, using general procedure

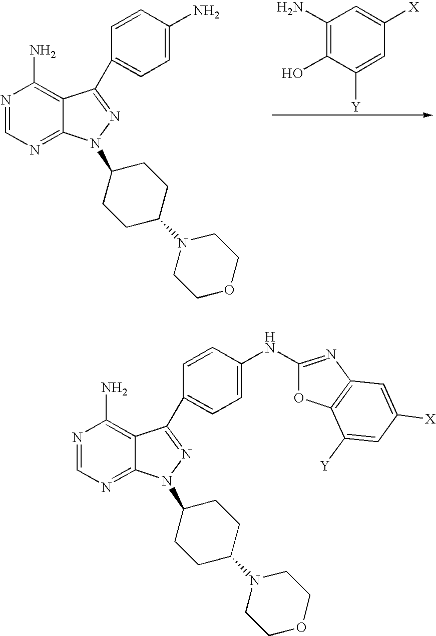

### Example #433

trans-3-[4-(5-tert-Butyl-7-methyl-benzoxazol-2-ylamino)-phenyl]-1-(4-morpholin-4-yl-cyclohexyl)-1H-pyrazolo[3,4-d]pyrimnidin-4-ylamine

### Example #434

trans-3-[4-(7-tert-Butyl-5-ethyl-benzoxazol-2-ylamino)-phenyl]-1-(4-morpholin-4-yl-cyclohexyl)-1H-pyrazolo[3,4-d]pyrimidin-4-ylamine

### Example #435

trans-3-[4-(5-Ethyl-7-methoxy-benzoxazol-2-ylamino)-phenyl]-1-(4-morpholin-4-yl-cyclohexyl)-1H-pyrazolo[3,4-d]pyrimidin-4-ylamine

### Example #436

trans-1-(2-{4-[4-Amino-1-(4-morpholin-4-yl-cyclohexyl)-1H-pyrazolo[3,4-d]pyrimidin-3-yl]-phenylamino}-7-methyl-benzoxazol-5-yl)-ethanone

### Example #437

trans-1-(2-{4-[4-Amino-1-(4-morpholin-4-yl-cyclohexyl)-1H-pyrazolo[3,4-d]pyrimidin-3-yl]-phenylamino}-5-fluoro-benzoxazol-7-yl)-ethanone

### Example #438

trans-3-[4-(7-Methoxy-5-propyl-benzoxazol-2-ylamino)-phenyl]-1-(4-morpholin-4-yl-cyclohexyl)-1H-pyrazolo[3,4-d]pyrimidin-4-ylamine

### Example #439

trans-2-{4-[4-Amino-1-(4-morpholin-4-yl-cyclohexyl)-1H-pyrazolo[3,4-d]pyrimidin-3-yl]-phenylamino}-5-bromo-benzoxazole-7-carbonitrile

### Example #440

trans-(2-{4-[4-Amino-1-(4-morpholin-4-yl-cyclohexyl)-1H-pyrazolo[3,4-d]pyrimidin-3-yl]-phenylamino}-7-ethoxy-benzoxazol-5-yl)-acetonitrile

### Example #441

trans-3-[4-(7-tert-Butyl-5-methyl-benzoxazol-2-ylamino)-phenyl]-1-(4-morpholin-4-yl-cyclohexyl)-1H-pyrazolo[3,4-d]pyrimidin-4-ylamine

### Example #442

trans-3-[4-(5-Chloro-7-methoxy-benzoxazol-2-ylamino)-phenyl]-1-(4-morpholin-4-yl-cyclohexyl)-1H-pyrazolo[3,4-d]pyrimidin-4-ylamine

### Example #443

trans-3-[4-(7-Chloro-5-methoxy-benzoxazol-2-ylamino)-phenyl]-1-(4-morpholin-4-yl-cyclohexyl)-1H-pyrazolo[3,4-d]pyrimidin-4-ylamine

### Example #444

trans-3-[4-(5-Fluoro-7-methoxy-benzoxazol-2-ylamino)-phenyl]-1-(4-morpholin-4-yl-cyclohexyl)-1H-pyrazolo[3,4-d]pyrimidin-4-ylamine

### Example #445

trans-2-{4-[4-Amino-1-(4-morpholin-4-yl-cyclohexyl)-1H-pyrazolo[3,4-d]pyrimidin-3-yl]-phenylamino}-5-chloro-benzoxazole-7-carboxylic acid amide

### Example #446

trans-(2-{4-[4-Amino-1-(4-morpholin-4-yl-cyclohexyl)-1H-pyrazolo[3,4-d]pyrimidin-3-yl]-phenylamino}-7-methoxy-benzoxazol-5-yl)-acetonitrile

The method used to determine the HPLC retention time is given in a lower-case letter in parentheses (see Table 1).

TABLE 16
Examples #433 through #446

| | | | NMR 300 MHz (d₆-DMSO)/LC-RT (method)/ | m/z |
|---|---|---|---|---|
| Y | X | Example # | Elemental analysis | (MH+) |
| Me | ^{t}Bu | 433 | 1.32(s, 9H), 1.47(m, 2H), 2.00(m, 6H), 2.35(m, | 581.3 |
| | | | 1H), 2.44(s, 3H), 2.51(m, 4H), 3.58(m, 4H), | |
| | | | 4.65(m, 1H), 6.82(br. s., 2H), 7.02(s, 1H), | |
| | | | 7.33(d, J=1.70Hz, 1H), 7.64(d, J=8.48Hz, | |
| | | | 2H), 7.93(d, J=8.48Hz, 2H), 8.23(s, 1H), | |
| | | | 10.84(s, 1H). | |
| ^{t}Bu | Et | 434 | 1.23(t, J=7.63Hz, 3H), 1.46(s, 9H), 1.42(m, | 595.4 |
| | | | 2H), 2.01(m, 6H), 2.36(m, 1H), 2.51(m, 4H), | |
| | | | 2.67(q, J=7.57Hz, 2H), 3.58(m, 4H), 4.64(m, | |
| | | | 1H), 6.79(br.s., 2H), 6.87(d, J=1.36Hz, 1H), | |
| | | | 7.19(d, J=1.36Hz, 1H), 7.65(d, J=8.48Hz, 2H), | |
| | | | 7.93(d, J=8.82Hz, 2H), 8.23(s, 1H), 10.77(s, | |
| | | | 1H). | |
| MeO | Et | 435 | 1.23(t, J=7.46Hz, 3H), 1.46(m, 2H), 2.00(m, | 569.3 |
| | | | 6H), 2.36(m, 1H), 2.50(m, 4H), 2.66(q, | |
| | | | J=7.57Hz, 2H), 3.58(m, 4H), 3.94(s, 3H), | |
| | | | 4.64(m, 1H), 6.71(s, 1H), 6.82(br. s., 2H), | |
| | | | 6.95(s, 1H), 7.65(d, J=8.82Hz, 2H), 7.89(d, | |
| | | | J=8.82Hz, 2H), 8.23(s, 1H), 10.83(s, 1H). | |
| Me | CH₃CO | 436 | 1.46(m, 2H), 1.98(m, 6H), 2.38(m, 1H), 2.50(m, | 567.3 |
| | | | 7H), 2.62(s, 3H), 3.58(m, 4H), 4.65(m, 1H), | |
| | | | 6.78(br. s., 2H), 7.67(m, 3H), 7.94(m, 3H), | |
| | | | 8.23(s, 1H), 11.10(s, 1H). | |
| CH₃CO | F | 437 | 1.46(m, 2H), 2.03(m, 6H), 2.34(m, 1H), 2.51(m, | 571.3 |
| | | | 4H), 2.76(s, 3H), 3.58(m, 4H), 4.65(m, 1H), | |
| | | | 6.79(br. s., 2H), 7.40(dd, J=10.34, 2.54Hz, | |
| | | | 1H), 7.67(m, 3H), 7.95(d, J=8.82Hz, 2H), | |
| | | | 8.23(s, 1H), 11.27(s, 1H). | |
| MeO | ⁿPr | 438 | 0.75-1.00(m, 5H) 1.25-1.75(m, 8H) 1.80-2.15(m, | 583 |
| | | | 6H) 2.55-2.65(m, 2H)3.45-3.65(m, 4H) 3.94(s, | |
| | | | 3H) 6.68(d, J=1.02Hz, 1H) 6.92(s, 1H) 7.65(d, | |
| | | | J=8.48Hz, 2H) 7.89(d, J=8.48Hz, 2H) | |
| | | | 8.23(s, 1H) 10.83(s, 1H) | |
| CN | Br | 439 | 11.50(s, 1H), 8.23(s, 1H), 8.04(d, J=1.7Hz, | 612, 614 |
| | | | 1H), 7.88(d, J=1.7Hz, 1H), 7.70(d, | |
| | | | J=8.9Hz, 1H), 7.67(d, J=8.9Hz, 1H), 4.64(s, | |
| | | | 1H), 3.57-3.60(m, 4H), 3.25-3.35(m, 2H), | |
| | | | 2.2-2.4(m, 1H), 1.8-2.1(m, 6H), 1.4-1.6(m, 2H). | |
| OEt | CH₂CN | 440 | EA for C₃₂H₃₅N₉O₃.0.6H₂O | 594 |
| | | | Calc: C, 63.58, H, 6.04, N, 20.85 | |
| | | | Found: C, 63.21, H, 6.02, N, 21.26 | |
| ^{t}Bu | Me | 441 | 2.07(h) | 581 |
| MeO | Cl | 442 | EA for C₂₉H₃₁N₈O₃Cl | 575 |
| | | | Calc: C, 60.57, H, 5.43, N, 19.49 | |
| | | | Found: C, 60.32, H, 5.45, N, 19.23 | |
| Cl | MeO | 443 | 1.47-1.51(m, 2H) 2.01-2.08(m, 6H) 2.36(t, | 575 |
| | | | J=11.36Hz, 1H) 3.30-3.33(m, 5H) 3.56-3.59(m, | |
| | | | 4H) 3.80(s, 3H) 4.61-4.69(m, 2H) 6.84(d, | |
| | | | J=2.37Hz, 1H) 7.10(d, J=2.37Hz, 1H) | |
| | | | 7.66(d, J=8.82Hz, 2H) 7.90(d, J=8.82Hz, 2H) | |
| | | | 8.23(s, 1H) 11.15(s, 1H) | |
| MeO | F | 444 | 1.46(m, 2H) 2.00(m, 6H) 2.35(m, 1H) 2.51(m, | 559.2 |
| | | | 4H) 3.58(m, 4H) 3.96(s, 3H) 4.64(m, 1H) | |
| | | | 6.79(dd, J=12.04, 2.20Hz, 1H) 6.96(dd, | |
| | | | J=8.82, 2.37Hz, 1H) 7.66(d, J=8.48Hz, 2H) | |
| | | | 7.88(d, J=8.82Hz, 2H) 8.23(s, 1H) 11.00(s, 1H) | |
| CONH₂ | Cl | 445 | EA for C₂₉H₃₀ClN₉O₃.0.5 CH₂Cl₂ | 588 |
| | | | Calc: C, 56.19, H, 4.96, N, 19.99 | |
| | | | Found: C, 55.89, H, 4.93, N, 20.32 | |
| MeO | CH₂CN | 446 | EA for C₃₁H₃₃N₉O₃.1 H₂O | 580 |
| | | | Calc: C, 62.30, H, 5.90, N, 21.09 | |
| | | | Found: C, 62.57, H, 5.93, N, 20.88 | |

### General Procedure FF: Ring Closure to Form Substituted aminobenzoxazoles in a One Step Protocol

A mixture of substituted 2-aminophenol (1-5 equivalents, preferably 1.15 equivalents), and substituted phenyl isothiocyanate (1-5 equivalents, preferably 1.0 equivalent) and anhydrous organic solvent (for example, dichloromethane, dioxane, DME, THF, or MTBE, preferably THF is stirred at ambient temperature under an inert atmosphere for 1-48 h (preferably 16 h). The reaction solution is cooled to about 0 to −30° C. (preferably about −15° C.) with a circulation bath. Then solid lithium hydroxide monohydrate (1-5 eq, preferably 2 eq.) was added in one portion. The reaction suspension was cooled to about 0 to −30° C. (preferably about −15° C.) again. Through an addition funnel, 30% aqueous hydrogen peroxide (1-10 eq., preferably 5 eq.) was added drop-wise at a rate so that the temperature is maintained between about 15 to 25° C. (preferably about 15° C.).

After 10 minutes to 3 hours (preferably 10 minutes), the reaction was complete, and a solution of sodium sulfite (Na₂SO₃, 2 L, 1M) was added to the stirring reaction mixture. The reaction mixture was transferred to a separatory funnel with an organic solvent. Layers were separated, and the organic layer was washed with solutions of brine and water. The combined aqueous washes were back extracted with organic solvent. The combined organic extracts were concentrated under reduced pressure. The residue was crystallized and dried in vacuum oven.

Other oxidants that can be used include oxygen (O₂), peracids (RCO₃H, R=aryl or alkyl, or perfluoroalkyl), chlorine (Cl₂), sodium periodate (NaIO₄), potassium periodate (KIO₄), tert-butyl peroxide (t-BuOOH), tert-butyl hypochlorite (t-BuOCl), sodium perborate (NaBO_{3—}nH₂O), sodium percarbonate (Na₂CO_{3—}1.5H2O2), urea hydrogen peroxide adduct (H₂NCONH₂_{2—}H₂O₂), sodium hypochlorite (NaOCl), potassium hypochlorite (KOCl), sodium hypobromite (NaBrO), potassium hypobromite (KBrO), sodium bromate (NaBrO₃), potassium bromate (KBrO₃), potassium permanganate (KMnO₄), and barium manganate (BaMnO₄)

Other bases that can be used include metal hydroxides (Na, K, or CsOH), metal carbonates (Li, Na, K, or Cs₂CO₃), metal bicarbonates (Li, Na, K, or CsHCO₃), metal alkoxides (MOR, R=Me, Et etc), metal phosphates (Li, Na, K, or Cs3PO4), metal dibasic phophates (Li, Na, K, or Cs₂HPO₄), and Tetraalkylammonium (R₄N, where R=Me, Et, Bu etc) of all the above.

### Illustration of General Procedure FF

### Preparation #55. (4-Bromo-2-fluorophenyl)(5-fluorobenzoxazol-2yl)amine

To a 5-L jacketed, 3-neck RB flask, equipped with a nitrogen inlet, a temperature probe, and a mechanical stirrer, was charged 2-amino4-fluorophenol (64.4 g, 507 mmol, 1.15 eq.), 2-fluoro-4-bromophenyl isothiocyanate (102.3 g, 440.8 mmol, 1.0 eq.), and anhydrous THF (1.5 L). The reaction mixture was stirred at room temperature overnight. Reaction (thiourea formation) was complete, shown by HPLC analysis.

The reaction solution was cooled to about −15° C. with a circulation bath. Then solid lithium hydroxide monohydrate (LiOH.H₂O, 37.0 g, 882 mmol, 2 eq.) was added in one portion. The reaction suspension was cooled to about −15° C. again. Through an addition funnel, 30% aqueous hydrogen peroxide (H₂O₂, 264 mL, 2.2 mol, 5 eq.) was added drop-wise at a speed of maintaining the internal temperature between about 15 to 25° C. The reaction is very exothermic at the early period of the addition and subsided toward the end of the addition. After the addition was complete, a sample was taken for HPLC analysis, and usually the reaction was complete.

A solution of sodium sulfite (Na₂SO₃, 2 L, 1M) was added to the stirring reaction mixture slowly while keeping temperature below 30° C. The solution was checked for residual peroxide using a peroxide test strip and showed no peroxide remained. The reaction mixture was transferred to a 6-L separatory funnel. The flask was rinsed with water (3×500 mL), EtOAc (4×500 mL) and the rinses were transferred to the separatory funnel. Layers were separated, and the organic layer was washed with solutions of brine and water (100+400 mL, 4 times), brine (1×500 mL). The combined aqueous washes were back extracted with EtOAc (2 L). The combined organic extracts were concentrated under reduced pressure. To the residue obtained was added acetonitrile (250 mL), and the suspension was rotated on rotovap for 30 minutes and left in refrigerator overnight. Solid was collected and washed with hexanes (1×300 mL), dried in vacuum oven. Product obtained (131.5 g, 92% yield), mp 188-189° C. ¹H NMR (400 MHz, DMSO-d₆) [symbol] 10.67 (s, 1H), 8.20 (t, J=8.8 Hz, 1H), 7.63 (d, J₁=10.7, 1H), 7.52-7.46 (m, 2H), 7.31 (dd, J₁=8.9, Jₛ=1.9, 1H), 6.99-6.94 (m, 1H); ¹³C NMR (100 MHz, DMSO-d₆) δ 159.9(C), 158.8(C), 157.6(C), 153.2(C), 150.7(C), 143.1(C), 142.5 and 142.4(C), 127.10 and 127.06(CH), 125.5 and 125.4(C), 122.4(CH), 118.4 and 118.2(CH), 114.0 and 113.9(C), 109.1 and 109.0(CH), 108.1 and 107.9(CH), 103.5 and 103.3(CH). HRMS: calcd for C₁₃H₈⁷⁹BrF₂N₂O 324.9788, found 324.9803 (MH⁺). Anal. calcd for C₁₃H₇BrF₂N₂O: C, 48.03; H, 2.17; Br, 24.58; F, 11.69; N, 8.62; Found: C, 47.83; H, 1.95; Br, 24.32; F, 11.80; N, 8.49.

The following examples were synthesized using general procedure FF:

### Preparation #56. (4-Bromo-2-fluorophenyl)(5-chlorobenzoxazol-2yl)amine

Yield 90%, mp 194-195° C. ¹H NMR (400 MHz, DMSO-d₆) [symbol] 10.71 (s, 1H),8.18(t, J=8.7 Hz, 1H), 7.63 (dd, J₁=10.6, J₂=2.3, 1H), 7.53-7.46 (m, 3H), 7.17 (dd, J₁=9.2, J₂=2.2, 1H). ¹³C NMR (100 MHz, DMSO-d₆) δ 158.4(C), 153.2(C), 150.8(C), 145.5(C), 142.8(C), 127.8(CH), 127.13 and 127.09(C), 125.4 and 125.2(C), 122.6(CH), 121.2(CH), 118.5 and 118.2(CH), 116.1(CH), 114.2 and 114.1(C), 109.8(CH). HRMS: calcd for C₁₃H₈⁷⁹BrClFN₂O 340.9493, found 340.9501 (MH⁺); calcd for C₁₃H₈⁸¹BrCFN₂O 342.9472, found 342.9470 (MH⁺). Anal. calcd for C₁₃H₇BrClFN₂O: C, 45.71; H, 2.07; Br, 23.39; Cl, 10.38; F, 5.56; N, 8.20; Found: C, 45.74; H, 2.05; Br, 23.71; Cl, 10.51; F, 5.53; N, 8.16.

### Prepartaion #57. (4-Bromo-2-fluorophenyl)(5-methylbenzoxazol-2yl)amine

Yield 89%, mp 185-186° C. ¹H NMR (400 MHz, DMSO-d₆) [symbol] 10.48 (s, 1H), 8.25 (t, J=8.5 Hz, 1H), 7.60 (dd, J₁=10.7, J₂=2.2, 1H), 7.46 (dd, J₁=9.3, J₂=1.0, 1H), 7.35 (d, J=8.2, 1H), 7.25 (s, 1H), 6.95 (d, J=8.2, 1H), 2.37 (s, 3H). ¹³C NMR (100 MHz, DMSO-d₆) δ 157.3(C), 153.0(C), 150.5(C), 144.8(C), 141.3(C), 132.8(C), 127.10 and 127.07(CH), 125.89 and 125.80(C), 122.15 and 122.05 (CH), 118.3 and 118.1(CH), 116.6(CH), 113.43 and 113.35(C), 108.1(CH), 21.1(CH₃). HRMS: calcd for C₁₄H₁₁⁷⁹BrFN₂O 319.9961, found 319.9959 (MH⁺); calcd for C₁₄H₁₁⁸¹BrFN₂O 321.9940, found 321.9949 (MH+). Anal. Calcd for C₁₄H₁₀BrFN₂O: C, 52.36; H, 3.14; Br, 24.88; F, 5.92; N, 8.72; Found: C, 52.14; H, 3.15; Br, 23.21; F, 5.91; N, 8.62.

### Preparation #58. (4-Bromo-2-fluorophenyl)[5-(trifluoromethyl)benzoxazol-2yl]amine

Yield 88%, mp 160-161° C. ¹H NMR (400 MHz, DMSO-d₆) [symbol] 10.83 (s, 1H), 8.22 (t, J=8.8 Hz, 1H), 7.83 (s, 1H), 7.76 (d, J=17.3 Hz, 1H), 7.61 (dd, J₁=10.6, J₂=2.2, 1H), 7.51-7.46 (m, 2H). ¹³C NMR (100 MHz, DMSO-d₆) [symbol] 158.7(C), 153.3(C), 150.8(C), 148.9(C), 142.0(C), 128.0 and 124.3(C), 127.12 and 127.09(CH), 125.29 and 125.25 and 125.21 and 125.13(C), 124.9 and 124.6(C), 122.6(CH), 119.9, 118.74 and 118.70(CH), 118.5(CH) and 118.3(CH), 114.4, 114.3, 113.2(CH), 109.4(CH). Anal. Calcd for C₁₄H₇BrF₄N₂O: C, 44.83; H, 1.88; Br, 21.30; F, 20.26; N, 7.47; O, 4.27. Found: C, 44.65; H, 1.71; Br, 21.14; F, 19.42; N, 7.44.

### Preparation #59. Ethyl(5-methylbenzoxazol-2-yl)amine

Yield 89%, mp 90-91° C. ¹H NMR (400 MHz, DMSO-d₆) δ (t, J=5.3 Hz, 1H), 7.16 (d, J=8.0 Hz, 1H), 7.03-7.02 (m, 1H), 6.76-6.73 (m, 1H), 3.34-3.27 (m, 2H), 2.31 (s, 3H), 1.18 (t, J=7.2, 3H). ¹³C NMR (100 MHz, DMSO-d₆) δ 161.7(C), 145.6 (C), 143.0 (C), 132.0 (C), 120.0(CH), 115.3 (CH), 107.4(CH), 37.1 (CH₂), 21.1 (CH₃), 14.8 (CH₃). HRMS: calcd for C₁₀H₁₂N₂O 176.0950, found 176.0948 (M⁺); Anal. Calcd for C₁₀H₁₂N₂O: C, 68.16; H, 6.86; N, 15.90. Found: C, 67.97; H, 6.86; N, 15.84.

### Example #447

Trans-4-(4-{4-Amino-5-[3-fluoro-4-(5-methyl-benzoxazol-2-ylamino)-phenyl]-pyrrolo[2,3-d]pyrimidin-7-yl)-cyclohexyl)-piperazin-2-one

Sodium trisacetoxyborohydride (46.8 mg, 0.22 mmol) was added to a suspension of 2-piperazinone (51.06 mg, 0.51 mmol) and 4-{4-amino-5-[3-fluoro-4-(5-methyl-benzoxazol-2-ylamino)-phenyl]-pyrrolo[2,3-d]pyrimidin-7-yl}-cyclohexanone (prepared from 4-chloro-3-iodopyrrolo[2,3-d]pyrimidine and 1,4-dioxa-spiro[4.5]decan-8-ol using general procedures A, C, B, K, and G) (80 mg, 0.17 mmol) in glacial acetic acid (0.03 mL, 0.51 mmol) and dichloromethane (5 mL). After about 18 h stirring at ambient temperature, the reaction was still heterogeneous hence NMP (2 mL) was added and the reaction was stirred for about a further 24 h. The raction was monitored by t.l.c. (using 10% MeOH in dichloromethane as the eluent) and quenched with saturated naqueous sodium hydrogencarbonate (10 mL). The procust was extracted into dichloromethane (3×50 mL), dried over anhydrous magnesium sulfate and evaporated to dryness to afford a yellow oil that was further purified by chromatography over silica gel using 0.1% NH₄OH and 5% MeOH in dichloromethane as the eluent. Additional purification using preparative RP-HPLC (5% to 85% acetonitrile/0.1M aqueous ammonium acetate, buffered to pH 4.5, over 20 min 1 mL/min, λ=254 nm; Deltapak C18, 300 Å, 5 μm, 150×3.9 mm column) afforded trans-4-(4-{4-amino-5-[3-fluoro-4-(5-methyl-benzoxazol-2-ylamino)-phenyl]-pyrrolo[2,3-d]pyrimidin-7-yl}-cyclohexyl)-piperazin-2-one (2 mg); ¹H NMR (DMSO-d₆, 400 MHz) δ 8.53 (1 H), 8.30 (1H), 7.33 (2H), 7.28 (2H), 7.00 (2H), 6.05(1H), 5.61 (2H), 4.69 (2H), 3.38 (2H), 3.34 (2H), 2.79 (2H), 2.45 (3H), 2.23 (2H), 2.17 (2H), 1.87 (2H), and 1.61 (2H); and m/z (M+H)⁺ 555.3.

### Example #448

Trans-4-(4-{4-Amino-3-[4-(5,7-dimethyl-benzoxazol-2-ylamino)-3-fluoro-phenyl]-pyrazolo[3,4-d]pyrimidin-1-yl}-cyclohexyl)-piperazin-2-one

Tetrakistriphenylphosphine (6 mg, 0.005 mmol) was added to a solution of 4-[4-(4-amino-3-iodo-pyrazolo[3,4-d]pyrimidin-1-yl)-cyclohexyl]-piperazin-2-one (prepared from 4-amino-3-iodo-pyrazolo[3,4-d]pyrimidine and 1,4-dioxa-spiro[4.5]decan-8-ol using general procedures A, K and J) (45 mg, 0.10 mmol), (5,7-dimethyl-benzoxazol-2-yl)-[2-fluoro-4-(4,4,5,5-tetramethyl-[1,3,2]dioxaborolan-2-yl)-phenyl]-amine (prepared using general procedures G and D) (49 mg, 0.13 mmol), and sodium carbonate (27 mg, 0.25 mmol) in DMF (5 mL) and water (2.5 mL) and heated to about 80° C. for about 12 h. Additional tetrakistriphenylphosphine (0.015 mmol), (5,7-dimethyl-benzoxazol-2-yl)-[2-fluoro-4-(4,4,5,5-tetramethyl-[1,3,2]dioxaborolan-2-yl)-phenyl]-amine (0.06 mmol) and sodium carbonate (0.25 mmol) was added and the reaction was heated at about 80° C. for about a further 16 h. The solvent was removed in vacuo and the residue was partitioned between dichloromethane (100 mL) and water (100 mL). The organic layer was separated and the aqueous layer was further extracted by dichloromethane (3×50 mL). The combined organic layers were dried over anhydrous magnesium sulfate and concentrated under reduced pressure. The product was purified by preparative RP-HPLC (5% to 85% acetonitrile/0.05M aqueous ammonium acetate, buffered to pH 4.5, over 20 min at 1.7 mL/min; λ=254 nm; Hypersil C18, 100 Å, 5 μm, 250×4.6 mm column) and triturated with ethyl acetate to afford trans-4-(4-{4-amino-3-[4-(5,7-dimethyl-benzoxazol-2-ylamino)-3-fluoro-phenyl]-pyrazolo[3,4-d]pyrimidin-1-yl}-cyclohexyl)-piperazin-2-one (4.2 mg) as an off-white solid; LC/MS (30% to 95% acetonitrile/0.01M aqueous ammonium acetate over 4.5 min at 0.8 mL/min; λ=190-700 nm; Genesis C18, 120 Å, 3 μm, 30×4.6 mm column; electrospray ionization method observing both positive and negative ions) Rₜ 2.30 min; m/z: (M+H)⁺ 570.4.

The contents of all references, patents and published patent applications, in their entirety, cited throughout this application are incorporated herein by reference.

We claim:

1. A compound of Formula (I), 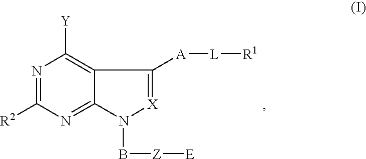
pharmaceutically acceptable salts thereof, metabolites thereof, isomers thereof, or pro-drugs thereof, wherein
X is N or CH;
A is optionally substituted phenyl,
or A is 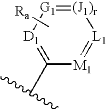
r is 1 and D₁, G₁, J₁, L₁ and M₁ are each independently selected from the group consisting of CRₐ and N, provided that at least two of D₁, G₁, J₁, L₁ and M₁ are CRₐ; or
r is 0, and one of D₁, G₁, L₁ and M₁ is NRₐ, one of D₁, G₁, L₁ and M₁ is CRₐ and the remainder are independently selected from the group consisting of CRₐ and N, wherein Rₐ is as defined below;
L is NH, optionally substituted alkyl, carbonyl, —O-optionally substituted alkyl, NH(optionally substituted aliphatic) or S;
R¹ is —C(═O)—N(R¹⁰⁰)₂ wherein R¹⁰⁰ for each occurrence is independently hydrogen or alkyl; 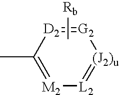
or R¹ is
or an optionally substituted group selected from the group consisting of an aliphatic group, benzimidazolyl, benzofuranyl, benzoisothiazolyl, benzoisoxazolyl, benzoxazolyl, benzothiazolyl, benzothienyl, cycloalkyl, 2,3-dihydrobenzofuranyl, 1,1-dioxybenzoisothiazolyl, furanyl, 1H-imidazo[1,2-a]imidazolyl, imidazo[1,2-a]pyridinyl, imidazo[1,2-a]pyrimidinyl, imidazo[2,1-b][1,3]thiazolyl, indazolyl, indolinyl, indolyl, isoquinolinyl, isothiazolyl, isoxazolyl, morpholinyl, naphthyl, oxadiazolyl, oxazolyl, phenylsulfonyl, phthalazinyl, piperidinyl, pyrazolyl, H-pyridinone, pyridinyl, pyrido-oxazolyl, pyrido-thiazolyl, pyrimido-oxazolyl, pyrimido-thiazolyl, pyrrolidinyl, pyrrolopyridinyl, pyrrolyl, quinolinyl, quinoxalinyl, quinazolinyl, tetrahydrofuranyl, tetrahydronaphthyl, tetrahydropyranyl, 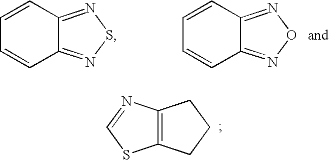
thiadiazolyl, thiazolyl thienyl,
wherein the foregoing optionally substituted groups are optionally substituted by one or more R_{b};
u is 1 and D₂, G₂, J₂, L₂ and M₂ are each independently selected from the group consisting of CRₐ and N, provided that at least two of D₂, G₂, J₂, L₂ and M₂ are CRₐ; or
u is 0, and one of D₂, G₂, L₂ and M₂ is NRₐ, one of D₂, G₂, L₂ and M₂ is CRₐ and the remainder are independently selected from the group consisting of CRₐ and N;
Rₐ and R_{b} each represent one or more substituents and for each occurrence is independently selected from the optionally substituted group consisting of an aliphatic group, alkoxy, alkylamino, aliphatic-carbonyl, aliphatic-cycloalkyl, aliphatic-heterocyclyl, alkyl-S—, alkyl-S(O)ₚ—, amido groups, amino, aminoalkyl, carboxamido, —CF₃, —CN, —C(O)-aliphatic, —C(O)-cycloalkyl, —C(O)-heterocyclyl, —C(O)H, C(O)OH, —C(O)O-aliphatic, C(O)O—C(O)O-heterocyclyl, cycloalkyl, cycloalkyl-aliphatic, cycloalkyl-S, cycloalkyl-S(O)ₚ, cycloalkylthio, dialkylaminoalkoxy, a halo, heterocyclyl, heterocycloalkoxy, heterocycloalkyl, heterocyclyloxy, heterocyclo-S, heterocyclo-S(O)ₚ, heterocyclothio, heterocycloalkyl-S, hydrogen, —NO₂, —OCF₃, —OH, tetrazolyl, trifluoromethylcarbonylamino, trifluoromethylsulfonamido, -Z¹⁰⁵-C(O)N(R)₂, -Z¹⁰⁵-N(R)—C(O)-Z²⁰⁰, -Z¹⁰⁵-N(R)—S(O)₂-Z²⁰⁰, -Z¹⁰⁵-N(R)—C(O)—N(R)-Z²⁰⁰, —N(R)-C(O)R, —N(R)—C(O)OR, O—R—C(O)-heterocyclyl-OR, R_{c} and —CH₂OR_{c};
where R_{c} for each occurrence is independently hydrogen, optionally substituted aliphatic , optionally substituted heterocyclyl-(C₁-C₆)—NR_{d}Rₑ, —W—(CH₂)ₜ—NR_{d}Rₑ, —W—(CH₂)ₜ—O-alkyl, —W—(CH₂)ₜ—S-alkyl, or —W—(CH₂)ₜ—OH;
Z¹⁰⁵ for each occurrence is independently a covalent bond or an aliphatic group;
Z²⁰⁰ for each occurrence is independently selected from an optionally substituted group selected from the group consisting of an aliphatic group, aliphatic-phenyland phenyl;
R_{d} and Rₑ for each occurrence are independently H, an aliphatic group, alkanoyl or SO₂-alkyl; or R_{d}, Rₑ and the nitrogen atom to which they are attached together form a five- or six-membered heterocyclic ring;
t for each occurrence is independently an integer from 2 to 6;
W for each occurrence is independently a bond or O, S, S(O), S(O)₂, or NR_{f}, wherein R_{f} for each occurrence is independently H or an aliphatic group; or
Rₐ is an optionally substituted cycloalkyl or heterocyclyl ring fused with the ring to which it is attached;
B is a bond or a) hydrogen; b) optionally substituted trityl; c) optionally substituted cycloalkyl; d) azaheterocyclyl substituted with an optionally substituted aliphatic group; e) azacycloalkyl which is substituted with one or more substituents selected from the optionally substituted group consisting of —(C₁-C₆)-alkyl, —(C₁-C₆)-alkyl-OR, —C(O)—(C₁-C₆)-alkyl-N(R)₂, —(C₁-C₆)-alkyl-N(R)₂, —(C₁-C₆)-alkyl-cycloalkyl, tetrahydrothienyl, and tetrahydrothiopyranyl; f) a group of the formula 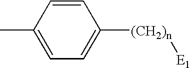
wherein E₁ is selected from an optionally substituted group consisting of amido, amino, imidazolyl, morpholinyl, piperazinyl, piperidinyl, pyrrolidinyl, or tetrahydrothiazolyl, and wherein E₁ is optionally substituted with one or more substituents selected from —(C₀-C₆)-alkyl-OR, —(C₁-C₆)-alkyl-C(O)OR, (C₁-C₆)alkyl-heterocylyl-(C₁-C₆)-alkyl-heterocycloalkyl, —(C₁-C₆)-alkyl-N(R)₂, cyclohexanone, alkoxyalkyl, and pyranyl, g) optionally substituted (C₁-C₆)-alkyl, h) optionally substituted cycloalkyl, i) optionally substituted alkoxyalkoxy, j) optionally substituted alkylamino, k) optionally substituted dialkylamino, l) alkylester, m) alkenyl, n) optionally substituted alkoxy, o) optionally substituted heterocyclyl, p) optionally substituted phenyl, q) optionally substituted 1,4-dioxa-spiro[4.5]decane, r) optionally substituted 1-oxa-2-aza-spiro[4.5]dec-2-ene, s) optionally substituted [1,3]dioxolane, t) —R²⁰⁰—O—(R²⁰⁰)₂—Si(R²⁰⁰)₃, u) a bond, provided that B, Z and E are not each a bond, v) alkoxyalkyl or w) phenylalkyl;
Z is a bond, carbonyl, R²⁰⁰—O—, amino, —O—, —S— or SO₂;
E is a bond or H, or is an optionally substituted group selected from the group consisting of alkoxy, alkoxy-aliphatic, alkoxyamino, alkoxyalkoxy, alkoxycarbonyl-aliphatic, aliphatic group, aliphatic-aminoaliphatic, aliphaticcarbonyl, alkylsulfonyl, amino, amino-aliphatic, amino-aliphatic-carbonyl, aminocarbonyl, aminocarbonyl-aliphatic, aminosulfonyl-aliphatic, CH₂—C(CH₃)₂(OH), —C(CH₃)₂N(CH₃)(H), cycloalkyl, di-aliphatic-amino, di-aliphatic-amino-aliphatic, di-aliphatic-amino-aliphatic-amino, di-aliphatic-aminocarbonyl, di-aliphatic-aminocarbonyl-aliphatic, heterocyclyl, heterocyclo-aliphatic, morpholinocarbonyl-aliphatic, phenyl, piperidinylalkoxy, tetrahydropyranyl-aliphatic, thiopyranyl, tetrahydrothiopyran-1,1-dioxide, triazolyl-aliphatic and urea; or
E is
—CH(R²⁰⁰)—C(O)—N(C₁-C₆)—N(R²⁰⁰)₂,
—N(R²⁰⁰)—(C₁-C₆)—C(O)—N(R²⁰⁰)₂,
—N(R²⁰⁰)—(C₁-C₆)—C(O)—OH,
—N(R²⁰⁰)—(C₁-C₆)—C(O)-morpholinyl,
—(C₁-C₆)—S—CH₃,
—C(R²⁰⁰)(CH₂OH)—(C₁-C₆)—OH,
—C(R²⁰⁰)₂—N(R²⁰⁰)₂,
—C(O)—OH,
—C(R²⁰⁰)₂(OH),
—C(R²⁰⁰)₂—O—(C₁-C₆)—C(R²⁰⁰)₂(OH),
—C(R²⁰⁰)₂C(R²⁰⁰)₂(OH),
wherein R²⁰⁰ is independently hydrogen or alkyl;
R² is H, —NH₂, —S(C₁-C₆)alkyl, —SO₂(C₁-C₆)alkyl, optionally substituted alkyl, —OR⁷, —N(H)SO₂R⁷, —N(R⁷)SO₂R⁷, —N(R⁷)C(O)N(H)R⁷, —N(R⁷)C(O)NR⁷, —N(H)C(O)R⁷, —N(R⁷)₂, —N(R⁷)C(O)R⁷, —NHC(O)NHR⁷, or —NHR⁷;
R⁷ is (C₁-C₆)-aliphatic optionally substituted by one or more substituents each independently selected from the group consisting of (C₁-C₆)alkoxy, heterocyclyl, hydroxyl, —NR⁵R⁶ optionally substituted phenyl, —C(O)R⁴ and heterocyclyl;
wherein any of said alkoxy, aliphatic and heterocyclyl may be optionally substituted;
wherein R⁵ and R⁶ are independently H or (C₁-C₆)alkyl, —NHS(O)₂R⁴, —NHC(O)R⁴ or —NHC(═NH)R⁴;
wherein R⁴ is selected from (C₁-C₆)alkyl and H;
Y is H, OR³ or N(R³)₂ wherein R³ is independently selected from H or an optionally substituted group consisting of aliphatic, —(CH₂)₂—C(O)—NH₂, —C(O)-aliphatic, —C(O)-cycloalkyl, and —C(O)-heterocyclyl;
where R for each occurrence is independently H or selected from an optionally substituted group consisting of aliphatic, heterocyclyl and heterocyclo-aliphatic;
n is an integer from 1 to 6; and
p is 1 or 2;
provided that 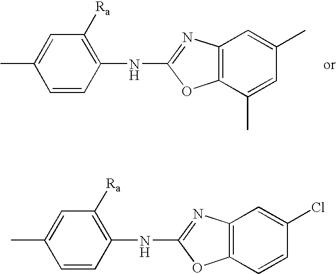
when A-L-R¹ is
then B-Z-E is not a pyrrolidinyl which is substituted with 2-methoxyethyl, N,N-dimethylaminomethyl, N,N-dimethylamino-1-oxoethyl, or 2-(N-methylamino)-1-oxopropyl;
when X is N; Y is NH₂; R² is H; L is NH; A is phenyl optionally substituted with fluoro or methoxy; B is cyclohexyl; Z is a bond and E is piperazinyl substituted with methyl, then R¹ is not:
phenyl optionally substituted with C₂H₄OH or chloro,
benzofuranyl optionally substituted with chloro,
imidazolyl optionally substituted with methyl,
benzoxazolyl optionally substituted with one or two methyls,
benzoxazolyl optionally substituted with one or two chloros,
benzoxazolyl optionally substituted with methoxy,
benzoxazolyl optionally substituted with ethyl,
benzoxazolyl optionally substituted with carbonitrile,
benzoxazolyl optionally substituted with isopropyl,
benzothiazolyl optionally substituted with one or two methyls,
benzothiazolyl optionally substituted with propyl,
benzothiazolyl optionally substituted with isopropyl,
benzothiazolyl optionally substituted with ethyl and phenyl,
thiazolyl substituted with ethyl,
thiazolyl optionally substituted phenyl,
thiazolyl optionally substituted with phenylmethyl,
thiazolyl optionally substituted with nitrophenyl,
thiazolyl optionally substituted with two methyls,
thiazolyl substituted with phenyl and methyl,
thiazolyl substituted with phenyl and propyl,
thiazolyl substituted with phenyl and isopropyl,
thiazolyl substituted with ethyl and methylphenyl,
benzoisothiazolyl optionally substituted with CF₃,
benzoisothiazolyl optionally substituted with one or two oxo,
benzoisoxazolyl substituted with CF₃,
indazolyl, or pyrimidinyl; or
when X is N; Y is NH₂; R is H; L is NH; A is phenyl optionally substituted with fluoro; R¹ is benzoxazolyl substituted with one or two methyls, benzothiazolyl or ethyl; Z is a bond; and E is COOH, piperazinyl substituted with methyl, piperazinyl substituted with oxo, or ethyl substituted with oxo;
then B is not ethyl, cyclohexyl, piperidinyl substituted with dimethylamino, or phenyl substituted with CN; or
when X is N; Y is NH₂; R²is H; L is NH; A is phenyl; B is a bond; Z is a bond; and R¹ is benzofuranyl, benzoisoxazolyl, piperidinyl, pyrrolyl, isooxazolyl substituted with phenyl, isoxazolyl substituted with trifluoromethyl, benzoxazolyl optionally substituted with one or two methyls, benzoxazolyl optionally substituted with ethyl, benzoxazolyl optionally substituted with chloro, or benzoxazolyl optionally substituted with isopropyl then
E is not:
piperidinyl optionally substituted with substituted alkyl,
piperazinyl,
pyrrolidinyl optionally substituted with methoxyethyl,
piperidinyl optionally substituted with dihydroxypropyl,
piperidinyl optionally substituted with hydroxyethyl,
piperidinyl optionally substituted with methoxyethyl,
piperidinyl optionally substituted with methylsulfanylethyl,
piperidinyl optionally substituted with optionally substituted ethyl,
piperidinyl optionally substituted with optionally substituted propyl,
imidazolyl optionally substituted with methyl,
imidazolyl optionally substituted with amino,
aminoalkylcarbonyl,
cyclohexanecarboxylate, or
pyrimidinyl substituted with CN; or
when X is N; Y is NH₂; R² is H; A is phenyl; R¹ is phenyl; B is cyclohexyl; Z is a bond; and E is piperazinyl substituted with methyl; then L is not methyl substituted by ═N—OCH₃, ═N—OH, NH₂ or CN; or
when X is N; Y is NH₂; R² is H; L is NH; A is phenyl; R¹ is benzoxazolyl substituted with two methyls; B is pyrrolidinyl optionally substituted with methylaminomethyl and ethyl, or pyrrolidinyl optionally substituted by dimethylamino and ethyl; and Z is carbonyl; then E is not dialkylamino, a bond or alkyl substituted with methylamino; or
when X is N; L is NH; A is phenyl; R¹ is benzoxazolyl optionally substituted with two methyls; B is cyclohexyl; and Z is a bond; then E is not dimethylamino or morpholino; or
when X is N; L is NH; A is phenyl; R¹ is benzoxazolyl optionally substituted with two methyls; B is cyclohexyl; and Z is NH; then E is not methoxyethyl or methyl; or
when X is N; Y is NH₂; R² is H; L is NH; A is phenyl; R¹ is benzoxazolyl substituted with two methyls; B is piperidinyl; and Z is a bond; then E is not a bond; or
when X is N; L is O-alkyl; A is phenyl; B is cyclohexyl or a bond; Z is a bond; and E is cyclopentyl or piperazinyl substituted with methyl; then R¹ is not phenyl optionally substituted with benzenesulfonamide or phenyl optionally substituted with benzylurea; or
when X is N, Y is NH₂, R² is H, L is NH, A is phenyl optionally substituted with fluoro, R¹ is benzoxazolyl substituted with ethyl, benzoxazolyl substituted with chloro, or benzoxazolyl substituted with one or two methyls; B is piperidinyl, azetidinyl, pyrrolyl, or cyclohexyl; and Z is a bond; then E is not:
methoxyethyl,
methoxypropyl,
methyl,
ethyl optionally substituted with hydroxyl,
piperazinyl substituted with oxo, or
imidazolyl optionally substituted with amino;
or
when X is N; Y is NH₂; R² is H; L is NH; A is phenyl; B is piperidinyl; Z is carbonyl; and R¹ is benzoxazolyl optionally substituted with two methyls or benzoxazolyl optionally substituted with chloro; then
E is not:
morpholinoalkyl,
dimethylaminomethyl,
piperidinyl optionally substituted with methyl,
isopropyl substituted with methylamine,
pyrrolidinyl,
ethyl optionally substituted with methyl and methylamino, or
ethyl optionally substituted with substituted alkyl; or
when X is N; Y is NH₂; R² is H; L is carbonyl; A is phenyl; Z is a bond; E is piperidinyl or pyridinyl; and B is a bond; then
R¹ is not:
oxazolyl,
isoxazolyl optionally substititued with methyl,
isoxazolyl optionally substituted with phenyl,
pyrazolyl optionally substituted with benzyl,
pyrazolyl optionally substituted with benzoyl,
pyrazolyl optionally substituted with methyl, or
pyrazolyl optionally substituted with ethanone; or
when X is N; Y is NH₂; R² is H; L is carbonyl; A is phenyl; Z is a bond; R¹ is phenyl; and B is cyclohexyl; then E is not piperazinyl substituted with methyl; or
when X is N; L is alkyl optionally substituted with OH; A is phenyl optionally substituted with methoxy; R¹ is benzoxazolyl or benzimidazolyl; B is cyclohexyl; and Z is a bond; then E is not piperazinyl substituted with methyl.

2. The compound, pharmaceutically acceptable salts thereof, metabolites thereof, isomers thereof, or pro-drugs thereof, of claim 1 wherein Y is —N(R³)₂.

3. The compound, pharmaceutically acceptable salts thereof, metabolites thereof, isomers thereof, or pro-drugs thereof, of claim 2 wherein:
X is N;
A is optionally substituted phenyl;
R¹ is optionally substituted benzoxazolyl or optionally substituted benzothiazolyl;
B is a bond or is selected from an optionally substituted group consisting of alkenyl, alkyl, alkoxyalkyl, (C₃-C₇)cycloalkyl, (C₃-C₇)cycloalkenyl, heterocyclyl, phenyl, 1,4-dioxa-spiro[4.5]dec-2-ene, 2,2-dipropyl[1,3]dixolane, 1-oxa-2-aza-spiro[4.5]dec-2-ene, 1,4-dioxa-spiro[4.5]decane and 2,2-dipropyl[1,3]dioxolane;
E is H or selected from an optionally substituted group consisting of alkoxy, alkoxyalkyl, alkoxyalkoxy, alkoxyamino, alkyl, alkylaminoalkyl, aminoalkyl, aminoalkylcarbonyl, aminocarbonyl, azetidinyl, benzimidazolyl, —C(CH₃)(CH₂OH)—CH₂—OH, —C(CH₃)₂, —NH(CH₃), —C(CH₃)₂—O—CH₂—C(CH₃)₂(OH), —CH₂—C(CH₃)₂(OH), —(CH₂)₂—S—CH₃, COOH, cycloalkyl, diazepanyl, dimethylamino, dimethylaminoalkyl, dimethylaminoalkylamino, dimethylaminocarbonyl, dimethylaminocarbonylalkyl, furanyl, imidazolinyl, imidazolyl, imidazolylalkyl, isoxazolyl, morpholinyl, morpholinylalkyl, —N(CH₃)—CH₂—C(═O)-morpholinyl, —N(CH₃)—CH₂—C(═O)—N(CH₃)₂, —N(CH₃)—CH₂—C(═O)—OH, oxodiazolyl, oxazolyl, piperazinyl, piperidinyl, pyranyl, pyrazinyl, pyrazolyl, pyridinyl, pyrrolidinyl, pyrrolyl, tetrahydropyranyl, tetrazolyl, thiadiazolyl, thiopyranyl, thienyl, triazolyl and triazolylalkyl;
R² is H, SCH₃, NH₂, or S(O)₂—CH₃; and
R³ for each occurrence is independently H or —(CH₂)₂—C(═O)NH₂.

4. The compound, pharmaceutically acceptable salts thereof, metabolites thereof, isomers thereof, or pro-drugs thereof, of claim 3 wherein:
A is optionally substituted by one or more substituents selected from the group consisting of alkyl, alkoxy, chloro and fluoro;
R¹ is optionally substituted by one or more substituents selected from the group consisting of alkyl, alkenyl, alkoxy, alkoxyalkoxy, alkoxycarbonylpiperidinylalkoxy, alkylcarbonyl, aminocarbonyl, bromo, CF₃, chloro, C(═O)—O(CH₃)₃, dialkylaminoalkoxy, dialkylaminocarbonyl, dialkylaminocarbonylalkoxy, fluoro, —OH, morpholinoalkoxy, NO₂, OCF₃, phenyl-S-alkoxy, optionally substituted piperidinylalkoxy, optionally substituted pyridinylalkoxy, optionally substituted pyrrollidinylalkoxy and optionally substituted thienylalkoxy;
B is a bond or an optionally substituted group selected from the group consisting of alkoxyalkyl, alkyl, azetidinyl, cycloalkenyl, cycloalkyl, isoxazolyl, phenyl, piperidinyl, pyranyl, pyridinyl, pyrrolidinyl, tetrahydropyranyl, tetrahydrothiopyranyl, thiopyranyl, 1,4-dioxa-spiro[4.5]dec-2-ene, [1,3]dioxolane, 1-oxa-2-aza-spiro[4.5]dec-2-ene, and 1,4-dioxa-spiro[4.5]decane;
E is H, dimethylaminoalkyl, dimethylaminocarbonyl or an optionally substituted group selected from the group consisting of alkyl, alkoxyalkyl, azetidinyl, benzimidizolyl, diazepanyl, furanyl, imidazolyidinyl, imidazolyl, isoxazolyl, morpholinyl, oxadiazolyl, oxazolyl, phenyl, piperidinyl, piperazinyl, pyrazinyl, pyrazolyl, pyridinyl, pyrrolidinyl, pyrrolyl, tetrahydrofuranyl, tetrahydropyranyl, tetrahydrothiopyran 1,1-dioxide, tetrazolyl, thiadiazolyl, thienyl, thiopyranyl, and triazolyl; and
wherein the group is optionally substituted by one or more substituents selected from the group consisting of alkoxy, alkoxyalkyl, alkyl, alkylcarbonyl, alkylsulfonyl, dialkylaminosulfonyl, fluoro, hydroxy, hydroxyalkyl, nitrile, oxo, S(O)₂CH₃, and S(O)₂CF₃.

5. The compound, pharmaceutically acceptable salts thereof, metabolites thereof, isomers thereof, or pro-drugs thereof, of claim 4 wherein L is NH, C(OH)H or carbonyl;
B is a bond or is selected from the optionally substituted group consisting of alkyl, azetidinyl, cycloalkyl, isoxazolyl, phenyl, piperidinyl, pyranyl, tetrahydropyranyl, tetrahydrothiopyranyl, 1,4-dioxa-spiro[4.5]dec-2-ene, [1,3]dioxolane, 1-oxa-2-aza-spiro[4.5]dec-2-ene, and 1,4-dioxa-spiro[4.5]decane;
wherein the group is substituted by one or more substituents selected from the group consisting of alkoxy, alkyl, CF₃, C≡N, cycloalkyl, fluoro, and hydroxyl.

6. The compound, pharmaceutically acceptable salts thereof, metabolites thereof, isomers thereof, or pro-drugs thereof, of claim 5 wherein R² is H.

7. The compound, pharmaceutically acceptable salts thereof, metabolites thereof, isomers thereof, or pro-drugs thereof, of claim 6 wherein R³ for each occurrence is H.

8. The compound, pharmaceutically acceptable salts thereof, metabolites thereof, isomers thereof, or pro-drugs thereof, of claim 7 wherein R¹ is benzoxazolyl or benzothiazolyl, each optionally substituted by one or more substituents selected from the group consisting of alkenyl, alkoxy, alkyl, bromo, CF₃, chloro, dimethylaminocarbonyl, fluoro, hydroxyl, OCF₃ and nitrile.

9. The compound, pharmaceutically acceptable salts thereof, metabolites thereof, isomers thereof, or prodrugs thereof, of claim 8 wherein A is phenyl optionally substituted by fluoro or alkoxy;
L is NH;
R¹ is benzoxazolyl optionally substituted by one or more substituents selected from the group consisting of CF₃, CH₃ and chloro;
Z is a bond, carbonyl, R²⁰⁰—O—, —O— or —S—; and
E is H or selected from the optionally substituted group consisting of alkoxyalkyl alkoxyamino, alkyl, COOH, cycloalkyl, diazepanyl, dimethylaminocarbonyl, furanyl, imidazolylalkyl, imidazolidinyl, imidazolyl, isoxazolyl, morpholinyl, —N(R²⁰⁰)—R²⁰⁰—C(═O)—N(R²⁰⁰)₂, —N(R²⁰⁰)—R²⁰⁰—C(═O)—OH, —N(R²⁰⁰)—R²⁰⁰—C(═O)-morpholinyl, OH, oxazolyl, piperazinyl, piperidinyl, pyrazinyl, pyrazolyl, pyridinyl, pyrrolidinyl, tetrahydrofuranyl, tetrahydropyranyl, tetrazolyl, thiadiazolyl, thienyl, and triazolyl;
wherein R²⁰⁰ is alkyl.

10. The compound of claim 9 wherein the compound is
3-[3-(fluoro-4-(5-trifluoromethyl-benzoxazol-2-ylamino)-phenyl]-1-[4-(2-methoxy-ethoxy)-cyclohexyl]-1H-pyrazolo[3,4-d]pyrimidin-4-ylamine,
3-[4-(7-chloro-5-methyl-benzoxazol-2-ylamino-phenyl]-1-[4-(2-methoxy-ethoxy)-cyclohexyl)-1H-pyrazolo[3,4-d]pyrimidin-4-ylamine, or
1-(4-{4-amino-3-[4-(5-chloro-benzoxazol-2-ylamino)-3-fluoro-phenyl]-pyrazolo[3,4-d]pyrimidin-1-yl )-cyclohexyloxy)-2-methyl-propan-2-ol.

11. The compound, pharmaceutically acceptable salts thereof, metabolites thereof, isomers thereof, or pro-drugs thereof, of claim 1 wherein:
X is CH;
A is optionally substituted phenyl;
R¹ is optionally substituted benzoxazolyl;
B is H or selected from the optionally substituted group consisting of alkoxyalkyl, alkyl, cycloalkyl and heterocyclyl;
E is H, or is selected from an optionally substituted group consisting of alkoxy, alkyl, alkylsulfonyl, aminocarbonylalkyl, diazepanyl, dimethylamino, morpholinyl, phenyl, piperazinyl, tetrazolyl and urea;
R² is H, NH₂, SCH₃, or SO₂CH₃; and
R³ for each occurrence is H.

12. The compound, pharmaceutically acceptable salts thereof, metabolites thereof, isomers thereof, or pro-drugs thereof, of claim 11 wherein:
A is optionally substituted by fluoro;
R¹ is an optionally substituted benzoxazolyl substituted by one or more substituents selected from the group consisting of alkoxy, alkyl, bromo, chloro, CF₃, dialkylaminoethoxy, fluoro, morpholinylalkoxy, morpholinylalkyl and nitrile;
B is H or is selected from the optionally substituted group consisting of cycloalkyl, alkyl, piperidinyl and pyrrolidinyl;
wherein the substituents are selected from the group consisting of alkyl, hydroxyl, oxo, nitrile and nitro;
E is H or selected from the optionally substituted group consisting of alkyl, alkoxy, alkoxyalkyl, alkylsulfonyl, aminocarbonylalkyl, diazepanyl, dimethylamino, morpholinyl, piperazinyl, phenyl, tetrazolyl and urea;
wherein the group is optionally substituted by one or more substituents selected from the group consisting of alkoxy, alkyl, alkylsulfonyl, cycloalkyl, hydroxyl, nitrile, nitro, NH₂ and oxo; and
Z is a bond, R²⁰⁰—O—, NH or —O—.

13. The compound, pharmaceutically acceptable salts thereof, metabolites thereof, isomers thereof, or pro-drugs thereof, of claim 12 wherein L is NH or N(alkenyl).

14. The compound, pharmaceutically acceptable salts thereof, metabolites thereof, isomers thereof, or pro-drugs thereof, of claim 13 wherein R is H.

15. The compound, pharmaceutically acceptable salts thereof, metabolites thereof, isomers thereof, or pro-drugs thereof, of claim 14 wherein R¹ is optionally substituted benzoxazolyl substituted by one or more substituents selected from the group consisting of alkyl, bromo, CF₃, chloro, fluoro and nitrile.

16. The compound, pharmaceutically acceptable salts thereof, metabolites thereof, isomers thereof, or pro-drugs thereof, of claim 15 wherein
A is phenyl or phenyl substituted by fluoro;
L is NH;
R¹ is benzoxazolyl substituted by one or more substituents selected from the group consisting of alkyl, bromo, CF₃ and chloro;
Z is a bond or —O—; and
E is optionally substituted alkyl, alkoxyalkyl, diazepanyl, piperazinyl or tetrazolyl.

17. The compound, pharmaceutically acceptable salts thereof, metabolites thereof, isomers thereof, or pro-drugs thereof, of claim 2 wherein:
X is CH;
A is optionally substituted phenyl;
R¹ is optionally substituted benzoxazolyl;
B is H or a bond or selected from the optionally substituted group consisting of alkyl and cycloalkyl;
Z is a bond, —R²⁰⁰—O—, amino or —O—;
E is H, a bond or an optionally substituted group selected from the group consisting of alkoxy, alkyl, alkylsulfonyl, aminocarbonylalkyl, dialkylamino, heterocyclyl, phenyl and urea;
R² is H, NH₂, —S(C₁-C₆)alkyl, or —SO₂(C₁-C₆)alkyl; and
R³ for each occurrence is H.

18. The compound, pharmaceutically acceptable salts thereof, metabolites thereof, isomers thereof, or pro-drugs thereof, of claim 17 wherein
A is optionally substituted by one or more fluoro;
R¹ is optionally substituted by one or more substituents selected from the group consisting of alkyl, alkoxy, aminoalkoxy, bromo, CF₃, chloro, fluoro, morpholinoalkoxy, morpholinoalkyl and nitrile;
E is H or an optionally substituted group selected from the group consisting of alkoxy, alkyl, alkylsulfonyl, aminocarbonylalkyl, diazapenyl, dimethylamino, morpholinyl, phenyl, piperazinyl, pyridinyl, pyrrolidinyl, tetrazolyl and urea;
wherein the optionally substituted group is optionally substituted by one or more alkoxy, alkyl, amino, bromo, cycloalkyl, dimethylamino, hydroxyl, oxo, nitrile, NO₂ or sulfonyl; and
R² is H.

19. The compound, pharmaceutically acceptable salts thereof, metabolites thereof, isomers thereof, or pro-drugs thereof, of claim 18 wherein
L is NH or N-alkenyl;
R¹ is substituted by one or more alkyl, bromo, CF₃, chloro, fluoro, or nitrile;
A is phenyl optionally substituted by fluoro;
B is a bond or is selected from the optionally substituted group consisting of alkyl, cycloalkenyl, cyclopentyl or cyclohexyl;
Z is a bond, —O— or —R²⁰⁰—O—; and
E is H, or selected from an optionally substituted group consisting of alkoxy, alkenyl, alkyl, cycloalkyl, diazapenyl, piperazinyl and tetrazolyl.

20. The compound, pharmaceutically acceptable salts thereof, metabolites thereof, isomers thereof, or pro-drugs thereof, of claim 19 wherein:
R¹ is substituted by alkyl, bromo or chloro; L is NH; B is cyclohexyl; Z is a bond or —R²⁰⁰—O—;
wherein R²⁰⁰ is alkyl;
E is alkoxy or optionally substituted piperazinyl; and Y is NH.

21. The compound of claim 20 wherein the compound is
4-(4-{4-Amino-5-[4-(5-chloro-benzoxazol-2-ylamino)-3-fluoro-phenyl]-pyrrolyl[2,3-d]pyrimidin-7-yl}-cyclohexyl-1-methyl-piperazin-2-one
5-[4-(5-Chloro-benzooxazol-2-ylamino)-phenyl]-7-[4-(2-methoxy-ethoxy)-cyclohexyll]-7H-pyrrolo[2,3-d]pyrimidin-4-ylamine; or
5-[4-(5-Bromo-7-methyl-benzooxazol-2-ylamino)-phenyl]-7-[4-(2-methoxyethoxy)-cyclohexyl]-7H-pyrrolo[2,3-d]pyrimidin-4-ylamine.

22. A method of treating a disease or condition in a patient in need thereof, comprising administering a compound according to claim 1 to said patient, wherein the disease or condition is selected from the group consisting of rheumatoid arthritis, thyroiditis, type 1 diabetes, multiple sclerosis, sarcoidosis, inflammatory bowel disease, Crohn's disease, myasthenia gravis, systemic lupus erythematosus, psoriasis, organ transplant rejection, benign and neoplastic proliferative diseases, lung cancer, breast cancer, stomach cancer, bladder cancer, colon cancer, pancreas cancer, ovarian cancer, prostate cancer, rectal cancer, hematopoietic malignancies, diabetic retinopathy, retinopathy of prematurity, choroidal neovascularization due to age-related macular degeneration, infantile hemangiomas, edema, ascites, effusions, exudates, cerebral edema, acute lung injury, adult respiratory distress syndrome, blood vessel proliferative disorders, fibrotic disorders, mesangial cell proliferative disorders, metabolic diseases, atherosclerosis, restenosis, psoriasis, hemangiomas, myocardial angiogenesis, coronary collaterals, cerebral collaterals, ischemic limb angiogenesis, ischemia/reperfusion injury, wound healing, peptic ulcer Helicobacter related diseases, virally-induced angiogenic disorders, fractures, Crow-Fukase syndrome (POEMS), preeclampsia, menometrorrhagia, cat scratch fever, rubeosis, neovascular glaucoma, retinopathies, malignant ascites, von Hippel Lindau disease, hematopoietic cancers, hyperproliferative disorders, burns, chronic lung disease, stroke, polyps, anaphylaxis, chronic inflammation, allergic inflammation, delayed-type hypersensitivity, ovarian hyperstimulation syndrome, angina, ankylosing spondylitis, asthma, congestive obstructive pulmonary disease (COPD), hepatitis C virus (HCV), idiopathic pulmonary fibrosis, myocardial infarct, psoriatic arthritis, restinosis and sciatica.

23. A pharmaceutical composition comprising a compound according to claim 1 and a pharmaceutically acceptable carrier or excipient.

24. A method of making an optionally substituted 2-aminobenzoxazole comprising the step of:
reacting an optionally substituted N-(2-hydroxyphenyl)thiourea with an oxidant and a base but not including a toxic metal until the reaction is substantially complete;
wherein the oxidant is selected from the group consisting of hydrogen peroxide, oxygen, peracids, chlorine, sodium periodate, potassium periodate, tert-butyl peroxide, tert-butyl hypochlorite, sodium perborate, sodium percarbonate, urea hydrogen peroxide adduct, sodium hypochlorite, potassium hypochlorite, sodium hypobromite, potassium hypobromite, sodium bromate, potassium bromate, potassium permanganate and barium manganate; and the base is selected from the group consisting of metal and tetraalkylammonium hydroxides, metal and tetraalkylammonium carbonates, metal and tetraalkylammonium bicarbonates, metal and tetraalkylammonium alkoxides, metal and tetraalkylammonium phosphates, metal and tetraalkylammonium dibasic phophates.
